(19) United States
(12) Patent Application Publication
(10) Pub. No.: US 2026/0146249 A1
(43) Pub. Date: May 28, 2026

(54) ANTISENSE OLIGONUCLEOTIDES FOR TREATING A DISEASE OR CONDITION ASSOCIATED WITH AN ABNORMAL PROCESSING OF APP

(71) Applicants: VICO Therapeutics B.V., Leiden (NL); KATHOLIEKE UNIVERSITEIT LEUVEN, Leuven (BE); VIB VZW, Gent (BE)

(72) Inventors: Sonia NAJAS SALES, Leiden (NL); Dennis Christian FRANKEN, Leiden (NL); Inmaculada SANJUAN RUIZ, Leuven (BE); Bart DE STROOPER, Leuven (BE); Lutgarde SERNEELS, Leuven (BE); Judith VAN DEUTEKOM, Leiden (NL)

(21) Appl. No.: 18/995,766

(22) PCT Filed: Jul. 19, 2023
(86) PCT No.: PCT/EP2023/070010

§ 371 Date: Jan. 16, 2025

(30) Foreign Application Priority Data
Jul. 25, 2022 (EP) 22382711.4

(51) Int. Cl. C12N 15/113; A61K 31/712; A61K 31/7125; A61P 25/28

(52) U.S. Cl. CPC C12N 15/1137; A61K 31/712; A61K 31/7125; A61P 25/28; C12N 2310/11; C12N 2310/315; C12N 2310/321

(57) ABSTRACT

The invention relates to the field of human genetics, more specifically to treatments for a disease or condition associated with an abnormal processing of the Amyloid Precursor Protein (APP), preferably familiar Alzheimer disease (FAD). The invention in particular relates to antisense oligonucleotides (AON's) that can be used for treating such diseases or conditions.

### RELATED APPLICATIONS

This application is a 35 U.S.C. § 371 filing of International Patent Application No. PCT/EP2023/070010, filed Jul. 19, 2023, which claims priority to European Patent Application No. 22382711.4, filed Jul. 25, 2022, the entire disclosures of which are hereby incorporated herein by reference.

### SEQUENCE LISTING

The instant application contains a Sequence Listing which has been submitted electronically in XML format and is hereby incorporated by reference in its entirety. Said XML file, created on Feb. 5, 2026, is named 761080_VCO9-004US_ST26.xml and is 918,211 bytes in size.

### FIELD

The invention relates to the field of human genetics, more specifically to treatments for a disease or condition associated with an abnormal (or altered) processing of the Amyloid Precursor Protein (APP), preferably familiar Alzheimer disease (FAD). The invention in particular relates to antisense oligonucleotides (AONs) that can be used for treating such diseases or conditions.

### BACKGROUND OF THE INVENTION

Neurocognitive disorders include a broad category of degenerative brain diseases marked by a progressive decline in memory, learning, perception, social, recognition, orientation, language, comprehension, and judgment (American Psychiatric Association, 2013, 5^{th}. Ed. Arlington). Neurocognitive disorders are a manifestation of impaired multiple molecular pathways and cellular functions, leading to synapse loss, cell death, inflammation, gliosis, and disruption of the functional networks that underlie cognition, sensation, and motor skills (Elahi & Miller, 2017 Nature Reviews. Neurology, 13:457-476). The continuous decline in physical function in patients ultimately leads to loss of autonomy and death. Alzheimer's disease (AD) is the most common neurocognitive and neurodegenerative disease, and the most frequent cause of dementia (60% to 80% of cases). AD involves both monogenic and complex forms of inheritance, and is characterized by the accumulation of insoluble amyloid-β plaques (also called Aβ plaques) in extracellular spaces and vascular walls, the aggregation of the microtubule protein Tau in neurofibrillary tangles in neurons, dystrophic neurites, loss of synapses, neurotransmitter alterations (acetylcholine deficiency and glutamate excitotoxicity), and micro- and astrogliosis. Molecular and cellular pathological events start decades before dementia becomes apparent. The age of onset of AD is over 65 years of age with risk polymorphisms in genes that regulate microglial immune activation, lipid metabolism and endocytosis. (Masters et al., 2015 Nature Reviews, 1:1-18; Congdon et al 2018, Nature Reviews Neurology, 14(7), 300-415); Long and Holtzman et al., 2019 Cell, 170(2), 312-339).

Familial Alzheimer's disease (FAD) is an Early-Onset AD (EOAD) (before 65 years of age) caused by mutations in genes involved in Aβ peptide production: APP (OMIM 104760), PSEN1 (OMIM 104311), or PSEN2 (OMIM 1600759) (Lanoiselée et al., 2017, PLOS Medicine, 14:3). FAD has an autosomal dominant inheritance pattern and a prevalence of 1% of all AD cases. In FAD patients Aβ plaques depositions are detected in asymptomatic patients and most likely drive the progression of Tau pathology. High rates of Tau accumulation can be seen in symptomatic FAD patients and Tau pathology has been suggested as an indicator of the onset of cognitive impairments (Gordon et al., Brain. 2019 Apr. 1; 142(4):1063-1076). In humans, it has been suggested that Aβ plaques deposition does not correlate with the degree of cognitive decline but may be required for progression of Tau pathology.

Aβ is produced by sequential cleavage of β-amyloid precursor protein (APP) by β-secretase and γ-secretase. The β-secretase enzyme (BACE1) cleaves APP at the N terminus of the Aβ sequence, releasing secreted APP-β and the membrane-bound C99 fragment. Following cleavage by BACE1, the γ-secretase complex binds to N-terminally cleaved APP fragment (C99) and intramembranous cleaves at the ε-site releasing C-terminal fragment (CTF) and Aβ49 or Aβ48. The γ-secretase complex then processes along the remaining Aβ C-terminal end, producing sequentially shorter peptides until the Aβ peptide is released from the complex (generally after producing peptides 38-, 40-, and 42-amino acids in length). Therefore, the processivity of the γ-secretase complex defines the length of the generated Aβ peptide. AB peptides are prone to aggregate into β sheet conformations in the form of higher-order oligomers, protofibrils, and fibrils, which are detectable in AD brain. Longer Aβ peptides (≥Aβ42) are more hydrophobic and show more capacity to aggregate and form Aβ plaques (L. Chávez-Gutiérrez and M. Szaruga, 2020, Seminars in Cell and Developmental Biology 105, 75-85; Long and Holtzman et al., 2019, Cell 170, 312-339) Importantly, γ-secretase complexes exhibit broad substrate specificity with close to 100 type 1 membrane proteins listed as potential substrates, besides APP (Wakabayashi et al., 2008, Physiology 23, 194-204).

The γ-secretase complex consists of four protein subunits: Presenilin (PSEN), presenilin enhancer 2 (PEN-2), Anterior pharynx-defective (APH), and Nicastrin. There are multiple isoforms of PSEN (PSEN1 or PSEN2) and APH (APH1A or APH1B); therefore up to four different γ-secretase complexes may exist in a single cell (De Strooper., 2003 Neuron, 38,9-12). PSEN1 is an aspartyl protease and is the catalytic subunit of γ-secretase, PEN-2 is required for γ-secretase maturation, APH stabilizes the complex and Nicastrin is thought to play a role in substrate binding (De Strooper et al., 1998, Nature 391, 387-390, De Strooper et al., 1999, Nature 398, 518-522, Wolfe at al., 1999, Nature 398, 513-517; N. Takasugi et al., 2003, Nature 422, 438-441; S. Shah et al., 2005, Cell 122, 435-447; R. Zhou et al., 2019, Science 363, 6428).

The proteins involved in Aβ peptide production form part of a membrane-embedded protease complex called the γ-secretase complex and are composed of the following four proteins with a 1:1:1:1 stoichiometry: Nicastrin (NCT), Anterior pharynx-defective 1 (APH1A or APH1B), Presenilin enhancer 2 (PEN-2) and Presenilin (PSEN-1 or PSEN-2). Presenilin-1 and -2 are the catalytic subunits of the complex (De Strooper., 2003Neuron, 38, 9-12). Presenilin 1 and Presenilin 2 are paralogs. γ-secretase complexes exhibit broad substrate specificity with close to 100 type 1 membrane proteins listed as potential substrates (Wakabayashi et al., 2008, Physiology 23, 194-204). One of the substrates of the γ-secretase complex is the Amyloid Precursor Protein (APP), which is converted into Aβ peptides by said complex (De Strooper et al., 1998, Nature 391, 387-390). APP may be processed through two distinct pathways: the non-amyloidogenic pathway and the amyloidogenic pathway.

Mutations in APP (OMIM 104760), PSEN1 (OMIM 104311) and PSEN2 (OMIM 1600759), have been found to be the main cause of the Familial Alzheimer's disease (FAD) (Lanoiselée et al., 2017, PLOS Medicine, 14:3). Currently, almost 300 mutations in PSEN1, over 10 in PSEN2 and over 18 in APP, have been linked to early onset FAD. Mutations in PSEN1 are not only significantly more prevalent than the ones in PSEN2 or APP, but are also associated with earlier clinical onsets and atypical cognitive presentations, such as motor impairments (L. Chávez-Gutiérrez and M. Szaruga, 2020, Seminars in Cell and Developmental Biology 105, 75-85); Mutations in PSEN1 have different effects:
1. some reduce or abolish γ-secretase activity,
2. others decrease the processivity of the γ-secretase activity, thereby shifting the γ-secretase activity towards generation of longer Aβ peptides species from APP,
3. others reduce the stability of the γ-secretase and the Aβ peptide during the sequential cleavage, or
4. reduce PSEN1 maturation.

It is strongly debated whether presenilin mutations cause Alzheimer's Disease by loss of function or gain of function (Xia et al., 2015, Neuron, 85 (5), 967-81; Veugelen et al., 2016, Neuron, 90 (2), 410-6). The most parsimonious explanation is that mutations in PSEN1 affect the activity of γ-secretase, leading to an increase in Aβ42/40 ratio, which increases neurotoxic oligomer assembly that causes synaptic dysfunction and neurodegeneration (Zoltowska & Berezovska, 2017, Molecular Neurobiology 55, 2275-2284, Chavez-Gutierrez et al., 2012. The EMBO Journal 31, 2261-2274; Szaruga et al., 2017, Cell 170, 443-456).

Until 2021, only symptomatic treatments were available for AD patients, mainly comprising cholinesterase inhibitors and NMDA inhibitors (Long and Holtzman et al., 2019, Cell 170 (2), 312-339). On Jun. 7, 2021, the U.S. Food and Drug Administration (FDA) granted accelerated approval of the first disease-modifying treatment for AD: Aduhelm (aducanumab). However, a post-approval trial is required to verify that the drug provides the expected clinical benefit and grant a continued approval for Aduhelm. Adulhelm is a monoclonal antibody that targets extracellular Aβ plaques in the brain. (https://www.fda.gov/news-events/press-announcements/fda-grants-accelerated-approval-alzheimers-drug)

Therefore, there is still a need for new and more efficient treatments for a disease or condition associated with an abnormal (or altered) processing of the Amyloid Precursor Protein (APP), preferably familiar Alzheimer disease (FAD). The inventors designed antisense oligonucleotides that could be used for such treatment. Such antisense oligonucleotides are expected not to have all drawbacks of existing treatments.

### LEGENDS TO THE FIGURES

FIGS. 1A-1D

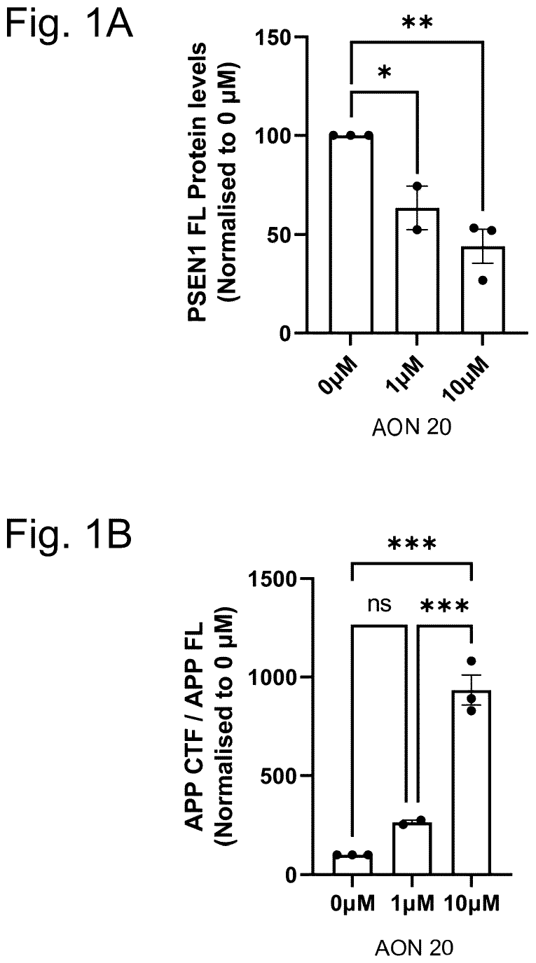

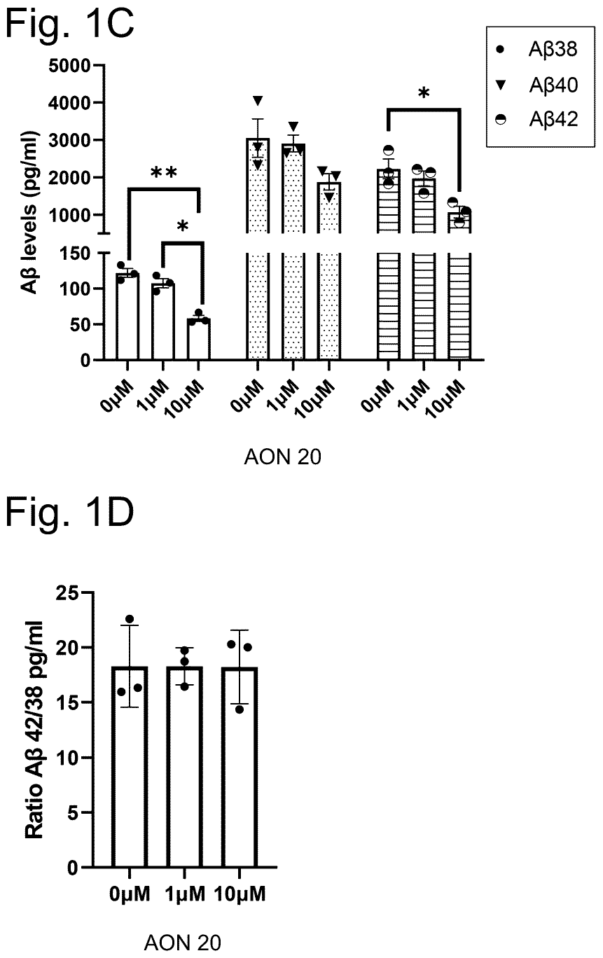

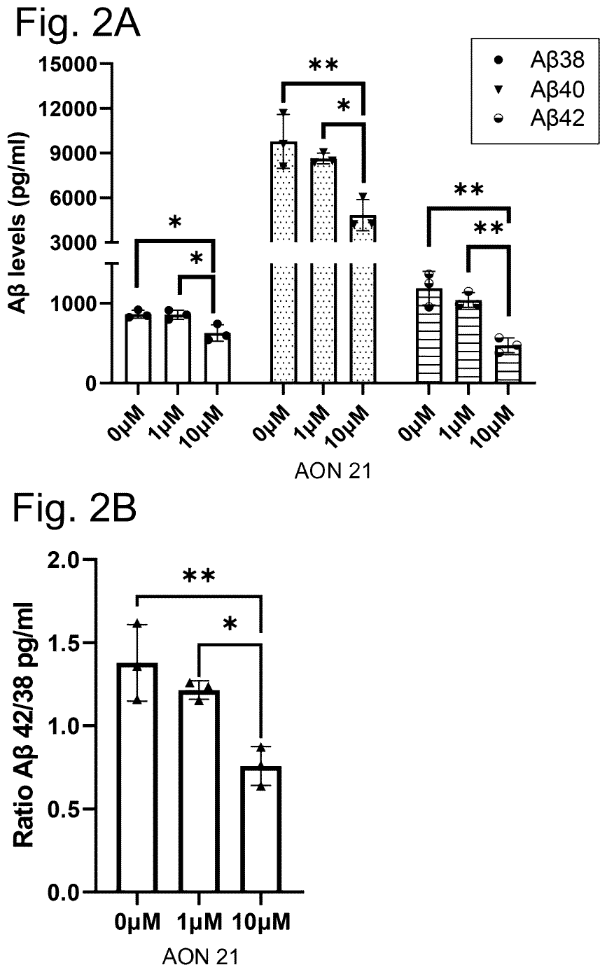

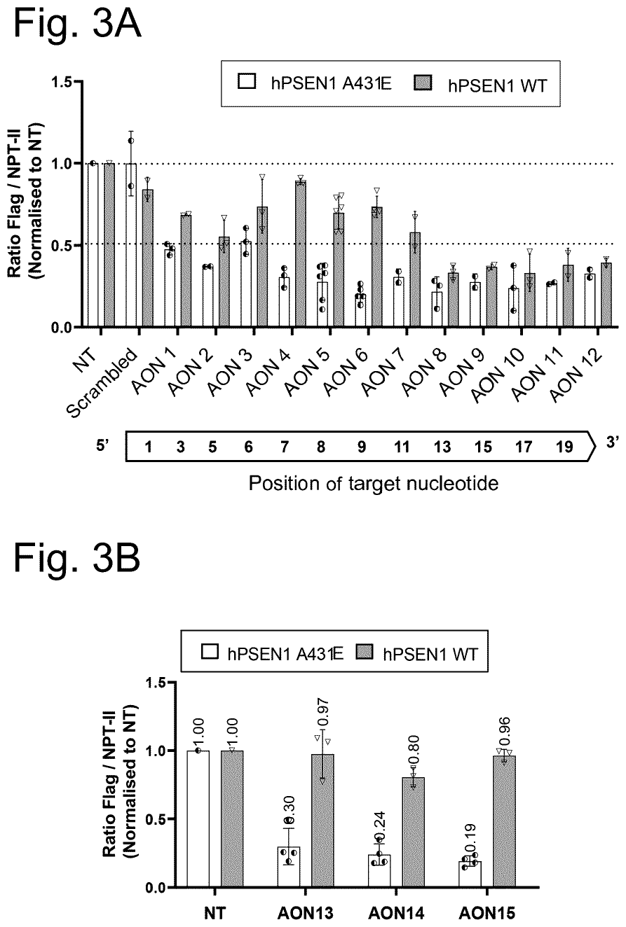

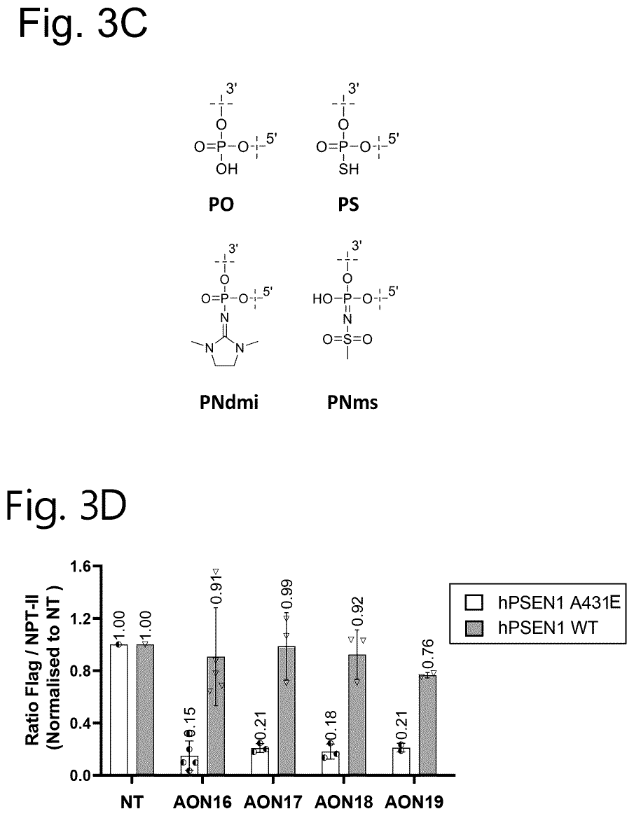

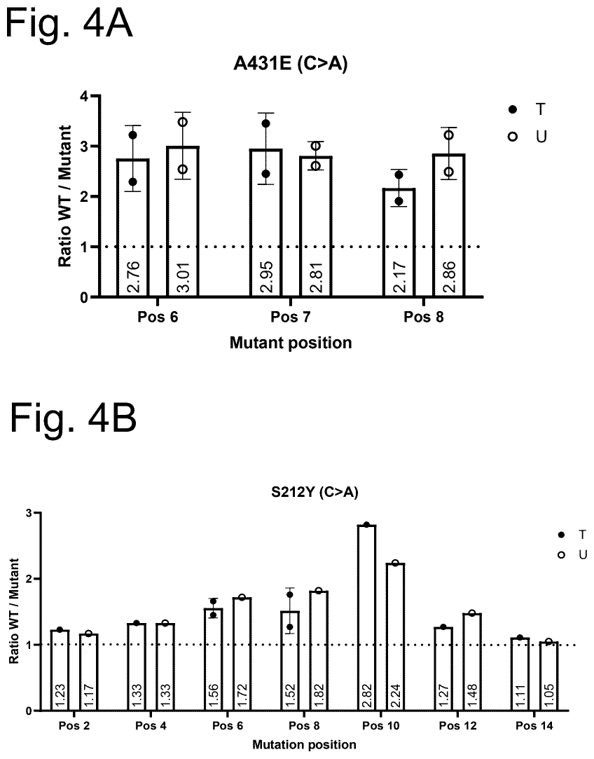

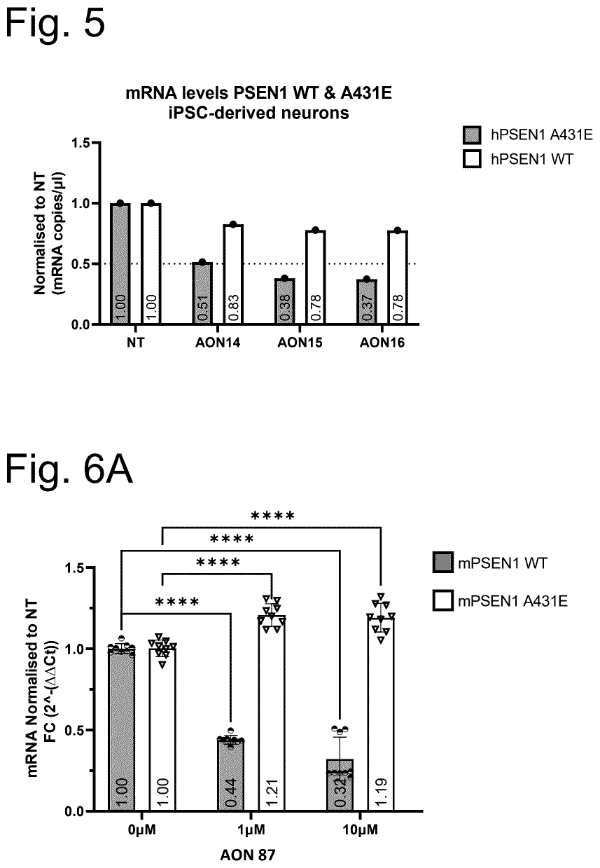

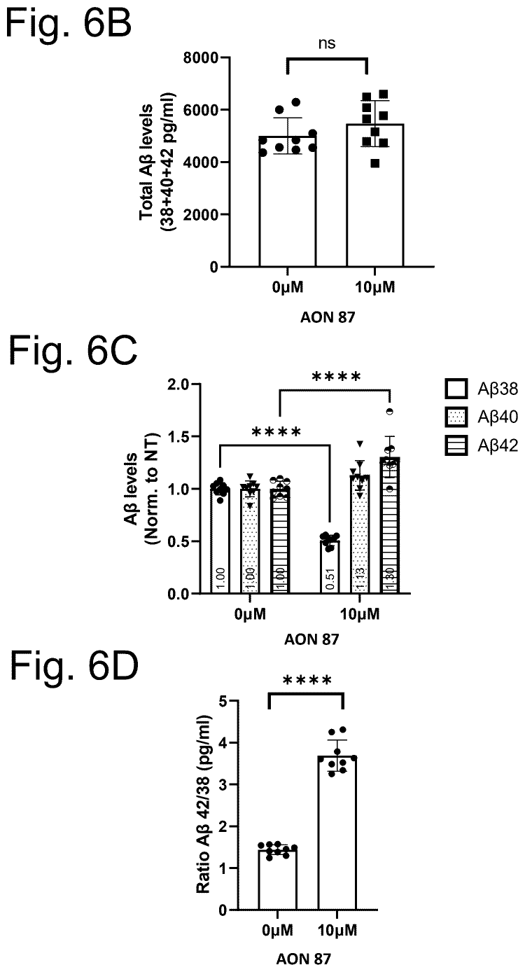

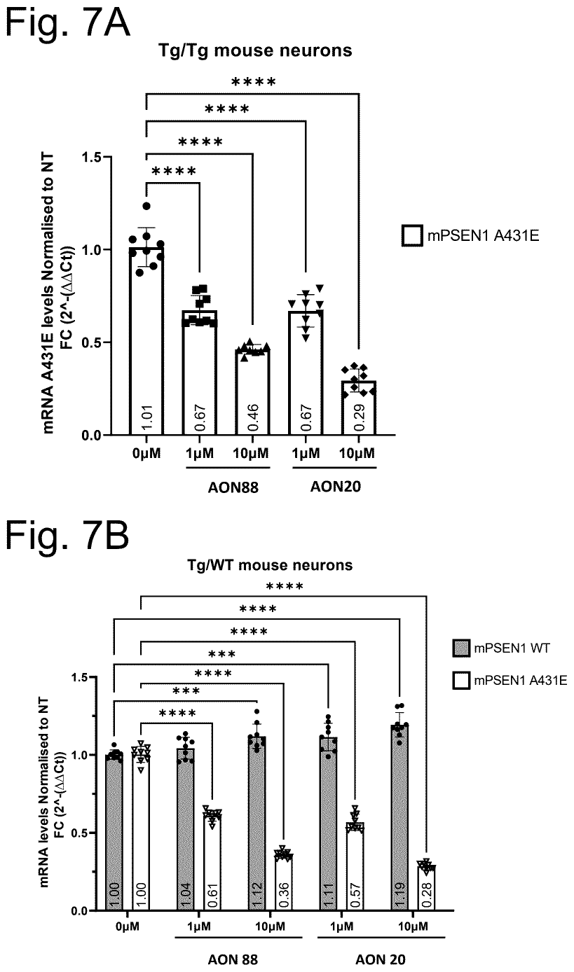

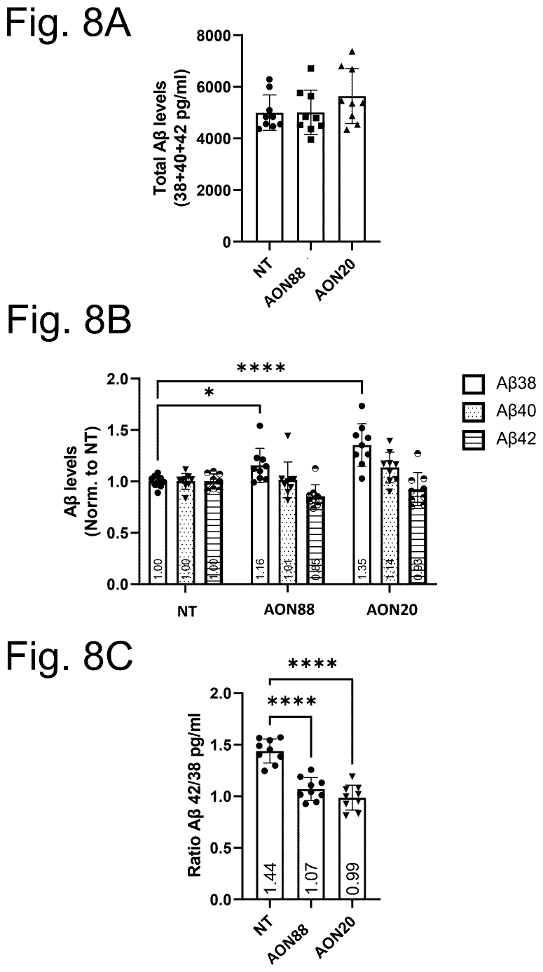

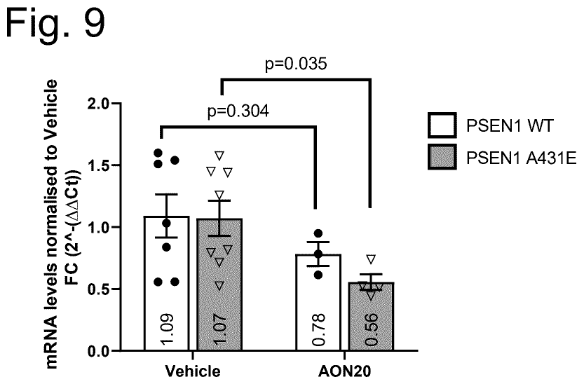

Analysis showing the effect on PSEN1 protein levels (FIG. 1A) and gamma-secretase activity (FIG. 1B-FIG. 1D) of AON20 (SEQ ID NO:118) treatment to Tg/Tg A431E mouse neurons. (FIG. 1A-FIG. 1B) Western blot analysis showing the protein levels of full-length PSEN1 (FIG. 1A) or the protein levels of CTF APP normalized by full-length APP (FIG. 1B) after 8 days treatment with AON20 at the indicated concentrations. Data in A and B is shown relative to the non-treated condition (0 μM). (FIG. 1C-FIG. 1D) ELISA analysis showing the profile of soluble Aβ38, Aβ40 and Aβ42 species in the supernatant of mouse Tg/Tg A431E neurons after 8 days treatment with AON20 at the indicated concentrations. Data is shown as Aβ levels (FIG. 1C) and as Aβ42/38 ratio (FIG. 1D). Each dot in the graph corresponds to one cell culture well, error bars indicate standard deviation (n=2-3). * p<0.05, ** p<0.01 (one-way ANOVA test). CTF=C-terminal fragment. FL=full length.

FIGS. 2A-2B

ELISA analysis showing the profile of soluble Aβ38, Aβ40, and Aβ42 species in the supernatant of mouse Tg/Wt A431E neurons after 7d treatment with AON21 (SEQ ID NO: 120) at the indicated concentrations. Data is shown as Aβ levels (FIG. 2A) and as Aβ42/38 ratio (FIG. 2B). Each dot in the graph corresponds to one cell culture well, error bars indicate standard deviation (n=3). * p<0.05, ** p<0.01 (one-way ANOVA test).

FIGS. 3A-3D

Wes analysis showing the effectivity and allele specificity of AONs targeting human A431E PSEN1 transfected to NIH3T3 cells overexpressing Flag-tagged human WT or A431E PSEN1. Data is shown as the ratio of Flag and NPT-II protein levels normalized to non-treated samples. AONs were transfected at 10 nM and cells were harvested 24 h after transfection. (FIG. 3A) Protein levels of WT and A431E PSEN1 after treatment with AONs wherein the sequence and the position of the nucleotide opposite the mutation shifted from the 5′ end to the 3′ end. (FIG. 3B) Protein levels of WT and A431E PSEN1 after treatment with derivatives from AON4, AON5, and AON6 (SEQ ID NO:8, 10 and 12) which contain a T (thymine) to U (uracil) substitution at the position opposite the mutation. (FIG. 3C) Backbone modifications used in AONs listed in Table 4. (FIG. 3D) Protein levels of WT and A431E PSEN1 after treatment with derivatives from AON14 (SEQ ID NO: 28) that incorporate PO and/or PNms linkages. NT=non-treated. Each dot in the graph corresponds to one cell culture well, error bars indicate standard deviation.

FIGS. 4A-4B

Analysis showing the effect of the position of the mutation within the AON and the effect of a T (thymine) to U (uracil) substitution opposite to the mutation position on the effectivity and allele selectivity of AONs targeting human A431E (FIG. 4A) or S212Y mutations (FIG. 4B). NIH3T3 cells were transfected with plasmids encoding for WT, A431E or S212Y human PSEN1. Three hours later, 10 nM of the indicated AONs were transfected and cells were harvested 24 h after transfection. (dd) PCR was used to measure WT and mutant mRNA levels. Data is shown as the ratio of WT and A431E (FIG. 4A) or S212Y (FIG. 4B) PSEN1 mRNA levels. AONs used are listed in Table 5. Each dot in the graph corresponds to one cell culture well (n=1 or 2), error bars indicate standard deviation.

FIG. 5

(dd) PCR analysis showing mRNA levels of WT and A431E PSEN1 in human neurons differentiated from patient-derived iPSC after 10 days of treatment with AON14 (SEQ ID NO:28), AON15 (SEQ ID NO:30), and AON16 (SEQ ID NO:33). mRNA copies of WT and A431E were quantified with a SNP assay and normalized to the mRNA copies of two housekeeping genes (TFRC and GAPDH). Data is shown relative to the non-treated condition (NT), n=1 cell culture well.

FIGS. 6A-6D

Analysis showing the effect on WT and A431E mouse Psen1 mRNA levels (FIG. 6A) and γ-secretase activity (FIG. 6B-FIG. 6D) of AON87 (SEQ ID NO: 202) treatment to Tg/Wt A431E primary mouse neurons. AON87 targets specifically mouse WT Psen1. (FIG. 6A) RT-qPCR analysis showing the mRNA levels of A431E and WT mouse Psen1 after 7 days of treatment with AON87 at the indicated concentrations. mRNA levels of A431E and WT Psen1 were normalized to the levels of two housekeeping genes (Actin and Rsp23). Data is shown relative to the non-treated condition (0 μM). (FIG. 6B-FIG. 6D) ELISA analysis showing the profile of soluble Aβ38, Aβ40, and Aβ42 species in the supernatant of Tg/Wt A431E primary mouse neurons after 7 days of treatment with AON87 at the indicated concentrations. Data is shown as total Aβ levels (FIG. 6B), as levels of Aβ38, Aβ40, and Aβ42 relative to the non-treated condition (FIG. 6C), and as the ratio Aβ42/38 (FIG. 6D). mRNA and soluble Aβ levels were measured in the same samples, each dot in the graph corresponds to one cell culture well (n=9), error bars indicate standard deviation. **** p<0.0001 (FIG. 6A and FIG. 6C, two-way ANOVA test with Dunnett's multiple comparisons test; FIG. 6B and FIG. 6D, t-test).

FIGS. 7A-7B

RT-qPCR analysis showing mRNA levels of WT and A431E Psen1 in Tg/Tg (FIG. 7A) and Tg/Wt (FIG. 7B) mouse primary neurons after 7 days of treatment with AON20 (SEQ ID NO: 118) and AON88 (SEQ ID NO:204) at the indicated concentrations. Data is shown relative to the non-treated condition (0 μM). Each dot in the graph corresponds to one cell culture well (n=9), error bars indicate standard deviation. *** p<0.001, **** p<0.0001 (one-way ANOVA test with Dunnett's multiple comparisons test).

FIGS. 8A-8C

ELISA analysis showing the profile of soluble Aβ38, Aβ40, and Aβ42 species in the supernatant of Tg/Wt primary mouse neurons after 7 days of treatment with AON20 (SEQ ID NO: 118) and AON88 (SEQ ID NO: 204) at 10 UM concentration. Data is shown as total Aβ levels (FIG. 8A), as levels of Aβ38, Aβ40, and Aβ42 relative to the non-treated condition (FIG. 8B), and as the ratio Aβ42/38 (FIG. 8C). Each dot in the graph corresponds to one cell culture well (n=9), error bars indicate standard deviation. *** p<0.001, **** p<0.0001 (FIG. 8A and FIG. 8C, one-way ANOVA test with Dunnett's multiple comparisons test; FIG. 8B, two-way ANOVA test with Dunnett's multiple comparisons test). NT: none treated.

FIG. 9

Analysis showing AON20 (SEQ ID NO:118) efficacy and allele selectivity in-vivo. Psen1 ^{wt/A431E}; APPKi mice receive a single intracerebroventricular injection of 150 μg of AON20. Animals were sacrificed after 2 weeks and hippocampal mRNA levels of WT and A431E Psen1 were measured by RT-qPCR. Results were normalised to Actin and GAPDH levels. Data is shown relative to vehicle condition. Each dot in the graph corresponds to one animal (n=7, vehicle; n=4, AON20), error bars indicate standard deviation. Statistical test: T-test.

### DESCRIPTION OF THE INVENTION

The invention targets the real underlying source of FAD by depleting the mutant form of the allele and in that way restoring the normal activity of the catalytic subunit of the γ-secretase complex using an oligonucleotide of the invention as defined herein. Such oligonucleotide preferentially targets and breaks down the allele carrying a mutation in PSEN1 or PSEN2, which will reduce the levels of mutant PSEN1 or PSEN2, allow replacement by the corresponding wild type proteins, increase the levels of functional γ-secretase, and thus reduce the production of toxic forms of Aβ peptides. This strategy is different from attempts to inhibit or downregulate the γ-secretase as such which causes severe (Notch-) signaling side effects and is also expected to be more effective than current approaches that focus on more downstream features of the disease such as Tau aggregation or more generic features of the disease like inflammation for the treatment of familial AD. This strategy is quite unique: the oligonucleotides are selected to have the highest possible efficiency and the highest possible specificity against the mutated allele of PSEN1 or PSEN2, while they are selected for the lowest possible efficiency and the lowest possible specificity against the wild type allele of PSEN1 or PSEN2. It means the oligonucleotides have been designed to specifically inactivate, delete, knock down, suppress the mutated allele of the PSEN1 or PSEN2 and primarily keep the wild type allele thereof intact.

### Antisense Oligonucleotide

The inventors surprisingly discovered antisense oligonucleotides that exhibit attractive therapeutic activities and that may be used for treating a disease or condition associated with an abnormal (or altered) processing of the Amyloid Precursor Protein (APP) preferably familiar Alzheimer disease (FAD).

Such antisense oligonucleotides are described below in more detail. Such antisense oligonucleotides will be referred to herein as antisense oligonucleotides according to the invention (or antisense oligonucleotide of the invention or oligonucleotide of the invention or oligonucleotide or AON).

In a first aspect, there is provided an antisense oligonucleotide which preferentially targets a mutated allele of a protein of the γ-secretase complex when present in a cell comprising (preferably expressing) said mutated allele. In a preferred embodiment, the protein of the γ-secretase complex whose allele is mutated is PSEN1 or PSEN2, preferably PSEN1. In a preferred embodiment, the antisense oligonucleotide is single-stranded.

In a second aspect, there is provided an antisense oligonucleotide comprising 15 to 30 or 20 to 30 nucleotides including a central region of 5 to 15 consecutive DNA nucleotides and/or DNA nucleotides analogues flanked in each end by wing regions comprising 1 to 5 RNA nucleotides analogues, which preferentially targets a mutated allele of a protein of the γ-secretase complex when present in a cell comprising and preferably expressing said mutated allele, wherein the mutation present in the mutated allele is targeted by the first, second, third, fourth, fifth, sixth, seventh, eight, nineth, tenth, eleventh, or twelfth nucleotide of the central region of the oligonucleotide. In a preferred embodiment, the protein of the γ-secretase complex whose allele is mutated is PSEN1 or PSEN2, preferably PSEN1. In a preferred embodiment, the antisense oligonucleotide is single-stranded.

As demonstrated in the example section of the present application, the antisense oligonucleotides of the second aspect are demonstrated to efficiently target a mutated allele of a protein of the γ-secretase complex, such as PSEN1.

In an embodiment, there is provided an antisense oligonucleotide comprising 15 to 30 or 20 to 30 nucleotides including a central region of 5 to 15 consecutive DNA nucleotides and/or DNA nucleotides analogues flanked in each end by wing regions comprising 1 to 5 RNA nucleotides analogues, which preferentially targets a mutated allele of a protein of the γ-secretase complex when present in a cell comprising and preferably expressing said mutated allele, wherein the mutation present in the mutated allele is targeted by the sixth, seventh, eighth, nineth, tenth, eleventh, twelfth, thirteenth, fourteenth, fifteenth, sixteenth, seventeenth, or eighteenth nucleotide in the oligonucleotide sequence starting from 5′ to 3′. In a preferred embodiment, the protein of the γ-secretase complex whose allele is mutated is PSEN1 or PSEN2, preferably PSEN1. In a preferred embodiment, the antisense oligonucleotide is single-stranded.

All definitions and/or embodiments described herein relate to both the first and the second aspects of the invention unless otherwise indicated.

In an embodiment, said cell is a cell, wherein said protein is endogenously expressed (mutated allele and preferably the wild type allele). In a preferred embodiment, the cell is a mammalian cell, preferably a murine or human cell. In a more preferred embodiment, the cell is representative for the disease FAD.

In a first embodiment, a cell which is representative for the disease FAD is a neuronal cell. Preferred neuronal cells are cells of the central nervous system. A preferred cell of the central nervous system may be from, or may be derived from, a region of the brain which is known to be affected in FAD. Such brain regions include the hippocampus, amygdala, cerebral cortex (e.g frontal lobe, parietal lobe, temporal lobe and/or occipital lobe) cerebellum, and the thalamus. Accordingly, in an embodiment, the activity of the antisense oligonucleotide is assessed in such neuronal cells. The assessment may also be carried out in vitro by culturing such cells and contacting them with the oligonucleotide.

In a second embodiment, a cell which is representative for the disease FAD is a human iPSC (induced pluripotent stem cell), which is triggered to differentiate or mature into neurons. As indicated in Example 5, the antisense oligonucleotides of the invention are able to successfully target proteins of the γ-secretase complex whose allele is mutated (PSEN1) in biologically relevant FAD systems, such as iPSC cells. Preferably, the iPSC is from a subject having an altered or diminished PSEN1 or PSEN2 activity, and is a FAD patient or is suspected to become a FAD patient due to his/her genetic background. Accordingly, in an embodiment, the activity of the antisense oligonucleotide is assessed using such neuronal cells derived from human iPSC.

The assessment may be carried out in vitro by culturing such cells and contacting them with the oligonucleotide.

Alternatively in another embodiment, said cell is a cell, wherein said protein is exogenously expressed (mutated allele or the wild type allele). In a preferred embodiment, the cell is a mammalian cell, preferably a murine or human cell. In this embodiment, a nucleic acid coding for said mutated protein is introduced into said cell. Said cell may be a non-neuronal cell, including but not limited to fibroblast, HeLa, or HEK293 cell. Said nucleic acid coding for said mutated protein may be introduced by viral or non-viral vectors, more preferably plasmids expressing the mutant protein or fusion-derivatives thereof.

Depending on the oligonucleotide used, the skilled person will know which cell is suitable to assess the activity of the oligonucleotide. As illustrated above, in an embodiment, said cell is a cell wherein said protein is endogenously expressed. Alternatively in another embodiment, a nucleic acid coding for said mutated protein is introduced into said cell. The skilled person may also assess the activity of the oligonucleotide in a cell exogenously and subsequently in a cell endogenously expressing the mutated protein.

The choice of the read out is a key feature of the invention as the use of a given read out may lead to the identification of oligonucleotide that are not optimal when later validated using a more relevant read out. Here, the inventors demonstrated that the use of the effect of the oligonucleotide on the mutated PSEN1 or PSEN2 transcript and/or the absence of effect on the wild type allele of said transcript and/or the effect of the oligonucleotide to normalize, reverse or correct abnormal Aβ42/38 and/or Aβ43/40 and/or Aβ43/37 and/or Aβ42/40 ratios, preferably the Aβ42/38 and/or Aβ43/40 ratios were crucial, more preferably Aβ42/38 ratios were crucial. In particular, the inventors have surprisingly found that antisense oligonucleotides of the invention have the biggest impact on Aβ38 peptide levels and the Aβ42/Aβ38 ratio while the effect on other Aβ-species (such as Aβ40) is less pronounced (Examples 2, 6, and 8). This observation confirms the need of using holistic readouts to evaluate the therapeutic benefit of the therapeutic effect of antisense oligonucleotides;

In a preferred embodiment, there is provided an antisense oligonucleotide which preferentially targets a mutated allele of a protein of the γ-secretase complex (preferably PSEN1) when present in a cell comprising (preferably expressing) said mutated allele. More preferably, the cell is a neuronal cell, even more preferably a human neuronal cell. Preferably, the cell expresses the endogenous mutated allele of a protein of the γ-secretase pathway. In a preferred embodiment,
the oligonucleotide is able to preferentially silence, inactivate, break down, knock down, reduce or decrease mutated PSEN1 or PSEN2 transcripts (preferably mutated PSEN1 transcripts) and even more preferably
the oligonucleotide is not (or minimally) able to silence, inactivate, break down, knock down, reduce or decrease wild-type PSEN1 or PSEN2 transcripts (preferably wild-type PSEN1 transcripts).

In a more preferred embodiment,
the oligonucleotide is able to preferentially silence, inactivate, break down, knock down, reduce or decrease mutated PSEN1 or PSEN2 transcripts (preferably mutated PSEN1 transcripts), and even more preferably the oligonucleotide is not (or minimally) able to silence, inactivate, break down, knock down, reduce or decrease wild-type PSEN1 or PSEN2 transcripts (preferably mutated PSEN1 transcripts) and/or
the oligonucleotide is able to normalize, reverse or correct abnormal Aβ42/38 and/or Aβ43/40 and/or Aβ43/37 and/or Aβ42/40 ratios, preferably the Aβ42/38 and/or Aβ43/40 ratios, and more preferably the Aβ42/38 ratio.

In an embodiment, the oligonucleotide:
1. preferentially silences or inactivates or breaks down mutated PSEN1 transcripts, and knocks down or reduces or decreases mutant PSEN1 protein expression and preferentially does not silence or inactivate or break down wild-type PSEN1 transcripts, and does not knock down or reduce or decrease wild-type PSEN1 protein expression, and/or
2. is able to normalize, correct, or reverse abnormal Aβ42/38 and/or Aβ43/40 and/or Aβ43/37 and/or Aβ42/40 ratios (preferably Aβ42/38 and/or Aβ43/40 ratios, and more preferably the Aβ42/38 ratio.

In an embodiment, the oligonucleotide:
1. preferentially silences or inactivates or breaks down mutated PSEN1 transcripts, and/or preferentially does not silence or inactivate or break down wild-type PSEN1 transcripts, and/or
2. is able to normalize, correct, or reverse abnormal Aβ42/38 and/or Aβ43/40 and/or Aβ43/37 and/or Aβ42/40 ratios (preferably Aβ42/38 and/or Aβ43/40 ratios, more preferably the Aβ42/38 ratio).

Each of these read outs have been extensively defined below.

In the context of the invention, an activity elicited or exhibited by an antisense oligonucleotide of the invention is to preferentially target a mutated allele of a protein of the γ-secretase complex when present in a cell comprising said mutant allele. In an embodiment, said protein is a human protein and/or is PSEN1.

The targeted mutated allele, especially the region or stretch of this mutated allele which is targeted may also be named the targeted region of the oligonucleotide.

This mutated allele of a human protein of the γ-secretase complex may be a disease-associated or disease-causing allele in a cell of a patient, in a tissue of a patient and/or in a patient as explained later herein. In an embodiment, the patient is a mammal. In a preferred embodiment, the patient is a human.

Throughout the application, the word “binds”, “specifically binds”, “targets”, “specifically targets”, “hybridizes” or “specifically hybridizes” could be used interchangeably when used in the context of an antisense oligonucleotide which is reverse complementary to a part of an allele, especially in the form of a transcript encoding this allele as identified herein. It follows that the expression “oligonucleotide” is synonymous with the expression “antisense oligonucleotide” in the context of the application.

Throughout the application, the word “preferentially binds”, “preferentially targets” or “preferentially hybridizes” could be used interchangeably when used in the context of an antisense oligonucleotide of the invention which is reverse complementary to a part of a mutated allele, especially in the form of a transcript encoding this mutated allele as identified herein. This “preferential binding/targeting/hybridizing” is later herein defined by comparison to the “specific binding/targeting/hybridizing” to a wild type/control/non mutated allele.

The word “transcript” refers to the pre-mRNA or mRNA encoded by an allele. In the context of the invention, a mutated allele is a gene encoding a mutated transcript, said mutated transcript encoding a mutated protein of the γ-secretase complex.

In this context, the wild type genomic DNA of mouse PSEN1 comprises SEQ ID NO: 145, the corresponding coding RNA comprises SEQ ID NO: 146 and the mouse PSEN1 protein comprises SEQ ID NO: 147.

In this context, the A431E genomic DNA of mouse A431E PSEN1 comprises SEQ ID NO: 148, the corresponding coding RNA comprises SEQ ID NO: 149 and the mouse A431E PSEN1 protein comprises SEQ ID NO: 150.

In this context, the wild type genomic DNA of human PSEN1 comprises SEQ ID NO: 151, the corresponding coding RNA comprises SEQ ID NO:152 or 169 and the human PSEN1 protein comprises SEQ ID NO: 153 or 159.

In this context, the A431E genomic DNA of human A431E PSEN1 comprises SEQ ID NO: 154, the corresponding coding RNA comprises SEQ ID NO: 155 or 170 and the human A431E PSEN1 protein comprises SEQ ID NO: 156 or 161.

In this context, the wild type genomic DNA of human PSEN2 comprises SEQ ID NO: 165, the corresponding coding RNA comprises SEQ ID NO: 171 or 172 and the human PSEN2 protein comprises SEQ ID NO: 166 or 168.

As used herein, “hybridization” refers to the pairing of complementary oligomeric compounds (e.g., an antisense compound and its target nucleic acid/target region). While not limited to a particular mechanism, the most common mechanism of pairing involves hydrogen bonding, which may be Watson-Crick, Hoogsteen or reversed Hoogsteen hydrogen bonding, between complementary nucleoside or nucleotide bases (nucleobases). For example, the natural base adenine is nucleobase complementary to the natural nucleobases thymine, 5-methyluracil and uracil which pair through the formation of hydrogen bonds. The natural base guanine is nucleobase complementary to the natural bases cytosine and 5-methyl-cytosine. Hybridization can occur under varying circumstances. In particular, hybridization of an oligonucleotide of the invention with a targeted pre-mRNA and/or mRNA can occur under varying circumstances. Similarly, binding of an oligonucleotide of the invention to a targeted pre-mRNA and/or mRNA (i.e. targeted region) can occur under varying circumstances. Preferably, said hybridization or said binding is assessed under physiological conditions in a cell, more preferably in a human cell. Preferred cells have been defined earlier herein. An oligonucleotide of the invention is preferably said to be able to bind to, or capable of binding to, or able to hybridize with, or capable of hybridizing with, when said binding or hybridization occurs under physiological conditions in a cell, preferably a human cell. Preferred cells have been defined earlier herein.

In the context of the invention, “hybridizes” or “binds” (or “preferentially hybridizes” or preferentially binds” or “preferentially binds” or “preferentially hybridizes”) is used under physiological conditions in a cell, preferably a human cell unless otherwise indicated.

In an embodiment, an antisense oligonucleotide is said to target a mutated allele encoding a mutated protein of the γ-secretase complex (preferably PSEN1) when said oligonucleotide is reverse complementary to at least 10 or at least 15 consecutive/contiguous bases of said mutated allele or of said mutated transcript encoding said mutated protein. This “at least 10 or at least 15 consecutive/contiguous bases of said mutated allele” may also be called the targeted region or the target region of the antisense oligonucleotide. In an embodiment, the length of the reverse complementary part may be at least 10, 11, 12, 13, 14, 15, 16, 17, 18, 19, 20, 21, 22, 23, 24, 25, 26, 27, 28, 29, 30, 31, 32, 33, 34, 35 nucleotides. Within the context of the invention, an oligonucleotide comprises up to 50 nucleotides and/or nucleotide analogues. An oligonucleotide may be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides and/or nucleotide analogues. The part of the oligonucleotide which is reverse complementary does not per se need to be 100% reverse complementary with the mutated allele or transcript of a mutated protein of the γ-secretase complex. In an embodiment, one or two mismatches may be present over a length of at least 15 nucleotides in total defining the part which is reverse complementary with said mutated allele or transcript.

An activity of the oligonucleotide of the invention is to preferentially target a mutated allele or mutated transcript encoding a mutated protein of the γ-secretase complex. This activity may mean that (the levels of) the targeted mutant allele (or corresponding transcript) is silenced or knocked down (or broken down) or inactivated or reduced or decreased. In other words, the levels of said mutated transcript are knocked down (or broken down), reduced or decreased. In this context, the protein of the γ-secretase complex is PSEN1. In a preferred embodiment, the protein is a human protein. More preferably, human PSEN1.

Therefore, a first way of assessing an activity of the antisense oligonucleotide of the invention is to assess whether the oligonucleotide is able to knock down (or break down) or reduce or decrease (the levels of) said mutated transcript (preferably PSEN1 or PSEN2 mutated allele or transcript). This reduction or decrease of the level of mutated transcript may be of at least 1%, 5%, 10%, 15%, 20%, 25%, 30%, 35%, 40%, 45%, 50%, 55%, 60%, 65%, 70%, 75%, 80%, 85%, 90%, 95% or 100% compared to the level of the same transcript at the onset of the treatment. In a preferred embodiment, the mutated transcript is no longer detectable. The reduction or decrease may be assessed in cells or tissues or subjects treated with said oligonucleotide and using Northern Blotting or (semi) quantitative RT-qPCR or RT-ddPCR analysis of mutant transcript copy numbers (preferably as carried out in the experimental part).

In a preferred embodiment, for this first way of assessing an activity of the antisense oligonucleotide of the invention, the level of the wild type allele or wild type transcript which is not targeted (or not specifically targeted) by the oligonucleotide is also assessed. Accordingly, the wild type allele or transcript is still detectable and the level of said wild type allele or transcript is not less than 30%, 40%, 50%, 60%, 70%, 80%, 90%, 95% or 100% of the level of the same allele or transcript at the onset of the treatment. In a preferred embodiment, the level of the wild type allele or transcript is the same as the one detected at the onset of the treatment. The presence of said transcript may be assessed in cells or tissues or subjects treated with said oligonucleotide and using Northern Blotting or (semi) quantitative RT-qPCR or RT-ddPCR analysis of mutant transcript copy numbers (preferably as carried out in the experimental part).

Therefore, in a preferred embodiment, the activity of the antisense oligonucleotide of the invention is assessed as follows:
whether the oligonucleotide is able to knock down or reduce or decrease the levels of a mutated PSEN1 or PSEN2 transcript and
whether the oligonucleotide is not (or minimally) able to knock down or reduce or decrease the levels of a wild type PSEN1 or PSEN2 transcript.

This reduction or decrease of the level of mutated transcript may be of at least 1%, 5%, 10%, 15%, 20%, 25%, 30%, 35%, 40%, 45%, 50%, 55%, 60%, 65%, 70%, 75%, 80%, 85%, 90%, 95% or 100% compared to the level of the same transcript at the onset of the treatment. In a preferred embodiment, the mutated transcript is no longer detectable.

The oligonucleotide is said not (or minimally) be able to knock down or reduce or decrease the wild type PSEN1 or PSEN2 allele or transcript when said wild type allele or transcript is still detectable is not less than 30%, 40%, 50%, 60%, 70%, 80%, 90%, 95% or 100% of the level of the same allele or transcript at the onset of the treatment.

In a more preferred embodiment, the oligonucleotide is able to knock down or reduce or decrease the levels of a mutated PSEN1 or PSEN2 transcript of at least 20 or at least 30 or at least 40 or at least 50 or at least 80 or at least 90 or at least 95% compared to the level of said mutated allele or transcript at the onset of the treatment and the levels of a wild type PSEN1 or PSEN2 transcript is still at least 50 or at least 80 or at least 90 or at least 95% of the level of said transcript of allele at the onset of the treatment.

The presence of a mutated or of a wild type allele or transcript is assessed as earlier described herein.

As explained in the background section, some mutations in PSEN1 or PSEN2 gene may shift γ-secretase complex activity towards the production of abnormal Aβ profiles, from short peptides (Aβ38, Aβ40) to longer peptides (Aβ42, Aβ43 and even longer up to Aβ48 or Aβ49, although there are no tools readily available to measure those species while the other ones are readily measurable with ELISA assays), as the result of an impairment of the carboxypeptidase-like activity of γ-secretase. The underlying mechanism is the destabilization of the substrate-enzyme complex i.e. the APP substrate interaction with the catalytic presenilin subunit in the γ-secretase complex is decreased because of the mutation of the PSEN1 or PSEN2 gene. This results in faster release of longer (less processed) forms of Aβ i.e. any peptide with a length up to 49 amino acids. The longer (larger or equal to Aβ49 or larger or equal to Aβ48 larger or equal to Aβ42 or larger or equal to Aβ43) forms of the Aβ peptide species are toxic because they are more hydrophobic and aggregate more easily and form more easily toxic oligomeric and fibrillic species. The processing of Aβ is progressive and there are two production lines, one starting with Aβ49>46>43>40>37 and one starting at Aβ48>45>42>38 (Takami et al., 2009, J Neurosci 29, 13042-52). In other words, Aβ42/38 and/or Aβ 43/40 and/or Aβ43/37 and/or Aβ 42/40 ratios are expected to increase in brain, cerebrospinal fluid (CSF) and/or plasma of FAD patients because of the PSEN1 or PSEN2 mutations. In an embodiment the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) in the plasma, and/or CSF and/or brain of FAD patients is increased of at least 1%, 5%, 10%, 15%, 20%, 25%, 30%, 35%, 40%, 45%, 50%, 55%, 60%, 65%, 70%, 75%, 80%, 85%, 90%, 95%, 100% compared to the level in healthy subjects. The Aβ42/38 and/or Aβ43/40 and/or Aβ43/37 and/or Aβ42/40 ratios (preferably the Aβ42/38 and/or Aβ42/40 ratios, more preferably the Aβ42/38 ratio) may be assessed by techniques known to the skilled person.

In an embodiment, the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) may be assessed using cell extract, cell medium, brain extract, CSF or plasma of FAD patients carrying PSEN1 or PSEN2 mutations.

In an embodiment, the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) may be assessed using cell extract, cell medium of cells endogenously or exogenously expressing a mutated PSEN1 or PSEN2.

In an embodiment it is assessed using ELISA, preferably as carried out in the experimental part.

In the in vivo situation (in transgenic mouse), these ratios in CSF, plasma and/or in brain extracts are first expected to increase and subsequently to decrease as a consequence of the aggregation of the Aβ peptides forming Aβ plaques.

In the in vivo situation (in FAD patients), these ratios in in CSF and/or plasma are first expected to increase and subsequently to decrease as a consequence of the aggregation of the Aβ peptides forming Aβ plaques.

This increase is typically observed in FAD patients with no detectable Aβ plaques in the brain. FAD patients with no detectable Aβ plaques in the brain may be called asymptomatic FAD patients. During the progression of the disease, Aβ plaques are formed and the ratios decreased in CSF samples. In plasma, these ratios are increased during all the progression of the disease. Therefore if the patient already has detectable Aβ plaques in the brain, the assay should be carried out in the plasma of said patients. The ELISA assay only assesses the soluble Aβ42/38 and/or Aβ43/40 and/or Aβ43/37 and/or Aβ42/40 ratios (preferably the Aβ42/38 and/or Aβ42/40 ratios, more preferably the Aβ42/38 ratio) and the effect of the aggregation process on the level of the ratios cannot be specified. Said ELISA assay primarily visualizes the first effect of the mutation of the γ-secretase complex activity, which is the generation of longer forms of the Aβ peptide species.

In an embodiment, an asymptomatic FAD subject or patient is selected or identified in view of his/her family history with several FAD patients having a PSEN1 or PSEN2 mutation. It is crucial to diagnose this type of subject when they are still asymptomatic (and therefore as early as possible) in order to start treatment as early as possible. The skilled person may use alternative in vitro assays that are able to assess the presence of aggregated Aβ peptides in brain extracts from a mouse.

Therefore, a second way of assessing an activity of the oligonucleotide of the invention is to determine the Aβ42/38 and/or Aβ43/40 and/or Aβ43/37 and/or Aβ42/40 ratios (preferably the Aβ42/38 and/or Aβ42/40 ratios, and more preferably the Aβ42/38 ratio) in cell cultures, tissues, plasma and/or CSF of treated FAD subjects. In an embodiment, the FAD patients are asymptomatic, with no detectable Aβ plaques in the brain. The oligonucleotide is expected to reduce the generation of long Aβ species leading to reduction of soluble Aβ42/38 and/or Aβ43/40 and/or Aβ43/37 and/or Aβ42/40 ratios (preferably the Aβ42/38 and/or Aβ42/40 ratios, more preferably the Aβ42/38 ratio). When the assessment is carried out in subjects, it is preferably carried out in the plasma and/or CSF of said subjects.

The normalization, correction, or reversion of abnormal Aβ42/38 and/or Aβ43/40 and/or Aβ43/37 and/or Aβ42/40 ratios (preferably the Aβ42/38 and/or Aβ42/40 ratios, more preferably the Aβ42/38 ratio) may mean a decrease of the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level at the onset of the treatment. The Aβ42/38 and/or Aβ43/40 and/or Aβ43/37 and/or Aβ42/40 ratios (preferably the Aβ42/38 and/or Aβ42/40 ratios, more preferably the Aβ42/38 ratio) may be assessed by techniques known to the skilled person. In an embodiment it is assessed using ELISA, preferably as carried out in the experimental part.

If the assessment is carried out in FAD patients with detectable Aβ plaques in the brain, it is preferably carried out in the plasma and not in the CSF of said patients.

The invention is uniquely using a read out which directly demonstrates the therapeutic effect of an oligonucleotide on a disease or condition associated with an abnormal (or altered) processing of the APP. This read out directly demonstrates the downstream consequences of the defects of the γ-secretase complex present in a cell, in a tissue, in plasma and/or in CSF of a FAD patient by assessing Aβ42/38 and/or Aβ43/40 and/or Aβ43/37 and/or Aβ42/40 ratios (preferably the Aβ42/38 and/or Aβ42/40 ratios, more preferably the Aβ42/38 ratio). In an embodiment, the FAD patient is preferably asymptomatic with no detectable Aβ plaques in the brain, as earlier defined herein. The effect of the oligonucleotide on this read out is expected to be representative of a therapeutic effect on a patient with a mutated γ-secretase complex (preferably PSEN1) that will develop FAD. It is expected that the used read out (i.e. Aβ42/38 and/or Aβ43/40 and/or Aβ43/37 and/or Aβ42/40 ratios (preferably the Aβ42/38 and/or Aβ42/40 ratios, more preferably the Aβ42/38 ratio)) is representative of the FAD disease and the normalization, correction or reversion of the abnormal increased soluble Aβ42/38 and/or Aβ43/40 and/or Aβ43/37 and/or Aβ42/40 ratios (preferably the Aβ42/38 and/or Aβ42/40 ratios, more preferably the Aβ42/38 ratio) is therefore predictive of a therapeutic activity of an oligonucleotide. This read out (any of the soluble Aβ42/38 and/or Aβ43/40 and/or Aβ43/37 and/or Aβ42/40 ratios (preferably the Aβ42/38 and/or Aβ42/40 ratios, more preferably the Aβ42/38 ratio), also called “a long/short” Aβ ratios) is more representative and more predictive than a read out limited to a single “long” Aβ peptide only (such as soluble Aβ42). The goal of the therapy is to restore a normal enzymatic activity of the γ-secretase complex or to switch the activity of said complex to a more normal activity. Therefore, the skilled person understands that long/short Aβ peptide ratios provide a better representation of the enzymatic activity of the γ-secretase complex than the use of just a single long form of the Aβ. In other words, in order to better understand, visualize or monitor the activity status of the γ-secretase complex (preferably PSEN1), it is preferred to assess the production of several species of the Aβ peptides and not only the production of a long form thereof.

In an embodiment, it follows that an antisense oligonucleotide of the invention may be considered to be active when:

The oligonucleotide is able to normalize, correct, or reverse abnormal Aβ42/38 and/or Aβ43/40 and/or Aβ43/37 and/or Aβ42/40 ratios (preferably the Aβ42/38 and/or Aβ42/40 ratios, more preferably the Aβ42/38 ratio), wherein the assessment is compared to the level of the same Aβ ratio at the onset of the treatment (or to corresponding level of the same Aβ ratios in healthy subjects). The assessment may be carried out in a cell, a tissue, plasma, or CSF or subject. When a subject is treated, said ratios are preferably assessed in plasma of said subjects. A subject may be an asymptomatic FAD subject. Alternatively, the subject may be a symptomatic FAD subject. If the subject is a symptomatic FAD subject, the assessment is preferably carried out using plasma. An alternative way of assessing the effect of the oligonucleotide on the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) is applying imaging technologies to assess Aβ plaque accumulation in the brain of treated subjects. Imaging technology may include PET scans on FAD patients. Imaging technology may also include histology on murine tissues.

In a preferred embodiment, an antisense oligonucleotide of the invention may be considered to be active when:
the oligonucleotide is able to preferentially silence, inactivate, breakdown, knock down or reduce or decrease or correct (the quantity or level of) mutated PSEN1 (or PSEN2) transcripts
the oligonucleotide is not (or minimally) able to silence, inactivate, breakdown, knock down or reduce or decrease or correct (the quantity or level of) wild-type PSEN1 (or PSEN2) transcripts, and
the oligonucleotide is able to normalize, correct, or reverse abnormal Aβ42/38 and/or Aβ43/40 and/or Aβ43/37 and/or Aβ42/40 ratios (preferably the Aβ42/38 and/or Aβ42/40 ratios, more preferably the Aβ42/38 ratio), preferably wherein the assessment is compared to the level of the same mutant transcript or the same Aβ ratio at the onset of the treatment (or to corresponding level of the same Aβ ratio in healthy subjects). The assessment may be carried out in a cell, a tissue, plasma, or CSF or subject. When a subject is treated, said ratios are preferably assessed in plasma of said subjects. A subject may be an asymptomatic FAD subject. Alternatively, the subject may be a symptomatic FAD subject. If the subject is a symptomatic FAD subject, the assessment is preferably carried out using plasma. An alternative way of assessing the effect of the oligonucleotide on the Aβ42/38 and/or Aβ43/40 and/or Aβ43/37 and/or Aβ42/40 ratios (preferably the Aβ42/38 and/or Aβ42/40 ratios, more preferably the Aβ42/38 ratio) is applying imaging technologies to assess Aβ plaque accumulation in the brain of treated subjects. Imaging technology may include PET scans on FAD patients. Imaging technology may also include histology on murine tissues.

In another preferred embodiment, an antisense oligonucleotide of the invention is considered to be active when:
the oligonucleotide is able to preferentially silence, inactivate, breakdown, knock down or reduce or decrease or correct (the quantity or level of) mutated PSEN1 (or PSEN2) proteins,
the oligonucleotide is not (or minimally) able to silence, inactivate, breakdown, knock down or reduce or decrease or correct (the quantity or level of) wild-type PSEN1 (or PSEN2) proteins, and
the oligonucleotide is able to normalize, correct, or reverse the abnormal increased soluble Aβ42/38 and/or Aβ43/40 and/or Aβ43/37 and/or Aβ42/40 ratios (preferably the Aβ42/38 and/or Aβ42/40 ratios, more preferably the Aβ42/38 ratio), preferably wherein the assessment is compared to the level of the same mutant protein or the same Aβ ratio at the onset of the treatment (or to corresponding level of the same Aβ ratio in healthy subjects). The assessment may be carried out in a cell, a tissue, plasma, or CSF or subject. A subject may be an asymptomatic FAD subject. Alternatively, the subject may be a symptomatic FAD subject. If the subject is a symptomatic FAD subject, the assessment is preferably carried out using plasma.

The silencing or inactivation or breaking down or reduction or decrease or knocking down of the mutated transcript is expected to induce a similar reduction or decrease of the levels of the mutated protein (preferably PSEN1).

In a more preferred embodiment, an antisense oligonucleotide of the invention may be considered to be active when:
the oligonucleotide is able to preferentially silence, inactivate, breakdown, knock down or reduce or decrease or correct (the quantity or level of) mutated PSEN1 (or PSEN2) transcripts,
the oligonucleotide is not (or minimally) able to silence, inactivate, breakdown, knock down or reduce or decrease or correct (the quantity or level of) wild-type PSEN1 (or PSEN2) transcripts,
the oligonucleotide is able to normalize, correct, or reverse abnormal Aβ42/38 and/or Aβ43/40 and/or Aβ43/37 and/or Aβ42/40 ratios (preferably the Aβ42/38 and/or Aβ42/40 ratios, more preferably the Aβ42/38 ratio) and,
the oligonucleotide is able to reduce the formation of Aβ plaques, preferably when compared to the level of the same (mutant) transcript or the same Aβ ratio or the quantity of Aβ plaques at the onset of the treatment (or to corresponding level of the same transcript, or Aβ ratio in healthy subjects).

The Aβ42/38 and/or Aβ43/40 and/or Aβ43/37 and/or Aβ42/40 ratios may be assessed using ELISA, preferably as carried out in the experimental part. The formation of Aβ plaques may be assessed using imaging technologies in the brain of treated subjects. Imaging technology may include PET scans on FAD patients. Imaging technology may also include histology on murine tissues.

The assessment may be carried out in a cell, a tissue, plasma, or CSF or subject. When a subject is treated, said ratios are preferably assessed in plasma of said subjects. A subject may be an asymptomatic FAD subject. Alternatively the subject may be a symptomatic FAD subject. If the subject is a symptomatic FAD subject, the assessment is preferably carried out using plasma.

In an embodiment, the antisense oligonucleotide of the invention preferentially targets a mutated allele (and not, or minimally, the WT allele) of protein PSEN1 or PSEN2 when present in a cell expressing said mutated allele. Preferred cells have been earlier defined herein. In a preferred embodiment, the antisense oligonucleotide of the invention preferentially targets a mutated allele (and not, or minimally, the WT allele) of protein PSEN1 (preferably human protein PSEN1) of the γ-secretase complex when present in a cell expressing said mutated allele. Preferred cells have been earlier defined herein.

Preferably, an antisense oligonucleotide of the invention is said to be active when,
it is able to silence, inactivate, knock down, reduce, break down, correct or decrease (the quantity or levels of) said mutated allele or transcript, and
the level of the corresponding wild-type allele or transcript is not less than 50%, 60%, 70%, 80%, 90%, 95% or 100% of the level of the same allele or transcript at the onset of the treatment.

The allele or transcript is preferably from the PSEN1 or PSEN2 gene, more preferably from the PSEN1 gene. In principle, the invention is not limited to a specific mutation found in PSEN1 or PSEN2. There are already several hundred mutations known for PSEN1 that are linked with FAD. Examples of mutations in PSEN1 that cause FAD and could be potentially targeted by the oligonucleotide of the invention: R35Q; E69D; A79V; V82L; 183T; M84V; L85P; P88L; V89L; V89L; C92S; V94M; V96F; V97L; T99A; F105C; F1051; F105L; F105V; R108Q; L113P; L113Q; Y115C; Y115D; Y115H; T1161; T116N; T116R; P117A; P117L; P117R; P117S; E120D; E120D; E120G; E120K; E123K; H131R; S132A; L134R; N135D; N135S; N135Y; A136G; M139I; M139I; M139K; M139L; M139T; M139V; V142F; 1143F; 1143M; 1143N; 1143T; 1143V; M146I; M146I; M146I; M146L; M146L; M146V; T1471; T147P; L150P; LI53V; Y154C; Y154N; Y156F; R157insIY; R157S; H163P; H163R; H163Y; A164V; W165C; W165C; W165G; L166H; L166P; L166R; L166V; S169L; S169P; S170F; S170P; L171P; L173F; L173F; L173W; L174del; L174M; L174R; F175S; F176L; F177L; F177S; S178P; G183V; E184D; E184G; V191A; 1202F; G206A; G206D; G206S; G206V; G209A; G209E; G209R; G209V; S212Y; 1213F; 1213L; 1213T; H214D; H214N; H214Y; G217D; G217R; L219F; L219P; L219R; R220P; Q222H; Q222P; Q222R; Q223R; L226F; L226R; I229F; S2301; S230N; S230R; A231P; A231T; A231V; L232P; M2331; M2331; M233L; M233L; M233T; M233V; L235P; L235R; L235V; F2371; F237L; 1238M; K239N; T245P; A246E; A246P; L248P; L248R; L250F; L250S; L250V; Y256S; D257A, A260V; V261F; V261L; L262F; L262V; C263F; C263R; P264L; G266S; P267A; P267L; P267S; R269G; R269H; L271V; V272A; E273A; E273G; T274R; A275V; R2781; R278K; R278S; R278T; E280A; E280G; E280K; L282F; L282R; L282V; F283L; P284L; P284S; A285V; L286P; L286V; T291A; T291P; K311R; E318G; D333G; R352C; R352_S353insR; T3541; R358Q; S365A; S365Y; R377M; R377W; G378E; G378V; G378fs; L381F; L381V; G384A; F386I; F386S; F388L; S3901; S390N; V391F; V391G; L392P; L392V; G394V; A396T; N405S; 1408T; A409T; C410Y; V4121; 1416T; G417S; L418F; L420R; L424F; L424H; L424R; L424V; A426P; A431E; A431V; A434C; A434T; L435F; P436Q; P436S; 1437V; 1439S; 1439V; or M457V.

Examples of mutations in PSEN2 that cause FAD and that could be targeted by an oligonucleotide of the invention: T18M; R29H; G34S; R62C; R62H; P69A; R71W; K82R; A85V; V101M; K115Efs; T122P; T122R; P123L; E126fs; E126K; S130L; V139M; N1411; N141Y; L143H; V1481; K161R; R163H; H169N; M174V; S 175C; G212V; V214L; Q228L; Y231C; 1235F; A237V; L238F; L238P; M2391; M239V; A252T; A258T; T301M; K306fs; P334A; P334R; P348L; A377V; V393M; T430M; or D439A

In an embodiment, the PSEN1 mutation is selected from the group consisting of the most frequent PSEN1 mutations: P117L, M139T, M139V, M146I, H163R, G206A, P264L, E280A, L392V and A431E, more preferably E280A and A431E. Most preferred mutation in PSEN1 is A431E. In an embodiment, the PSEN1 mutation is selected from the group consisting of the most frequent PSEN1 mutations: P117L, M139T, M139V, M146I, H163R, G206A, P264L, E280A, L392V, A431E, and S212Y, more preferably E280A and A431E. Most preferred mutation in PSEN1 is A431E

In an embodiment, the PSEN1 mutation is selected from the group consisting of the mutations caused by a change of a guanine, thymine or cytosine to an adenosine in the coding sequence resulting in one of the following mutations in the PSEN1 protein: R35Q, V94M, F1051, R108Q, L113Q, T116N, P117T, P117Q, E120K, E123K, M139K, M139I, V142I, M146I, V151M, Y154N, L166H, L174M, 1180N, G206S, G206D, G209R, G209E, S212Y, H214N, G217D, S230N, A231T, M2331, F2371, A246E, Y256N, V261I, G266S, R269H, V272D, T274K, R278K, E280K, R358Q, A360T, S365Y, G378E, F386I, F386L, S390N, A396T, A409T, C410Y, G417S, L424H, A431E, A434T, P436Q. Most preferred mutation in PSEN1 is A431E. For all these mutations, the oligonucleotide may be designed as follows: when the base of the nucleotide present in the oligonucleotide and targeting the mutation is a thymine, said thymine is replaced by an uracil. Examples are provided of oligonucleotides targeting the A431E mutated allele of PSEN1. Such examples are disclosed in SEQ ID NO: 26, 28, 30, 33, 35, 37, 39. Examples are also provided of oligonucleotides targeting the S212Y mutated allele of PSEN1. Such examples are disclosed in SEQ ID NO: 188, 190, 192, 194, 198, 200.

Preferred oligonucleotides targeting this mutation via a thymine have this thymine be replaced by a uracil and this uracil is at the first, second or third position of their central part. Said preferred oligonucleotides targeting this mutation via a thymine have this thymine replaced by a uracil at the sixth, seventh, or eighth nucleotide in the oligonucleotide sequence starting from 5′ to 3′. Other preferred oligonucleotides targeting this mutation via a thymine have this thymine replaced by a uracil and this uracil is at the first, second, third, or seventh position of the central part of the oligonucleotide. Said preferred oligonucleotides targeting this mutation via a thymine have this thymine replaced by a uracil at the sixth, seventh, eighth, or twelfth nucleotide in the oligonucleotide sequence starting from 5′ to 3′. This replacement increases mutant allele selectivity (See examples 3-4), possibly due to a slightly decreased melting temperature (Tm). Indeed, examples 3 and 4 indicate that antisense oligonucleotides with a thymine-uracil replacement at the above-mentioned preferred nucleotide positions exhibit an increased mutant allele selectivity of PSEN1. Examples are provided of oligonucleotides targeting the A431E or S212Y mutated allele of PSEN1. Preferred examples are found in SEQ ID NO: 28, 30, 33, 37, 39, 47, 97, 99, 115, 192, 194, 198.

As used herein, the term “central part” or “central region” of an antisense oligonucleotide refers to a specific section or segment of the oligonucleotide sequence. The precise definition and boundaries of the central part can vary depending on the context of the invention. In context of the first aspect of the invention, the central part may be determined based on the nucleotide position relative to the ends of the oligonucleotide. In context of the second aspect of the invention, the central part refers to the distinct region of the gapmer present between two flanking regions/wings. With respect to both aspects, the skilled person understands that the central part is designed to specifically target a mutated allele of a protein of the γ-secretase complex.

As known to the skilled person an oligonucleotide is a polymer of nucleotides and/or a polymer of nucleotides analogues. The expression “nucleotide analogue” may be replaced by “derived from a nucleotide”. An oligonucleotide comprises or consists of repeating monomers. Within the context of the invention, an oligonucleotide comprises up to 50 nucleotides and/or nucleotide analogues. Said oligonucleotide may have 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides. Such oligonucleotide may also be identified as an oligonucleotide having from 10 to 50 nucleotides or from 12 to 50 nucleotides. Attractive results were obtained with oligonucleotides having a length ranged from 15 to 30 nucleotides, 15, 16, 17, 18, 19, 20, 21, 22, 23, 24, 25, 26, 27, 28, 29 or 30 nucleotides.

In an embodiment, the antisense oligonucleotide of the invention comprises nucleotides and/or nucleotides analogues. In another embodiment, an antisense oligonucleotide of the invention comprises 15 to 30 nucleotides including a central region of DNA nucleotides (and/or DNA nucleotide analogues) flanked at each end by wing regions comprising RNA nucleotides analogues. In a preferred embodiment, the antisense oligonucleotide comprising 15 to 30 nucleotides including a central region of DNA nucleotides flanked at each end by wing regions comprising RNA nucleotides analogues relates to the antisense oligonucleotide of the second aspect of the invention. A DNA nucleotide analogue may be present in the central region of DNA nucleotides as long as the natural recruitment of RNaseH to the AON-target RNA duplex is not significantly reduced or hindered or completely abolished and/or its on target activity is not significantly reduced. In this context, “its activity is not significantly reduced” means that the activity of an oligonucleotide with one or more DNA analogues nucleotides in the central part is better or similar or not less than 50%, 60%, 70%, 80%, or 90% compared to oligonucleotide with only DNA nucleotides in the central part. Accordingly, an antisense oligonucleotide as defined herein is able to recruit RNaseH to silence, inactivate, knock down, break down, decrease or reduce (the levels or expression of) the targeted mutant allele.

In another embodiment, an antisense oligonucleotide of the invention comprises 15 to 30 nucleotides including a central region of consecutive DNA nucleotides (and/or DNA nucleotide analogues) flanked at each end by wing regions comprising RNA nucleotides analogues. In another embodiment, an antisense oligonucleotide of the invention comprises 15 to 30 nucleotides including a central region of consecutive DNA nucleotides (and/or DNA nucleotide analogues) flanked at each end by wing regions comprising consecutive RNA nucleotides analogues. In a preferred embodiment, an antisense oligonucleotide of the invention comprises 15 to 30 nucleotides including a central region of 5 to 15 consecutive DNA nucleotides (and/or DNA nucleotide analogues) flanked at each end by wing regions comprising 1 to 5 RNA nucleotide analogues. In a preferred embodiment, an antisense oligonucleotide of the invention comprises 15 to 30 nucleotides including a central region of 5 to 15 consecutive DNA nucleotide analogues flanked in each end by wing regions comprising 1 to 5 RNA nucleotide analogues. In a preferred embodiment, the antisense oligonucleotide comprising 15 to 30 nucleotides including a central region of (consecutive) DNA nucleotides (and/or DNA nucleotide analogues) flanked at each end by wing regions comprising (consecutive) RNA nucleotides analogues relates to the antisense oligonucleotide of the second aspect of the invention. In another preferred embodiment, the antisense oligonucleotide comprising 15 to 30 nucleotides including a central region of 5 to 15 (consecutive) DNA nucleotides (and/or DNA nucleotide analogues) flanked at each end by wing regions comprising 1 to 5 RNA nucleotides analogues relates to the antisense oligonucleotide of the second aspect of the invention.

In an embodiment, the central region comprises 5, 6, 7, 8, 9, 10, 11, 12, 13, 14 or 15 DNA nucleotides and/or DNA nucleotide analogues. Preferably, the length of the central region is 9, 10 or 11 DNA nucleotides and/or DNA nucleotide analogues. In another embodiment, one wing region comprises 1, 2, 3, 4 or 5 RNA nucleotide analogues. Preferably, the length of one wing region is 2 or 4 RNA nucleotide analogues. In another embodiment, each wing region comprises 1, 2, 3, 4 or 5 RNA nucleotide analogues. Preferably, the length of each wing region is 2 or 5 RNA nucleotide analogues. It is also encompassed by the invention that each wing region is not identical in length and/or in the type of chemistry used in the RNA nucleotide analogues.

In an embodiment, the central region comprises 5, 6, 7, 8, 9, 10, 11, 12, 13, 14 or 15 consecutive DNA nucleotides and/or DNA nucleotide analogues. Preferably, the length of the central region is 9, 10 or 11 consecutive DNA nucleotides and/or DNA nucleotide analogues. In another embodiment, one wing region comprises 1, 2, 3, 4 or 5 consecutive RNA nucleotide analogues. Preferably, the length of one wing region is 2 or 5 consecutive RNA nucleotide analogues. In an embodiment, each wing region comprises 1, 2, 3, 4 or 5 consecutive RNA nucleotide analogues. Preferably, the length of each wing region is 2 or 5 consecutive RNA nucleotide analogues. It is also encompassed by the invention that each wing region is not identical in length and/or in the type chemistry used in the RNA nucleotide analogues.

In an embodiment, the central region is 10 consecutive DNA nucleotides and/or DNA nucleotide analogues, and each wing region is 5 consecutive RNA nucleotide analogues. In another embodiment, the central region is 10 consecutive DNA nucleotides and/or DNA nucleotide analogues, and each wing region is 4 consecutive RNA nucleotide analogues. In another embodiment, the central region is 10 consecutive DNA nucleotides and/or DNA nucleotide analogues, and each wing region is 3 consecutive RNA nucleotide analogues. In another embodiment, the central region is 10 consecutive DNA nucleotides and/or DNA nucleotide analogues, and each wing region is 2 consecutive RNA nucleotide analogues. In another embodiment, the central region is 10 consecutive DNA nucleotides and/or DNA nucleotide analogues, and each wing region is 1 consecutive RNA nucleotide analogue.

In an embodiment, the central region is 11 consecutive DNA nucleotides and/or DNA nucleotide analogues, and each wing region is 5 consecutive RNA nucleotide analogues. In another embodiment, the central region is 11 consecutive DNA nucleotides and/or DNA nucleotide analogues, and each wing region is 4 consecutive RNA nucleotide analogues. In another embodiment, the central region is 11 consecutive DNA nucleotides and/or DNA nucleotide analogues, and each wing region is 3 consecutive RNA nucleotide analogues. In another embodiment, the central region is 11 consecutive DNA nucleotides and/or DNA nucleotide analogues, and each wing region is 2 consecutive RNA nucleotide analogues. In another embodiment, the central region is 11 consecutive DNA nucleotides and/or DNA nucleotide analogues, and each wing region is 1 consecutive RNA nucleotide analogue.

In an embodiment, the central region is 9 consecutive DNA nucleotides and/or DNA nucleotide analogues, and each wing region is 5 consecutive RNA nucleotide analogues. In another embodiment, the central region is 9 consecutive DNA nucleotides and/or DNA nucleotide analogues, and each wing region is 4 consecutive RNA nucleotide analogues. In another embodiment, the central region is 9 consecutive DNA nucleotides and/or DNA nucleotide analogues, and each wing region is 3 consecutive RNA nucleotide analogues. In another embodiment, the central region is 9 consecutive DNA nucleotides and/or DNA nucleotide analogues, and each wing region is 2 consecutive RNA nucleotide analogues. In another embodiment, the central region is 9 consecutive DNA nucleotides and/or DNA nucleotide analogues, and each wing region is 1 consecutive RNA nucleotide analogue.

In an embodiment, the central region is 12 consecutive DNA nucleotides and/or DNA nucleotide analogues, and each wing region is 5 consecutive RNA nucleotide analogues. In another embodiment, the central region is 12 consecutive DNA nucleotides and/or DNA nucleotide analogues, and each wing region is 4 consecutive RNA nucleotide analogues. In another embodiment, the central region is 12 consecutive DNA nucleotides and/or DNA nucleotide analogues, and each wing region is 3 consecutive RNA nucleotide analogues. In another embodiment, the central region is 12 consecutive DNA nucleotides and/or DNA nucleotide analogues, and each wing region is 2 consecutive RNA nucleotide analogues. In another embodiment, the central region is 12 consecutive DNA nucleotides and/or DNA nucleotide analogues, and each wing region is 1 consecutive RNA nucleotide analogue.

In a preferred embodiment, the mutation in the allele or transcript of PSEN1 or PSEN2 is targeted by a nucleotide of the oligonucleotide being present in the central part of the oligonucleotide; the central part being 5 to 15 nucleotides and each wing being 1 to 5 nucleotides. In a preferred embodiment, the antisense oligonucleotide comprising a nucleotide, present in the central part of the oligonucleotide, which targets the mutation in the allele or transcript of PSEN1 or PSEN2 relates to the antisense oligonucleotide of the second aspect of the invention. It was surprisingly found that the efficacy and selectivity of the oligonucleotide for the mutated PSEN1 or PSEN2 allele or transcript is optimal when the nucleotide of the oligonucleotide targeting the mutated nucleotide of PSEN1 or PSEN2 is located in the central part of the oligonucleotide. In particular, as shown in Examples 3 and 4, antisense oligonucleotides have an increased selectivity towards the mutant PSEN1 allele when the nucleotide targeting the mutated nucleotide of PSEN1 is located within the first five nucleotides of the central part of the oligonucleotide starting from 5′ to 3′ (i.e, nucleotides located at position 6, 7, 8, 9, or 10 in the oligonucleotide sequence starting from 5′ to 3′).

In an embodiment, said nucleotide is the first, second, third, fourth, fifth, sixth, seventh, eighth, nineth, tenth, eleventh or twelfth nucleotide in the central part of the oligonucleotide starting from 5′ to 3′. In said embodiment, said nucleotide is the sixth, seventh, eighth, nineth, tenth, eleventh, twelfth, thirteenth, fourteenth, fifteenth, sixteenth, seventeenth, or eighteenth nucleotide in the oligonucleotide sequence starting from 5′ to 3′. In a preferred embodiment, said nucleotide is the first, second, third, fourth, or fifth nucleotide in the central part of the oligonucleotide starting from 5′ to 3′. Accordingly, in said preferred embodiment, said nucleotide is the sixth, seventh, eighth, nineth, or tenth nucleotide in the oligonucleotide sequence starting from 5′ to 3′. In an embodiment, said nucleotide is the first, second, third, or fifth nucleotide in the central part of the oligonucleotide starting from 5′ to 3′. In said preferred embodiment, said nucleotide is the sixth, seventh, eight, or tenth nucleotide in the oligonucleotide sequence part starting from 5′ to 3′. In an embodiment, when the base of said nucleotide present in the oligonucleotide and targeting the mutation is a thymine, said thymine is replaced by an uracil. Examples are provided of oligonucleotides targeting the A431E mutated allele of PSEN1. Such examples are disclosed in SEQ ID NO: 26, 28, 30, 33, 35, 37, 39.

Examples are also provided of oligonucleotides targeting the S212Y mutated allele of PSEN1. Such examples are disclosed in SEQ ID NO: 188, 190, 192, 194, 198, 200.

Preferred oligonucleotides targeting this mutation via a thymine have this thymine be replaced by an uracil and this uracil is at the first, second or third position of their central part. Accordingly, preferred oligonucleotides targeting this mutation have the thymine replaced by an uracil at the sixth, seventh, or eight position of the oligonucleotide sequence starting from 5′ to 3′. Other preferred oligonucleotides targeting this mutation via a thymine have this thymine be replaced by an uracil and this uracil is at the first, second, third, or seventh position of the central part of the oligonucleotide. Said preferred oligonucleotides have the thymine replaced by an uracil at the sixth, seventh, eighth, or twelfth position of the oligonucleotide sequence starting from 5′ to 3′. This replacement increases mutant allele selectivity, possibly due to a slight decreased Tm. Preferred examples are found in SEQ ID NO: 28, 30, 33, 37, 39, 47, 97, 99, 115, 192, 194, 198.

Therefore, in a preferred embodiment, the oligonucleotide is as follows:
it comprises a central part of 5 to 15 nucleotides and two wings, each of 1 to 5 nucleotides and
the mutation present in the mutated allele or transcript of PSEN1 or PSEN2 is targeted by the first, second, third, fourth, fifth, sixth, seventh, eighth, nineth, tenth, eleventh or twelfth nucleotide of the central part of the oligonucleotide.

In a preferred embodiment, the oligonucleotide is as follows:
it comprises a central part of 5 to 15 nucleotides and two wings, each of 1 to 5 nucleotides and
the mutation present in the mutated allele or transcript of PSEN1 or PSEN2 is targeted by the sixth, seventh, eighth, nineth, tenth, eleventh, twelfth, thirteenth, fourteenth, fifteenth, sixteenth, seventeenth, or eighteenth nucleotide in the oligonucleotide sequence starting from 5′ to 3′.

Accordingly, in a preferred embodiment, the antisense oligonucleotide relates to the antisense oligonucleotide of the second aspect of the invention.

In another preferred embodiment, the base of the nucleotide of the oligonucleotide targeting the mutation present in the PSEN1 or PSEN2 allele or transcript is a RNA base. In a more preferred embodiment, said RNA base is a uracil. More preferably, the target mutation is a C to A mutations, but also the target mutation might be a G or T to A mutations.

In an embodiment, the antisense oligonucleotide of the invention comprises nucleotides and nucleotide analogues. Nucleotides may be RNA or DNA nucleotides. The most common naturally occurring nucleotides in RNA are adenosine monophosphate, cytidine monophosphate, guanosine monophosphate, thymidine monophosphate, and uridine monophosphate. These consist of a pentose sugar ribose, a 5′-linked phosphate group which is linked via a phosphate ester, and a 1′-linked base. The same holds for DNA wherein deoxyribose is present instead of ribose. The sugar connects the base and the phosphate linkage, and is therefore often referred to as the scaffold of the nucleotide. A modification in the pentose sugar is therefore often referred to as a scaffold modification. A sugar modification may therefore be called a scaffold modification. For several modifications, the original pentose sugar may be replaced in its entirety by another moiety that similarly connects the base and the phosphate. It is therefore understood that while a pentose sugar is often a scaffold, a scaffold is not necessarily a pentose sugar.

A base, sometimes called a nucleobase, is generally adenine, cytosine, guanine, thymine, or uracil, or a derivative thereof. Cytosine, thymine, and uracil are pyrimidine bases, and are generally linked to the scaffold through their 1-nitrogen. Adenine and guanine are purine bases, and are generally linked to the scaffold through their 9-nitrogen. A base present in an oligonucleotide is usually complementary to the target region. If such a base is a modified base or if a base analogue is being used, said modified base or base analogue should keep the same base pair specificity as the base it replaces. “Base pairing” refers to the binding of two bases (or nucleobases) to each other by hydrogen bonds. Specifically, a nucleobase analogue replacing cytosine is capable of base pairing with guanine, a nucleobase analogue replacing guanine is capable of base pairing with cytosine, a nucleobase analogue replacing adenine is capable of base pairing with uracil and a nucleobase replacing uracil is capable of base pairing with adenine.

A nucleotide is generally connected to neighboring nucleotides through condensation of its 5′-phosphate moiety to the 3′-hydroxyl moiety of the neighboring nucleotide monomer. Similarly, its 3′-hydroxyl moiety is generally connected to the 5′-phosphate of a neighboring nucleotide monomer. This forms phosphodiester bonds. The phosphodiesters and the scaffold form an alternating copolymer. The bases are grafted to this copolymer, namely to the scaffold moieties. Because of this characteristic, the alternating copolymer formed by linked monomers of an oligonucleotide is often called the backbone of the oligonucleotide. Because the phosphodiester bonds connect neighboring monomers together, they are often referred to as backbone linkages. It is understood that when a phosphate group is modified so that it is instead an analogous moiety such as a phosphorothioate (PS), such a moiety is still referred to as the backbone linkage of the monomer. This is referred to as a backbone linkage modification. In general terms, the backbone of an oligonucleotide is thus comprised of alternating scaffolds and backbone linkages. In an embodiment, the backbone of the oligonucleotide (central part and the wings) is fully modified, preferably being a PS backbone.

In an embodiment the antisense oligonucleotide comprises a central part comprising DNA nucleotides (or DNA nucleotides analogues) and modified RNA parts in each wing. In a preferred embodiment, the antisense oligonucleotide comprising a central part comprising DNA nucleotides (or DNA nucleotides analogues) and modified RNA parts in each wing relates to the antisense oligonucleotide of the second aspect of the invention. A RNA or DNA nucleotide may be modified and may therefore be considered as an RNA or DNA nucleotide analogue when it comprises a modified base and/or a modified sugar. In addition, the internucleoside linkage linking two adjacent nucleosides may be modified compared to the phosphodiester bond linking two nucleosides. A “modified internucleoside linkage” may be replaced by the wording “backbone linkage modification” as explained earlier herein.

In an embodiment, the antisense oligonucleotide of the invention comprises a DNA central part (or region) flanked at each end (wing) by modified RNA parts (regions). In another embodiment, the antisense oligonucleotide of the invention comprises a modified DNA central part (or region) flanked at each end (wing) by modified RNA parts (regions). In a preferred embodiment, the antisense oligonucleotide of the invention comprising a modified DNA central part flanked at each end by modified RNA parts relates to the antisense oligonucleotide of the second aspect of the invention.

In an embodiment, the oligonucleotide of the invention, consisting of a 5′ wing, a 3′ wing, and a central part (gap) is single-stranded. Such structure (a 5′ wing, a 3′ wing, and a central part (gap)) may also be called gapmer. In a preferred embodiment, the single-stranded antisense oligonucleotide consisting of a 5′ wing, a 3′ wing, and a central part relates to the antisense oligonucleotide of the second aspect of the invention. Said structure is attractive as it allows the oligonucleotide to bind towards target mRNA and to recruit RNaseH. The recruited RNaseH has at least one of the following effects on the targeted mutant allele: to silence, inactivate, knock down, break down, decrease or reduce its levels. In contrast, double-stranded siRNAs are not able to recruit RNaseH but rather depend on Argonaute proteins for silencing target mRNA. Double-stranded siRNAs are loaded into the RNA-induced silencing complex (RISC). Upon RISC loading, the less thermodynamically stable 5′-end of the siRNA is incorporated and guides the RISC to the complementary target mRNA. The mRNA target dissociates from the intact siRNA after Argonaute-dependent cleavage, freeing RISC to regenerate and cleave additional mRNA targets.

The presence of the wings provides the antisense oligonucleotide stability and resistance properties to degradation by exonucleases. Implementation of chemical modifications in the gap and/or wings, comprising base, scaffold, and/or linkage modifications, may improve safety, bio-distribution, stability, cellular uptake, intracellular trafficking, target binding affinity, duplex stability and the efficiency of the oligonucleotide of the invention compared to an oligonucleotide consisting of non-modified DNA and/or non-modified RNA analogues. This effect may be at least due to the presence of the modified RNA wings and/or the modified DNA central part. Finetuning of DNA and/or RNA base, scaffold, and/or linkage modifications at specific positions in the oligonucleotide (precision chemistry) may result in oligonucleotides with most favorable characteristics for clinical application.

In an embodiment, the central part/region (gap) of the antisense oligonucleotide comprises DNA nucleotides or DNA nucleotide analogues. In a preferred embodiment, the base and sugar of this part of the antisense oligonucleotide are not modified. However, in a more preferred embodiment, an internucleoside linkage (or the whole backbone) in this central part is modified. In an embodiment, the modified internucleoside linkage is a phosphorothioate or a phosphoramidate internucleoside linkage. A nucleotide of the central part/region of said antisense oligonucleotide may have at least one internucleoside linkage modification and/or at least one base modification compared to an antisense oligonucleotide with a full non-modified DNA central part.

In an embodiment, a wing of the antisense oligonucleotide comprises modified RNA nucleotides and/or modified internucleoside linkages. In an embodiment, both wings of the antisense oligonucleotide comprise modified RNA nucleotides and/or modified internucleoside linkages. A wing of said antisense oligonucleotide may have at least one internucleoside linkage modification and/or at least one sugar modification and/or at least one base modification compared to a non-modified RNA-based antisense oligonucleotide. In an embodiment, the modified internucleoside linkage is a phosphorothioate or a phosphoramidate internucleoside linkage.

A base modification in the central part and/or in the wing of the oligonucleotide includes a modified version of the natural purine and pyrimidine bases (e.g. adenine, uracil, guanine, cytosine, and thymine), such as hypoxanthine (e.g. inosine), orotic acid, agmatidine, lysidine, pseudouracil, 2-thiopyrimidine (e.g. 2-thiouracil, 2-thiothymine), G-clamp and its derivatives, 5-substituted pyrimidine (e.g. 5-halouracil, 5-propynyluracil, 5-propynylcytosine, 5-aminomethyluracil, 5-hydroxymethyluracil, 5-aminomethylcytosine, 5-hydroxymethylcytosine, Super T), 7-deazaguanine, 7-deazaadenine, 2,6-diaminopurine, 7-aza-2,6-diaminopurine, 8-aza-7-deazaguanine, 8-aza-7-deazaadenine, 8-aza-7-deaza-2,6-diaminopurine, Super G, Super A, and N4-ethylcytosine, or derivatives thereof; N2-cyclopentylguanine (cPent-G), N2-cyclopentyl-2-aminopurine (cPent-AP), and N2-propyl-2-aminopurine (Pr-AP), or derivatives thereof; and degenerate or universal bases, like 2,6-difluorotoluene or absent bases like abasic sites (e.g. 1-deoxyribose, 1,2-dideoxyribose, 1-deoxy-2-O-methylribose; or pyrrolidine derivatives in which the ring oxygen has been replaced with nitrogen (azaribose)). Examples of derivatives of Super A, Super G and Super T can be found in U.S. Pat. No. 6,683,173 (Epoch Biosciences), which is incorporated here entirely by reference. cPent-G, cPent-AP and Pr-AP were shown to reduce immunostimulatory effects when incorporated in siRNA (Peacock H. et al.).

A preferred modified base is 5-methylcytosine.

Depending on the length of each wing, an antisense oligonucleotide of the invention may comprise 1, 2, 3, 4, 5, 6, 7, 8, 9, 10 base modifications. It is also encompassed by the invention to introduce more than one distinct base modification in said wing of said antisense oligonucleotide.

Depending on the length of the central part, an antisense oligonucleotide of the invention may comprise 1, 2, 3, 4, 5, 6, 7, 8, 9, 10, 11, 12, 13, 14 or 15 base modifications. It is also encompassed by the invention to introduce more than one distinct base modification in the central part of said antisense oligonucleotide.

Depending on the length of the antisense oligonucleotide, an antisense oligonucleotide of the invention may comprise at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more base modifications. It is also encompassed by the invention to introduce more than one distinct base modification in said antisense oligonucleotide.

A modified sugar in a nucleotide of a wing and/or in the central part of the antisense oligonucleotide is synonymous of a scaffold modification of the oligonucleotide.

A scaffold modification can include a modified version of the ribosyl moiety, such as 2′-O-modified RNA such as 2′-O-alkyl or 2′-O-(substituted)alkyl e.g. 2′-O-methyl, 2′-O-(2-cyanoethyl), 2′-O-(2-methoxy)ethyl (2′-MOE), 2′-O-(2-thiomethyl)ethyl, 2′-O-butyryl, 2′-O-propargyl, 2′-O-acetalester (such as e.g. Biscans et al. Bioorg. Med. Chem. 2015, 23, 5360), 2′-O-allyl, 2′-O-(2S-methoxypropyl), 2′-O-(N-(aminoethyl)carbamoyl)methyl) (2′-AECM), 2′-O-(2-carboxyethyl) and carbamoyl derivatives (Yamada et al. Org. Biomol. Chem. 2014, 12, 6457), 2′-O-(2-amino) propyl, 2′-O-(2-(dimethylamino)propyl), 2′-O-(2-amino)ethyl, 2′-O-(2-(dimethylamino)ethyl); 2′-deoxy (DNA); 2′-O-(haloalkoxy)methyl (Arai K. et al. Bioorg. Med. Chem. 2011, 21, 6285) e.g. 2′-O-(2-chloroethoxy)methyl (MCEM), 2′-O-(2,2-dichloroethoxy)methyl (DCEM); 2′-O-alkoxycarbonyl e.g. 2′-O-[2-(methoxycarbonyl)ethyl] (MOCE), 2′-O-[2-(N-methylcarbamoyl)ethyl] (MCE), 2′-O-[2-(N,N-dimethylcarbamoyl)ethyl] (DCME), 2′-O-[2-(methylthio)ethyl] (2′-MTE), 2′-(ω-O-serinol); 2′-halo e.g. 2′-F, FANA (2′-F arabinosyl nucleic acid); 2′,4′-difluoro-2′-deoxy; carbasugar and azasugar modifications; 3′-O-substituted e.g. 3′-O-methyl, 3′-O-butyryl, 3′-O-propargyl; 4′-substituted e.g. 4′-aminomethyl-2′-O-methyl or 4′-aminomethyl-2′-fluoro; 5′-subtituted e.g. 5′-methyl or CNA (Østergaard et al. ACS Chem. Biol. 2014, 22, 6227); and their derivatives.

A scaffold modification can include a bicyclic nucleic acid monomer (BNA) which may be a bridged nucleic acid monomer. Each occurrence of said BNA may result in a monomer that is independently chosen from the group consisting of a conformationally restricted nucleotide (CRN) monomer, a locked nucleic acid (LNA) monomer, a xylo-LNA monomer, an α-LNA monomer, an α-L-LNA monomer, a β-D-LNA monomer, a 2′-amino-LNA monomer, a 2′-(alkylamino)-LNA monomer, a 2′-(acylamino)-LNA monomer, a 2′-N-substituted-2′-amino-LNA monomer, a 2′-thio-LNA monomer, a (2′-O,4′-C) constrained ethyl (cEt) BNA monomer, a (2′-O,4′-C) constrained methoxyethyl (cMOE) BNA monomer, a 2′,4′-BNA^{NC} (N—H) monomer, a 2′,4′-BNA^{NC} (N-Me) monomer, a 2′,4′-BNA^{NC} (N-Bn) monomer, an ethylene-bridged nucleic acid (ENA) monomer, a carba LNA (cLNA) monomer, a 3,4-dihydro-2H-pyran nucleic acid (DpNA) monomer, a 2′-C-bridged bicyclic nucleotide (CBBN) monomer, a heterocyclic-bridged BNA monomer (such as triazolyl or tetrazolyl-linked), an amido-bridged BNA monomer, an urea-bridged BNA monomer, a sulfonamide-bridged BNA monomer, a bicyclic carbocyclic nucleotide monomer, a TriNA monomer, an α-L-TriNA monomer, a bicyclo DNA (bcDNA) monomer, an abcDNA monomer, an F-bcDNA monomer, a tricyclo DNA (tcDNA) monomer, an F-tcDNA monomer, an oxetane nucleotide monomer, a locked PMO monomer derived from 2′-amino-LNA, a guanidine-bridged nucleic acid (GuNA) monomer, a spirocyclopropylene-bridged nucleic acid (scpBNA) monomer, and derivatives thereof.

A preferred sugar modification is selected from:
2′-O-modified RNA, more preferably 2′-O-alkyl or 2′-O-(substituted)alkyl, even more preferably 2′-O-methyl (2′-OMe) or 2′-O-(2-methoxy)ethyl (2′-MOE)
(BNA), more preferably a (CRN) monomer or a locked nucleic acid (LNA) monomer.

More preferred sugar modifications are 2′-OMe, 2′-MOE, and a locked nucleic acid (LNA).

Depending on the length of each wing, an antisense oligonucleotide of the invention may comprise 1, 2, 3, 4, 5, 6, 7, 8, 9, 10 sugar modifications. It is also encompassed by the invention to introduce more than one distinct sugar modification in said wing of said antisense oligonucleotide.

Depending on the length of the central part, an antisense oligonucleotide of the invention may comprise 1, 2, 3, 4, 5, 6, 7, 8, 9, 10, 11, 12, 13, 14, 15 sugar modifications. It is also encompassed by the invention to introduce more than one distinct sugar modification in said wing of said antisense oligonucleotide.

Depending on the length of the antisense oligonucleotide, an antisense oligonucleotide of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 or more sugar modifications. It is also encompassed by the invention to introduce more than one distinct sugar modification in said antisense oligonucleotide.

Antisense oligonucleotides according to the invention can comprise backbone linkage modifications in their wing and/or in their central part. A backbone linkage modification can be, but is not limited to, a modified version of the phosphodiester present in RNA, such as phosphorothioate (PS), chirally pure phosphorothioate, (R)-phosphorothioate, (S)-phopshorothioate, phosphorodithioate (PS2), phosphonoacetate (PACE), phosphonoacetamide (PACA), thiophosphonoacetate (thioPACE), thiophosphonoacetamide, phosphorothioate prodrug, H-phosphonate, methyl phosphonate, methyl phosphonothioate, methyl phosphate, methyl phosphorothioate, ethyl phosphate, ethyl phosphorothioate, boranophosphate, boranophosphorothioate, methyl boranophosphate, methyl boranophosphorothioate, methyl boranophosphonate, methyl boranophosphonothioate, phosphate, phosphotriester, aminoalkylphosphotriester, and their derivatives. Another modification includes phosphoryl guanidines, acylphosphoramidates, sulfonylphosphoramidates, phosphoramidite, phosphoramidate, N3′→P5′ phosphoramidate, phosphordiamidate, phosphorothiodiamidate, sulfamate, dimethylenesulfoxide, amide, sulfonate, siloxane, sulfide, sulfone, formacetyl, thioformacetyl, methylene formacetyl, alkenyl, methylenehydrazino, sulfonamide, amide, triazole, oxalyl, carbamate, methyleneimino (MMI), and thioacetamido nucleic acid (TANA); and their derivatives. Examples of chirally pure phosphorothioate linkages are described in e.g. WO2014/010250 or WO2017/062862 (WaVe Life Sciences). Examples of phosphoryl guanidine linkages are described in WO2016/028187 (Noogen). Various salts, mixed salts and free acid forms are also included, as well as 3′→3′ and 2′→5′ linkages.

Preferred backbone linkage modifications are phosphorothioate and phosphoramidate.

More preferred backbone linkage modifications are phosphorothioate and phosphoramidate (preferably a mesyl-phosphoramidate).

Depending on its length, an antisense oligonucleotide of the invention may comprise 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, or more backbone linkage modifications. It is also encompassed by the invention to introduce more than one distinct backbone modification in said antisense oligonucleotide.

In an embodiment, the antisense oligonucleotide is such that a DNA nucleotide in its central part is not modified.

In another embodiment, the antisense oligonucleotide is such that a DNA nucleotide in its central part is modified and that at least part of the backbone (i.e. internucleoside linkage) in its central part comprises phosphorothioate and/or phosphoramidate (preferably a mesyl-phosphoramidate).

In an embodiment, the antisense oligonucleotide is such that the RNA nucleotide analogues in each of its wings have been modified comprising a modified internucleoside linkage (preferably a phosphorothioate and/or phosphoramidite (preferably a mesyl-phosphoramidate)) and/or a modified sugar (preferably a 2′-MOE, 2′-OMe and/or a locked nucleic acid (LNA) monomer) and/or a modified base (preferably a 5-methylcytosine).

In an embodiment, the antisense oligonucleotide is such that:
the DNA nucleotides in its central part have not been modified and
the RNA nucleotide analogues in each of its wings have been modified comprising a modified internucleoside linkage (preferably a phosphorothioate or phosphoramidate (more preferably a mesyl-phosphoramidate) and/or a modified sugar (preferably a 2′-MOE, 2′-OMe and/or a locked nucleic acid (LNA) monomer) and/or a modified base (preferably a 5-methylcytosine).

In an embodiment, the antisense oligonucleotide is such that:
a DNA nucleotide in its central part has been modified and that the backbone (i.e. at least one internucleoside linkage) in its central comprises phosphorothioate or phosphoramidate (more preferably a mesyl-phosphoramidate) and
the RNA nucleotide analogues in each of its wings have been modified comprising a modified internucleoside linkage (preferably a phosphorothioate or phosphoramidate (more preferably a mesyl-phosphoramidate) and/or a modified sugar (preferably a 2′-MOE, 2′-OMe and/or a locked nucleic acid (LNA) monomer) and/or a modified base (preferably a 5-methylcytosine).

In an embodiment, the antisense oligonucleotide comprises 15 to 30 or 20 to 30 nucleotides including a central region of 5 to 15 consecutive DNA nucleotides flanked at each end by wing regions comprising 1 to 5 RNA nucleotides analogues. Preferably, the backbone of the oligonucleotide is fully modified (central region and each wing). More preferably the backbone is phosphorothioate and/or phosphoramidite (more preferably mesyl-phosphoramidate).

In an embodiment, the antisense oligonucleotide is as follows:
1. its DNA nucleotides in its central part have not been modified and the RNA nucleotide analogues in each of its wings have been modified comprising a modified internucleoside linkage (preferably a phosphorothioate or phosphoramidite (more preferably mesyl-phosphoramidate)) and/or a modified sugar (preferably a 2′-MOE, 2′-OMe and/or a locked nucleic acid (LNA) monomer) and/or a modified base (preferably a 5-methylcytosine),
or
1. a DNA nucleotide in its central part has been modified and that the backbone (i.e. at least one internucleoside linkage) in its central part comprises phosphorothioate and/or phosphoramidate (more preferably mesyl-phosphoramidate) and
   the RNA nucleotide analogues in each of its wings have been modified comprising a modified internucleoside linkage (preferably a phosphorothioate or phosphoramidate (more preferably mesyl-phosphoramidate) and/or a modified sugar (preferably a 2′-MOE, 2′-OMe and/or a locked nucleic acid (LNA) monomer) and/or a modified base (preferably a 5-methylcytosine).

In an embodiment, the antisense oligonucleotide is as follows:
1. its DNA nucleotides in its central part have not been modified and the RNA nucleotide analogues in each of its wings have been modified comprising a modified internucleoside linkage (preferably a phosphorothioate and/or phosphoramidite (more preferably mesyl-phosphoramidate)) and/or a modified sugar (preferably a 2′-MOE and/or a modified base (preferably a 5-methylcytosine),
or
2. a DNA nucleotide in its central part has been modified and that the backbone (i.e. at least one internucleoside linkage) in its central part has been modified comprises a phosphorothioate and/or phosphoramidate (more preferably mesyl-phosphoramidate) and
   the RNA nucleotide analogues in each of its wings have been modified comprising a modified internucleoside linkage (preferably a phosphorothioate and/or phosphoramidite (more preferably mesyl-phosphoramidate)) and/or a modified sugar (preferably a 2′-MOE and/or a modified base (preferably a 5-methylcytosine).

In a preferred embodiment, the antisense oligonucleotide is as follows:
its DNA nucleotides in its central part have been modified and its backbone (i.e. at least one internucleoside linkage) in its central part comprises phosphorothioate linkages, and the RNA nucleotide analogues in each of its wings have been modified comprising a modified backbone which is phosphorothioate linkages, and preferably at least one modified sugar (preferably a 2′-MOE) and/or at least one modified base (preferably a 5-methylcytosine).

In an embodiment, the invention provides human antisense oligonucleotides, which preferentially target a mutated allele of a human protein of the γ-secretase pathway, preferably the human protein of the γ-secretase pathway is PSEN1. In this embodiment, the antisense oligonucleotide targets, hybridizes, binds and/or is reverse complementary to a mutated allele of a human protein of the γ-secretase pathway, preferably PSEN1.

In an embodiment, the base sequence of the oligonucleotide comprises any of SEQ ID NO:1, 3, 5, 7, 9, 11, 13, 15, 17, 19, 21, 23, 25, 27, 29, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 174, 176, 178, 180, 182, 184, 186, 188, 190, 192, 194, 196, 198, 200, 202, 204, 219, 221, 223, 225, 227, 229, 231, 233, 235, 237, 239, 241, 243, 245, 247, 249, 251, 253, 255, 257, 259, 261, 263, 265, 267, 269, 271, 273, 275, 277. More preferred oligonucleotides comprising one of these base sequences are disclosed below.

In an embodiment the antisense oligonucleotide comprises the following base sequence:

(SEQ ID NO: 1)
TCTTTCTTGAAAATGGCAAG(AON1)

In this embodiment, the antisense oligonucleotide has a length of 20 to 50 nucleotides and comprises the base sequence SEQ ID NO: 1. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 1 are 5-methylcytosines.

In an embodiment, the oligonucleotide comprising SEQ ID NO:1 has at least one of its internucleoside linkages which is modified, preferably as a phosphorothioate or phosphoramidate (preferably mesyl-phosphoramidate). More preferably, the oligonucleotide comprising SEQ ID NO: 1 is fully modified with a PS backbone.

In an embodiment one wing or preferably each of the wings of the oligonucleotide comprising SEQ ID NO:1 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 1 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:1 is represented by SEQ ID NO:2:

(SEQ ID NO: 2)
eTₛeC*ₛeTₛeTₛeTₛdCₛdTₛdTₛdGₛdAₛdAₛdAₛdAₛdTₛdGₛeGₛe
C*ₛeAₛeAₛeG

wherein A means adenosine, G means guanine, T means thymine, C means cytosine, C* means 5-methylcytosine, U means Uracil, e means 2′-MOE, d means DNA, subscript s means a PS internucleoside linkage, subscript o means PO linkage, subscript PNms means PNms linkage, and subscript PNdmi means PNdmi linkage.

This nomenclature is used for all oligonucleotides of the present application, unless otherwise indicated.

In an embodiment the antisense oligonucleotide comprises the following base sequence:

(SEQ ID NO: 3)
ATTCTTTCTTGAAAATGGCA(AON2)

In this embodiment, the antisense oligonucleotide has a length of 20 to 50 nucleotides and comprises the base sequence SEQ ID NO:3. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 3 are 5-methylcytosines.

In an embodiment, the oligonucleotide comprising SEQ ID NO:3 has at least one of its internucleoside linkages which is modified, preferably as a phosphorothioate or phosphoramidate (preferably mesyl-phosphoramidate). More preferably, the oligonucleotide comprising SEQ ID NO: 3 is fully modified with a PS backbone.

In an embodiment one wing or preferably each of the wings of the oligonucleotide comprising SEQ ID NO:3 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:3 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:3 is represented by SEQ ID NO:4:

(SEQ ID NO: 4)
eAₛeTₛeTₛeC*ₛeTₛdTₛdTₛdCₛdTₛdTₛdGₛdAₛdAₛdAₛdAₛeTₛe
GₛeGₛeC*ₛeA

In an embodiment the antisense oligonucleotide comprises the following base sequence:

(SEQ ID NO: 5)
CAATTCTTTCTTGAAAATGG(AON3)

In this embodiment, the antisense oligonucleotide has a length of 20 to 50 nucleotides and comprises the base sequence SEQ ID NO:5. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 5 are 5-methylcytosines.

In an embodiment, the oligonucleotide comprising SEQ ID NO:5 has at least one of its internucleoside linkages which is modified, preferably as a phosphorothioate or phosphoramidate (preferably mesyl-phosphoramidate). More preferably, the oligonucleotide comprising SEQ ID NO: 5 is fully modified with a PS backbone.

In an embodiment one wing or preferably each of the wings of the oligonucleotide comprising SEQ ID NO:5 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:5 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:5 is represented by SEQ ID NO: 6:

(SEQ ID NO: 6)
eC*ₛeAₛeAₛeTₛeTₛdCₛdTₛdTₛdTₛdCₛdTₛdTₛdGₛdAₛdAₛeAₛe
AₛeTₛeGₛeG

In an embodiment the antisense oligonucleotide comprises the following base sequence:

(SEQ ID NO: 7)
(AON4)GCAATTCTTTCTTGAAAATG

In this embodiment, the antisense oligonucleotide has a length of 20 to 50 nucleotides and comprises the base sequence SEQ ID NO:7. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 7 are 5-methylcytosines.

In an embodiment, the oligonucleotide comprising SEQ ID NO:7 has at least one of its internucleoside linkages which is modified, preferably as a phosphorothioate or phosphoramidate (preferably mesyl-phosphoramidate). More preferably, the oligonucleotide comprising SEQ ID NO: 7 is fully modified with a PS backbone.

In an embodiment one wing or preferably each of the wings of the oligonucleotide comprising SEQ ID NO:7 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:7 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:7 is represented by SEQ ID NO:8:

(SEQ ID NO: 8)
eGₛeC*ₛeAₛeAₛeTₛdTₛdCₛdTₛdTₛdTₛdCₛdTₛdTₛdGₛdAₛeAₛe
AₛeAₛeTₛeG

In an embodiment the antisense oligonucleotide comprises the following base sequence:

(SEQ ID NO: 9)
(AON5)GGCAATTCTTTCTTGAAAAT

In this embodiment, the antisense oligonucleotide has a length of 20 to 50 nucleotides and comprises the base sequence SEQ ID NO:9. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 9 are 5-methylcytosines.

In an embodiment, the oligonucleotide comprising SEQ ID NO:9 has at least one of its internucleoside linkages which is modified, preferably as a phosphorothioate or phosphoramidate (preferably mesyl-phosphoramidate). More preferably, the oligonucleotide comprising SEQ ID NO: 9 is fully modified with a PS backbone.

In an embodiment one wing or preferably each of the wings of the oligonucleotide comprising SEQ ID NO:9 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:9 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:9 is represented by SEQ ID NO:10:

(SEQ ID NO: 10)
eGₛeGₛeC*ₛeAₛeAₛdTₛdTₛdCₛdTₛdTₛdTₛdCₛdTₛdTₛdGₛeAₛe
AₛeAₛeAₛeT

In an embodiment the antisense oligonucleotide comprises the following base sequence:

(SEQ ID NO: 11)
(AON6)TGGCAATTCTTTCTTGAAAA

In this embodiment, the antisense oligonucleotide has a length of 20 to 50 nucleotides and comprises the base sequence SEQ ID NO: 11. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 11 are 5-methylcytosines.

In an embodiment, the oligonucleotide comprising SEQ ID NO:11 has at least one of its internucleoside linkages which is modified, preferably as a phosphorothioate or phosphoramidate (preferably mesyl-phosphoramidate). More preferably, the oligonucleotide comprising SEQ ID NO: 11 is fully modified with a PS backbone.

In an embodiment one wing or preferably each of the wings of the oligonucleotide comprising SEQ ID NO:11 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:11 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:11 is represented by SEQ ID NO: 12:

(SEQ ID NO: 12)
eTₛeGₛeGₛeC*ₛeAₛdAₛdTₛdTₛdCₛdTₛdTₛdTₛdCₛdTₛdTₛeGₛe
AₛeAₛeAₛeA

In an embodiment the antisense oligonucleotide comprises the following base sequence:

(SEQ ID NO: 13)
(AON7)CTGGCAATTCTTTCTTGAAA

In this embodiment, the antisense oligonucleotide has a length of 20 to 50 nucleotides and comprises the base sequence SEQ ID NO: 13. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 13 are 5-methylcytosines.

In an embodiment, the oligonucleotide comprising SEQ ID NO:13 has at least one of its internucleoside linkages which is modified, preferably as a phosphorothioate or phosphoramidate (preferably mesyl-phosphoramidate). More preferably, the oligonucleotide comprising SEQ ID NO: 13 is fully modified with a PS backbone.

In an embodiment one wing or preferably each of the wings of the oligonucleotide comprising SEQ ID NO:13 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 13 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 13 is represented by SEQ ID NO: 14:

(SEQ ID NO: 14)
eC*ₛeTₛeGₛeGₛeC*ₛdAₛdAₛdTₛdTₛdCₛdTₛdTₛdTₛdCₛdTₛe
TₛeGₛeAₛeAₛeA

In an embodiment the antisense oligonucleotide comprises the following base sequence:

(SEQ ID NO: 15)
(AON8)AGCTGGCAATTCTTTCTTGA

In this embodiment, the antisense oligonucleotide has a length of 20 to 50 nucleotides and comprises the base sequence SEQ ID NO: 15. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 15 are 5-methylcytosines.

In an embodiment, the oligonucleotide comprising SEQ ID NO:15 has at least one of its internucleoside linkages which is modified, preferably as a phosphorothioate or phosphoramidate (preferably mesyl-phosphoramidate). More preferably, the oligonucleotide comprising SEQ ID NO: 15 is fully modified with a PS backbone.

In an embodiment one wing or preferably each of the wings of the oligonucleotide comprising SEQ ID NO: 15 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 15 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 15 is represented by SEQ ID NO: 16:

(SEQ ID NO: 16)
eAₛeGₛeC*ₛeTₛeGₛdGₛdCₛdAₛdAₛdTₛdTₛdCₛdTₛdTₛdTₛeC*ₛ
eTₛeTₛeGₛeA

In an embodiment the antisense oligonucleotide comprises the following base sequence:

(SEQ ID NO: 17)
(AON9)AGAGCTGGCAATTCTTTCTT

In this embodiment, the antisense oligonucleotide has a length of 20 to 50 nucleotides and comprises the base sequence SEQ ID NO: 17. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 17 are 5-methylcytosines.

In an embodiment, the oligonucleotide comprising SEQ ID NO:17 has at least one of its internucleoside linkages which is modified, preferably as a phosphorothioate or phosphoramidate (preferably mesyl-phosphoramidate). More preferably, the oligonucleotide comprising SEQ ID NO: 17 is fully modified with a PS backbone.

In an embodiment one wing or preferably each of the wings of the oligonucleotide comprising SEQ ID NO:17 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 17 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:17 is represented by SEQ ID NO:18:

(SEQ ID NO: 18)
eAₛeGₛeAₛeGₛeC*ₛdTₛdGₛdGₛdCₛdAₛdAₛdTₛdTₛdCₛdTₛeTₛe
TₛeC*ₛeTₛeT

In an embodiment the antisense oligonucleotide comprises the following base sequence:

(SEQ ID NO: 19)
GAAGAGCTGGCAATTCTTTC(AON10)

In this embodiment, the antisense oligonucleotide has a length of 20 to 50 nucleotides and comprises the base sequence SEQ ID NO: 19. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 19 are 5-methylcytosines.

In an embodiment, the oligonucleotide comprising SEQ ID NO:19 has at least one of its internucleoside linkages which is modified, preferably as a phosphorothioate or phosphoramidate (preferably mesyl-phosphoramidate). More preferably, the oligonucleotide comprising SEQ ID NO: 19 is fully modified with a PS backbone.

In an embodiment one wing or preferably each of the wings of the oligonucleotide comprising SEQ ID NO:19 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 19 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 19 is represented by SEQ ID NO:20:

(SEQ ID NO: 20)
eGₛeAₛeAₛeGeAₛdGₛdCₛdTₛdGₛdGₛdCₛdAₛdAₛdTₛdTₛeC*ₛe
TₛeTₛeTₛeC*

In an embodiment the antisense oligonucleotide comprises the following base sequence:

(SEQ ID NO: 21)
(AON11)TGGAAGAGCTGGCAATTCTT

In this embodiment, the antisense oligonucleotide has a length of 20 to 50 nucleotides and comprises the base sequence SEQ ID NO:21. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 21 are 5-methylcytosines.

In an embodiment, the oligonucleotide comprising SEQ ID NO:21 has at least one of its internucleoside linkages which is modified, preferably as a phosphorothioate or phosphoramidate (preferably mesyl-phosphoramidate). More preferably, the oligonucleotide comprising SEQ ID NO: 21 is fully modified with a PS backbone.

In an embodiment one wing or preferably each of the wings of the oligonucleotide comprising SEQ ID NO:21 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:21 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:21 is represented by SEQ ID NO:22:

(SEQ ID NO: 22)
eTₛeGₛeGₛeAₛeAₛdGₛdAₛdGₛdCₛdTₛdGₛdGₛdCₛdAₛdAₛeTₛe
TₛeC*ₛeTₛeT

In an embodiment the antisense oligonucleotide comprises the following base sequence:

(SEQ ID NO: 23)
ATTGGAAGAGCTGGCAATTC(AON12)

In this embodiment, the antisense oligonucleotide has a length of 20 to 50 nucleotides and comprises the base sequence SEQ ID NO:23. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 23 are 5-methylcytosines.

In an embodiment, the oligonucleotide comprising SEQ ID NO:23 has at least one of its internucleoside linkages which is modified, preferably as a phosphorothioate or phosphoramidate (preferably mesyl-phosphoramidate). More preferably, the oligonucleotide comprising SEQ ID NO: 23 is fully modified with a PS backbone.

In an embodiment one wing or preferably each of the wings of the oligonucleotide comprising SEQ ID NO:23 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:23 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:23 is represented by SEQ ID NO:24:

(SEQ ID NO: 24)
eAₛeTₛeTₛeGₛeGₛdAₛdAₛdGₛdAₛdGₛdCₛdTₛdGₛdGₛdCₛeAₛe
AₛeTₛeTₛeC*

In an embodiment the antisense oligonucleotide comprises the following base sequence:

(SEQ ID NO: 25)
GCAATUCTTTCTTGAAAATG(AON13)

In this embodiment, the antisense oligonucleotide has a length of 20 to 50 nucleotides and comprises the base sequence SEQ ID NO:25. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 25 are 5-methylcytosines.

In an embodiment, the oligonucleotide comprising SEQ ID NO:25 has at least one of its internucleoside linkages which is modified, preferably as a phosphorothioate or phosphoramidate (preferably mesyl-phosphoramidate). More preferably, the oligonucleotide comprising SEQ ID NO: 25 is fully modified with a PS backbone.

In an embodiment one wing or preferably each of the wings of the oligonucleotide comprising SEQ ID NO:25 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:25 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:25 is represented by SEQ ID NO:26:

(SEQ ID NO: 26)
eGₛeC*ₛeAₛeAₛeTₛdUₛdCₛdTₛdTₛdTₛdCₛdTₛdTₛdGₛdAₛeAₛe
AₛeAₛeTₛeG

In an embodiment the antisense oligonucleotide comprises the following base sequence:

(SEQ ID NO: 27)
GGCAATUCTTTCTTGAAAAT(AON14)

In this embodiment, the antisense oligonucleotide has a length of 20 to 50 nucleotides and comprises the base sequence SEQ ID NO:27. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 27 are 5-methylcytosines.

In an embodiment, the oligonucleotide comprising SEQ ID NO:27 has at least one of its internucleoside linkages which is modified, preferably as a phosphorothioate or phosphoramidate (preferably mesyl-phosphoramidate). More preferably, the oligonucleotide comprising SEQ ID NO: 27 is fully modified with a PS backbone.

In an embodiment one wing or preferably each of the wings of the oligonucleotide comprising SEQ ID NO:27 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:27 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:27 is represented by SEQ ID NO:28:

(SEQ ID NO: 28)
eGₛeGₛeC*ₛeAₛeAₛdTₛdUₛdCₛdTₛdTₛdTₛdCₛdTₛdTₛdGₛeAₛeAₛeAₛ
eAₛeT

In an embodiment the antisense oligonucleotide comprises the following base sequence:

(SEQ ID NO: 29)
TGGCAATUCTTTCTTGAAAA (AON15)

In this embodiment, the antisense oligonucleotide has a length of 20 to 50 nucleotides and comprises the base sequence SEQ ID NO:29. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C and preferably all Cs of the oligonucleotide comprising SEQ ID NO: 29 are 5-methylcytosines.

In an embodiment, the oligonucleotide comprising SEQ ID NO:29 has at least one of its internucleoside linkages which is modified, preferably as a phosphorothioate or phosphoramidate (preferably mesyl-phosphoramidate). More preferably, the oligonucleotide comprising SEQ ID NO: 29 is fully modified with a PS backbone.

In an embodiment one wing or preferably each of the wings of the oligonucleotide comprising SEQ ID NO:29 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:29 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:29 is represented by SEQ ID NO:30:

(SEQ ID NO: 30)
eTₛeGₛeGₛeC*ₛeAₛdAₛdTₛdUₛdCₛdTₛdTₛdTₛdCₛdTₛdTₛeGₛeAₛeAₛ
eAₛeA

In an embodiment the antisense oligonucleotide comprises the following base sequence:

(SEQ ID NO: 32)
GGCAATUCTTTCTTGAAAAT (AON16)

In this embodiment, the antisense oligonucleotide has a length of 20 to 50 nucleotides and comprises the base sequence SEQ ID NO:32. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:32 is 5-methylcytosine. In another embodiment, the C of the oligonucleotide comprising SEQ ID NO:32 present in the gap of said oligonucleotide (C at position 8 and at position 12 of SEQ ID NO:32) are not methylated. In another embodiment, all Cs of the oligonucleotide comprising SEQ ID NO: 32 are 5-methylcytosines.

In an embodiment, the oligonucleotide comprising SEQ ID NO:32 has at least one of its internucleoside linkages which is modified, preferably as a phosphorothioate or phosphoramidate (preferably mesyl-phosphoramidate). In an embodiment, the oligonucleotide comprising SEQ ID NO: 32 has 1, 2 or 3 phosphosdiester linkages in each of its wings, preferably 3 phosphodiester linkages in each of its wings. In another embodiment, the oligonucleotide comprising SEQ ID NO: 32 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO: 32 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 32 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:32 is represented by SEQ ID NO:33:

(SEQ ID NO: 33)
eGₛeGₒeC*ₒeAₒeAₛdTₛdUₛdCₛdTₛdTₛdTₛdCₛdTₛdTₛdGₛeAₒeAₒeAₒ
eAₛeT

In an embodiment the antisense oligonucleotide comprises the following base sequence:

(SEQ ID NO: 34)
GGCAATUCTTTCTTGAAAAT (AON17)

In this embodiment, the antisense oligonucleotide has a length of 20 to 50 nucleotides and comprises the base sequence SEQ ID NO:34. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:34 is 5-methylcytosine. In another embodiment, the C of the oligonucleotide comprising SEQ ID NO:32 present in the gap of said oligonucleotide (C at position 8 and at position 12 of SEQ ID NO:34) are not methylated. In another embodiment, all Cs of the oligonucleotide comprising SEQ ID NO: 34 are 5-methylcytosines.

In an embodiment, the oligonucleotide comprising SEQ ID NO:34 has at least one of its internucleoside linkages which is modified, preferably as a phosphorothioate or phosphoramidate (preferably mesyl-phosphoramidate). In an embodiment, the oligonucleotide comprising SEQ ID NO: 34 has 1, 2 or 3 phosphosdiesters linkage in each of its wings, preferably 3 phosphodiesters linkage in each of its wings. In another embodiment, the oligonucleotide comprising SEQ ID NO: 34 is fully modified with a PS backbone.

In an embodiment, the oligonucleotide comprising SEQ ID NO:34 has at least one of its internucleoside linkages which is modified, preferably as a PNms linkage, preferably at least two of them. In an embodiment, a PNms linkage is present in the central region of the oligonucleotide and not in its wings.

In an embodiment one wing or preferably each of the wings of the oligonucleotide comprising SEQ ID NO:34 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:34 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:34 is represented by SEQ ID NO:35:

(SEQ ID NO: 35)
eGₛeGₛeC*ₛeAₛeAₛdTₛdU_{PNms}dC_{PNms}dTₛdTₛdTₛdCₛdTₛdTₛdGₛeAₛe
AₛeAₛeAₛeT

In an embodiment the antisense oligonucleotide comprises the following base sequence:

(SEQ ID NO: 36)
GGCAATUCTTTCTTGAAAAT (AON18)

In this embodiment, the antisense oligonucleotide has a length of 20 to 50 nucleotides and comprises the base sequence SEQ ID NO:36. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:36 is 5-methylcytosine. In another embodiment, the C of the oligonucleotide comprising SEQ ID NO:36 present in the gap of said oligonucleotide (C at position 8 and at position 12 of SEQ ID NO:36) are not methylated. In another embodiment, all Cs of the oligonucleotide comprising SEQ ID NO: 36 are 5-methylcytosines.

In an embodiment, the oligonucleotide comprising SEQ ID NO:36 has at least one of its internucleoside linkages which is modified, preferably as a phosphorothioate or phosphoramidate (preferably mesyl-phosphoramidate). In an embodiment, the oligonucleotide comprising SEQ ID NO: 36 has 1, 2 or 3 phosphosdiesters linkage in each of its wings, preferably 3 phosphodiesters linkage in each of its wings. In another embodiment, the oligonucleotide comprising SEQ ID NO: 36 is fully modified with a PS backbone.

In an embodiment, the oligonucleotide comprising SEQ ID NO:36 has at least one of its internucleoside linkages which is modified, preferably as a PNms linkage, preferably at least two of them. In an embodiment, a PNms linkage is present in the central region of the oligonucleotide and not in its wings.

In an embodiment one wing or preferably each of the wings of the oligonucleotide comprising SEQ ID NO:36 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:36 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:36 is represented by SEQ ID NO:37:

(SEQ ID NO: 37)
eGₛeGₛeC*ₛeAₛeAₛdTₛdUₛdCP_{Nms}dT_{PNms}dTₛdTₛdCₛdTₛdTₛdGₛeAₛe
AₛeAₛeAₛeT

In an embodiment the antisense oligonucleotide comprises the following base sequence:

(SEQ ID NO: 38)
GGCAATUCTTTCTTGAAAAT (AON19)

In this embodiment, the antisense oligonucleotide has a length of 20 to 50 nucleotides and comprises the base sequence SEQ ID NO:38. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:38 is 5-methylcytosine. In another embodiment, the C of the oligonucleotide comprising SEQ ID NO:38 present in the gap of said oligonucleotide (C at position 8 and at position 12 of SEQ ID NO:38) are not methylated. In another embodiment, all Cs of the oligonucleotide comprising SEQ ID NO: 38 are 5-methylcytosines.

In an embodiment, the oligonucleotide comprising SEQ ID NO:38 has at least one of its internucleoside linkages which is modified, preferably as a phosphorothioate or phosphoramidate (preferably mesyl-phosphoramidate). More preferably, the oligonucleotide comprising SEQ ID NO: 38 has 1, 2 or 3 phosphosdiesters linkage in each of its wings, preferably 3 phosphodiesters linkage in each of its wings. In an embodiment, the oligonucleotide comprising SEQ ID NO: 38 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

In an embodiment, the oligonucleotide comprising SEQ ID NO:38 has at least one of its internucleoside linkages which is modified, preferably as a PNms linkage, preferably at least two of them. In an embodiment, a PNms linkage is present in the central region of the oligonucleotide and not in its wings.

In an embodiment one wing or preferably each of the wings of the oligonucleotide comprising SEQ ID NO:38 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:38 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:38 is represented by SEQ ID NO:39:

(SEQ ID NO: 39)
eGₛeGₒeC*ₒeAₒeAₛdTₛdUₛdC_{PNms}dT_{PNms}dTₛdTₛdCₛdTₛdTₛdGₛeAₒe
AₒeAₒeAₛeT

In an embodiment the antisense oligonucleotide comprises the following base sequence:

(SEQ ID NO: 40)
GGCAAATCTTTCTTGAAAAT (AON22)

In this embodiment, the antisense oligonucleotide has a length of 20 to 50 nucleotides and comprises the base sequence SEQ ID NO:40. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:40 is 5-methylcytosine. In another embodiment, the C of the oligonucleotide comprising SEQ ID NO:40 present in the gap of said oligonucleotide (C at position 8 and at position 12 of SEQ ID NO:40) are not methylated. In another embodiment, all Cs of the oligonucleotide comprising SEQ ID NO: 40 are 5-methylcytosines.

In an embodiment, the oligonucleotide comprising SEQ ID NO:40 has at least one of its internucleoside linkages which is modified, preferably as a phosphorothioate or phosphoramidate (preferably mesyl-phosphoramidate). More preferably, the oligonucleotide comprising SEQ ID NO: 40 has 1, 2 or 3 phosphosdiesters linkage in each of its wings, preferably 3 phosphodiesters linkage in each of its wings. In an embodiment, the oligonucleotide comprising SEQ ID NO:40 is fully modified with a PS backbone.

In an embodiment one wing or preferably each of the wings of the oligonucleotide comprising SEQ ID NO:40 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:40 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:40 is represented by SEQ ID NO:41:

(SEQ ID NO: 41)
eGₛeGₛeC*ₛeAₛeAₛdAₛdTₛdCₛdTₛdTₛdTₛdCₛdTₛdTₛdGₛeAₛeAₛeAₛ
eAₛeT

In an embodiment the antisense oligonucleotide comprises the following base sequence:

(SEQ ID NO: 42)
GGCAATTGTTTCTTGAAAAT (AON23)

In this embodiment, the antisense oligonucleotide has a length of 20 to 50 nucleotides and comprises the base sequence SEQ ID NO:42. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:42 is 5-methylcytosine. In another embodiment, the C of the oligonucleotide comprising SEQ ID NO:42 present in the gap of said oligonucleotide (C at position 12 of SEQ ID NO:42) are not methylated. In another embodiment, all Cs of the oligonucleotide comprising SEQ ID NO:42 are 5-methylcytosines.

In an embodiment, the oligonucleotide comprising SEQ ID NO:42 has at least one of its internucleoside linkages which is modified, preferably as a phosphorothioate or phosphoramidate (preferably mesyl-phosphoramidate). More preferably, the oligonucleotide comprising SEQ ID NO: 42 has 1, 2 or 3 phosphosdiesters linkage in each of its wings, preferably 3 phosphodiesters linkage in each of its wings. In an embodiment, the oligonucleotide comprising SEQ ID NO:42 is fully modified with a PS backbone.

In an embodiment one wing or preferably each of the wings of the oligonucleotide comprising SEQ ID NO:42 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:42 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:42 is represented by SEQ ID NO:43:

(SEQ ID NO: 43)
eGₛeGₛeC*ₛeAₛeAₛdTₛdTₛdGₛdTₛdTₛdTₛdCₛdTₛdTₛdGₛeAₛeAₛe
AₛeAₛeT

In an embodiment the antisense oligonucleotide comprises the following base sequence:

(SEQ ID NO: 44)
GGCAATTGATTCTTGAAAAT (AON24)

In this embodiment, the antisense oligonucleotide has a length of 20 to 50 nucleotides and comprises the base sequence SEQ ID NO:44. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:44 is 5-methylcytosine. In another embodiment, the C of the oligonucleotide comprising SEQ ID NO:44 present in the gap of said oligonucleotide (C at position 12 of SEQ ID NO:44) are not methylated.

In another embodiment, all Cs of the oligonucleotide comprising SEQ ID NO:44 are 5-methylcytosines.

In an embodiment, the oligonucleotide comprising SEQ ID NO:44 has at least one of its internucleoside linkages which is modified, preferably as a phosphorothioate or phosphoramidate (preferably mesyl-phosphoramidate). More preferably, the oligonucleotide comprising SEQ ID NO: 44 has 1, 2 or 3 phosphosdiesters linkage in each of its wings, preferably 3 phosphodiesters linkage in each of its wings. In an embodiment, the oligonucleotide comprising SEQ ID NO:44 is fully modified with a PS backbone.

In an embodiment one wing or preferably each of the wings of the oligonucleotide comprising SEQ ID NO:44 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:44 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:44 is represented by SEQ ID NO:45:

(SEQ ID NO: 45)
eGₛeGₛeC*ₛeAₛeAₛdTₛdTₛdGₛdAₛdTₛdTₛdCₛdTₛdTₛdGₛeAₛeAₛeAₛ
eAₛeT

In an embodiment the antisense oligonucleotide comprises the following base sequence:

(SEQ ID NO: 46)
GCAATUCTTTCTTGAAAATG (AON25)

In this embodiment, the antisense oligonucleotide has a length of 20 to 50 nucleotides and comprises the base sequence SEQ ID NO:46. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:46 is 5-methylcytosine. In another embodiment, the C of the oligonucleotide comprising SEQ ID NO:46 present in the gap of said oligonucleotide (C at position 7 and C at position 11 of SEQ ID NO:46) are not methylated. In another embodiment, all Cs of the oligonucleotide comprising SEQ ID NO: 46 are 5-methylcytosines.

In an embodiment, the oligonucleotide comprising SEQ ID NO:46 has at least one of its internucleoside linkages which is modified, preferably as a phosphorothioate or phosphoramidate (preferably mesyl-phosphoramidate). More preferably, the oligonucleotide comprising SEQ ID NO: 46 has 1, 2 or 3 phosphosdiesters linkage in each of its wings, preferably 3 phosphodiesters linkage in each of its wings. In an embodiment, the oligonucleotide comprising SEQ ID NO:46 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO: 46 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 46 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:46 is represented by SEQ ID NO:47:

(SEQ ID NO: 47)
eGₛeC*ₒeAₒeAₒeTₛdUₛdCₛdTₛdTₛdTₛdCₛdTₛdTₛdGₛdAₛeAₒeAₒeAₒ
eTₛeG

In an embodiment the antisense oligonucleotide comprises the following base sequence:

(SEQ ID NO: 48)
GGCAATTCTAACTTGAAAAT (AON26)

In this embodiment, the antisense oligonucleotide has a length of 20 to 50 nucleotides and comprises the base sequence SEQ ID NO:48. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:48 is 5-methylcytosine. In another embodiment, the C of the oligonucleotide comprising SEQ ID NO:48 present in the gap of said oligonucleotide (C at position 8 and at position 12 of SEQ ID NO:48) are not methylated. In another embodiment, all Cs of the oligonucleotide comprising SEQ ID NO: 48 are 5-methylcytosines.

In an embodiment, the oligonucleotide comprising SEQ ID NO:48 has at least one of its internucleoside linkages which is modified, preferably as a phosphorothioate or phosphoramidate (preferably mesyl-phosphoramidate). More preferably, the oligonucleotide comprising SEQ ID NO: 48 has 1, 2 or 3 phosphosdiesters linkage in each of its wings, preferably 3 phosphodiesters linkage in each of its wings. In an embodiment, the oligonucleotide comprising SEQ ID NO:48 is fully modified with a PS backbone.

In an embodiment one wing or preferably each of the wings of the oligonucleotide comprising SEQ ID NO:48 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:48 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:48 is represented by SEQ ID NO:49:

(SEQ ID NO: 49)
eGₛeGₛeC*ₛeAₛeAₛdTₛdTₛdCₛdTₛdAₛdAₛdCₛdTₛdTₛdGₛeAₛeAₛeAₛ
eAₛeT

In an embodiment the antisense oligonucleotide comprises the following base sequence:

(SEQ ID NO: 50)
GGCAATTCTTAGTTGAAAAT (AON27)

In this embodiment, the antisense oligonucleotide has a length of 20 to 50 nucleotides and comprises the base sequence SEQ ID NO:50. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:50 is 5-methylcytosine. In another embodiment, the C of the oligonucleotide comprising SEQ ID NO:50 present in the gap of said oligonucleotide (C at position 8 of SEQ ID NO:50) are not methylated. In another embodiment, all Cs of the oligonucleotide comprising SEQ ID NO:50 are 5-methylcytosines.

In an embodiment, the oligonucleotide comprising SEQ ID NO:50 has at least one of its internucleoside linkages which is modified, preferably as a phosphorothioate or phosphoramidate (preferably mesyl-phosphoramidate). More preferably, the oligonucleotide comprising SEQ ID NO: 50 has 1, 2 or 3 phosphosdiesters linkage in each of its wings, preferably 3 phosphodiesters linkage in each of its wings. In an embodiment, the oligonucleotide comprising SEQ ID NO:50 is fully modified with a PS backbone.

In an embodiment one wing or preferably each of the wings of the oligonucleotide comprising SEQ ID NO:50 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:50 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:50 is represented by SEQ ID NO:51:

(SEQ ID NO: 51)
eGₛeGₛeC*ₛeAₛeAₛdTₛdTₛdCₛdTₛdTₛdAₛdGₛdTₛdTₛdGₛeAₛeAₛeAₛ
eAₛeT

In an embodiment the antisense oligonucleotide comprises the following base sequence:

(SEQ ID NO: 52)
CAATTCTTTCTTGAAAAT (AON28)

In this embodiment, the antisense oligonucleotide has a length of 18 to 50 nucleotides and comprises the base sequence SEQ ID NO:52. The length may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:52 is 5-methylcytosine. In another embodiment, the C of the oligonucleotide comprising SEQ ID NO:52 present in the gap of said oligonucleotide (C at position 6 and at position 10 of SEQ ID NO:52) are not methylated. In another embodiment, and preferably all Cs of the oligonucleotide comprising SEQ ID NO:52 are 5-methylcytosines.

In an embodiment, the oligonucleotide comprising SEQ ID NO:52 has at least one of its internucleoside linkages which is modified, preferably as a phosphorothioate or phosphoramidate (preferably mesyl-phosphoramidate). More preferably, the oligonucleotide comprising SEQ ID NO: 52 has 1, 2 or 3 phosphosdiesters linkage in each of its wings, preferably 3 phosphodiesters linkage in each of its wings. In an embodiment, the oligonucleotide comprising SEQ ID NO:52 is fully modified with a PS backbone.

In an embodiment one wing or preferably each of the wings of the oligonucleotide comprising SEQ ID NO:52 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:52 comprises 1, 2, 3, 4 or 5 nucleotides. In a more preferred embodiment, one wing or even more preferably both wings comprise 4 nucleotides. Most preferably both wings comprise 4 nucleotides and are MOE.

A most preferred oligonucleotide comprising SEQ ID NO:52 is represented by SEQ ID NO:53:

(SEQ ID NO: 53)
eC*ₛeAₛeAₛeTₛdTₛdCₛdTₛdTₛdTₛdCₛdTₛdTₛdGₛdAₛeAₛeAₛeAₛeT

In an embodiment the antisense oligonucleotide comprises the following base sequence:

(SEQ ID NO: 54)
GCAATTCTTTCTTGAAAA (AON29)

In this embodiment, the antisense oligonucleotide has a length of 18 to 50 nucleotides and comprises the base sequence SEQ ID NO:54. The length may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:54 is 5-methylcytosine. In another embodiment, the C of the oligonucleotide comprising SEQ ID NO:54 present in the gap of said oligonucleotide (C at position 7 and at position 11 of SEQ ID NO:54) are not methylated. In another embodiment, and preferably all Cs of the oligonucleotide comprising SEQ ID NO:54 are 5-methylcytosines.

In an embodiment, the oligonucleotide comprising SEQ ID NO:54 has at least one of its internucleoside linkages which is modified, preferably as a phosphorothioate or phosphoramidate (preferably mesyl-phosphoramidate). More preferably, the oligonucleotide comprising SEQ ID NO: 54 has 1, 2 or 3 phosphosdiesters linkage in each of its wings, preferably 3 phosphodiesters linkage in each of its wings. In an embodiment, the oligonucleotide comprising SEQ ID NO:54 is fully modified with a PS backbone.

In an embodiment one wing or preferably each of the wings of the oligonucleotide comprising SEQ ID NO:54 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:54 comprises 1, 2, 3, 4 or 5 nucleotides. In a more preferred embodiment, one wing or even more preferably both wings comprise 4 nucleotides. Most preferably both wings comprise 4 nucleotides and are MOE.

A most preferred oligonucleotide comprising SEQ ID NO:54 is represented by SEQ ID NO:55:

(SEQ ID NO: 55)
eGₛeC*ₛeAₛeAₛdTₛdTₛdCₛdTₛdTₛdTₛdCₛdTₛdTₛdGₛeAₛeAₛeAₛeA

In an embodiment the antisense oligonucleotide comprises the following base sequence:

(SEQ ID NO: 56)
GGCAATTCTTTCTTGAAA (AON30)

In this embodiment, the antisense oligonucleotide has a length of 18 to 50 nucleotides and comprises the base sequence SEQ ID NO:56. The length may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:56 is 5-methylcytosine. In another embodiment, the C of the oligonucleotide comprising SEQ ID NO:56 present in the gap of said oligonucleotide (C at position 8 and at position 12 of SEQ ID NO:56) are not methylated. In another embodiment, all Cs of the oligonucleotide comprising SEQ ID NO: 56 are 5-methylcytosines.

In an embodiment, the oligonucleotide comprising SEQ ID NO:56 has at least one of its internucleoside linkages which is modified, preferably as a phosphorothioate or phosphoramidate (preferably mesyl-phosphoramidate). More preferably, the oligonucleotide comprising SEQ ID NO: 56 has 1, 2 or 3 phosphosdiesters linkage in each of its wings, preferably 3 phosphodiesters linkage in each of its wings. In an embodiment, the oligonucleotide comprising SEQ ID NO:56 is fully modified with a PS backbone.

In an embodiment one wing or preferably each of the wings of the oligonucleotide comprising SEQ ID NO:56 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:56 comprises 1, 2, 3, 4 or 5 nucleotides. In a more preferred embodiment, one wing or even more preferably both wings comprise 4 nucleotides. Most preferably both wings comprise 4 nucleotides and are MOE.

A most preferred oligonucleotide comprising SEQ ID NO:56 is represented by SEQ ID NO:57:

(SEQ ID NO: 57)
eGₛeGₛeC*ₛeAₛdAₛdTₛdTₛdCₛdTₛdTₛdTₛdCₛdTₛdTₛeGₛeAₛeAₛeA

In an embodiment the antisense oligonucleotide comprises the following base sequence:

(SEQ ID NO: 58)
AATTCTTTCTTGAAAA (AON31)

In this embodiment, the antisense oligonucleotide has a length of 16 to 50 nucleotides and comprises the base sequence SEQ ID NO:58. The length may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:58 is 5-methylcytosine. In another embodiment, the C of the oligonucleotide comprising SEQ ID NO:58 present in the gap of said oligonucleotide (C at positions 5 and 9 of SEQ ID NO:58) are not methylated. In another embodiment, all Cs of the oligonucleotide comprising SEQ ID NO:58 are 5-methylcytosines.

In an embodiment, the oligonucleotide comprising SEQ ID NO:58 has at least one of its internucleoside linkages which is modified, preferably as a phosphorothioate or phosphoramidate (preferably mesyl-phosphoramidate). More preferably, the oligonucleotide comprising SEQ ID NO: 58 has 1, 2 or 3 phosphosdiesters linkage in each of its wings, preferably 3 phosphodiesters linkage in each of its wings. In an embodiment, the oligonucleotide comprising SEQ ID NO:58 is fully modified with a PS backbone.

In an embodiment one wing or preferably each of the wings of the oligonucleotide comprising SEQ ID NO:58 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:58 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:58 is represented by SEQ ID NO:59:

(SEQ ID NO: 59)
eAₛeAₛeTₛdTₛdCₛdTₛdTₛdTₛdCₛdTₛdTₛdGₛdAₛeAₛeAₛeA

In an embodiment the antisense oligonucleotide comprises the following base sequence:

(SEQ ID NO: 60)
CAATTCTTTCTTGAAA (AON32)

In this embodiment, the antisense oligonucleotide has a length of 16 to 50 nucleotides and comprises the base sequence SEQ ID NO:60. The length may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:60 is 5-methylcytosine. In another embodiment, the C of the oligonucleotide comprising SEQ ID NO:60 present in the gap of said oligonucleotide (C at position 6 and position 10 of SEQ ID NO:60) are not methylated. In another embodiment, all Cs of the oligonucleotide comprising SEQ ID NO:60 are 5-methylcytosines.

In an embodiment, the oligonucleotide comprising SEQ ID NO:60 has at least one of its internucleoside linkages which is modified, preferably as a phosphorothioate or phosphoramidate (preferably mesyl-phosphoramidate). More preferably, the oligonucleotide comprising SEQ ID NO: 60 has 1, 2 or 3 phosphosdiesters linkage in each of its wings, preferably 3 phosphodiesters linkage in each of its wings. In an embodiment, the oligonucleotide comprising SEQ ID NO:60 is fully modified with a PS backbone.

In an embodiment one wing or preferably each of the wings of the oligonucleotide comprising SEQ ID NO:60 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:60 comprises 1, 2, 3, 4 or 5 nucleotides. In a more preferred embodiment, one wing or even more preferably both wings comprise 3 nucleotides. Most preferably both wings comprise 3 nucleotides and are MOE.

A most preferred oligonucleotide comprising SEQ ID NO:60 is represented by SEQ ID NO:61:

(SEQ ID NO: 61)
eC*ₛeAₛeAₛdTₛdTₛdCₛdTₛdTₛdTₛdCₛdTₛdTₛdGₛeAₛeAₛeA

In an embodiment the antisense oligonucleotide comprises the following base sequence:

(SEQ ID NO: 62)
GCAATTCTTTCTTGAA (AON33)

In this embodiment, the antisense oligonucleotide has a length of 16 to 50 nucleotides and comprises the base sequence SEQ ID NO:62. The length may be 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:62 is 5-methylcytosine. In another embodiment, the C of the oligonucleotide comprising SEQ ID NO:62 present in the gap of said oligonucleotide (C at position 7 and position 11 of SEQ ID NO:62) are not methylated. In another embodiment, all Cs of the oligonucleotide comprising SEQ ID NO:62 are 5-methylcytosines.

In an embodiment, the oligonucleotide comprising SEQ ID NO:62 has at least one of its internucleoside linkages which is modified, preferably as a phosphorothioate or phosphoramidate (preferably mesyl-phosphoramidate). More preferably, the oligonucleotide comprising SEQ ID NO: 62 has 1, 2 or 3 phosphosdiesters linkage in each of its wings, preferably 3 phosphodiesters linkage in each of its wings. In an embodiment, the oligonucleotide comprising SEQ ID NO:62 is fully modified with a PS backbone.

In an embodiment one wing or preferably each of the wings of the oligonucleotide comprising SEQ ID NO:62 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:62 comprises 1, 2, 3, 4 or 5 nucleotides. In a more preferred embodiment, one wing or even more preferably both wings comprise 3 nucleotides. Most preferably both wings comprise 3 nucleotides and are MOE.

A most preferred oligonucleotide comprising SEQ ID NO:62 is represented by SEQ ID NO:63:

(SEQ ID NO: 63)
eGₛeC*ₛeAₛdAₛdTₛdTₛdCₛdTₛdTₛdTₛdCₛdTₛdTₛeGₛeAₛeA

In an embodiment the antisense oligonucleotide comprises the following base sequence:

(SEQ ID NO: 64)
ATTCTTTCTTGAAAATGGCA (AON34)

In this embodiment, the antisense oligonucleotide has a length of 20 to 50 nucleotides and comprises the base sequence SEQ ID NO:64. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:64 is 5-methylcytosine. In another embodiment, the C of the oligonucleotide comprising SEQ ID NO:64 present in the gap of said oligonucleotide (C at position 8 of SEQ ID NO:64) are not methylated. In another embodiment, and preferably all Cs of the oligonucleotide comprising SEQ ID NO:64 are 5-methylcytosines.

In an embodiment, the oligonucleotide comprising SEQ ID NO:64 has at least one of its internucleoside linkages which is modified, preferably as a phosphorothioate or phosphoramidate (preferably mesyl-phosphoramidate). More preferably, the oligonucleotide comprising SEQ ID NO: 64 has 1, 2 or 3 phosphosdiesters linkage in each of its wings, preferably 3 phosphodiesters linkage in each of its wings. In an embodiment, the oligonucleotide comprising SEQ ID NO:64 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

In an embodiment one wing or preferably each of the wings of the oligonucleotide comprising SEQ ID NO:64 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:64 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:64 is represented by SEQ ID NO:65:

(SEQ ID NO: 65)
eAₛeTₒeTₒeC*ₒeTₛdTₛdTₛdCₛdTₛdTₛdGₛdAₛdAₛdAₛdAₛeTₒeGₒeGₒ
eC*ₛeA

In an embodiment the antisense oligonucleotide comprises the following base sequence:

(SEQ ID NO: 66)
ATTCTTTCTTGAAAATGGCA (AON35)

In this embodiment, the antisense oligonucleotide has a length of 20 to 50 nucleotides and comprises the base sequence SEQ ID NO:66. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:66 is 5-methylcytosine. In another embodiment, the C of the oligonucleotide comprising SEQ ID NO:66 present in the gap of said oligonucleotide (C at position 8 of SEQ ID NO:66) are not methylated. In another embodiment, all Cs of the oligonucleotide comprising SEQ ID NO:66 are 5-methylcytosines.

In an embodiment, the oligonucleotide comprising SEQ ID NO:66 has at least one of its internucleoside linkages which is modified, preferably as a phosphorothioate or phosphoramidate (preferably mesyl-phosphoramidate). More preferably, the oligonucleotide comprising SEQ ID NO: 66 has 1, 2 or 3 phosphosdiesters linkage in each of its wings, preferably 3 phosphodiesters linkage in each of its wings. In an embodiment, the oligonucleotide comprising SEQ ID NO:66 is fully modified with a PS backbone.

In an embodiment one wing or preferably each of the wings of the oligonucleotide comprising SEQ ID NO:66 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:66 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, one internucleoside linkage modification of one wing or preferably of each of the wings of the oligonucleotide comprising SEQ ID NO:66 is a dmi-Phosphoramidate (PNdmi) linkage. Preferably, one wing or each of the wings of the oligonucleotide comprising SEQ ID NO: 66 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 66 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

A most preferred oligonucleotide comprising SEQ ID NO:66 is represented by SEQ ID NO:67:

(SEQ ID NO: 67)
eAₛeTP_{Ndmi}eT_{PNdmi}eC*_{PNdmi}eTₛdTₛdTₛdCₛdTₛdTₛdGₛdAₛdAₛdAₛdAₛ
eT_{PNdmi}eG_{PNdmi}eG_{PNdmi}eC*ₛeA

In an embodiment the antisense oligonucleotide comprises the following base sequence:

(SEQ ID NO: 68)
GGCAATTCTTTCTTGAAAAT (AON36)

In this embodiment, the antisense oligonucleotide has a length of 20 to 50 nucleotides and comprises the base sequence SEQ ID NO:68. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:68 is 5-methylcytosine. In another embodiment, the C of the oligonucleotide comprising SEQ ID NO:68 present in the gap of said oligonucleotide (C at position 8 and position 12 of SEQ ID NO:68) are not methylated. In another embodiment, all Cs of the oligonucleotide comprising SEQ ID NO:68 are 5-methylcytosines.

In an embodiment, the oligonucleotide comprising SEQ ID NO:68 has at least one of its internucleoside linkages which is modified, preferably as a phosphorothioate or phosphoramidate (preferably mesyl-phosphoramidate). More preferably, the oligonucleotide comprising SEQ ID NO: 68 has 1, 2 or 3 phosphosdiesters linkage in each of its wings, preferably 3 phosphodiesters linkage in each of its wings. In an embodiment, the oligonucleotide comprising SEQ ID NO:68 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

In an embodiment one wing or preferably each of the wings of the oligonucleotide comprising SEQ ID NO:68 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:68 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:68 is represented by SEQ ID NO:69:

(SEQ ID NO: 69)
eGₛeGₒeC*ₒeAₒeAₛdTₛdTₛdCₛdTₛdTₛdTₛdCₛdTₛdTₛdGₛeAₒeAₒeAₒ
eAₛeT

In an embodiment the antisense oligonucleotide comprises the following base sequence:

(SEQ ID NO: 70)
GGCAATTCTTTCTTGAAAAT (AON37)

In this embodiment, the antisense oligonucleotide has a length of 20 to 50 nucleotides and comprises the base sequence SEQ ID NO:70. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:70 is 5-methylcytosine. In another embodiment, the C of the oligonucleotide comprising SEQ ID NO:70 present in the gap of said oligonucleotide (C at position 8 and position 12 of SEQ ID NO:70) are not methylated. In another embodiment, all Cs of the oligonucleotide comprising SEQ ID NO:70 are 5-methylcytosines.

In an embodiment, the oligonucleotide comprising SEQ ID NO:70 has at least one of its internucleoside linkages which is modified, preferably as a phosphorothioate or phosphoramidate (preferably mesyl-phosphoramidate). More preferably, the oligonucleotide comprising SEQ ID NO: 70 has 1, 2 or 3 phosphosdiesters linkage in each of its wings, preferably 3 phosphodiesters linkage in each of its wings. In an embodiment, the oligonucleotide comprising SEQ ID NO:70 is fully modified with a PS backbone.

In an embodiment, one internucleoside linkage modification of one wing or preferably of each of the wings of the oligonucleotide comprising SEQ ID NO:70 is a dmi-Phosphoramidate (PNdmi) linkage. Preferably, one wing or each of the wings of the oligonucleotide comprising SEQ ID NO: 70 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 70 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:70 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:70 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:70 is represented by SEQ ID NO:71:

(SEQ ID NO: 71)
eGₛeG_{PNdmi}eC*_{PNdmi}eA_{PNdmi}eAₛdTₛdTₛdCₛdTₛdTₛdTₛdCₛdTₛdTₛdGₛ
eA_{PNdmi}eA_{PNdmi}eA_{PNdmi}eAₛeT

In an embodiment the antisense oligonucleotide comprises the following base sequence:

(SEQ ID NO: 72)
GGCAATTCTTTCTTGAAAAT (AON38)

In this embodiment, the antisense oligonucleotide has a length of 20 to 50 nucleotides and comprises the base sequence SEQ ID NO:72. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:72 is 5-methylcytosine. In another embodiment, the C of the oligonucleotide comprising SEQ ID NO:72 present in the gap of said oligonucleotide (C at position 8 and position 12 of SEQ ID NO:72) are not methylated. In another embodiment, all Cs of the oligonucleotide comprising SEQ ID NO:72 are 5-methylcytosines.

In an embodiment, the oligonucleotide comprising SEQ ID NO:72 has at least one of its internucleoside linkages which is modified, preferably as a phosphorothioate or phosphoramidate (preferably mesyl-phosphoramidate). More preferably, the oligonucleotide comprising SEQ ID NO: 72 has 1, 2 or 3 phosphosdiesters linkage in each of its wings, preferably 3 phosphodiesters linkage in each of its wings. In an embodiment, the oligonucleotide comprising SEQ ID NO:72 is fully modified with a PS backbone.

In an embodiment one wing or preferably each of the wings of the oligonucleotide comprising SEQ ID NO:72 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:72 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

A most preferred oligonucleotide comprising SEQ ID NO:72 is represented by SEQ ID NO:73:

(SEQ ID NO: 73)
eGₛeGₒeC*ₛeAₒeAₛdTₛdTₛdCₛdTₛdTₛdTₛdCₛdTₛdTₛdGₛeAₒeAₛeAₒ
eAₛeT

In an embodiment the antisense oligonucleotide comprises the following base sequence:

(SEQ ID NO: 74)
AGCTGGCAATTCTTTCTTGA (AON39)

In this embodiment, the antisense oligonucleotide has a length of 20 to 50 nucleotides and comprises the base sequence SEQ ID NO:74. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:74 is 5-methylcytosine. In another embodiment, the C of the oligonucleotide comprising SEQ ID NO:74 present in the gap of said oligonucleotide (C at position 7 and position 12 of SEQ ID NO:74) are not methylated. In another embodiment, and preferably all Cs of the oligonucleotide comprising SEQ ID NO:74 are 5-methylcytosines.

In an embodiment, the oligonucleotide comprising SEQ ID NO:74 has at least one of its internucleoside linkages which is modified, preferably as a phosphorothioate or phosphoramidate (preferably mesyl-phosphoramidate). More preferably, the oligonucleotide comprising SEQ ID NO: 74 has 1, 2 or 3 phosphosdiesters linkage in each of its wings, preferably 3 phosphodiesters linkage in each of its wings. In an embodiment, the oligonucleotide comprising SEQ ID NO:74 is fully modified with a PS backbone.

In an embodiment one wing or preferably each of the wings of the oligonucleotide comprising SEQ ID NO:74 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:74 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

A most preferred oligonucleotide comprising SEQ ID NO:74 is represented by SEQ ID NO:75:

(SEQ ID NO: 75)
eAₛeGₒeC*ₒeTₒeGₛdGₛdCₛdAₛdAₛdTₛdTₛdCₛdTₛdTₛdTₛeC*ₒeTₒe
TₒeGₛeA

In an embodiment the antisense oligonucleotide comprises the following base sequence:

(SEQ ID NO: 76)
AGCTGGCAATTCTTTCTTGA (AON40)

In this embodiment, the antisense oligonucleotide has a length of 20 to 50 nucleotides and comprises the base sequence SEQ ID NO:76. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:76 is 5-methylcytosine. In another embodiment, the C of the oligonucleotide comprising SEQ ID NO:76 present in the gap of said oligonucleotide (C at position 7 and position 12 of SEQ ID NO:76) are not methylated. In another embodiment, all Cs of the oligonucleotide comprising SEQ ID NO:76 are 5-methylcytosines.

In an embodiment, the oligonucleotide comprising SEQ ID NO:76 has at least one of its internucleoside linkages which is modified, preferably as a phosphorothioate or phosphoramidate (preferably mesyl-phosphoramidate). More preferably, the oligonucleotide comprising SEQ ID NO: 76 has 1, 2 or 3 phosphosdiesters linkage in each of its wings, preferably 3 phosphodiesters linkage in each of its wings. In an embodiment, the oligonucleotide comprising SEQ ID NO:76 is fully modified with a PS backbone.

In an embodiment, one internucleoside linkage modification of one wing or preferably of each of the wings of the oligonucleotide comprising SEQ ID NO:76 is a dmi-Phosphoramidate (PNdmi) linkage. Preferably, one wing or each of the wings of the oligonucleotide comprising SEQ ID NO: 76 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 76 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:76 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:76 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:76 is represented by SEQ ID NO:77:

(SEQ ID NO: 77)
eAₛeG_{PNdmi}eC*_{PNdmi}eT_{PNdmi}eGₛdGₛdCₛdAₛdAₛdTₛdTₛdCₛdTₛdTₛdTₛ
eC*_{PNdmi}eT_{PNdmi}eT_{PNdmi}eGₛeA

In an embodiment the antisense oligonucleotide comprises the following base sequence:

(SEQ ID NO: 78)
AGCTGGCAATTCTTTCTTGA (AON41)

In this embodiment, the antisense oligonucleotide has a length of 20 to 50 nucleotides and comprises the base sequence SEQ ID NO:78. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:78 is 5-methylcytosine. In another embodiment, the C of the oligonucleotide comprising SEQ ID NO:78 present in the gap of said oligonucleotide (C at position 7 and position 12 of SEQ ID NO:78) are not methylated. In another embodiment, all Cs of the oligonucleotide comprising SEQ ID NO:78 are 5-methylcytosines.

In an embodiment, the oligonucleotide comprising SEQ ID NO:78 has at least one of its internucleoside linkages which is modified, preferably as a phosphorothioate or phosphoramidate (preferably mesyl-phosphoramidate). More preferably, the oligonucleotide comprising SEQ ID NO: 78 has 1, 2 or 3 phosphosdiesters linkage in each of its wings, preferably 3 phosphodiesters linkage in each of its wings. In an embodiment, the oligonucleotide comprising SEQ ID NO:78 is fully modified with a PS backbone.

In an embodiment one wing or preferably each of the wings of the oligonucleotide comprising SEQ ID NO:78 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:78 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

A most preferred oligonucleotide comprising SEQ ID NO:78 is represented by SEQ ID NO:79:

(SEQ ID NO: 79)
eAₛeGₒeC*ₛeTₒeGₛdGₛdCₛdAₛdAₛdTₛdTₛdCₛdTₛdTₛdTₛeC*ₒeTₛe
TₒeGₛeA

In an embodiment the antisense oligonucleotide comprises the following base sequence:

(SEQ ID NO: 80)
GAAGAGCTGGCAATTCTTTC (AON42)

In this embodiment, the antisense oligonucleotide has a length of 20 to 50 nucleotides and comprises the base sequence SEQ ID NO:80. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:80 is 5-methylcytosine. In another embodiment, the C of the oligonucleotide comprising SEQ ID NO:80 present in the gap of said oligonucleotide (C at position 7 and position 11 of SEQ ID NO:80) are not methylated. In another embodiment, all Cs of the oligonucleotide comprising SEQ ID NO:80 are 5-methylcytosines.

In an embodiment, the oligonucleotide comprising SEQ ID NO:80 has at least one of its internucleoside linkages which is modified, preferably as a phosphorothioate or phosphoramidate (preferably mesyl-phosphoramidate). More preferably, the oligonucleotide comprising SEQ ID NO: 80 has 1, 2 or 3 phosphosdiesters linkage in each of its wings, preferably 3 phosphodiesters linkage in each of its wings. In an embodiment, the oligonucleotide comprising SEQ ID NO:80 is fully modified with a PS backbone.

In an embodiment one wing or preferably each of the wings of the oligonucleotide comprising SEQ ID NO:80 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:80 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

A most preferred oligonucleotide comprising SEQ ID NO:80 is represented by SEQ ID NO:81:

(SEQ ID NO: 81)
eGₛeAₒeAₒeGₒeAₛdGₛdCₛdTₛdGₛdGₛdCₛdAₛdAₛdTₛdTₛeC*ₒeTₒeTₒ
eTₛeC*

In an embodiment the antisense oligonucleotide comprises the following base sequence:

(SEQ ID NO: 82)
GAAGAGCTGGCAATTCTTTC (AON43)

In this embodiment, the antisense oligonucleotide has a length of 20 to 50 nucleotides and comprises the base sequence SEQ ID NO:82. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:82 is 5-methylcytosine. In another embodiment, the C of the oligonucleotide comprising SEQ ID NO:82 present in the gap of said oligonucleotide (C at position 7 and position 11 of SEQ ID NO:82) are not methylated. In another embodiment, all Cs of the oligonucleotide comprising SEQ ID NO:82 are 5-methylcytosines.

In an embodiment, the oligonucleotide comprising SEQ ID NO:82 has at least one of its internucleoside linkages which is modified, preferably as a phosphorothioate or phosphoramidate (preferably mesyl-phosphoramidate). More preferably, the oligonucleotide comprising SEQ ID NO: 82 has 1, 2 or 3 phosphosdiesters linkage in each of its wings, preferably 3 phosphodiesters linkage in each of its wings. In an embodiment, the oligonucleotide comprising SEQ ID NO:82 is fully modified with a PS backbone.

In an embodiment, one internucleoside linkage modification of one wing or preferably of each of the wings of the oligonucleotide comprising SEQ ID NO:82 is a dmi-Phosphoramidate (PNdmi) linkage. Preferably, one wing or each of the wings of the oligonucleotide comprising SEQ ID NO: 82 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 82 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:82 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:82 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:82 is represented by SEQ ID NO:83:

(SEQ ID NO: 83)
eGₛeA_{PNdmi}eA_{PNdmi}eG_{PNdmi}eAₛdGₛdCₛdTₛdGₛdGₛdCₛdAₛdAₛdTₛdTₛ
eC*_{PNdmi}eT_{PNdmi}eT_{PNdmi}eTₛeC*

In an embodiment the antisense oligonucleotide comprises the following base sequence:

(SEQ ID NO: 84)
TGGCAATUCTTTCTTGAAAA (AON44)

In this embodiment, the antisense oligonucleotide has a length of 20 to 50 nucleotides and comprises the base sequence SEQ ID NO:84. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:84 is 5-methylcytosine. In another embodiment, the C of the oligonucleotide comprising SEQ ID NO:84 present in the gap of said oligonucleotide (C at position 9 and position 13 of SEQ ID NO:84) are not methylated. In another embodiment, all Cs of the oligonucleotide comprising SEQ ID NO:84 are 5-methylcytosines.

In an embodiment, the oligonucleotide comprising SEQ ID NO:84 has at least one of its internucleoside linkages which is modified, preferably as a phosphorothioate or phosphoramidate (preferably mesyl-phosphoramidate). More preferably, the oligonucleotide comprising SEQ ID NO: 84 has 1, 2 or 3 phosphosdiesters linkage in each of its wings, preferably 3 phosphodiesters linkage in each of its wings. In an embodiment, the oligonucleotide comprising SEQ ID NO:84 is fully modified with a PS backbone.

In an embodiment one wing or preferably each of the wings of the oligonucleotide comprising SEQ ID NO:84 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:84 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, at least one internucleoside linkages in one wing (or in both wings) is not modified and is thus a phosphodiester internucleoside linkage. A preferred internucleoside linkage modification in one wing (or in both wings) is phosphorothioate A most preferred oligonucleotide comprising SEQ ID NO:84 is represented by SEQ ID NO:85:

(SEQ ID NO: 85)
eTₛeGₒeGₒeC*ₒeAₛdAₛdTₛdUₛdCₛdTₛdTₛdTₛdCₛdTₛdTₛeGₒeAₒeAₒ
eAₛeA

In an embodiment the antisense oligonucleotide comprises the following base sequence:

(SEQ ID NO: 86)
GCAATUCTTTCTTGAAAATG (AON45)

In this embodiment, the antisense oligonucleotide has a length of 20 to 50 nucleotides and comprises the base sequence SEQ ID NO:86. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:86 is 5-methylcytosine. In another embodiment, the C of the oligonucleotide comprising SEQ ID NO:86 present in the gap of said oligonucleotide (C at position 7 and position 11 of SEQ ID NO:86) are not methylated. In another embodiment, all Cs of the oligonucleotide comprising SEQ ID NO:86 are 5-methylcytosines.

In an embodiment, the oligonucleotide comprising SEQ ID NO:86 has at least one of its internucleoside linkages which is modified, preferably as a phosphorothioate or phosphoramidate (preferably mesyl-phosphoramidate). More preferably, the oligonucleotide comprising SEQ ID NO: 86 has 1, 2 or 3 phosphosdiesters linkage in each of its wings, preferably 3 phosphodiesters linkage in each of its wings. In an embodiment, the oligonucleotide comprising SEQ ID NO:86 is fully modified with a PS backbone.

In an embodiment, one internucleoside linkage modification of one wing or preferably of each of the wings of the oligonucleotide comprising SEQ ID NO:86 is a dmi-Phosphoramidate (PNdmi) linkage. Preferably, one wing or each of the wings of the oligonucleotide comprising SEQ ID NO: 86 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 86 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:86 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:86 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:86 is represented by SEQ ID NO:87:

(SEQ ID NO: 87)
eGₛeC*_{PNdmi}eAₒeA_{PNdmi}eTₛdUₛdCₛdTₛdTₛdTₛdCₛdTₛdTₛdGₛdAₛeAₒe
AₒeAₒeT_{PNdmi}eG

In an embodiment the antisense oligonucleotide comprises the following base sequence:

(SEQ ID NO: 88)
GGCAATUCTTTCTTGAAAAT (AON46)

In this embodiment, the antisense oligonucleotide has a length of 20 to 50 nucleotides and comprises the base sequence SEQ ID NO:88. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:88 is 5-methylcytosine. In another embodiment, the C of the oligonucleotide comprising SEQ ID NO:88 present in the gap of said oligonucleotide (C at position 8 and position 12 of SEQ ID NO:88) are not methylated. In another embodiment, all Cs of the oligonucleotide comprising SEQ ID NO:88 are 5-methylcytosines.

In an embodiment, the oligonucleotide comprising SEQ ID NO:88 has at least one of its internucleoside linkages which is modified, preferably as a phosphorothioate or phosphoramidate (preferably mesyl-phosphoramidate). More preferably, the oligonucleotide comprising SEQ ID NO: 88 has 1, 2 or 3 phosphosdiesters linkage in each of its wings, preferably 3 phosphodiesters linkage in each of its wings. In an embodiment, the oligonucleotide comprising SEQ ID NO:88 is fully modified with a PS backbone.

In an embodiment, one internucleoside linkage modification of one wing or preferably of each of the wings of the oligonucleotide comprising SEQ ID NO:88 is a dmi-Phosphoramidate (PNdmi) linkage. Preferably, one wing or each of the wings of the oligonucleotide comprising SEQ ID NO: 88 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 88 comprises two distinct modified internucleoside linkages, preferably dmi-Phosphoramidate and phosphorothioate.

In an embodiment, the oligonucleotide hasthree distinct internucleoside linkages, preferably phosphodiester, dmi-Phosphoramidate and phosphorothioate.

In an embodiment one wing or preferably each of the wings of the oligonucleotide comprising SEQ ID NO:88 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:88 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:88 is represented by SEQ ID NO:89:

(SEQ ID NO: 89)
eGₛeG_{PNdmi}eC*ₒeA_{PNdmi}eAₛdTₛdUₛdCₛdTₛdTₛdTₛdCₛdTₛdTₛdGₛeAₒ
eAₒeAₒeA_{PNdmi}eT

In an embodiment the antisense oligonucleotide comprises the following base sequence:

(SEQ ID NO: 90)
TGGCAATUCTTTCTTGAAAA (AON47)

In this embodiment, the antisense oligonucleotide has a length of 20 to 50 nucleotides and comprises the base sequence SEQ ID NO:90. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:90 is 5-methylcytosine. In another embodiment, the C of the oligonucleotide comprising SEQ ID NO:90 present in the gap of said oligonucleotide (C at position 9 and position 13 of SEQ ID NO:90) are not methylated. In another embodiment, all Cs of the oligonucleotide comprising SEQ ID NO:90 are 5-methylcytosines.

In an embodiment, the oligonucleotide comprising SEQ ID NO:90 has at least one of its internucleoside linkages which is modified, preferably as a phosphorothioate or phosphoramidate (preferably mesyl-phosphoramidate). More preferably, the oligonucleotide comprising SEQ ID NO: 90 has 1, 2 or 3 phosphosdiesters linkage in each of its wings, preferably 3 phosphodiesters linkage in each of its wings. In an embodiment, the oligonucleotide comprising SEQ ID NO:90 is fully modified with a PS backbone.

In an embodiment, one internucleoside linkage modification of one wing or preferably of each of the wings of the oligonucleotide comprising SEQ ID NO:90 is a dmi-Phosphoramidate (PNdmi) linkage. Preferably, one wing or each of the wings of the oligonucleotide comprising SEQ ID NO: 90 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 90 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:90 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:90 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:90 is represented by SEQ ID NO:91:

(SEQ ID NO: 91)
eTₛeG_{PNdmi}eGₒeC*_{PNdmi}eAₛdAₛdTₛdUₛdCₛdTₛdTₛdTₛdCₛdTₛdTₛeGₒ
eAₒeAₒeA_{PNdmi}eA

In an embodiment the antisense oligonucleotide comprises the following base sequence:

(SEQ ID NO: 92)
GGCAATUCTTTCTTGAAA (AON48)

In this embodiment, the antisense oligonucleotide has a length of 18 to 50 nucleotides and comprises the base sequence SEQ ID NO:92. The length may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:92 is 5-methylcytosine. In another embodiment, the C of the oligonucleotide comprising SEQ ID NO:92 present in the gap of said oligonucleotide (C at position 8 and position 12 of SEQ ID NO:92) are not methylated. In another embodiment, all Cs of the oligonucleotide comprising SEQ ID NO:92 are 5-methylcytosines.

In an embodiment, the oligonucleotide comprising SEQ ID NO:92 has at least one of its internucleoside linkages which is modified, preferably as a phosphorothioate or phosphoramidate (preferably mesyl-phosphoramidate). More preferably, the oligonucleotide comprising SEQ ID NO: 92 has 1, 2 or 3 phosphosdiesters linkage in each of its wings, preferably 3 phosphodiesters linkage in each of its wings. In an embodiment, the oligonucleotide comprising SEQ ID NO:92 is fully modified with a PS backbone.

In an embodiment one wing or preferably each of the wings of the oligonucleotide comprising SEQ ID NO:92 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:92 comprises 1, 2, 3, 4 or 5 nucleotides. In a more preferred embodiment, one wing or even more preferably both wings comprise 4 nucleotides. Most preferably both wings comprise 4 nucleotides and are MOE.

A most preferred oligonucleotide comprising SEQ ID NO:92 is represented by SEQ ID NO:93:

(SEQ ID NO: 93)
eGₛeGₛeC*ₛeAₛdAₛdTₛdUₛdCₛdTₛdTₛdTₛdCₛdTₛdTₛeGₛeAₛeAₛeA

In an embodiment the antisense oligonucleotide comprises the following base sequence:

(SEQ ID NO: 94)
GGCAATUCTTTCTTGAAA (AON49)

In this embodiment, the antisense oligonucleotide has a length of 18 to 50 nucleotides and comprises the base sequence SEQ ID NO:94. The length may be 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:94 is 5-methylcytosine. In another embodiment, the C of the oligonucleotide comprising SEQ ID NO:94 present in the gap of said oligonucleotide (C at position 8 and position 12 of SEQ ID NO:94) are not methylated. In another embodiment, all Cs of the oligonucleotide comprising SEQ ID NO:94 are 5-methylcytosines.

In an embodiment, the oligonucleotide comprising SEQ ID NO:94 has at least one of its internucleoside linkages which is modified, preferably as a phosphorothioate or phosphoramidate (preferably mesyl-phosphoramidate). More preferably, the oligonucleotide comprising SEQ ID NO: 94 has 1, 2 or 3 phosphosdiesters linkage in each of its wings, preferably 3 phosphodiesters linkage in each of its wings. In an embodiment, =the oligonucleotide comprising SEQ ID NO:94 is fully modified with a PS backbone.

In an embodiment, one internucleoside linkage modification of one wing or preferably of each of the wings of the oligonucleotide comprising SEQ ID NO:94 is a dmi-Phosphoramidate (PNdmi) linkage. Preferably, one wing or each of the wings of the oligonucleotide comprising SEQ ID NO: 94 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 94 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioate an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:94 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:94 comprises 1, 2, 3, 4 or 5 nucleotides. In a more preferred embodiment, one wing or even more preferably both wings comprise 4 nucleotides. Most preferably both wings comprise 4 nucleotides and are MOE.

A most preferred oligonucleotide comprising SEQ ID NO:94 is represented by SEQ ID NO:95:

(SEQ ID NO: 95)
eGₛeG_{PNdmi}eC*ₒeAₛdAₛdTₛdUₛdCₛdTₛdTₛdTₛdCₛdTₛdTₛeGₒeAₒe
A_{PNdmi}eA

In an embodiment the antisense oligonucleotide comprises the following base sequence:

(SEQ ID NO: 96)
GCAATUCTTTCTTGAAAATG (AON50)

In this embodiment, the antisense oligonucleotide has a length of 20 to 50 nucleotides and comprises the base sequence SEQ ID NO:96. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:96 is 5-methylcytosine. In another embodiment, the C of the oligonucleotide comprising SEQ ID NO:96 present in the gap of said oligonucleotide (C at position 7 and position 11 of SEQ ID NO:96) are not methylated. In another embodiment, and preferably all Cs of the oligonucleotide comprising SEQ ID NO:96 are 5-methylcytosines.

In an embodiment, the oligonucleotide comprising SEQ ID NO:96 has at least one of its internucleoside linkages which is modified, preferably as a PNms linkage, preferably at least two of them. In an embodiment, a PNms linkage is present in the central region of the oligonucleotide and not in its wings. In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO:96 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:96 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:96 is represented by SEQ ID NO:97:

(SEQ ID NO: 97)
eGₛeC*ₒeAₒeAₒeTₛdU_{PNms}dC_{PNms}dTₛdTₛdTₛdCₛdTₛdTₛdGₛdAₛeAₒ
eAₒeAₒeTₛeG

In an embodiment the antisense oligonucleotide comprises the following base sequence:

(SEQ ID NO: 98)
GCAATUCTTTCTTGAAAATG (AON51)

In this embodiment, the antisense oligonucleotide has a length of 20 to 50 nucleotides and comprises the base sequence SEQ ID NO:98. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:98 is 5-methylcytosine. In another embodiment, the C of the oligonucleotide comprising SEQ ID NO:98 present in the gap of said oligonucleotide (C at position 7 and position 11 of SEQ ID NO:98) are not methylated. In another embodiment, all Cs of the oligonucleotide comprising SEQ ID NO:98 are 5-methylcytosines.

In an embodiment, the oligonucleotide comprising SEQ ID NO:98 has at least one of its internucleoside linkages which is modified, preferably as a PNms linkage, preferably at least two of them. In an embodiment, a PNms linkage is present in the central region of the oligonucleotide and not in its wings.

In an embodiment, at least one internucleoside linkages in one wing (or in both wings) is not modified and is thus a phosphodiester internucleoside linkage. A preferred internucleoside linkage modification in one wing (or in both wings) is phosphorothioate.

In an embodiment, one wing or preferably each wing of the oligonucleotide comprising SEQ ID NO: 98 comprises one modified internucleoside linkage, preferably phosphorothioate.

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO:98 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:98 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:98 is represented by SEQ ID NO:99:

(SEQ ID NO: 99)
eGₛeC*ₒeAₒeAₒeTₛdUₛdC_{PNms}dT_{PNms}dTₛdTₛdCₛdTₛdTₛdGₛdAₛeAₒe
AₒeAₒeTₛeG

In an embodiment the antisense oligonucleotide comprises the following base sequence:

(SEQ ID NO: 100)
GCAATUCTTTCTTGAAAATG (AON52)

In this embodiment, the antisense oligonucleotide has a length of 20 to 50 nucleotides and comprises the base sequence SEQ ID NO: 100. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:100 is 5-methylcytosine. In another embodiment, the C of the oligonucleotide comprising SEQ ID NO: 100 present in the gap of said oligonucleotide (C at position 7 and position 11 of SEQ ID NO: 100) are not methylated. In another embodiment, all Cs of the oligonucleotide comprising SEQ ID NO: 100 are 5-methylcytosines.

In an embodiment, the oligonucleotide comprising SEQ ID NO: 100 has at least one of its internucleoside linkages which is modified, preferably as a phosphorothioate or phosphoramidate (preferably mesyl-phosphoramidate). More preferably, the oligonucleotide comprising SEQ ID NO: 100 has 1, 2 or 3 phosphosdiesters linkage in each of its wings, preferably 3 phosphodiesters linkage in each of its wings. In an embodiment, the oligonucleotide comprising SEQ ID NO: 100 is fully modified with a PS backbone.

In an embodiment, one internucleoside linkage modification of one wing or preferably of each of the wings or in the central part of the oligonucleotide comprising SEQ ID NO: 100 is a mesyl-phosphoramidate (PNms) linkage. Preferably, one wing or each of the wings of the oligonucleotide comprising SEQ ID NO:100 comprises 1 or 2 or 3 or 4 or 5 PNms linkages, more preferably 2 or 3 or 4, most preferably 2. Preferably, the central part of the oligonucleotide comprising SEQ ID NO: 100 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 100 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 100 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 100 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:100 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 100 is represented by SEQ ID NO: 101:

(SEQ ID NO: 101)
eGₛeC*_{PNms}eA_{PNms}eAₒeTₛdUₛdC_{PNms}dT_{PNms}dTₛdTₛdCₛdTₛdTₛdGₛdAₛ
eAₒeAₒeAₒeTₛeG

In an embodiment the antisense oligonucleotide comprises the following base sequence:

(SEQ ID NO: 102)
GCAATUCTTTCTTGAAAATG (AON53)

In this embodiment, the antisense oligonucleotide has a length of 20 to 50 nucleotides and comprises the base sequence SEQ ID NO: 102. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:102 is 5-methylcytosine. In another embodiment, the C of the oligonucleotide comprising SEQ ID NO: 102 present in the gap of said oligonucleotide (C at position 7 and position 11 of SEQ ID NO: 102) are not methylated. In another embodiment, all Cs of the oligonucleotide comprising SEQ ID NO: 102 are 5-methylcytosines.

In an embodiment, the oligonucleotide comprising SEQ ID NO:102 has at least one of its internucleoside linkages which is modified, preferably as a phosphorothioate or phosphoramidate (preferably mesyl-phosphoramidate). More preferably, the oligonucleotide comprising SEQ ID NO: 102 has 1, 2 or 3 phosphosdiesters linkage in each of its wings, preferably 3 phosphodiesters linkage in each of its wings. In an embodiment, the oligonucleotide comprising SEQ ID NO: 102 is fully modified with a PS backbone.

In an embodiment, the oligonucleotide comprising SEQ ID NO: 102 has at least one of its internucleoside linkages which is modified, preferably as a PNms linkage, preferably at least two of them. In an embodiment, a PNms linkage is present in the central region of the oligonucleotide and not in one wing (or in both wings).

In an embodiment, the oligonucleotide comprising SEQ ID NO:102 has at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 102 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 102 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 102 is represented by SEQ ID NO: 103:

(SEQ ID NO: 103)
eGₛeC*ₒeAₒeAₒeTₛdUₛdC_{PNms}dT_{PNms}dTₛdTₛdCₛdTₛdTₛdGₛdAₛeAₛeAₛ
eAₛeTₛeG

In an embodiment the antisense oligonucleotide comprises the following base sequence:

(SEQ ID NO: 104)
GCAATUCTTTCTTGAAAATG (AON54)

In this embodiment, the antisense oligonucleotide has a length of 20 to 50 nucleotides and comprises the base sequence SEQ ID NO: 104. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:104 is 5-methylcytosine. In another embodiment, the C of the oligonucleotide comprising SEQ ID NO: 104 present in the gap of said oligonucleotide (C at position 7 and position 11 of SEQ ID NO: 104) are not methylated. In another embodiment, all Cs of the oligonucleotide comprising SEQ ID NO: 104 are 5-methylcytosines.

In an embodiment, the oligonucleotide comprising SEQ ID NO:104 has at least one of its internucleoside linkages which is modified, preferably as a phosphorothioate or phosphoramidate (preferably mesyl-phosphoramidate). More preferably, the oligonucleotide comprising SEQ ID NO: 104 has 1, 2 or 3 phosphosdiesters linkage in each of its wings, preferably 3 phosphodiesters linkage in each of its wings. In an embodiment, the oligonucleotide comprising SEQ ID NO: 104 is fully modified with a PS backbone.

In an embodiment, the oligonucleotide comprising SEQ ID NO: 104 has at least one of its internucleoside linkages which is modified, preferably as a PNms linkage, preferably at least 2, 3, 4, 5, of them. In an embodiment, a PNms linkage is present in the central region of the oligonucleotide and not in its wings.

In an embodiment, the oligonucleotide comprising SEQ ID NO:104 has at least one internucleoside linkages in one wing (or in both wings) which is not modified and is thus a phosphodiester internucleoside linkage. A preferred internucleoside linkage modification in one wing (or in both wings) is phosphorothioate

In an embodiment, the oligonucleotide has distinct internucleoside linkages, preferably phosphodiester, mesyl-phosphoramidate and phosphorothioate.

In an embodiment one wing or preferably each of the wings of the oligonucleotide comprising SEQ ID NO: 104 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 104 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:104 is represented by SEQ ID NO: 105:

(SEQ ID NO: 105)
eGₛeC*ₛeAₛeAₛeTₛdUₛdC_{PNms}dT_{PNms}dTₛdTₛdCₛdTₛdTₛdGₛdAₛeAₒe
AₒeAₒeTₛeG

In an embodiment the antisense oligonucleotide comprises the following base sequence:

(SEQ ID NO: 106)
GGCAATUCTTTCTTGAAAAT (AON55)

In this embodiment, the antisense oligonucleotide has a length of 20 to 50 nucleotides and comprises the base sequence SEQ ID NO: 106. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:106 is 5-methylcytosine. In another embodiment, the C of the oligonucleotide comprising SEQ ID NO: 106 present in the gap of said oligonucleotide (C at position 8 and position 12 of SEQ ID NO: 106) are not methylated. In another embodiment, all Cs of the oligonucleotide comprising SEQ ID NO: 106 are 5-methylcytosines.

In an embodiment, the oligonucleotide comprising SEQ ID NO: 106 has at least one of its internucleoside linkages which is modified, preferably as a phosphorothioate or phosphoramidate (preferably mesyl-phosphoramidate). More preferably, the oligonucleotide comprising SEQ ID NO: 106 has 1, 2 or 3 phosphosdiesters linkage in each of its wings, preferably 3 phosphodiesters linkage in each of its wings. In an embodiment, the oligonucleotide comprising SEQ ID NO: 106 is fully modified with a PS backbone.

In an embodiment, the oligonucleotide comprising SEQ ID NO:106 has at least one of its internucleoside linkages which is modified, preferably as a PNms linkage, preferably at least 2, 3, 4, 5 of them. In an embodiment, a PNms linkage is present in the central region of the oligonucleotide and not in one of its wings (or not in both wings).

In an embodiment, the oligonucleotide comprising SEQ ID NO:106 has at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 106 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 106 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 106 is represented by SEQ ID NO: 107:

(SEQ ID NO: 107)
eGₛeGₒeC*ₒeAₒeAₛdTₛdU_{PNms}dCP_{Nms}dTₛdTₛdTₛdCₛdTₛdTₛdGₛeAₒe
AₒeAₒeAₛeT

In an embodiment the antisense oligonucleotide comprises the following base sequence:

(SEQ ID NO: 108)
GGCAATUCTTTCTTGAAAAT (AON56)

In this embodiment, the antisense oligonucleotide has a length of 20 to 50 nucleotides and comprises the base sequence SEQ ID NO: 108. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 108 is 5-methylcytosine. In another embodiment, the C of the oligonucleotide comprising SEQ ID NO: 108 present in the gap of said oligonucleotide (C at position 8 and position 12 of SEQ ID NO: 108) are not methylated. In another embodiment, all Cs of the oligonucleotide comprising SEQ ID NO: 108 are 5-methylcytosines.

In an embodiment, the oligonucleotide comprising SEQ ID NO: 108 has at least one of its internucleoside linkages which is modified, preferably as a phosphorothioate or phosphoramidate (preferably mesyl-phosphoramidate). More preferably, the oligonucleotide comprising SEQ ID NO: 108 has 1, 2 or 3 phosphosdiesters linkage in each of its wings, preferably 3 phosphodiesters linkage in each of its wings. In an embodiment, the oligonucleotide comprising SEQ ID NO: 108 is fully modified with a PS backbone.

In an embodiment, one internucleoside linkage modification of one wing or preferably of each of the wings or in the central part of the oligonucleotide comprising SEQ ID NO: 108 is a mesyl-phosphoramidate (PNms) linkage. Preferably, one wing or each of the wings of the oligonucleotide comprising SEQ ID NO: 108 comprises 1 or 2 or 3 or 4 or 5 PNms linkages, more preferably 2 or 3 or 4, most preferably 2. Preferably, the central part of the oligonucleotide comprising SEQ ID NO: 108 comprises 1 or 2 or 3 or 4 or 5 PNms linkages, more preferably 2 or 3 or 4, most preferably 2. In an embodiment, one wing or preferably each wing of the oligonucleotide comprising SEQ ID NO: 108 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 108 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 108 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 108 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 108 is represented by SEQ ID NO: 109:

(SEQ ID NO: 109)
eGₛeG_{PNms}eC*_{PNms}eAₒeAₛdTₛdUₛdC_{PNms}dT_{PNms}dTₛdTₛdCₛdTₛdTₛdGₛe
AₒeAₒeAₒeAₛeT

In an embodiment the antisense oligonucleotide comprises the following base sequence:

(SEQ ID NO: 110)
GGCAATUCTTTCTTGAAAAT (AON57)

In this embodiment, the antisense oligonucleotide has a length of 20 to 50 nucleotides and comprises the base sequence SEQ ID NO: 110. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:110 is 5-methylcytosine. In another embodiment, the C of the oligonucleotide comprising SEQ ID NO: 110 present in the gap of said oligonucleotide (C at position 8 and position 12 of SEQ ID NO: 110) are not methylated. In another embodiment, all Cs of the oligonucleotide comprising SEQ ID NO: 110 are 5-methylcytosines.

In an embodiment, the oligonucleotide comprising SEQ ID NO:110 has at least one of its internucleoside linkages which is modified, preferably as a phosphorothioate or phosphoramidate (preferably mesyl-phosphoramidate). More preferably, the oligonucleotide comprising SEQ ID NO: 110 has 1, 2 or 3 phosphosdiesters linkage in each of its wings, preferably 3 phosphodiesters linkage in each of its wings. In an embodiment, the oligonucleotide comprising SEQ ID NO: 110 is fully modified with a PS backbone.

In an embodiment, one internucleoside linkage modification of one wing or preferably of each of the wings of the oligonucleotide comprising SEQ ID NO: 110 is a mesyl-phosphoramidate (PNms) linkage. Preferably, one wing or each of the wings of the oligonucleotide comprising SEQ ID NO: 110 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 110 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 110 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO:110 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 110 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 110 is represented by SEQ ID NO: 111:

(SEQ ID NO: 111)
eGₛeG_{PNms}eC*_{PNms}eAₒeAₛdTₛdUₛdCₛdTₛdTₛdTₛdCₛdTₛdTₛdGₛeAₒe
AₒeAₒeAₛeT

In an embodiment the antisense oligonucleotide comprises the following base sequence:

(SEQ ID NO: 112)
GGCAATUCTTTCTTGAAAAT (AON58)

In this embodiment, the antisense oligonucleotide has a length of 20 to 50 nucleotides and comprises the base sequence SEQ ID NO: 112. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:112 is 5-methylcytosine. In another embodiment, the C of the oligonucleotide comprising SEQ ID NO: 112 present in the gap of said oligonucleotide (C at position 8 and position 12 of SEQ ID NO: 112) are not methylated. In another embodiment, and preferably all Cs of the oligonucleotide comprising SEQ ID NO:112 are 5-methylcytosines.

In an embodiment, the oligonucleotide comprising SEQ ID NO:112 has at least one of its internucleoside linkages which is modified, preferably as a phosphorothioate or phosphoramidate (preferably mesyl-phosphoramidate). More preferably, the oligonucleotide comprising SEQ ID NO: 112 has 1, 2 or 3 phosphosdiesters linkage in each of its wings, preferably 3 phosphodiesters linkage in each of its wings. In an embodiment, the oligonucleotide comprising SEQ ID NO: 112 is fully modified with a PS backbone.

In an embodiment, one internucleoside linkage modification of one wing or preferably of each of the wings of the oligonucleotide comprising SEQ ID NO: 112 is a mesyl-phosphoramidate (PNms) linkage. Preferably, one wing or each of the wings of the oligonucleotide comprising SEQ ID NO: 112 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 112 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 112 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 112 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:112 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 112 is represented by SEQ ID NO: 113:

(SEQ ID NO: 113)
eGₛeGₒeC*_{PNms}eA_{PNms}eAₛdTₛdUₛdCₛdTₛdTₛdTₛdCₛdTₛdTₛdGₛeAₒe
AₒeAₒeAₛeT

In an embodiment the antisense oligonucleotide comprises the following base sequence:

(SEQ ID NO: 114)
GGCAATUCTTTCTTGAAAAT (AON59)

In this embodiment, the antisense oligonucleotide has a length of 20 to 50 nucleotides and comprises the base sequence SEQ ID NO: 114. The length may be 20, 21, 22, 23, 24, 25, 26, 27,
In an embodiment, at least one C of the oligonucleotide comprising SEQ ID NO:114 is 5-methylcytosine. In another embodiment, the C of the oligonucleotide comprising SEQ ID NO: 114 present in the gap of said oligonucleotide (C at position 8 and position 12 of SEQ ID NO: 114) are not methylated. In another embodiment, and preferably all Cs of the oligonucleotide comprising SEQ ID NO: 114 are 5-methylcytosines.

In an embodiment, the oligonucleotide comprising SEQ ID NO: 114 has at least one of its internucleoside linkages which is modified, preferably as a phosphorothioate or phosphoramidate (preferably mesyl-phosphoramidate). More preferably, the oligonucleotide comprising SEQ ID NO: 114 has 1, 2 or 3 phosphosdiesters linkage in each of its wings, preferably 3 phosphodiesters linkage in each of its wings. In an embodiment, the oligonucleotide comprising SEQ ID NO: 114 is fully modified with a PS backbone.

In an embodiment, one internucleoside linkage modification of one wing or preferably of each of the wings of the oligonucleotide comprising SEQ ID NO: 114 is a mesyl-phosphoramidate (PNms) linkage. Preferably, one wing or each of the wings of the oligonucleotide comprising SEQ ID NO: 114 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 114 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 114 and no PNms linkage is present in the wings of said oligonucleotide.

In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO:114 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:114 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 114 is represented by SEQ ID NO: 115:

(SEQ ID NO: 115)
eGₛeGₒeC*ₒeAₒeAₛdTₛdUₛdCₛdTₛdTₛdTₛdCₛdTₛdTₛdGₛeAₒeAₒe
A_{PNms}eA_{PNms}eT

In an embodiment the antisense oligonucleotide comprises the following base sequence:

(SEQ ID NO: 116)
GGCAATUCTTTCTTGAAAAT (AON60)

In this embodiment, the antisense oligonucleotide has a length of 20 to 50 nucleotides and comprises the base sequence SEQ ID NO: 116. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:116 is 5-methylcytosine. In another embodiment, the C of the oligonucleotide comprising SEQ ID NO: 116 present in the gap of said oligonucleotide (C at position 8 and position 12 of SEQ ID NO: 116) are not methylated. In another embodiment, all Cs of the oligonucleotide comprising SEQ ID NO: 116 are 5-methylcytosines.

In an embodiment, the oligonucleotide comprising SEQ ID NO:116 has at least one of its internucleoside linkages which is modified, preferably as a phosphorothioate or phosphoramidate (preferably mesyl-phosphoramidate). More preferably, the oligonucleotide comprising SEQ ID NO: 116 has 1, 2 or 3 phosphosdiesters linkage in each of its wings, preferably 3 phosphodiesters linkage in each of its wings. In an embodiment, the oligonucleotide comprising SEQ ID NO: 116 is fully modified with a PS backbone.

In an embodiment, one internucleoside linkage modification of one wing or preferably of each of the wings of the oligonucleotide comprising SEQ ID NO: 116 is a mesyl-phosphoramidate (PNms) linkage. Preferably, one wing or each of the wings of the oligonucleotide comprising SEQ ID NO: 116 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 116 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 116 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO:116 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:116 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:116 is represented by SEQ ID NO: 117:

(SEQ ID NO: 117)
eGₛeGₒeC*ₒeAₒeAₛdTₛdUₛdCₛdTₛdTₛdTₛdCₛdTₛdTₛdGₛeAₒeA_{PNms}e
A_{PNms}eAₒeT.

In an embodiment the antisense oligonucleotide comprises the following base sequence:

(SEQ ID NO: 121)
GCAATUCTTTCTTGAAAATG (AON61)

In this embodiment, the antisense oligonucleotide has a length of 20 to 50 nucleotides and comprises the base sequence SEQ ID NO: 121. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 121 is 5-methylcytosine. In another embodiment, the C of the oligonucleotide comprising SEQ ID NO: 121 present in the gap of said oligonucleotide (C at position 7 and position 11 of SEQ ID NO: 121) are not methylated. In another embodiment, all Cs of the oligonucleotide comprising SEQ ID NO: 121 are 5-methylcytosines.

In an embodiment, the oligonucleotide comprising SEQ ID NO:121 has at least one of its internucleoside linkages which is modified, preferably as a phosphorothioate or phosphoramidate (preferably mesyl-phosphoramidate). More preferably, the oligonucleotide comprising SEQ ID NO: 121 has 1, 2 or 3 phosphosdiesters linkage in each of its wings, preferably 3 phosphodiesters linkage in each of its wings. In an embodiment, the oligonucleotide comprising SEQ ID NO: 121 is fully modified with a PS backbone.

In an embodiment, in the gap of the oligonucleotide comprising SEQ ID NO: 121, at least one sugar modification is present, preferably at least one 2-Omethyl sugar modification is present in the gap of said oligonucleotide. Most preferably, at position 7 of SEQ ID NO: 121, a 2-Omethyl sugar modification is present.

In an embodiment one wing or preferably each of the wings of the oligonucleotide comprising SEQ ID NO: 121 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 121 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:121 is represented by SEQ ID NO: 122:

(SEQ ID NO: 122)
eGₛeC*ₒeAₒeAₒeTₛdUₛmCₛdTₛdTₛdTₛdCₛdTₛ
dTₛdGₛdAₛeAₒeAₒeAₒeTₛeG

In an embodiment the antisense oligonucleotide comprises the following base sequence:

(SEQ ID NO: 123)
GGCAATUCTTTCTTGAAAAT
(AON62)

In this embodiment, the antisense oligonucleotide has a length of 20 to 50 nucleotides and comprises the base sequence SEQ ID NO: 123. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 123 is 5-methylcytosine. In another embodiment, the C of the oligonucleotide comprising SEQ ID NO: 123 present in the gap of said oligonucleotide (C at position 8 and position 12 of SEQ ID NO: 123) are not methylated. In another embodiment, all Cs of the oligonucleotide comprising SEQ ID NO: 123 are 5-methylcytosines.

In an embodiment, the oligonucleotide comprising SEQ ID NO: 123 has at least one of its internucleoside linkages which is modified, preferably as a phosphorothioate or phosphoramidate (preferably mesyl-phosphoramidate). More preferably, the oligonucleotide comprising SEQ ID NO: 123 has 1, 2 or 3 phosphosdiesters linkage in each of its wings, preferably 3 phosphodiesters linkage in each of its wings. In an embodiment, the oligonucleotide comprising SEQ ID NO: 123 is fully modified with a PS backbone.

In an embodiment, in the gap of the oligonucleotide comprising SEQ ID NO:123, at least one sugar modification is present, preferably at least one 2-Omethyl sugar modification is present in the gap of said oligonucleotide. Most preferably, at position 7 of SEQ ID NO: 123, a 2-Omethyl sugar modification is present.

In an embodiment one wing or preferably each of the wings of the oligonucleotide comprising SEQ ID NO:123 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 123 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 123 is represented by SEQ ID NO: 124:

(SEQ ID NO: 124)
eGₛeGₒeC*ₒeAₒeAₛdTₛmUₛdCₛdTₛdTₛdTₛdCₛdTₛ
dTₛdGₛeAₒeAₒeAₒeAₛeT

In an embodiment the antisense oligonucleotide comprises the following base sequence:

(SEQ ID NO: 125)
GCAATUCTTTCTTGAAAATG
(AON63)

In this embodiment, the antisense oligonucleotide has a length of 20 to 50 nucleotides and comprises the base sequence SEQ ID NO: 125. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 125 is 5-methylcytosine. In another embodiment, the C of the oligonucleotide comprising SEQ ID NO: 125 present in the gap of said oligonucleotide (C at position 7 and position 11 of SEQ ID NO: 125) are not methylated. In another embodiment, all Cs of the oligonucleotide comprising SEQ ID NO: 125 are 5-methylcytosines.

In an embodiment, the oligonucleotide comprising SEQ ID NO: 125 has at least one of its internucleoside linkages which is modified, preferably as a phosphorothioate or phosphoramidate (preferably mesyl-phosphoramidate). More preferably, the oligonucleotide comprising SEQ ID NO: 125 has 1, 2 or 3 phosphosdiesters linkage in each of its wings, preferably 3 phosphodiesters linkage in each of its wings. In an embodiment, the oligonucleotide comprising SEQ ID NO: 125 is fully modified with a PS backbone.

In an embodiment, one internucleoside linkage modification of one wing or preferably of each of the wings of the oligonucleotide comprising SEQ ID NO: 125 is a dmi-Phosphoramidate (PNdmi) linkage. Preferably, one wing or each of the wings of the oligonucleotide comprising SEQ ID NO: 125 comprises 1 or 2 or 3 or 4 or 5 PNdmi linkages, more preferably 1 or 2 or 3, most preferably 1

In an embodiment, one wing or preferably each wing of the oligonucleotide comprising SEQ ID NO: 125 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:125 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 125 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:125 is represented by SEQ ID NO: 126:

(SEQ ID NO: 126)
eGₛeC*ₒeAₒeAₒeTₛdUₛdCₛdTₛdTₛdTₛdCₛdTₛdTₛ
dGₛdAₛeAₒeAₒeAₒeT_{PNdmi}eG

In an embodiment the antisense oligonucleotide comprises the following base sequence:

(SEQ ID NO: 127)
GGCAATUCTTTCTTGAAAAT(AON64)

In this embodiment, the antisense oligonucleotide has a length of 20 to 50 nucleotides and comprises the base sequence SEQ ID NO: 127. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 127 present in the gap of said oligonucleotide (C at position 8 and position 12 of SEQ ID NO: 127) are not methylated. In another embodiment, all Cs of the oligonucleotide comprising SEQ ID NO: 127 are 5-methylcytosines.

In an embodiment, the oligonucleotide comprising SEQ ID NO:127 has at least one of its internucleoside linkages which is modified, preferably as a phosphorothioate or phosphoramidate (preferably mesyl-phosphoramidate). More preferably, the oligonucleotide comprising SEQ ID NO: 127 has 1, 2 or 3 phosphosdiesters linkage in each of its wings, preferably 3 phosphodiesters linkage in each of its wings. In an embodiment, the oligonucleotide comprising SEQ ID NO: 127 is fully modified with a PS backbone.

In an embodiment, one internucleoside linkage modification of one wing or preferably of each of the wings of the oligonucleotide comprising SEQ ID NO: 127 is a dmi-Phosphoramidate (PNdmi) linkage. Preferably, one wing or each of the wings of the oligonucleotide comprising SEQ ID NO: 127 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 127 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO: 127 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 127 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 127 is represented by SEQ ID NO: 128:

(SEQ ID NO: 128)
eGₛeGₒeC*ₒeAₒeAₛdTₛdUₛdCₛdTₛdTₛdTₛdCₛ
dTₛdTₛdGₛeAₒeAₒeAₒeA_{PNdmi}eT

In an embodiment the antisense oligonucleotide comprises the following base sequence:

(SEQ ID NO: 129)
GCAATUCTTTCTTGAAAATG(AON65)

In this embodiment, the antisense oligonucleotide has a length of 20 to 50 nucleotides and comprises the base sequence SEQ ID NO: 129. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 129 present in the gap of said oligonucleotide (C at position 7 and position 11 of SEQ ID NO: 129) are not methylated. In another embodiment, all Cs of the oligonucleotide comprising SEQ ID NO: 129 are 5-methylcytosines.

In an embodiment, the oligonucleotide comprising SEQ ID NO: 129 has at least one of its internucleoside linkages which is modified, preferably as a phosphorothioate or phosphoramidate (preferably mesyl-phosphoramidate). More preferably, the oligonucleotide comprising SEQ ID NO: 129 has 1, 2 or 3 phosphosdiesters linkage in each of its wings, preferably 3 phosphodiesters linkage in each of its wings. In an embodiment, the oligonucleotide comprising SEQ ID NO: 129 is fully modified with a PS backbone.

In an embodiment, one internucleoside linkage modification of one wing or preferably of each of the wings of the oligonucleotide comprising SEQ ID NO: 129 is a mesyl-phosphoramidate (PNms) linkage. Preferably, one wing or each of the wings of the oligonucleotide comprising SEQ ID NO: 129 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 129 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 129 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, in the gap of the oligonucleotide comprising SEQ ID NO:129, at least one sugar modification is present, preferably at least one 2-Omethyl sugar modification is present in the gap of said oligonucleotide. Most preferably, at position 7 of SEQ ID NO: 129, a 2-Omethyl sugar modification is present.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 129 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 129 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:129 is represented by SEQ ID NO: 130:

(SEQ ID NO: 130)
eGₛeC*ₒeAₒeAₒeTₛdU_{PNms}mC_{PNms}dTₛdTₛdTₛdCₛdTₛ
dTₛdGₛdAₛeAₒeAₒeAₒeTₛeG

In an embodiment the antisense oligonucleotide comprises the following base sequence:

(SEQ ID NO: 131)
GCAATUCTTTCTTGAAAATG
(AON66)

In this embodiment, the antisense oligonucleotide has a length of 20 to 50 nucleotides and comprises the base sequence SEQ ID NO: 131. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 131 present in the gap of said oligonucleotide (C at position 7 and position 11 of SEQ ID NO:131) are not methylated. In another embodiment, all Cs of the oligonucleotide comprising SEQ ID NO: 131 are 5-methylcytosines.

In an embodiment, the oligonucleotide comprising SEQ ID NO:131 has at least one of its internucleoside linkages which is modified, preferably as a phosphorothioate or phosphoramidate (preferably mesyl-phosphoramidate). More preferably, the oligonucleotide comprising SEQ ID NO: 131 has 1, 2 or 3 phosphosdiesters linkage in each of its wings, preferably 3 phosphodiesters linkage in each of its wings. In an embodiment, the oligonucleotide comprising SEQ ID NO: 131 is fully modified with a PS backbone.

In an embodiment, one internucleoside linkage modification of one wing or preferably of each of the wings of the oligonucleotide comprising SEQ ID NO: 131 is a mesyl-phosphoramidate (PNms) linkage. Preferably, one wing or each of the wings of the oligonucleotide comprising SEQ ID NO: 131 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 131 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 131 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment, in the gap of the oligonucleotide comprising SEQ ID NO:131, at least one sugar modification is present, preferably at least one 2-Omethyl sugar modification is present in the gap of said oligonucleotide. Most preferably, at position 7 of SEQ ID NO: 131, a 2-Omethyl sugar modification is present.

In an embodiment one wing or preferably each of the wings of the oligonucleotide comprising SEQ ID NO: 131 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 131 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:131 is represented by SEQ ID NO:132:

(SEQ ID NO: 132)
eGₛeC*ₒeAₒeAₒeTₛdUₛmC_{PNms}dT_{PNms}dTₛdTₛdCₛdTₛ
dTₛdGₛdAₛeAₒeAₒeAₒeTₛeG

In an embodiment the antisense oligonucleotide comprises the following base sequence:

(SEQ ID NO: 133)
GGCAATUCTTTCTTGAAAAT
(AON67)

In this embodiment, the antisense oligonucleotide has a length of 20 to 50 nucleotides and comprises the base sequence SEQ ID NO: 133. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:133 is 5-methylcytosine. In another embodiment, the C of the oligonucleotide comprising SEQ ID NO: 133 present in the gap of said oligonucleotide (C at position 8 and position 12 of SEQ ID NO: 133) are not methylated. In another embodiment, all Cs of the oligonucleotide comprising SEQ ID NO: 133 are 5-methylcytosines.

In an embodiment, the oligonucleotide comprising SEQ ID NO: 133 has at least one of its internucleoside linkages which is modified, preferably as a phosphorothioate or phosphoramidate (preferably mesyl-phosphoramidate). More preferably, the oligonucleotide comprising SEQ ID NO: 133 is fully modified with a PS backbone.

In an embodiment, one internucleoside linkage modification of one wing or preferably of each of the wings of the oligonucleotide comprising SEQ ID NO: 133 is a mesyl-phosphoramidate (PNms) linkage. Preferably, one wing or each of the wings of the oligonucleotide comprising SEQ ID NO: 133 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 133 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 133 and no PNms linkage is present in the wings of said oligonucleotide.

In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 133 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:133 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 133 is represented by SEQ ID NO: 134:

(SEQ ID NO: 134)
eGₛeGₒeC*ₒeAₒeAₛdTₛmU_{PNms}dC_{PNms}dTₛdTₛdTₛdCₛ
dTₛdTₛdGₛeAₒeAₒeAₒeAₛeT

In an embodiment the antisense oligonucleotide comprises the following base sequence:

(SEQ ID NO: 135)
GGCAATUCTTTCTTGAAAAT
(AON68)

In this embodiment, the antisense oligonucleotide has a length of 20 to 50 nucleotides and comprises the base sequence SEQ ID NO: 135. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 135 is 5-methylcytosine. In another embodiment, the C of the oligonucleotide comprising SEQ ID NO: 135 present in the gap of said oligonucleotide (C at position 8 and position 12 of SEQ ID NO: 135) are not methylated. In another embodiment, all Cs of the oligonucleotide comprising SEQ ID NO: 135 are 5-methylcytosines.

In an embodiment, the oligonucleotide comprising SEQ ID NO: 135 has at least one of its internucleoside linkages which is modified, preferably as a phosphorothioate or phosphoramidate (preferably mesyl-phosphoramidate). More preferably, the oligonucleotide comprising SEQ ID NO: 135 has 1, 2 or 3 phosphosdiesters linkage in each of its wings, preferably 3 phosphodiesters linkage in each of its wings. In an embodiment, the oligonucleotide comprising SEQ ID NO:135 is fully modified with a PS backbone.

In an embodiment, one internucleoside linkage modification of one wing or preferably of each of the wings of the oligonucleotide comprising SEQ ID NO: 135 is a mesyl-phosphoramidate (PNms) linkage. Preferably, one wing or each of the wings of the oligonucleotide comprising SEQ ID NO: 135 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 135 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 135 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 135 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 135 comprises 1, 2, 3, 4 or 5 nucleotides. In a more preferred embodiment, one wing or even more preferably both wings comprise 5 nucleotides. Most preferably both wings comprise 5 nucleotides and are MOE.

In an embodiment, in the gap of the oligonucleotide comprising SEQ ID NO:135, at least one sugar modification is present, preferably at least one 2-Omethyl sugar modification is present in the gap of said oligonucleotide. Most preferably, at position 7 of SEQ ID NO: 135, a 2-Omethyl sugar modification is present.

A most preferred oligonucleotide comprising SEQ ID NO:135 is represented by SEQ ID NO: 136:

(SEQ ID NO: 136)
eGₛeGₒeC*ₒeAₒeAₛdTₛmUₛdC_{PNms}dT_{PNms}dTₛdTₛ
dCₛdTₛdTₛdGₛeAₒeAₒeAₒeAₛeT

In an embodiment the antisense oligonucleotide comprises the following base sequence:

(SEQ ID NO: 137)
GCAATUCTTTCTTGAAAATG (AON69)

In this embodiment, the antisense oligonucleotide has a length of 20 to 50 nucleotides and comprises the base sequence SEQ ID NO: 137. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:137 is 5-methylcytosine. In another embodiment, the C of the oligonucleotide comprising SEQ ID NO: 137 present in the gap of said oligonucleotide (C at position 7 and position 11 of SEQ ID NO: 137) are not methylated. In another embodiment, all Cs of the oligonucleotide comprising SEQ ID NO: 137 are 5-methylcytosines.

In an embodiment, the oligonucleotide comprising SEQ ID NO: 137 has at least one of its internucleoside linkages which is modified, preferably as a phosphorothioate or phosphoramidate (preferably mesyl-phosphoramidate). More preferably, the oligonucleotide comprising SEQ ID NO: 137 has 1, 2 or 3 phosphosdiesters linkage in each of its wings, preferably 3 phosphodiesters linkage in each of its wings. In an embodiment, the oligonucleotide comprising SEQ ID NO: 137 is fully modified with a PS backbone.

In an embodiment, one internucleoside linkage modification of one wing or preferably of each of the wings of the oligonucleotide comprising SEQ ID NO: 137 is a mesyl-phosphoramidate (PNms) linkage. Preferably, one wing or each of the wings of the oligonucleotide comprising SEQ ID NO: 137 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 137 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 137 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, one internucleoside linkage modification of one wing or preferably of each of the wings of the oligonucleotide comprising SEQ ID NO: 137 is a dmi-Phosphoramidate (PNdmi) linkage. Preferably, one wing or each of the wings of the oligonucleotide comprising SEQ ID NO: 137 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 137 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment one wing or preferably each of the wings of the oligonucleotide comprising SEQ ID NO: 137 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 137 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:137 is represented by SEQ ID NO: 138:

(SEQ ID NO: 138)
eGₛeC*ₒeAₒeAₒeTₛdU_{PNms}dC_{PNms}dTₛdTₛdTₛdCₛdTₛdTₛdGₛdAₛeAₒe
AₒeAₒeT_{PNdmi}eG

In an embodiment the antisense oligonucleotide comprises the following base sequence:

(SEQ ID NO: 139)
GCAATUCTTTCTTGAAAATG (AON70)

In this embodiment, the antisense oligonucleotide has a length of 20 to 50 nucleotides and comprises the base sequence SEQ ID NO: 139. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 139 is 5-methylcytosine. In another embodiment, the C of the oligonucleotide comprising SEQ ID NO: 139 present in the gap of said oligonucleotide (C at position 7 and position 11 of SEQ ID NO: 139) are not methylated. In another embodiment, all Cs of the oligonucleotide comprising SEQ ID NO: 139 are 5-methylcytosines.

In an embodiment, the oligonucleotide comprising SEQ ID NO:139 has at least one of its internucleoside linkages which is modified, preferably as a phosphorothioate or phosphoramidate (preferably mesyl-phosphoramidate). More preferably, the oligonucleotide comprising SEQ ID NO: 139 has 1, 2 or 3 phosphosdiesters linkage in each of its wings, preferably 3 phosphodiesters linkage in each of its wings. In an embodiment, the oligonucleotide comprising SEQ ID NO: 139 has 1, 2 or 3 phosphosdiesters linkage in each of its wings, preferably 3 phosphodiesters linkage in each of its wings. In an embodiment, the oligonucleotide comprising SEQ ID NO: 139 is fully modified with a PS backbone.

In an embodiment, one internucleoside linkage modification of one wing or preferably of each of the wings of the oligonucleotide comprising SEQ ID NO: 139 is a mesyl-phosphoramidate (PNms) linkage. Preferably, one wing or each of the wings of the oligonucleotide comprising SEQ ID NO: 139 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 139 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 139 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment, one internucleoside linkage modification of one wing or preferably of each of the wings of the oligonucleotide comprising SEQ ID NO: 139 is a dmi-Phosphoramidate (PNdmi) linkage. Preferably, one wing or each of the wings of the oligonucleotide comprising SEQ ID NO: 139 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 139 comprises two distinct modified internucleoside linkages, preferably dmi-Phosphoramidate and phosphorothioate.

In an embodiment one wing or preferably each of the wings of the oligonucleotide comprising SEQ ID NO: 139 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 139 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 139 is represented by SEQ ID NO: 140:

(SEQ ID NO: 140)
eGₛeC*ₒeAₒeAₒeTₛdUₛdC_{PNms}dT_{PNms}dTₛdTₛdCₛdTₛdTₛdGₛdAₛeAₒe
AₒeAₒeT_{PNdmi}eG

In an embodiment the antisense oligonucleotide comprises the following base sequence:

(SEQ ID NO: 141)
GGCAATUCTTTCTTGAAAAT (AON71)

In this embodiment, the antisense oligonucleotide has a length of 20 to 50 nucleotides and comprises the base sequence SEQ ID NO: 141. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 141 is 5-methylcytosine. In another embodiment, the C of the oligonucleotide comprising SEQ ID NO: 141 present in the gap of said oligonucleotide (C at position 8 and position 12 of SEQ ID NO: 141) are not methylated. In another embodiment, all Cs of the oligonucleotide comprising SEQ ID NO: 141 are 5-methylcytosines.

In an embodiment, one internucleoside linkage modification of one wing or preferably of each of the wings of the oligonucleotide comprising SEQ ID NO: 141 is a mesyl-phosphoramidate (PNms) linkage. Preferably, one wing or each of the wings of the oligonucleotide comprising SEQ ID NO: 141 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 141 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 141 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment, one internucleoside linkage modification of one wing or preferably of each of the wings of the oligonucleotide comprising SEQ ID NO: 141 is a dmi-Phosphoramidate (PNdmi) linkage. Preferably, one wing or each of the wings of the oligonucleotide comprising SEQ ID NO: 141 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 141 comprises two distinct modified internucleoside linkages, preferably dmi-Phosphoramidate and phosphorothioate.

In an embodiment, the oligonucleotide comprising SEQ ID NO:141 has at least one of its internucleoside linkages which is modified, preferably as a phosphorothioate or phosphoramidate (preferably mesyl-phosphoramidate). More preferably, the oligonucleotide comprising SEQ ID NO: 141 has 1, 2 or 3 phosphosdiesters linkage in each of its wings, preferably 3 phosphodiesters linkage in each of its wings. In an embodiment, the oligonucleotide comprising SEQ ID NO: 141 has 1, 2 or 3 phosphosdiesters linkage in each of its wings, preferably 3 phosphodiesters linkage in each of its wings. In an embodiment, the oligonucleotide comprising SEQ ID NO: 141 is fully modified with a PS backbone.

In an embodiment one wing or preferably each of the wings of the oligonucleotide comprising SEQ ID NO: 141 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 141 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:141 is represented by SEQ ID NO: 142:

(SEQ ID NO: 142)
eGₛeGₒeC*ₒeAₒeAₛdTₛdU_{PNms}dC_{PNms}dTₛdTₛdTₛdCₛdTₛdTₛdGₛeAₒe
AₒeAₒeA_{PNdmi}eT

In an embodiment the antisense oligonucleotide comprises the following base sequence:

(SEQ ID NO: 143)
GGCAATUCTTTCTTGAAAAT (AON72)

In this embodiment, the antisense oligonucleotide has a length of 20 to 50 nucleotides and comprises the base sequence SEQ ID NO: 143. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:143 is 5-methylcytosine. In another embodiment, the C of the oligonucleotide comprising SEQ ID NO: 143 present in the gap of said oligonucleotide (C at position 8 and position 12 of SEQ ID NO: 143) are not methylated. In another embodiment, all Cs of the oligonucleotide comprising SEQ ID NO: 143 are 5-methylcytosines.

In an embodiment, one internucleoside linkage modification of one wing or preferably of each of the wings of the oligonucleotide comprising SEQ ID NO: 143 is a mesyl-phosphoramidate (PNms) linkage. Preferably, one wing or each of the wings of the oligonucleotide comprising SEQ ID NO: 143 comprises 1 or 2 or 3 or 4 or 5 PNms linkages, more preferably 2 or 3 or 4, most preferably 2.

In an embodiment, one wing or preferably each wing of the oligonucleotide comprising SEQ ID NO: 143 comprises two distinct modified internucleoside linkages, preferably mesyl-phosphoramidate and phosphorothioate.

In an embodiment, 1, 2, 3, 4, 5 PNms linkages are present in the central part of the oligonucleotide comprising SEQ ID NO: 143 and no PNms linkage is present in the wings of said oligonucleotide. In this embodiment, the internucleoside linkage in the wings of said oligonucleotide are chosen from phophodiesters linkage, mesyl-phosphoramidate and phosphorothioate.

In an embodiment, one internucleoside linkage modification of one wing or preferably of each of the wings of the oligonucleotide comprising SEQ ID NO: 143 is a dmi-Phosphoramidate (PNdmi) linkage. Preferably, one wing or each of the wings of the oligonucleotide comprising SEQ ID NO: 143 comprises 1 or 2 or 3 or 4 or 5 PNdmi linkages, more preferably 1 or 2 or 3, most preferably 1.

In an embodiment, one wing or preferably each wing of the oligonucleotide comprising SEQ ID NO: 143 comprises two distinct modified internucleoside linkages, preferably dmi-Phosphoramidate and phosphorothioate.

In an embodiment, at least one internucleoside linkages in one wing (or in both wings) is not modified and is thus a phosphodiester internucleoside linkage. A preferred internucleoside linkage modification in one wing (or in both wings) is phosphorothioateln an embodiment, the oligonucleotide has three distinct internucleoside linkages, preferably phosphodiester, mesyl-phosphoramidate and phosphorothioate.

In an embodiment, the oligonucleotide comprising SEQ ID NO:143 has at least one of its internucleoside linkages which is modified, preferably as a phosphorothioate or phosphoramidate (preferably mesyl-phosphoramidate). More preferably, the oligonucleotide comprising SEQ ID NO: 143 has 1, 2 or 3 phosphosdiesters linkage in each of its wings, preferably 3 phosphodiesters linkage in each of its wings. In an embodiment, the oligonucleotide comprising SEQ ID NO: 143 is fully modified with a PS backbone.

In an embodiment one wing or preferably each of the wings of the oligonucleotide comprising SEQ ID NO: 143 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 143 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 143 is represented by SEQ ID NO: 144:

(SEQ ID NO: 144)
eGₛeGₒeC*ₒeAₒeAₛdTₛdUₛdC_{PNms}dT_{PNms}dTₛdTₛdCₛdTₛdTₛdGₛeAₒe
AₒeAₒeA_{PNdmi}eT

In an embodiment the antisense oligonucleotide comprises the following base sequence:

(SEQ ID NO: 173)
GTAAATCATTCCCACCACAC (AON73)

In this embodiment, the antisense oligonucleotide has a length of 20 to 50 nucleotides and comprises the base sequence SEQ ID NO: 173. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 173 is 5-methylcytosine. In another embodiment, the C of the oligonucleotide comprising SEQ ID NO: 173 present in the gap of said oligonucleotide (C at positions 7, 11, 12, 13, 15 of SEQ ID NO: 173) are not methylated. In another embodiment, all Cs of the oligonucleotide comprising SEQ ID NO: 173 are 5-methylcytosines.

In an embodiment, the oligonucleotide comprising SEQ ID NO: 173 has at least one of its internucleoside linkages which is modified, preferably as a phosphorothioate or phosphoramidate (preferably mesyl-phosphoramidate). More preferably, the oligonucleotide comprising SEQ ID NO: 173 is fully modified with a PS backbone.

In an embodiment one wing or preferably each of the wings of the oligonucleotide comprising SEQ ID NO: 173 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 173 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 173 is represented by SEQ ID NO: 174:

(SEQ ID NO: 174)
eGₛeTₛeAₛeAₛeAₛdTₛdCₛdAₛdTₛdTₛdCₛdCₛdCₛdAₛdCₛeC*ₛeAₛe
C*ₛeAₛeC*.

In an embodiment the antisense oligonucleotide comprises the following base sequence:

(SEQ ID NO: 175)
ATGTAAATCATTCCCACCAC (AON74)

In this embodiment, the antisense oligonucleotide has a length of 20 to 50 nucleotides and comprises the base sequence SEQ ID NO: 175. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 175 is 5-methylcytosine. In another embodiment, the C of the oligonucleotide comprising SEQ ID NO: 175 present in the gap of said oligonucleotide (C at positions 9, 13-15 of SEQ ID NO: 175) are not methylated. In another embodiment, all Cs of the oligonucleotide comprising SEQ ID NO: 175 are 5-methylcytosines.

In an embodiment, the oligonucleotide comprising SEQ ID NO: 175 has at least one of its internucleoside linkages which is modified, preferably as a phosphorothioate or phosphoramidate (preferably mesyl-phosphoramidate). More preferably, the oligonucleotide comprising SEQ ID NO: 175 is fully modified with a PS backbone.

In an embodiment one wing or preferably each of the wings of the oligonucleotide comprising SEQ ID NO:175 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 175 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 175 is represented by SEQ ID NO: 176:

(SEQ ID NO: 176)
eAₛeTₛeGₛeTₛeAₛdAₛdAₛdTₛdCₛdAₛdTₛdTₛdCₛdCₛdCₛeAₛeC*ₛe
C*ₛeAₛeC*.

In an embodiment the antisense oligonucleotide comprises the following base sequence:

(SEQ ID NO: 177)
GAATGTAAATCATTCCCACC (AON75)

In this embodiment, the antisense oligonucleotide has a length of 20 to 50 nucleotides and comprises the base sequence SEQ ID NO: 177. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 177 is 5-methylcytosine. In another embodiment, the C of the oligonucleotide comprising SEQ ID NO: 177 present in the gap of said oligonucleotide (C at position 11 and position 15 of SEQ ID NO: 177) are not methylated. In another embodiment, all Cs of the oligonucleotide comprising SEQ ID NO: 177 are 5-methylcytosines.

In an embodiment, the oligonucleotide comprising SEQ ID NO: 177 has at least one of its internucleoside linkages which is modified, preferably as a phosphorothioate or phosphoramidate (preferably mesyl-phosphoramidate). More preferably, the oligonucleotide comprising SEQ ID NO: 177 is fully modified with a PS backbone.

In an embodiment one wing or preferably each of the wings of the oligonucleotide comprising SEQ ID NO: 177 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 177 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 177 is represented by SEQ ID NO: 178:

(SEQ ID NO: 178)
eGₛeAₛeAₛeTₛeGₛdTₛdAₛdAₛdAₛdTₛdCₛdAₛdTₛdTₛdCₛeC*ₛeC*ₛe
AₛeC*ₛeC*

In an embodiment the antisense oligonucleotide comprises the following base sequence:

(SEQ ID NO: 179)
GTGAATGTAAATCATTCCCA (AON76).

In this embodiment, the antisense oligonucleotide has a length of 20 to 50 nucleotides and comprises the base sequence SEQ ID NO: 179. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 179 is 5-methylcytosine. In another embodiment, the C of the oligonucleotide comprising SEQ ID NO: 179 present in the gap of said oligonucleotide (C at position 13 of SEQ ID NO:179) are not methylated. In another embodiment, all Cs of the oligonucleotide comprising SEQ ID NO: 179 are 5-methylcytosines.

In an embodiment, the oligonucleotide comprising SEQ ID NO: 179 has at least one of its internucleoside linkages which is modified, preferably as a phosphorothioate or phosphoramidate (preferably mesyl-phosphoramidate). More preferably, the oligonucleotide comprising SEQ ID NO: 179 is fully modified with a PS backbone.

In an embodiment one wing or preferably each of the wings of the oligonucleotide comprising SEQ ID NO: 179 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 179 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 179 is represented by SEQ ID NO: 180:

(SEQ ID NO: 180)
eGₛeTₛeGₛeAₛeAₛdTₛdGₛdTₛdAₛdAₛdAₛdTₛdCₛdAₛdTₛeTₛe
C*ₛeC*ₛeC*ₛeA

In an embodiment the antisense oligonucleotide comprises the following base sequence:

(SEQ ID NO: 181)
CAGTGAATGTAAATCATTCC(AON77).

In this embodiment, the antisense oligonucleotide has a length of 20 to 50 nucleotides and comprises the base sequence SEQ ID NO: 181. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:181 is 5-methylcytosine. In another embodiment, the C of the oligonucleotide comprising SEQ ID NO: 181 present in the gap of said oligonucleotide (C at position 15 of SEQ ID NO: 181) are not methylated. In another embodiment, all Cs of the oligonucleotide comprising SEQ ID NO:181 are 5-methylcytosines.

In an embodiment, the oligonucleotide comprising SEQ ID NO: 181 has at least one of its internucleoside linkages which is modified, preferably as a phosphorothioate or phosphoramidate (preferably mesyl-phosphoramidate). More preferably, the oligonucleotide comprising SEQ ID NO: 181 is fully modified with a PS backbone.

In an embodiment one wing or preferably each of the wings of the oligonucleotide comprising SEQ ID NO:181 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 181 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:181 is represented by SEQ ID NO: 182:

(SEQ ID NO: 182)
eC*ₛeAₛeGₛeTₛeGₛdAₛdAₛdTₛdGₛdTₛdAₛdAₛdAₛdTₛdCₛeAₛe
TₛeTₛeC*ₛeC*

In an embodiment the antisense oligonucleotide comprises the following base sequence:

(SEQ ID NO: 183)
TCCAGTGAATGTAAATCATT(AON78).

In this embodiment, the antisense oligonucleotide has a length of 20 to 50 nucleotides and comprises the base sequence SEQ ID NO: 183. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 183 is 5-methylcytosine. In another embodiment, all Cs of the oligonucleotide comprising SEQ ID NO: 183 are 5-methylcytosines.

In an embodiment, the oligonucleotide comprising SEQ ID NO: 183 has at least one of its internucleoside linkages which is modified, preferably as a phosphorothioate or phosphoramidate (preferably mesyl-phosphoramidate). More preferably, the oligonucleotide comprising SEQ ID NO: 183 is fully modified with a PS backbone.

In an embodiment one wing or preferably each of the wings of the oligonucleotide comprising SEQ ID NO: 183 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 183 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 183 is represented by SEQ ID NO: 184:

(SEQ ID NO: 184)
eTₛeC*ₛeC*ₛeAₛeGₛdTₛdGₛdAₛdAₛdTₛdGₛdTₛdAₛdAₛdAₛe
TₛeC*ₛeAₛeTₛeT

In an embodiment the antisense oligonucleotide comprises the following base sequence:

(SEQ ID NO: 185)
TTTCCAGTGAATGTAAATCA(AON79).

In this embodiment, the antisense oligonucleotide has a length of 20 to 50 nucleotides and comprises the base sequence SEQ ID NO: 185. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 185 is 5-methylcytosine. In another embodiment, all Cs of the oligonucleotide comprising SEQ ID NO: 185 are 5-methylcytosines.

In an embodiment, the oligonucleotide comprising SEQ ID NO:185 has at least one of its internucleoside linkages which is modified, preferably as a phosphorothioate or phosphoramidate (preferably mesyl-phosphoramidate). More preferably, the oligonucleotide comprising SEQ ID NO: 185 is fully modified with a PS backbone.

In an embodiment one wing or preferably each of the wings of the oligonucleotide comprising SEQ ID NO: 185 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 185 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 185 is represented by SEQ ID NO: 186:

(SEQ ID NO: 186)
eTₛeTₛeTₛeC*ₛeC*ₛdAₛdGₛdTₛdGₛdAₛdAₛdTₛdGₛdTₛdAₛe
AₛeAₛeTₛeC*ₛeA

In an embodiment the antisense oligonucleotide comprises the following base sequence:

(SEQ ID NO: 187)
GUAAATCATTCCCACCACAC(AON 80).

In this embodiment, the antisense oligonucleotide has a length of 20 to 50 nucleotides and comprises the base sequence SEQ ID NO: 187. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 187 is 5-methylcytosine. In another embodiment, the C of the oligonucleotide comprising SEQ ID NO: 187 present in the gap of said oligonucleotide (C at positions 7, 11-13, and 15 of SEQ ID NO: 187) are not methylated. In another embodiment, all Cs of the oligonucleotide comprising SEQ ID NO: 187 are 5-methylcytosines.

In an embodiment, the oligonucleotide comprising SEQ ID NO: 187 has at least one of its internucleoside linkages which is modified, preferably as a phosphorothioate or phosphoramidate (preferably mesyl-phosphoramidate). More preferably, the oligonucleotide comprising SEQ ID NO: 187 is fully modified with a PS backbone.

In an embodiment one wing or preferably each of the wings of the oligonucleotide comprising SEQ ID NO: 187 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 187 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:187 is represented by SEQ ID NO: 188:

(SEQ ID NO: 188)
eGₛeUₛeAₛeAₛeAₛdTₛdCₛdAₛdTₛdTₛdCₛdCₛdCₛdAₛdCₛeC*ₛe
AₛeC*ₛeAₛeC*

In an embodiment the antisense oligonucleotide comprises the following base sequence:

(SEQ ID NO: 189)
ATGUAAATCATTCCCACCAC(AON 81.

In this embodiment, the antisense oligonucleotide has a length of 20 to 50 nucleotides and comprises the base sequence SEQ ID NO: 189. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:189 is 5-methylcytosine. In another embodiment, the C of the oligonucleotide comprising SEQ ID NO: 189 present in the gap of said oligonucleotide (C at positions 9, 13-15, of SEQ ID NO: 189) are not methylated. In another embodiment, all Cs of the oligonucleotide comprising SEQ ID NO: 189 are 5-methylcytosines.

In an embodiment, one wing or preferably each wing of the oligonucleotide comprising SEQ ID NO: 189 comprises one distinct modified internucleoside linkages, preferably phosphorothioate.

In an embodiment, the oligonucleotide comprising SEQ ID NO: 189 has at least one of its internucleoside linkages which is modified, preferably as a phosphorothioate or phosphoramidate (preferably mesyl-phosphoramidate). More preferably, the oligonucleotide comprising SEQ ID NO: 189 is fully modified with a PS backbone.

In an embodiment one wing or preferably each of the wings of the oligonucleotide comprising SEQ ID NO: 189 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:189 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 189 is represented by SEQ ID NO: 190:

(SEQ ID NO: 190)
eAₛeTₛeGₛeUₛeAₛdAₛdAₛdTₛdCₛdAₛdTₛdTₛdCₛdCₛdCₛeAₛeC
*ₛeC*ₛeAₛeC*

In an embodiment the antisense oligonucleotide comprises the following base sequence:

(SEQ ID NO: 191)
GAATGUAAATCATTCCCACC(AON 82.

In this embodiment, the antisense oligonucleotide has a length of 20 to 50 nucleotides and comprises the base sequence SEQ ID NO: 191. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 191 is 5-methylcytosine. In another embodiment, the C of the oligonucleotide comprising SEQ ID NO: 191 present in the gap of said oligonucleotide (C at position 11 and at position 15 of SEQ ID NO: 191) are not methylated. In another embodiment, all Cs of the oligonucleotide comprising SEQ ID NO: 191 are 5-methylcytosines.

In an embodiment, one wing or preferably each wing of the oligonucleotide comprising SEQ ID NO: 191 comprises one distinct modified internucleoside linkages, preferably phosphorothioate. In an embodiment, the oligonucleotide comprising SEQ ID NO: 191 has at least one of its internucleoside linkages which is modified, preferably as a phosphorothioate or phosphoramidate (preferably mesyl-phosphoramidate). More preferably, the oligonucleotide comprising SEQ ID NO: 191 is fully modified with a PS backbone.

In an embodiment one wing or preferably each of the wings of the oligonucleotide comprising SEQ ID NO: 191 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 191 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 191 is represented by SEQ ID NO: 192:

(SEQ ID NO: 192)
eGₛeAₛeAₛeTₛeGₛdUₛdAₛdAₛdAₛdTₛdCₛdAₛdTₛdTₛdCₛeC*ₛe
C*ₛeAₛeC*ₛeC*

In an embodiment the antisense oligonucleotide comprises the following base sequence:

(SEQ ID NO: 193)
GTGAATGUAAATCATTCCCA(AON83.

In this embodiment, the antisense oligonucleotide has a length of 20 to 50 nucleotides and comprises the base sequence SEQ ID NO: 193. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 193 is 5-methylcytosine. In another embodiment, the C of the oligonucleotide comprising SEQ ID NO: 193 present in the gap of said oligonucleotide (C at position 13 of SEQ ID NO: 193) are not methylated. In another embodiment, all Cs of the oligonucleotide comprising SEQ ID NO:193 are 5-methylcytosines.

In an embodiment, one wing or preferably each wing of the oligonucleotide comprising SEQ ID NO: 193 comprises one distinct modified internucleoside linkages, preferably phosphorothioate. In an embodiment, the oligonucleotide comprising SEQ ID NO:193 has at least one of its internucleoside linkages which is modified, preferably as a phosphorothioate or phosphoramidate (preferably mesyl-phosphoramidate). More preferably, the oligonucleotide comprising SEQ ID NO: 193 is fully modified with a PS backbone.

In an embodiment one wing or preferably each of the wings of the oligonucleotide comprising SEQ ID NO: 193 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:193 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 193 is represented by SEQ ID NO: 194:

(SEQ ID NO: 194)
eGₛeTₛeGₛeAₛeAₛdTₛdGₛdUₛdAₛdAₛdAₛdTₛdCₛdAₛdTₛeTₛeC
*ₛeC*ₛeC*ₛeA

In an embodiment the antisense oligonucleotide comprises the following base sequence:

(AON 84 (SEQ ID NO: 195)
CAGTGAATGUAAATCATTCC.

In this embodiment, the antisense oligonucleotide has a length of 20 to 50 nucleotides and comprises the base sequence SEQ ID NO: 195. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 195 is 5-methylcytosine. In another embodiment, the C of the oligonucleotide comprising SEQ ID NO: 195 present in the gap of said oligonucleotide (C at position 15 of SEQ ID NO: 195) are not methylated. In another embodiment, all Cs of the oligonucleotide comprising SEQ ID NO:195 are 5-methylcytosines.

In an embodiment, one wing or preferably each wing of the oligonucleotide comprising SEQ ID NO: 195 comprises one distinct modified internucleoside linkages, preferably phosphorothioate. In an embodiment, the oligonucleotide comprising SEQ ID NO:195 has at least one of its internucleoside linkages which is modified, preferably as a phosphorothioate or phosphoramidate (preferably mesyl-phosphoramidate). More preferably, the oligonucleotide comprising SEQ ID NO: 195 is fully modified with a PS backbone.

In an embodiment one wing or preferably each of the wings of the oligonucleotide comprising SEQ ID NO: 195 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 195 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 195 is represented by SEQ ID NO: 196:

(SEQ ID NO: 196)
eC*ₛeAₛeGₛeTₛeGₛdAₛdAₛdTₛdGₛdUₛdAₛdAₛdAₛdTₛdCₛeAₛeTₛe
TₛeC*ₛeC*

In an embodiment the antisense oligonucleotide comprises the following base sequence:

(AON 85 (SEQ ID NO: 197)
TCCAGTGAATGUAAATCATT.

In this embodiment, the antisense oligonucleotide has a length of 20 to 50 nucleotides and comprises the base sequence SEQ ID NO: 197. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 197 is 5-methylcytosine. In another embodiment, all Cs of the oligonucleotide comprising SEQ ID NO: 197 are 5-methylcytosines.

In an embodiment, one wing or preferably each wing of the oligonucleotide comprising SEQ ID NO: 197 comprises one distinct modified internucleoside linkages, preferably phosphorothioate.

In an embodiment, the oligonucleotide comprising SEQ ID NO:197 has at least one of its internucleoside linkages which is modified, preferably as a phosphorothioate or phosphoramidate (preferably mesyl-phosphoramidate). More preferably, the oligonucleotide comprising SEQ ID NO: 197 is fully modified with a PS backbone.

In an embodiment one wing or preferably each of the wings of the oligonucleotide comprising SEQ ID NO: 197 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:197 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:197 is represented by SEQ ID NO: 198:

(SEQ ID NO: 198)
eTₛeC*ₛeC*ₛeAₛeGₛdTₛdGₛdAₛdAₛdTₛdGₛdUₛdAₛdAₛdAₛeTₛeC*ₛ
eAₛeTₛeT

In an embodiment the antisense oligonucleotide comprises the following base sequence:

(AON 86 (SEQ ID NO: 199)
TTTCCAGTGAATGUAAATCA.

In this embodiment, the antisense oligonucleotide has a length of 20 to 50 nucleotides and comprises the base sequence SEQ ID NO: 199. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO: 199 is 5-methylcytosine. In another embodiment, all Cs of the oligonucleotide comprising SEQ ID NO: 199 are 5-methylcytosines.

In an embodiment, one wing or preferably each wing of the oligonucleotide comprising SEQ ID NO: 199 comprises one distinct modified internucleoside linkages, preferably phosphorothioate. In an embodiment, the oligonucleotide comprising SEQ ID NO: 199 has at least one of its internucleoside linkages which is modified, preferably as a phosphorothioate or phosphoramidate (preferably mesyl-phosphoramidate). More preferably, the oligonucleotide comprising SEQ ID NO: 199 is fully modified with a PS backbone.

In an embodiment one wing or preferably each of the wings of the oligonucleotide comprising SEQ ID NO: 199 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 199 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO: 199 is represented by SEQ ID NO:200:

(SEQ ID NO: 200)
eTₛeTₛeTₛeC*ₛeC*ₛdAₛdGₛdTₛdGₛdAₛdAₛdTₛdGₛdUₛdAₛeAₛeAₛe
TₛeC*ₛeA

In an embodiment the antisense oligonucleotide comprises the following base sequence:

(AON 88 (SEQ ID NO: 203)
AGCGCCGGCAATTCTTTCTTG.

In this embodiment, the antisense oligonucleotide has a length of 20 to 50 nucleotides and comprises the base sequence SEQ ID NO:203. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:203 is 5-methylcytosine. In another embodiment, the C of the oligonucleotide comprising SEQ ID NO:203 present in the gap of said oligonucleotide (C at positions 6, 9, 14 of SEQ ID NO:203) are not methylated. In another embodiment, all Cs of the oligonucleotide comprising SEQ ID NO:203 are 5-methylcytosines.

In an embodiment, one wing or preferably each wing of the oligonucleotide comprising SEQ ID NO: 203 comprises one distinct modified internucleoside linkages, preferably phosphorothioate. In an embodiment, the oligonucleotide comprising SEQ ID NO:203 has at least one of its internucleoside linkages which is modified, preferably as a phosphorothioate or phosphoramidate (preferably mesyl-phosphoramidate). More preferably, the oligonucleotide comprising SEQ ID NO: 203 is fully modified with a PS backbone.

In an embodiment one wing or preferably each of the wings of the oligonucleotide comprising SEQ ID NO:203 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:203 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:203 is represented by SEQ ID NO:204:

(SEQ ID NO: 204)
eAₛeGₒeC*ₒeGₒeC*ₛdCₛdGₛdGₛdCₛdAₛdAₛdTₛdTₛdCₛdTₛdTₛeTₒ
eC*ₒeTₒeTₛeG

In an embodiment the antisense oligonucleotide comprises the following base sequence:

(SEQ ID NO: 218)
GCAATUCTTTCTTGAAAATG (AON89)

In this embodiment, the antisense oligonucleotide has a length of 20 to 50 nucleotides and comprises the base sequence SEQ ID NO:218. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:218 is 5-methylcytosine. In another embodiment, the C of the oligonucleotide comprising SEQ ID NO:218 present in the gap of said oligonucleotide (C at position 7 and at position 11 of SEQ ID NO:218) are not methylated. In another embodiment, all Cs of the oligonucleotide comprising SEQ ID NO: 218 are 5-methylcytosines.

In an embodiment, the oligonucleotide comprising SEQ ID NO:218 has at least one of its internucleoside linkages which is modified, preferably as a phosphorothioate or phosphoramidate (preferably mesyl-phosphoramidate). In an embodiment, the oligonucleotide comprising SEQ ID NO: 218 has 1, 2 or 3 phosphosdiester linkages in each of its wings, preferably 3 phosphodiester linkages in each of its wings. In another embodiment, the oligonucleotide comprising SEQ ID NO: 218 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:218 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 218 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:218 is represented by SEQ ID NO:219:

(SEQ ID NO: 219)
eGₛeC*ₒeAₛeAₒeTₛdUₛdCₛdTₛdTₛdTₛdCₛdTₛdTₛdGₛdAₛeAₒeAₛeAₒ
eTₛeG

In an embodiment the antisense oligonucleotide comprises the following base sequence:

(SEQ ID NO: 220)
GGCAATUCTTTCTTGAAAAT (AON90)

In this embodiment, the antisense oligonucleotide has a length of 20 to 50 nucleotides and comprises the base sequence SEQ ID NO:220. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:220 is 5-methylcytosine. In another embodiment, the C of the oligonucleotide comprising SEQ ID NO:220 present in the gap of said oligonucleotide (C at position 8 and at position 12 of SEQ ID NO:220) are not methylated. In another embodiment, all Cs of the oligonucleotide comprising SEQ ID NO: 220 are 5-methylcytosines.

In an embodiment, the oligonucleotide comprising SEQ ID NO:220 has at least one of its internucleoside linkages which is modified, preferably as a phosphorothioate or phosphoramidate (preferably mesyl-phosphoramidate). In an embodiment, the oligonucleotide comprising SEQ ID NO: 220 has 1, 2 or 3 phosphosdiester linkages in each of its wings, preferably 3 phosphodiester linkages in each of its wings. In another embodiment, the oligonucleotide comprising SEQ ID NO: 220 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:220 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 220 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:220 is represented by SEQ ID NO:221:

(SEQ ID NO: 221)
eGₛeGₒeC*ₛeAₒeAₛdTₛdUₛdCₛdTₛdTₛdTₛdCₛdTₛdTₛdGₛeAₒeAₛeAₒ
eAₛeT

In an embodiment the antisense oligonucleotide comprises the following base sequence:

(SEQ ID NO: 222)
TGGCAATUCTTTCTTGAAAA (AON91)

In this embodiment, the antisense oligonucleotide has a length of 20 to 50 nucleotides and comprises the base sequence SEQ ID NO:222. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:222 is 5-methylcytosine. In another embodiment, the C of the oligonucleotide comprising SEQ ID NO:222 present in the gap of said oligonucleotide (C at position 9 and at position 13 of SEQ ID NO:222) are not methylated. In another embodiment, all Cs of the oligonucleotide comprising SEQ ID NO: 222 are 5-methylcytosines.

In an embodiment, the oligonucleotide comprising SEQ ID NO:222 has at least one of its internucleoside linkages which is modified, preferably as a phosphorothioate or phosphoramidate (preferably mesyl-phosphoramidate). In an embodiment, the oligonucleotide comprising SEQ ID NO: 222 has 1, 2 or 3 phosphosdiester linkages in each of its wings, preferably 3 phosphodiester linkages in each of its wings. In another embodiment, the oligonucleotide comprising SEQ ID NO: 222 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:222 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 222 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:222 is represented by SEQ ID NO:223:

(SEQ ID NO: 223)
eTₛeGₒeGₛeC*ₒeAₛdAₛdTₛdUₛdCₛdTₛdTₛdTₛdCₛdTₛdTₛeGₒeAₛeAₒ
eAₛeA

In an embodiment the antisense oligonucleotide comprises the following base sequence:

(SEQ ID NO: 224)
GCAATUCTTTCTTGAAAATG (AON92)

In this embodiment, the antisense oligonucleotide has a length of 20 to 50 nucleotides and comprises the base sequence SEQ ID NO:224. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:224 is 5-methylcytosine. In another embodiment, the C of the oligonucleotide comprising SEQ ID NO:224 present in the gap of said oligonucleotide (C at position 7 and at position 11 of SEQ ID NO:224) are not methylated. In another embodiment, all Cs of the oligonucleotide comprising SEQ ID NO: 224 are 5-methylcytosines.

In an embodiment, the oligonucleotide comprising SEQ ID NO:224 has at least one of its internucleoside linkages which is modified, preferably as a phosphorothioate or phosphoramidate (preferably mesyl-phosphoramidate). In an embodiment, the oligonucleotide comprising SEQ ID NO: 224 has 1, 2 or 3 phosphosdiester linkages in each of its wings, preferably 3 phosphodiester linkages in each of its wings. In another embodiment, the oligonucleotide comprising SEQ ID NO: 224 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:224 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 224 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:224 is represented by SEQ ID NO:225:

(SEQ ID NO: 225)
eGₛeC*ₛeAₛeAₛeTₛdUₛdC*ₛdTₛdTₛdTₛdC*ₛdTₛdTₛdGₛdAₛeAₛeAₛ
eAₛeTₛeG

In an embodiment the antisense oligonucleotide comprises the following base sequence:

(SEQ ID NO: 226)
GGCAATUCTTTCTTGAAAAT (AON93)

In this embodiment, the antisense oligonucleotide has a length of 20 to 50 nucleotides and comprises the base sequence SEQ ID NO:226. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:226 is 5-methylcytosine. In another embodiment, the C of the oligonucleotide comprising SEQ ID NO:226 present in the gap of said oligonucleotide (C at position 8 and at position 12 of SEQ ID NO:226) are not methylated. In another embodiment, all Cs of the oligonucleotide comprising SEQ ID NO: 226 are 5-methylcytosines.

In an embodiment, the oligonucleotide comprising SEQ ID NO:226 has at least one of its internucleoside linkages which is modified, preferably as a phosphorothioate or phosphoramidate (preferably mesyl-phosphoramidate). In an embodiment, the oligonucleotide comprising SEQ ID NO: 226 has 1, 2 or 3 phosphosdiester linkages in each of its wings, preferably 3 phosphodiester linkages in each of its wings. In another embodiment, the oligonucleotide comprising SEQ ID NO: 226 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:226 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 226 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:226 is represented by SEQ ID NO:227:

(SEQ ID NO: 227)
eGₛeGₛeC*ₛeAₛeAₛdTₛdUₛdC*ₛdTₛdTₛdTₛdC*ₛdTₛdTₛdGₛeAₛeAₛ
eAₛeAₛeT

In an embodiment the antisense oligonucleotide comprises the following base sequence:

(SEQ ID NO: 228)
TGGCAATUCTTTCTTGAAAA (AON94)

In this embodiment, the antisense oligonucleotide has a length of 20 to 50 nucleotides and comprises the base sequence SEQ ID NO:228. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:228 is 5-methylcytosine. In another embodiment, the C of the oligonucleotide comprising SEQ ID NO:228 present in the gap of said oligonucleotide (C at position 9 and at position 13 of SEQ ID NO:228) are not methylated. In another embodiment, all Cs of the oligonucleotide comprising SEQ ID NO: 228 are 5-methylcytosines.

In an embodiment, the oligonucleotide comprising SEQ ID NO:228 has at least one of its internucleoside linkages which is modified, preferably as a phosphorothioate or phosphoramidate (preferably mesyl-phosphoramidate). In an embodiment, the oligonucleotide comprising SEQ ID NO: 228 has 1, 2 or 3 phosphosdiester linkages in each of its wings, preferably 3 phosphodiester linkages in each of its wings. In another embodiment, the oligonucleotide comprising SEQ ID NO: 228 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:228 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 228 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:228 is represented by SEQ ID NO:229:

(SEQ ID NO: 229)
eTₛeGₛeGₛeC*ₛeAₛdAₛdTₛdUₛdC*ₛdTₛdTₛdTₛdC*ₛdTₛdTₛeGₛeAₛ
eAₛeAₛeA

In an embodiment the antisense oligonucleotide comprises the following base sequence:

(SEQ ID NO: 230)
GCAATUCTTTCTTGAAAATG (AON95)

In this embodiment, the antisense oligonucleotide has a length of 20 to 50 nucleotides and comprises the base sequence SEQ ID NO:230. The length may be 20, 21, 22, 23, 24, 25, 26, 27,
In an embodiment, at least one C of the oligonucleotide comprising SEQ ID NO:230 is 5-methylcytosine. In another embodiment, the C of the oligonucleotide comprising SEQ ID NO:230 present in the gap of said oligonucleotide (C at position 7 and at position 11 of SEQ ID NO:230) are not methylated. In another embodiment, all Cs of the oligonucleotide comprising SEQ ID NO: 230 are 5-methylcytosines.

In an embodiment, the oligonucleotide comprising SEQ ID NO:230 has at least one of its internucleoside linkages which is modified, preferably as a phosphorothioate or phosphoramidate (preferably mesyl-phosphoramidate). In an embodiment, the oligonucleotide comprising SEQ ID NO: 230 has 1, 2 or 3 phosphosdiester linkages in each of its wings, preferably 3 phosphodiester linkages in each of its wings. In another embodiment, the oligonucleotide comprising SEQ ID NO: 230 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:230 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 230 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:230 is represented by SEQ ID NO:231:

(SEQ ID NO: 231)
eGₛeC*ₒeAₒeAₒeTₛdUₛdC*ₛdTₛdTₛdTₛdC*ₛdTₛdTₛdGₛdAₛeAₒeAₒ
eAₒeTₛeG

In an embodiment the antisense oligonucleotide comprises the following base sequence:

(SEQ ID NO: 232)
GGCAATUCTTTCTTGAAAAT (AON96)

In this embodiment, the antisense oligonucleotide has a length of 20 to 50 nucleotides and comprises the base sequence SEQ ID NO:232. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:232 is 5-methylcytosine. In another embodiment, the C of the oligonucleotide comprising SEQ ID NO:232 present in the gap of said oligonucleotide (C at position 8 and at position 12 of SEQ ID NO:232) are not methylated. In another embodiment, all Cs of the oligonucleotide comprising SEQ ID NO: 232 are 5-methylcytosines.

In an embodiment, the oligonucleotide comprising SEQ ID NO:232 has at least one of its internucleoside linkages which is modified, preferably as a phosphorothioate or phosphoramidate (preferably mesyl-phosphoramidate). In an embodiment, the oligonucleotide comprising SEQ ID NO: 232 has 1, 2 or 3 phosphosdiester linkages in each of its wings, preferably 3 phosphodiester linkages in each of its wings. In another embodiment, the oligonucleotide comprising SEQ ID NO: 232 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:232 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 232 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:232 is represented by SEQ ID NO:233:

(SEQ ID NO: 233)
eGₛeGₒeC*ₒeAₒeAₛdTₛdUₛdC*ₛdTₛdTₛdTₛdC*ₛdTₛdTₛdGₛeAₒeAₒ
eAₒeAₛeT

In an embodiment the antisense oligonucleotide comprises the following base sequence:

(SEQ ID NO: 234)
TGGCAATUCTTTCTTGAAAA (AON97)

In this embodiment, the antisense oligonucleotide has a length of 20 to 50 nucleotides and comprises the base sequence SEQ ID NO:234. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:234 is 5-methylcytosine. In another embodiment, the C of the oligonucleotide comprising SEQ ID NO:234 present in the gap of said oligonucleotide (C at position 9 and at position 13 of SEQ ID NO:234) are not methylated. In another embodiment, all Cs of the oligonucleotide comprising SEQ ID NO: 234 are 5-methylcytosines.

In an embodiment, the oligonucleotide comprising SEQ ID NO:234 has at least one of its internucleoside linkages which is modified, preferably as a phosphorothioate or phosphoramidate (preferably mesyl-phosphoramidate). In an embodiment, the oligonucleotide comprising SEQ ID NO: 234 has 1, 2 or 3 phosphosdiester linkages in each of its wings, preferably 3 phosphodiester linkages in each of its wings. In another embodiment, the oligonucleotide comprising SEQ ID NO: 234 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:234 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 234 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:234 is represented by SEQ ID NO:235:

(SEQ ID NO: 235)
eTₛeGₒeGₒeC*ₒeAₛdAₛdTₛdUₛdC*ₛdTₛdTₛdTₛdC*ₛdTₛdTₛeGₒeAₒ
eAₒeAₛeA

In an embodiment the antisense oligonucleotide comprises the following base sequence:

(SEQ ID NO: 236)
GCAATUCTTTCTTGAAAATG (AON98)

In this embodiment, the antisense oligonucleotide has a length of 20 to 50 nucleotides and comprises the base sequence SEQ ID NO:236. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:236 is 5-methylcytosine. In another embodiment, the C of the oligonucleotide comprising SEQ ID NO:236 present in the gap of said oligonucleotide (C at position 7 and at position 11 of SEQ ID NO:236) are not methylated. In another embodiment, all Cs of the oligonucleotide comprising SEQ ID NO: 236 are 5-methylcytosines.

In an embodiment, the oligonucleotide comprising SEQ ID NO:236 has at least one of its internucleoside linkages which is modified, preferably as a phosphorothioate or phosphoramidate (preferably mesyl-phosphoramidate). In an embodiment, the oligonucleotide comprising SEQ ID NO: 236 has 1, 2 or 3 phosphosdiester linkages in each of its wings, preferably 3 phosphodiester linkages in each of its wings. In another embodiment, the oligonucleotide comprising SEQ ID NO: 236 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:236 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 236 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:236 is represented by SEQ ID NO:237:

(SEQ ID NO: 237)
eGₛeC*ₒeAₛeAₒeTₛdUₛdC*ₛdTₛdTₛdTₛdC*ₛdTₛdTₛdGₛdAₛeAₒeAₛ
eAₒeTₛeG

In an embodiment the antisense oligonucleotide comprises the following base sequence:

(SEQ ID NO: 238)
GGCAATUCTTTCTTGAAAAT (AON99)

In this embodiment, the antisense oligonucleotide has a length of 20 to 50 nucleotides and comprises the base sequence SEQ ID NO:238. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:238 is 5-methylcytosine. In another embodiment, the C of the oligonucleotide comprising SEQ ID NO:238 present in the gap of said oligonucleotide (C at position 8 and at position 12 of SEQ ID NO:238) are not methylated. In another embodiment, all Cs of the oligonucleotide comprising SEQ ID NO: 238 are 5-methylcytosines.

In an embodiment, the oligonucleotide comprising SEQ ID NO:238 has at least one of its internucleoside linkages which is modified, preferably as a phosphorothioate or phosphoramidate (preferably mesyl-phosphoramidate). In an embodiment, the oligonucleotide comprising SEQ ID NO: 238 has 1, 2 or 3 phosphosdiester linkages in each of its wings, preferably 3 phosphodiester linkages in each of its wings. In another embodiment, the oligonucleotide comprising SEQ ID NO: 238 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:238 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 238 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:238 is represented by SEQ ID NO:239:

(SEQ ID NO: 239)
eGₛeGₒeC*ₛeAₒeAₛdTₛdUₛdC*ₛdTₛdTₛdTₛdC*ₛdTₛdTₛdGₛeAₒeAₛ
eAₒeAₛeT

In an embodiment the antisense oligonucleotide comprises the following base sequence:

(SEQ ID NO: 240)
TGGCAATUCTTTCTTGAAAA (AON100)

In this embodiment, the antisense oligonucleotide has a length of 20 to 50 nucleotides and comprises the base sequence SEQ ID NO:240. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:240 is 5-methylcytosine. In another embodiment, the C of the oligonucleotide comprising SEQ ID NO:240 present in the gap of said oligonucleotide (C at position 9 and at position 13 of SEQ ID NO:240) are not methylated. In another embodiment, all Cs of the oligonucleotide comprising SEQ ID NO: 240 are 5-methylcytosines.

In an embodiment, the oligonucleotide comprising SEQ ID NO:240 has at least one of its internucleoside linkages which is modified, preferably as a phosphorothioate or phosphoramidate (preferably mesyl-phosphoramidate). In an embodiment, the oligonucleotide comprising SEQ ID NO: 240 has 1, 2 or 3 phosphosdiester linkages in each of its wings, preferably 3 phosphodiester linkages in each of its wings. In another embodiment, the oligonucleotide comprising SEQ ID NO: 240 is fully modified with a PS backbone.

In an embodiment, at least one internucleoside linkages in one wing (or in both wings) is not modified and is thus a phosphodiester internucleoside linkage. A preferred internucleoside linkage modification in one wing (or in both wings) is phosphorothioate. In an embodiment one wing or preferably each of the wings of the oligonucleotide comprising SEQ ID NO:240 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO: 240 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:240 is represented by SEQ ID NO:241:

(SEQ ID NO: 241)
eTₛeGₒeGₛeC*ₒeAₛdAₛdTₛdUₛdC*ₛdTₛdTₛdTₛdC*ₛdTₛdTₛe
GₒeAₛeAₒeAₛeA

In an embodiment the antisense oligonucleotide comprises the following base sequence:

(SEQ ID NO: 242)
GCAATUCTTTCTTGAAAATG (AON101)

In this embodiment, the antisense oligonucleotide has a length of 20 to 50 nucleotides and comprises the base sequence SEQ ID NO:242. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:242 is 5-methylcytosine. In another embodiment, the C of the oligonucleotide comprising SEQ ID NO:242 present in the gap of said oligonucleotide (C at position 7 and position 11 of SEQ ID NO:242) are not methylated. In another embodiment, all Cs of the oligonucleotide comprising SEQ ID NO:242 are 5-methylcytosines.

In an embodiment, the oligonucleotide comprising SEQ ID NO:242 has at least one of its internucleoside linkages which is modified, preferably as a phosphorothioate or phosphoramidate (preferably mesyl-phosphoramidate). More preferably, the oligonucleotide comprising SEQ ID NO: 242 has 1, 2 or 3 phosphosdiesters linkage in each of its wings, preferably 3 phosphodiesters linkage in each of its wings. In an embodiment, the oligonucleotide comprising SEQ ID NO:242 is fully modified with a PS backbone.

In an embodiment, one internucleoside linkage modification of one wing or preferably of each of the wings of the oligonucleotide comprising SEQ ID NO:242 is a dmi-Phosphoramidate (PNdmi) linkage. Preferably, one wing or each of the wings of the oligonucleotide comprising SEQ ID NO: 242 comprises 1 or 2 or 3 or 4 or 5 PNdmi linkages, more preferably 2 or 3 or 4, most preferably 1.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 242 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:242 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:242 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:242 is represented by SEQ ID NO:243:

(SEQ ID NO: 243)
eG_{PNdmi}eC*ₒeAₛeAₒeTₛdUₛdCₛdTₛdTₛdTₛdCₛdTₛdTₛdGₛdAₛ
eAₒeAₛeAₒeT_{PNdmi}eG

In an embodiment the antisense oligonucleotide comprises the following base sequence:

(SEQ ID NO: 244)
GCAATUCTTTCTTGAAAATG (AON102)

In this embodiment, the antisense oligonucleotide has a length of 20 to 50 nucleotides and comprises the base sequence SEQ ID NO:244. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:244 is 5-methylcytosine. In another embodiment, the C of the oligonucleotide comprising SEQ ID NO:244 present in the gap of said oligonucleotide (C at position 7 and position 11 of SEQ ID NO:244) are not methylated. In another embodiment, all Cs of the oligonucleotide comprising SEQ ID NO:244 are 5-methylcytosines.

In an embodiment, the oligonucleotide comprising SEQ ID NO:244 has at least one of its internucleoside linkages which is modified, preferably as a phosphorothioate or phosphoramidate (preferably mesyl-phosphoramidate). More preferably, the oligonucleotide comprising SEQ ID NO: 244 has 1, 2 or 3 phosphosdiesters linkage in each of its wings, preferably 3 phosphodiesters linkage in each of its wings. In an embodiment, the oligonucleotide comprising SEQ ID NO:244 is fully modified with a PS backbone.

In an embodiment, one internucleoside linkage modification of one wing or preferably of each of the wings of the oligonucleotide comprising SEQ ID NO:244 is a dmi-Phosphoramidate (PNdmi) linkage. Preferably, one wing or each of the wings of the oligonucleotide comprising SEQ ID NO: 244 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 244 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:244 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:244 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:244 is represented by SEQ ID NO:245:

(SEQ ID NO: 245)
eG_{PNdmi}eC*_{PNdmi}eA_{PNdmi}eAₒeTₛdUₛdCₛdTₛdTₛdTₛdCₛdTₛd
TₛdGₛdAₛeAₒeA_{PNdmi}eA_{PNdmi}eT_{PNdmie}G

In an embodiment the antisense oligonucleotide comprises the following base sequence:

(SEQ ID NO: 246)
GCAATUCTTTCTTGAAAATG (AON103)

In this embodiment, the antisense oligonucleotide has a length of 20 to 50 nucleotides and comprises the base sequence SEQ ID NO:246. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:246 is 5-methylcytosine. In another embodiment, the C of the oligonucleotide comprising SEQ ID NO:246 present in the gap of said oligonucleotide (C at position 7 and position 11 of SEQ ID NO:246) are not methylated. In another embodiment, all Cs of the oligonucleotide comprising SEQ ID NO:246 are 5-methylcytosines.

In an embodiment, the oligonucleotide comprising SEQ ID NO:246 has at least one of its internucleoside linkages which is modified, preferably as a phosphorothioate or phosphoramidate (preferably mesyl-phosphoramidate). More preferably, the oligonucleotide comprising SEQ ID NO: 246 has 1, 2 or 3 phosphosdiesters linkage in each of its wings, preferably 3 phosphodiesters linkage in each of its wings. In an embodiment, the oligonucleotide comprising SEQ ID NO:246 is fully modified with a PS backbone.

In an embodiment, one internucleoside linkage modification of one wing or preferably of each of the wings of the oligonucleotide comprising SEQ ID NO:246 is a dmi-Phosphoramidate (PNdmi) linkage. Preferably, one wing or each of the wings of the oligonucleotide comprising SEQ ID NO: 246 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 246 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:246 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:246 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:246 is represented by SEQ ID NO:247:

(SEQ ID NO: 247)
eG_{PNdmie}C*ₒeA_{PNdmi}eAₒeTₛdUₛdCₛdTₛdTₛdTₛdCₛdTₛdTₛd
GₛdAₛeAₒeA_{PNdmi}eAₒeT_{PNdmi}eG

In an embodiment the antisense oligonucleotide comprises the following base sequence:

(SEQ ID NO: 248)
GCAATUCTTTCTTGAAAATG (AON104)

In this embodiment, the antisense oligonucleotide has a length of 20 to 50 nucleotides and comprises the base sequence SEQ ID NO:248. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:248 is 5-methylcytosine. In another embodiment, the C of the oligonucleotide comprising SEQ ID NO:248 present in the gap of said oligonucleotide (C at position 7 and position 11 of SEQ ID NO:248) are not methylated. In another embodiment, all Cs of the oligonucleotide comprising SEQ ID NO:248 are 5-methylcytosines.

In an embodiment, the oligonucleotide comprising SEQ ID NO:248 has at least one of its internucleoside linkages which is modified, preferably as a phosphorothioate or phosphoramidate (preferably mesyl-phosphoramidate). More preferably, the oligonucleotide comprising SEQ ID NO: 248 has 1, 2 or 3 phosphosdiesters linkage in each of its wings, preferably 3 phosphodiesters linkage in each of its wings. In an embodiment, the oligonucleotide comprising SEQ ID NO:248 is fully modified with a PS backbone.

In an embodiment, one internucleoside linkage modification of one wing or preferably of each of the wings of the oligonucleotide comprising SEQ ID NO:248 is a dmi-Phosphoramidate (PNdmi) linkage. Preferably, one wing or each of the wings of the oligonucleotide comprising SEQ ID NO: 248 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 248 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:248 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:248 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:248 is represented by SEQ ID NO:249:

(SEQ ID NO: 249)
eG_{PNdmi}eC*ₒeA_{PNdmi}eAₒeT_{PNdmi}dUₛdCₛdTₛdTₛdTₛdCₛdTₛd
TₛdGₛdA_{PNdmi}eAₒeA_{PNdmi}eAₒeT_{PNdmi}eG

In an embodiment the antisense oligonucleotide comprises the following base sequence:

(SEQ ID NO: 250)
GCAATUCTTTCTTGAAAATG (AON105)

In this embodiment, the antisense oligonucleotide has a length of 20 to 50 nucleotides and comprises the base sequence SEQ ID NO:250. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:250 is 5-methylcytosine. In another embodiment, the C of the oligonucleotide comprising SEQ ID NO:250 present in the gap of said oligonucleotide (C at position 7 and position 11 of SEQ ID NO:250) are not methylated. In another embodiment, all Cs of the oligonucleotide comprising SEQ ID NO:250 are 5-methylcytosines.

In an embodiment, the oligonucleotide comprising SEQ ID NO:250 has at least one of its internucleoside linkages which is modified, preferably as a phosphorothioate or phosphoramidate (preferably mesyl-phosphoramidate). More preferably, the oligonucleotide comprising SEQ ID NO: 250 has 1, 2 or 3 phosphosdiesters linkage in each of its wings, preferably 3 phosphodiesters linkage in each of its wings. In an embodiment, the oligonucleotide comprising SEQ ID NO:250 is fully modified with a PS backbone.

In an embodiment, one internucleoside linkage modification of one wing or preferably of each of the wings of the oligonucleotide comprising SEQ ID NO:250 is a dmi-Phosphoramidate (PNdmi) linkage. Preferably, one wing or each of the wings of the oligonucleotide comprising SEQ ID NO: 250 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 250 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:250 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:250 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:250 is represented by SEQ ID NO:251:

(SEQ ID NO: 251)
eG_{PNdmi}eC*_{PNdmi}eAₛeAₒeTₛdUₛdCₛdTₛdTₛdTₛdCₛdTₛdTₛd
GₛdAₛeAₒeAₛeA_{PNdmi}eT_{PNdmi}eG

In an embodiment the antisense oligonucleotide comprises the following base sequence:

(SEQ ID NO: 252)
GCAATUCTTTCTTGAAAATG (AON106)

In this embodiment, the antisense oligonucleotide has a length of 20 to 50 nucleotides and comprises the base sequence SEQ ID NO:252. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:252 is 5-methylcytosine. In another embodiment, the C of the oligonucleotide comprising SEQ ID NO:252 present in the gap of said oligonucleotide (C at position 7 and position 11 of SEQ ID NO:252) are not methylated. In another embodiment, all Cs of the oligonucleotide comprising SEQ ID NO:252 are 5-methylcytosines.

In an embodiment, the oligonucleotide comprising SEQ ID NO:252 has at least one of its internucleoside linkages which is modified, preferably as a phosphorothioate or phosphoramidate (preferably mesyl-phosphoramidate). More preferably, the oligonucleotide comprising SEQ ID NO: 252 has 1, 2 or 3 phosphosdiesters linkage in each of its wings, preferably 3 phosphodiesters linkage in each of its wings. In an embodiment, the oligonucleotide comprising SEQ ID NO:252 is fully modified with a PS backbone.

In an embodiment, one internucleoside linkage modification of one wing or preferably of each of the wings of the oligonucleotide comprising SEQ ID NO:252 is a dmi-Phosphoramidate (PNdmi) linkage. Preferably, one wing or each of the wings of the oligonucleotide comprising SEQ ID NO: 252 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 252 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:252 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:252 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:252 is represented by SEQ ID NO:253:

(SEQ ID NO: 253)
eG_{PNdmi}eC*_{PNdmi}eAₛeA_{PNdmi}eTₛdUₛdCₛdTₛdTₛdTₛdCₛdTₛ
dTₛdGₛdAₛeA_{PNdmi}eAₛeA_{PNdmi}eT_{PNdmi}eG

In an embodiment the antisense oligonucleotide comprises the following base sequence:

(SEQ ID NO: 254)
GGCAATUCTTTCTTGAAAAT (AON107)

In this embodiment, the antisense oligonucleotide has a length of 20 to 50 nucleotides and comprises the base sequence SEQ ID NO:254. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:254 is 5-methylcytosine. In another embodiment, the C of the oligonucleotide comprising SEQ ID NO:254 present in the gap of said oligonucleotide (C at position 8 and position 12 of SEQ ID NO:254) are not methylated. In another embodiment, all Cs of the oligonucleotide comprising SEQ ID NO:254 are 5-methylcytosines.

In an embodiment, the oligonucleotide comprising SEQ ID NO:254 has at least one of its internucleoside linkages which is modified, preferably as a phosphorothioate or phosphoramidate (preferably mesyl-phosphoramidate). More preferably, the oligonucleotide comprising SEQ ID NO: 254 has 1, 2 or 3 phosphosdiesters linkage in each of its wings, preferably 3 phosphodiesters linkage in each of its wings. In an embodiment, the oligonucleotide comprising SEQ ID NO:254 is fully modified with a PS backbone.

In an embodiment, one internucleoside linkage modification of one wing or preferably of each of the wings of the oligonucleotide comprising SEQ ID NO:254 is a dmi-Phosphoramidate (PNdmi) linkage. Preferably, one wing or each of the wings of the oligonucleotide comprising SEQ ID NO: 254 comprises 1 or 2 or 3 or 4 or 5 PNdmi linkages, more preferably 2 or 3 or 4, most preferably 1.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 254 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:254 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:254 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:254 is represented by SEQ ID NO:255:

(SEQ ID NO: 255)
eG_{PNdmi}eGₒeC*ₛeAₒeAₛdTₛdUₛdCₛdTₛdTₛdTₛdCₛdTₛdTₛdGₛeAₒeAₛ
eAₒeA_{PNdmi}eT

In an embodiment the antisense oligonucleotide comprises the following base sequence:

(SEQ ID NO: 256)
GGCAATUCTTTCTTGAAAAT (AON108)

In this embodiment, the antisense oligonucleotide has a length of 20 to 50 nucleotides and comprises the base sequence SEQ ID NO:256. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:256 is 5-methylcytosine. In another embodiment, the C of the oligonucleotide comprising SEQ ID NO:256 present in the gap of said oligonucleotide (C at position 8 and position 12 of SEQ ID NO:256) are not methylated. In another embodiment, all Cs of the oligonucleotide comprising SEQ ID NO:256 are 5-methylcytosines.

In an embodiment, the oligonucleotide comprising SEQ ID NO:256 has at least one of its internucleoside linkages which is modified, preferably as a phosphorothioate or phosphoramidate (preferably mesyl-phosphoramidate). More preferably, the oligonucleotide comprising SEQ ID NO: 256 has 1, 2 or 3 phosphosdiesters linkage in each of its wings, preferably 3 phosphodiesters linkage in each of its wings. In an embodiment, the oligonucleotide comprising SEQ ID NO:256 is fully modified with a PS backbone.

In an embodiment, one internucleoside linkage modification of one wing or preferably of each of the wings of the oligonucleotide comprising SEQ ID NO:256 is a dmi-Phosphoramidate (PNdmi) linkage. Preferably, one wing or each of the wings of the oligonucleotide comprising SEQ ID NO: 256 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 256 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:256 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:256 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:256 is represented by SEQ ID NO:257:

(SEQ ID NO: 257)
eG_{PNdmi}eG_{PNdmi}eC*_{PNdmi}eAₒeAₛdTₛdUₛdCₛdTₛdTₛdTₛdCₛdTₛdTₛdGₛ
eAₒeA_{PNdmi}eA_{PNdmi}eA_{PNdmi}eT

In an embodiment the antisense oligonucleotide comprises the following base sequence:

(SEQ ID NO: 258)
GGCAATUCTTTCTTGAAAAT (AON109)

In this embodiment, the antisense oligonucleotide has a length of 20 to 50 nucleotides and comprises the base sequence SEQ ID NO:258. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:258 is 5-methylcytosine. In another embodiment, the C of the oligonucleotide comprising SEQ ID NO:258 present in the gap of said oligonucleotide (C at position 8 and position 12 of SEQ ID NO:258) are not methylated. In another embodiment, all Cs of the oligonucleotide comprising SEQ ID NO:258 are 5-methylcytosines.

In an embodiment, the oligonucleotide comprising SEQ ID NO:258 has at least one of its internucleoside linkages which is modified, preferably as a phosphorothioate or phosphoramidate (preferably mesyl-phosphoramidate). More preferably, the oligonucleotide comprising SEQ ID NO: 258 has 1, 2 or 3 phosphosdiesters linkage in each of its wings, preferably 3 phosphodiesters linkage in each of its wings. In an embodiment, the oligonucleotide comprising SEQ ID NO:258 is fully modified with a PS backbone.

In an embodiment, one internucleoside linkage modification of one wing or preferably of each of the wings of the oligonucleotide comprising SEQ ID NO:258 is a dmi-Phosphoramidate (PNdmi) linkage. Preferably, one wing or each of the wings of the oligonucleotide comprising SEQ ID NO: 258 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 258 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:258 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:258 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:258 is represented by SEQ ID NO:259:

(SEQ ID NO: 259)
eG_{PNdmi}eGₒeC*_{PNdmi}eAₒeAₛdTₛdUₛdCₛdTₛdTₛdTₛdCₛdTₛdTₛdGₛeAₒ
eA_{PNdmi}eAₒeA_{PNdmi}eT

In an embodiment the antisense oligonucleotide comprises the following base sequence:

(SEQ ID NO: 260)
GGCAATUCTTTCTTGAAAAT (AON110)

In this embodiment, the antisense oligonucleotide has a length of 20 to 50 nucleotides and comprises the base sequence SEQ ID NO:260. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:260 is 5-methylcytosine. In another embodiment, the C of the oligonucleotide comprising SEQ ID NO:260 present in the gap of said oligonucleotide (C at position 8 and position 12 of SEQ ID NO:260) are not methylated. In another embodiment, all Cs of the oligonucleotide comprising SEQ ID NO:260 are 5-methylcytosines.

In an embodiment, the oligonucleotide comprising SEQ ID NO:260 has at least one of its internucleoside linkages which is modified, preferably as a phosphorothioate or phosphoramidate (preferably mesyl-phosphoramidate). More preferably, the oligonucleotide comprising SEQ ID NO: 260 has 1, 2 or 3 phosphosdiesters linkage in each of its wings, preferably 3 phosphodiesters linkage in each of its wings. In an embodiment, the oligonucleotide comprising SEQ ID NO:260 is fully modified with a PS backbone.

In an embodiment, one internucleoside linkage modification of one wing or preferably of each of the wings of the oligonucleotide comprising SEQ ID NO:260 is a dmi-Phosphoramidate (PNdmi) linkage. Preferably, one wing or each of the wings of the oligonucleotide comprising SEQ ID NO: 260 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 260 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:260 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:260 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:260 is represented by SEQ ID NO:261:

(SEQ ID NO: 261)
eG_{PNdmi}eGₒeC*_{PNdmi}eAₒeA_{PNdmi}dTₛdUₛdCₛdTₛdTₛdTₛdCₛdTₛdTₛ
dG_{PNdmi}eAₒeA_{PNdmi}eAₒeA_{PNdmi}eT

In an embodiment the antisense oligonucleotide comprises the following base sequence:

(SEQ ID NO: 262)
GGCAATUCTTTCTTGAAAAT (AON111)

In this embodiment, the antisense oligonucleotide has a length of 20 to 50 nucleotides and comprises the base sequence SEQ ID NO:262. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:262 is 5-methylcytosine. In another embodiment, the C of the oligonucleotide comprising SEQ ID NO:262 present in the gap of said oligonucleotide (C at position 8 and position 12 of SEQ ID NO:262) are not methylated. In another embodiment, all Cs of the oligonucleotide comprising SEQ ID NO:262 are 5-methylcytosines.

In an embodiment, the oligonucleotide comprising SEQ ID NO:262 has at least one of its internucleoside linkages which is modified, preferably as a phosphorothioate or phosphoramidate (preferably mesyl-phosphoramidate). More preferably, the oligonucleotide comprising SEQ ID NO: 262 has 1, 2 or 3 phosphosdiesters linkage in each of its wings, preferably 3 phosphodiesters linkage in each of its wings. In an embodiment, the oligonucleotide comprising SEQ ID NO:262 is fully modified with a PS backbone.

In an embodiment, one internucleoside linkage modification of one wing or preferably of each of the wings of the oligonucleotide comprising SEQ ID NO:262 is a dmi-Phosphoramidate (PNdmi) linkage. Preferably, one wing or each of the wings of the oligonucleotide comprising SEQ ID NO: 262 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 262 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:262 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:262 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:262 is represented by SEQ ID NO:263:

(SEQ ID NO: 263)
eG_{PNdmi}eG_{PNdmi}eC*ₛeAₒeAₛdTₛdUₛdCₛdTₛdTₛdTₛdCₛdTₛdTₛdGₛeAₒe
AₛeA_{PNdmi}eA_{PNdmi}eT

In an embodiment the antisense oligonucleotide comprises the following base sequence:

(SEQ ID NO: 264)
GGCAATUCTTTCTTGAAAAT (AON112)

In this embodiment, the antisense oligonucleotide has a length of 20 to 50 nucleotides and comprises the base sequence SEQ ID NO:264. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:264 is 5-methylcytosine. In another embodiment, the C of the oligonucleotide comprising SEQ ID NO:264 present in the gap of said oligonucleotide (C at position 8 and position 12 of SEQ ID NO:264) are not methylated. In another embodiment, all Cs of the oligonucleotide comprising SEQ ID NO:264 are 5-methylcytosines.

In an embodiment, the oligonucleotide comprising SEQ ID NO:264 has at least one of its internucleoside linkages which is modified, preferably as a phosphorothioate or phosphoramidate (preferably mesyl-phosphoramidate). More preferably, the oligonucleotide comprising SEQ ID NO: 264 has 1, 2 or 3 phosphosdiesters linkage in each of its wings, preferably 3 phosphodiesters linkage in each of its wings. In an embodiment, the oligonucleotide comprising SEQ ID NO:264 is fully modified with a PS backbone.

In an embodiment, one internucleoside linkage modification of one wing or preferably of each of the wings of the oligonucleotide comprising SEQ ID NO:264 is a dmi-Phosphoramidate (PNdmi) linkage. Preferably, one wing or each of the wings of the oligonucleotide comprising SEQ ID NO: 264 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 264 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:264 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:264 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:264 is represented by SEQ ID NO:265:

(SEQ ID NO: 265)
eG_{PNdmi}eG_{PNdmi}eC*ₛeA_{PNdmi}eAₛdTₛdUₛdCₛdTₛdTₛdTₛdCₛdTₛdTₛdGₛe
A_{PNdmi}eAₛeA_{PNdmi}eA_{PNdmi}eT

In an embodiment the antisense oligonucleotide comprises the following base sequence:

(SEQ ID NO: 266)
TGGCAATUCTTTCTTGAAAA (AON113)

In this embodiment, the antisense oligonucleotide has a length of 20 to 50 nucleotides and comprises the base sequence SEQ ID NO:266. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:266 is 5-methylcytosine. In another embodiment, the C of the oligonucleotide comprising SEQ ID NO:266 present in the gap of said oligonucleotide (C at position 9 and position 13 of SEQ ID NO:266) are not methylated. In another embodiment, all Cs of the oligonucleotide comprising SEQ ID NO:266 are 5-methylcytosines.

In an embodiment, the oligonucleotide comprising SEQ ID NO:266 has at least one of its internucleoside linkages which is modified, preferably as a phosphorothioate or phosphoramidate (preferably mesyl-phosphoramidate). More preferably, the oligonucleotide comprising SEQ ID NO: 266 has 1, 2 or 3 phosphosdiesters linkage in each of its wings, preferably 3 phosphodiesters linkage in each of its wings. In an embodiment, the oligonucleotide comprising SEQ ID NO:266 is fully modified with a PS backbone.

In an embodiment, one internucleoside linkage modification of one wing or preferably of each of the wings of the oligonucleotide comprising SEQ ID NO:266 is a dmi-Phosphoramidate (PNdmi) linkage. Preferably, one wing or each of the wings of the oligonucleotide comprising SEQ ID NO: 266 comprises 1 or 2 or 3 or 4 or 5 PNdmi linkages, more preferably 2 or 3 or 4, most preferably 1.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 266 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:266 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:266 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:266 is represented by SEQ ID NO:267:

(SEQ ID NO: 267)
eT_{PNdmi}eGₒeGₛeC*ₒeAₛdAₛdTₛdUₛdCₛdTₛdTₛdTₛdCₛdTₛdTₛeGₒeAₛe
AₒeA_{PNdmi}eA

In an embodiment the antisense oligonucleotide comprises the following base sequence:

(SEQ ID NO: 268)
TGGCAATUCTTTCTTGAAAA (AON114)

In this embodiment, the antisense oligonucleotide has a length of 20 to 50 nucleotides and comprises the base sequence SEQ ID NO:268. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:268 is 5-methylcytosine. In another embodiment, the C of the oligonucleotide comprising SEQ ID NO:268 present in the gap of said oligonucleotide (C at position 9 and position 13 of SEQ ID NO:268) are not methylated. In another embodiment, all Cs of the oligonucleotide comprising SEQ ID NO:268 are 5-methylcytosines.

In an embodiment, the oligonucleotide comprising SEQ ID NO:268 has at least one of its internucleoside linkages which is modified, preferably as a phosphorothioate or phosphoramidate (preferably mesyl-phosphoramidate). More preferably, the oligonucleotide comprising SEQ ID NO: 268 has 1, 2 or 3 phosphosdiesters linkage in each of its wings, preferably 3 phosphodiesters linkage in each of its wings. In an embodiment, the oligonucleotide comprising SEQ ID NO:268 is fully modified with a PS backbone.

In an embodiment, one internucleoside linkage modification of one wing or preferably of each of the wings of the oligonucleotide comprising SEQ ID NO:262 is a dmi-Phosphoramidate (PNdmi) linkage. Preferably, one wing or each of the wings of the oligonucleotide comprising SEQ ID NO: 268 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 268 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:268 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:268 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:268 is represented by SEQ ID NO:269:

(SEQ ID NO: 269)
eT_{PNdmi}eG_{PNdmi}eG_{PNdmi}eC*ₒeAₛdAₛdTₛdUₛdCₛdTₛdTₛdTₛdCₛdTₛdTₛe
GₒeA_{PNdmi}eA_{PNdmi}eA_{PNdmi}eA

In an embodiment the antisense oligonucleotide comprises the following base sequence:

(SEQ ID NO: 270)
TGGCAATUCTTTCTTGAAAA
(AON115)

In this embodiment, the antisense oligonucleotide has a length of 20 to 50 nucleotides and comprises the base sequence SEQ ID NO:270. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:270 is 5-methylcytosine. In another embodiment, the C of the oligonucleotide comprising SEQ ID NO:270 present in the gap of said oligonucleotide (C at position 8 and position 12 of SEQ ID NO:270) are not methylated. In another embodiment, all Cs of the oligonucleotide comprising SEQ ID NO:270 are 5-methylcytosines.

In an embodiment, the oligonucleotide comprising SEQ ID NO:270 has at least one of its internucleoside linkages which is modified, preferably as a phosphorothioate or phosphoramidate (preferably mesyl-phosphoramidate). More preferably, the oligonucleotide comprising SEQ ID NO: 270 has 1, 2 or 3 phosphosdiesters linkage in each of its wings, preferably 3 phosphodiesters linkage in each of its wings. In an embodiment, the oligonucleotide comprising SEQ ID NO:270 is fully modified with a PS backbone.

In an embodiment, one internucleoside linkage modification of one wing or preferably of each of the wings of the oligonucleotide comprising SEQ ID NO:270 is a dmi-Phosphoramidate (PNdmi) linkage. Preferably, one wing or each of the wings of the oligonucleotide comprising SEQ ID NO: 270 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 270 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:270 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:270 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:270 is represented by SEQ ID NO:271:

(SEQ ID NO: 271)
eT_{PNdmi}eGₒeG_{PNdmi}eC*ₒeAₛdAₛdTₛdUₛdCₛdTₛdTₛ
dTₛdCₛdTₛdTₛeGₒeA_{PNdmi}eAₒeA_{PNdmi}eA

In an embodiment the antisense oligonucleotide comprises the following base sequence:

(SEQ ID NO: 272)
TGGCAATUCTTTCTTGAAAA(AON116)

In this embodiment, the antisense oligonucleotide has a length of 20 to 50 nucleotides and comprises the base sequence SEQ ID NO:272. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:272 is 5-methylcytosine. In another embodiment, the C of the oligonucleotide comprising SEQ ID NO:272 present in the gap of said oligonucleotide (C at position 9 and position 13 of SEQ ID NO:272) are not methylated. In another embodiment, all Cs of the oligonucleotide comprising SEQ ID NO:272 are 5-methylcytosines.

In an embodiment, the oligonucleotide comprising SEQ ID NO:272 has at least one of its internucleoside linkages which is modified, preferably as a phosphorothioate or phosphoramidate (preferably mesyl-phosphoramidate). More preferably, the oligonucleotide comprising SEQ ID NO: 272 has 1, 2 or 3 phosphosdiesters linkage in each of its wings, preferably 3 phosphodiesters linkage in each of its wings. In an embodiment, the oligonucleotide comprising SEQ ID NO:272 is fully modified with a PS backbone.

In an embodiment, one internucleoside linkage modification of one wing or preferably of each of the wings of the oligonucleotide comprising SEQ ID NO:272 is a dmi-Phosphoramidate (PNdmi) linkage. Preferably, one wing or each of the wings of the oligonucleotide comprising SEQ ID NO: 272 comprises 1 or 2 or 3 or 4 or 5 PNdmi linkages, more preferably 2 or 3 or 4, most preferably 3.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 272 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:272 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:272 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:272 is represented by SEQ ID NO:273:

(SEQ ID NO: 273)
eT_{PNdmi}eGₒeG_{PNdmi}eC*ₒeA_{PNdmi}dAₛdTₛdUₛdCₛdTₛdTₛ
dTₛdCₛdTₛdT_{PNdmi}eGₒeA_{PNdmi}eAₒeA_{PNdmi}eA

In an embodiment the antisense oligonucleotide comprises the following base sequence:

(SEQ ID NO: 274)
TGGCAATUCTTTCTTGAAAA(AON117)

In this embodiment, the antisense oligonucleotide has a length of 20 to 50 nucleotides and comprises the base sequence SEQ ID NO:274. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:274 is 5-methylcytosine. In another embodiment, the C of the oligonucleotide comprising SEQ ID NO:274 present in the gap of said oligonucleotide (C at position 9 and position 13 of SEQ ID NO:274) are not methylated. In another embodiment, all Cs of the oligonucleotide comprising SEQ ID NO:274 are 5-methylcytosines.

In an embodiment, the oligonucleotide comprising SEQ ID NO:274 has at least one of its internucleoside linkages which is modified, preferably as a phosphorothioate or phosphoramidate (preferably mesyl-phosphoramidate). More preferably, the oligonucleotide comprising SEQ ID NO: 274 has 1, 2 or 3 phosphosdiesters linkage in each of its wings, preferably 3 phosphodiesters linkage in each of its wings. In an embodiment, the oligonucleotide comprising SEQ ID NO:274 is fully modified with a PS backbone.

In an embodiment, one internucleoside linkage modification of one wing or preferably of each of the wings of the oligonucleotide comprising SEQ ID NO:274 is a dmi-Phosphoramidate (PNdmi) linkage. Preferably, one wing or each of the wings of the oligonucleotide comprising SEQ ID NO: 274 comprises 1 or 2 or 3 or 4 or 5 PNdmi linkages, more preferably 2 or 3 or 4, most preferably 2.

In an embodiment, at least one internucleoside linkages in one wing (or in both wings) is not modified and is thus a phosphodiester internucleoside linkage. A preferred internucleoside linkage modification in one wing (or in both wings) is phosphorothioate

In an embodiment, one wing or preferably each wing of the oligonucleotide comprising SEQ ID NO: 274 comprises two distinct modified internucleoside linkages, preferably dmi-Phosphoramidate and phosphorothioate.

In an embodiment, the oligonucleotide has three distinct internucleoside linkages, preferably phosphodiester, dmi-Phosphoramidate and phosphorothioate.

In an embodiment one wing or preferably each of the wings of the oligonucleotide comprising SEQ ID NO:274 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:274 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:274 is represented by SEQ ID NO:275:

SEQ ID NO: 275)
eT_{PNdmi}eG_{PNdmi}eGₛeC*ₒeAₛdAₛdTₛdUₛdCₛdTₛdTₛ
dTₛdCₛdTₛdTₛeGₒeAₛeA_{PNdmi}eA_{PNdmi}eA

In an embodiment the antisense oligonucleotide comprises the following base sequence:

(SEQ ID NO: 276)
TGGCAATUCTTTCTTGAAAA
(AON118)

In this embodiment, the antisense oligonucleotide has a length of 20 to 50 nucleotides and comprises the base sequence SEQ ID NO:276. The length may be 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 nucleotides.

Each nucleotide may be a nucleotide analogue as identified earlier herein. If a base is modified or if a base analogue is being used, said modified base or base analogue should keep the same base pair specificity as the base it replaces.

In an embodiment, at least one C of the oligonucleotide comprising SEQ ID NO:276 is 5-methylcytosine. In another embodiment, the C of the oligonucleotide comprising SEQ ID NO:276 present in the gap of said oligonucleotide (C at position 9 and position 13 of SEQ ID NO:276) are not methylated. In another embodiment, all Cs of the oligonucleotide comprising SEQ ID NO:276 are 5-methylcytosines.

In an embodiment, the oligonucleotide comprising SEQ ID NO:276 has at least one of its internucleoside linkages which is modified, preferably as a phosphorothioate or phosphoramidate (preferably mesyl-phosphoramidate). More preferably, the oligonucleotide comprising SEQ ID NO: 276 has 1, 2 or 3 phosphosdiesters linkage in each of its wings, preferably 3 phosphodiesters linkage in each of its wings. In an embodiment, the oligonucleotide comprising SEQ ID NO:276 is fully modified with a PS backbone.

In an embodiment, one internucleoside linkage modification of one wing or preferably of each of the wings of the oligonucleotide comprising SEQ ID NO:276 is a dmi-Phosphoramidate (PNdmi) linkage. Preferably, one wing or each of the wings of the oligonucleotide comprising SEQ ID NO: 276 comprises 1 or 2 or 3 or 4 or 5 PNdmi linkages, more preferably 2 or 3 or 4, most preferably 3.

In an embodiment, one wing or preferably each wing of the oligonucleotide comprising SEQ ID NO: 276 comprises two distinct modified internucleoside linkages, preferably dmi-Phosphoramidate and phosphorothioate.

In an embodiment, the oligonucleotide has two distinct internucleoside linkages, preferably dmi-Phosphoramidate and phosphorothioate.

In an embodiment one wing or preferably each of the wings of the oligonucleotide comprising SEQ ID NO:276 has at least one internucleoside linkage modification and/or at least one sugar modification and/or at least one base modification compared to an RNA-based antisense oligonucleotide. In a preferred embodiment, one wing or more preferably both wings of the oligonucleotide comprising SEQ ID NO:276 comprises 1, 2, 3, 4 or 5 nucleotides. In a more preferred embodiment, one wing or even more preferably both wings comprise 5 nucleotides. Most preferably both wings comprise 5 nucleotides and are MOE.

A most preferred oligonucleotide comprising SEQ ID NO:276 is represented by SEQ ID NO:277:

(SEQ ID NO: 277)
eT_{PNdmi}eG_{PNdmi}eGₛeC*_{PNdmi}eAₛdAₛdTₛdUₛdCₛdTₛdTₛ
dTₛdCₛdTₛdTₛeG_{PNdmi}eAₛeA_{PNdmi}eA_{PNdmi}eA

Preferred oligonucleotides comprise SEQ ID NO: 2, 4, 6, 8, 7, 8, 9, 10, 11, 12, 14, 16, 18, 20, 22, 24, 25, 26, 27, 28, 29, 30, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117,122, 124, 126, 128, 130, 132, 134, 136, 138, 140, 142, 144, 174, 176, 178, 180, 182, 184, 186, 188, 190, 192, 194, 196, 198, 200, 204, 217, 219, 221, 223, 225, 227, 229, 231, 233, 235, 237, 239, 241, 243, 245, 247, 249, 251, 253, 255, 257, 259, 261, 263, 264, 265, 267, 269, 271, 273, 275, 277 preferably SEQ ID NO: 7, 8, 9, 10, 11, 12, 25, 26, 27, 28, 29, 30, 33, 34, 35, 36, 37, 38, 39, 47, 85, 97, 99, 115, 192, 194, 196, 198, 200, 221, 223 more preferably SEQ ID NO: 8, 10, 12, 26, 28, 30, 35, 37, 39, 47, 85, 97, 99, 115, 221, 223 and most preferably SEQ ID NO:

26, 28, 30, 35, 37, 39, 47, 85, 97, 99, 115, 221, 223.

### Composition

In a further aspect, there is provided a composition comprising the antisense oligonucleotide of the invention, wherein the composition comprises a pharmaceutically acceptable excipient. This composition preferably comprises or consists of or essentially consists of an antisense oligonucleotide as described above.

All preferred features relating to each of these antisense oligonucleotides have been disclosed in the section entitled “antisense oligonucleotide”.

In a preferred embodiment, said composition is for use as a medicament. Said composition is therefore a pharmaceutical composition. A pharmaceutical composition usually comprises a pharmaceutically accepted carrier, diluent and/or excipient. In a preferred embodiment, a composition of the current invention comprises an antisense oligonucleotide as defined herein and optionally further comprises a pharmaceutically acceptable formulation, filler, preservative, solubilizer, carrier, diluent, excipient, salt, adjuvant and/or solvent. Such pharmaceutically acceptable carrier, filler, preservative, solubilizer, diluent, salt, adjuvant, solvent and/or excipient may for instance be found in Remington: The Science and Practice of Pharmacy, 20th Edition. Baltimore, MD: Lippincott Williams & Wilkins, 2000.

A pharmaceutical composition may be further formulated to further aid in enhancing the stability, solubility, absorption, bioavailability, pharmacokinetics and cellular uptake of said antisense oligonucleotide, in particular formulations comprising excipients capable of forming complexes, nanoparticles, microparticles, nanotubes, nanogels, hydrogels, poloxamers or pluronics, polymersomes, colloids, microbubbles, vesicles, micelles, lipoplexes, and/or liposomes. Examples of nanoparticles include polymeric nanoparticles, gold nanoparticles, magnetic nanoparticles, silica nanoparticles, lipid nanoparticles, sugar particles, protein nanoparticles and peptide nanoparticles.

A preferred composition comprises at least one excipient that may further aid in enhancing the targeting and/or delivery of said antisense oligonucleotide to the central nerve system, and/or the spinal cord, and/or into brain tissue and/or to a neuronal tissue and/or a cell. A cell may be a neuronal cell.

Many of these excipients are known in the art (e.g. see Bruno, 2011) and may be categorized as a first type of excipient. Examples of first type of excipients include polymers (e.g. polyethyleneimine (PEI), polypropyleneimine (PPI), dextran derivatives, butylcyanoacrylate (PBCA), hexylcyanoacrylate (PHCA), poly(lactic-co-glycolic acid) (PLGA), polyamines (e.g. spermine, spermidine, putrescine, cadaverine), chitosan, poly(amido amines) (PAMAM), poly(ester amine), polyvinyl ether, polyvinyl pyrrolidone (PVP), polyethylene glycol (PEG) cyclodextrins, hyaluronic acid, colominic acid, and derivatives thereof), dendrimers (e.g. poly(amidoamine)), lipids {e.g. 1,2-dioleoyl-3-dimethylammonium propane (DODAP), dioleoyldimethylammonium chloride (DODAC), phosphatidylcholine derivatives [e.g 1,2-distearoyl-sn-glycero-3-phosphocholine (DSPC)], lyso-phosphatidylcholine derivaties [e.g. 1-stearoyl-2-lyso-sn-glycero-3-phosphocholine (S-LysoPC)], sphingomyeline, 2-{3-[bis-(3-amino-propyl)-amino]-propylamino}-N-ditetracedyl carbamoyl methylacetamide (RPR209120), phosphoglycerol derivatives [e.g. 1,2-dipalmitoyl-sn-glycero-3-phosphoglycerol sodium salt (DPPG-Na), phosphaticid acid derivatives [1,2-distearoyl-sn-glycero-3-phosphaticid acid, sodium salt (DSPA), phosphatidylethanolamine derivatives [e.g. dioleoyl-phosphatidylethanolamine (DOPE), 1,2-distearoyl-sn-glycero-3-phosphoethanolamine (DSPE),2-diphytanoyl-sn-glycero-3-phosphoethanolamine (DPhyPE),], N-[1-(2,3-dioleoyloxy) propyl]-N, N, N-trimethylammonium (DOTAP), N-[1-(2,3-dioleyloxy) propyl]-N,N,N-trimethylammonium (DOTMA), 1,3-di-oleoyloxy-2-(6-carboxy-spermyl)-propylamid (DOSPER), (1,2-dimyristyolxypropyl-3-dimethylhydroxy ethyl ammonium (DMRIE), (N1-cholesteryloxycarbonyl-3,7-diazanonane-1,9-diamine (CDAN), dimethyldioctadecylammonium bromide (DDAB), 1-palmitoyl-2-oleoyl-sn-glycerol-3-phosphocholine (POPC), (b-L-arginyl-2,3-L-diaminopropionic acid-N-palmityl-N-olelyl-amide trihydrochloride (AtuFECT01), N,N-dimethyl-3-aminopropane derivatives [e.g. 1,2-distearoyloxy-N, N-dimethyl-3-aminopropane (DSDMA), 1,2-dioleyloxy-N,N-dimethyl-3-aminopropane (DoDMA), 1,2-dilinoleyloxy-N, N-3-dimethylaminopropane (DLinDMA), 2,2-dilinoleyl-4-dimethylaminomethyl [1,3]-dioxolane (DLin-K-DMA), phosphatidylserine derivatives [1,2-dioleyl-sn-glycero-3-phospho-L-serine, sodium salt (DOPS)], cholesterol}proteins (e.g. albumin, gelatins, atellocollagen), and peptides (e.g. protamine, PepFects, NickFects, polyarginine, polylysine, CADY, MPG).

Another preferred composition may comprise at least one excipient categorized as a second type of excipient. A second type of excipient may comprise or contain a conjugate group as described herein to enhance targeting and/or delivery of the antisense oligonucleotide of the invention to a tissue and/or cell and/or into a tissue and/or cell, as for example neuronal tissue or cell. Neuronal tissues or cells may be all the neuronal tissues or cells known to be affected in FAD. Such neuronal tissues or cells may be derived from or may be from the hippocampus, amygdala, cerebellar cortex (frontal lobe, parietal lobe, temporal lobe, occipital lobe), cerebellum, corpus callosum or the thalamus, beyond others. Both types of excipients may be combined together into one single composition as identified herein. A conjugate group may be selected from a ligand, including transferrin-receptor targeting moieties like antibody fragments, aptamers, peptides, sugars or lipids, ethylene glycol moieties.

The skilled person may select, combine and/or adapt one or more of the above or other alternative excipients and delivery systems to formulate and deliver an antisense oligonucleotide for use in the present invention.

Such a pharmaceutical composition of the invention may be administered in an effective concentration at set times to an animal, preferably a mammal. More preferred mammal is a human being. An antisense oligonucleotide or a composition as defined herein for use according to the invention may be suitable for direct administration to a cell, tissue and/or an organ in vivo of individuals affected by or at risk of developing a disease or condition as identified herein, and may be administered directly in vivo, ex vivo or in vitro. Administration may be via systemic and/or parenteral routes, for example intravenous, subcutaneous, intraventricular, intrathecal, intra-cerebrospinal, intramuscular, intranasal, enteral, intravitreal, intracerebral, epidural or oral route.

Preferably, such a pharmaceutical composition of the invention may be encapsulated in the form of an emulsion, suspension, pill, tablet, capsule or soft-gel for oral delivery, or in the form of aerosol or dry powder or device for delivery to the cerebrospinal fluid.

In an embodiment, the pharmaceutical composition is a cerebrospinal composition or an artificial cerebrospinal composition also called an artificial cerebrospinal fluid (aCSF). Such composition comprises a buffer solution representative of the cerebrospinal fluid. Preferably such composition comprises the following ion concentrations: Na⁺ 150 mM, K⁺ 3.0 mM, Ca²⁺ 1.4 mM, Mg²⁺ 0.8 mM, P 1.0 mM and Cl⁻ 155 mM. It is commonly used as a vehicle solution for administration of therapeutic agents to the central nervous system (CNS). The exact composition may slightly differ between suppliers. The oligonucleotide may be formulated in such composition and may be delivered using a device.

In an embodiment an antisense oligonucleotide of the invention may be used together with another compound already known to be used for the treatment of said disease. Such combined use may be a sequential use: each component is administered in a distinct composition. Alternatively, each compound may be used together in a single composition.

In a further aspect, there is therefore provided an antisense oligonucleotide or a composition for use as a medicament.

In an embodiment, an antisense oligonucleotide or a composition for use as a medicament is preferably for treating and/or delaying and/or curing and/or preventing and/or ameliorating a disease or condition associated with an abnormal processing (or altered processing) of the Amyloid Precursor Protein (APP), preferably familiar Alzheimer disease (FAD). In such disease or condition with an abnormal processing (or altered processing) of the APP, one allele of a protein of the γ-secretase complex is mutated compared to the wild-type protein (human PSEN1 protein is represented by SEQ ID NO: 153 or 159 and is encoded by SEQ ID NO: 152 or 169 and genomic DNA is represented by SEQ ID NO: 151). In an embodiment, an allele of PSEN1, preferably an allele of human PSEN1 is mutated. Known mutations have been listed earlier herein.

A subject or a patient having a disease or condition associated with an abnormal processing of the APP may be identified as such (may be diagnosed) by the presence of at least one of the following symptoms and/or parameters:
increased Aβ42/38 and/or Aβ43/40 and/or Aβ43/37 and/or Aβ42/40 ratios (preferably increased Aβ 42/40 and/or Aβ 42/38 ratios, more preferably an increased Aβ42/38 ratio) in the plasma of symptomatic FAD patients,
increased Aβ42/38 and/or Aβ43/40 and/or Aβ43/37 and/or Aβ42/40 ratios (preferably increased Aβ 42/40 and/or Aβ 42/38 ratios, more preferably an increased Aβ42/38 ratio) in the cerebrospinal fluid or plasma of FAD patients with no detectable Aβ plaques in the brain (i.e. so called asymptomatic patients)
decreased Aβ42/38 and/or Aβ43/40 and/or Aβ43/37 and/or Aβ42/40 ratios (preferably decreased Aβ 42/40 and/or Aβ 42/38 ratios, more preferably a decreased Aβ42/38 ratio) in the cerebrospinal fluid of patients with Aβ plaques in the brain
presence of Aβ plaques in the brain (measured with positron emission tomography (PET) using ligands specific for fibrilar Aβ)
an increased level of Tau and/or phosphorylated Tau 181 (p-Tau181) in the cerebrospinal fluid and the plasma
presence of neurofibrillary tangles in the brain (measured with positron emission tomography (PET) using ligands specific for Tau)
reduce of cerebral metabolic rate for glucose (assessed with in vivo imaging using 18[F]fluoro-2-deoxy-D-glucose PET (FDG-PET)
neuronal degeneration and especially an atrophy or degeneration of at least one of the following brain regions: hippocampus, amygdala, cortex, cerebellum, frontal lobe, parietal lobe, temporal lobe, occipital lobe, frontal lobe, corpus callosum and the thalamus assessed with neuroimaging techniques such as MRI
an increased SNAP25 level in cerebrospinal fluid
an increased level of neurofilament light (NFL) in cerebrospinal fluid and the plasma
cognitive impairments (assessed by MMSE and Alzheimer's Disease Assessment Scale-Cognitive subscale)

In this context, increased Aβ 42/40 and/or Aβ 42/38 ratios has been already defined earlier herein.

In this context, an increased level of Tau and/or phosphorylated Tau 181 (p-Tau181) in the cerebrospinal fluid and/or the plasma may mean an increased level of Tau and/or phosphorylated Tau 181 (p-Tau181) which is increased of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level in a healthy subject. The level of Tau may be assessed by techniques known to the skilled person.

In an embodiment it is assessed using ELISA, preferably as carried out in the experimental part. Level of Tau and neurofibrillary tangles formed by Tau aggregations may also be assessed using PET imaging.

An atrophy or degeneration of a brain region and especially of at least one of the following brain regions listed above may be assessed by a physician using common known techniques such as MRI imaging.

In this context, a decreased metabolic rate for glucose in the brain may mean a decreased level of metabolic rate which is decreased of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level in a healthy subject. The metabolic rate for glucose in the brain may be assessed by techniques known to the skilled person.

In this context, neuronal degeneration and especially an atrophy or degeneration of at least one of the following brain regions: hippocampus, amygdala, cortex, cerebellum, frontal lobe, parietal lobe, temporal lobe, occipital lobe, frontal lobe, corpus callosum and the thalamus may be assessed with neuroimaging techniques such as MRI. For example to investigate neurodegeneration we may use the hippocampal and ventricular volumes as a parameter. The white matter hyperintensity (WMH) volume, a cerebrovascular disease marker may also be used.

In this context, an increased level of NFL in the cerebrospinal fluid and the plasma is indicative of a disease or condition associated with neurodegeneration. Increased NFL levels may mean an increased level of NFL which is increased of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level in a healthy subject. The level of NFL may be assessed by techniques known to the skilled person. In an embodiment it is assessed using ELISA.

In this context, an increased level of SNAP25 in the cerebrospinal fluid is indicative of a disease or condition associated with synaptic damage and neurodegeneration. Increased SNAP25 levels may mean an increased level of SNAP25 which is increased of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level in a healthy subject. The level of SNAP25 may be assessed by techniques known to the skilled person. In an embodiment it is assessed using ELISA.

In an embodiment, an antisense oligonucleotide or a composition for use according to the invention is able to induce a therapeutic effect. A therapeutic effect may mean that the antisense oligonucleotide elicits at least one of the following:
the oligonucleotide is able to preferentially silence, inactivate, break down, knock down, reduce and/or decrease (the levels of) mutated PSEN1 or PSEN2 transcripts (preferably PSEN1 transcripts) and preferably the oligonucleotide is not (or minimally) able to silence, inactivate, break down, knock down, reduce and/or decrease (the levels of) wild-type PSEN1 or PSEN2 transcripts (preferably PSEN1 transcripts),
the oligonucleotide is able to normalize, correct, or reverse abnormal Aβ42/38 and/or Aβ43/40 and/or Aβ43/37 and/or Aβ42/40 ratios (preferably Aβ42/38 and/or Aβ43/40 ratios, more preferably the Aβ42/38 ratio), and
the oligonucleotide is able to alleviate one or more symptom(s) and/or characteristic(s) and/or to improve a parameter linked with or associated with a disease or condition associated with an abnormal processing of the Amyloid Precursor Protein (APP) preferably familiar Alzheimer disease (FAD).

The silencing, inactivation, knocking down, breaking down, decrease or the reduction of the (levels of) the mutated allele (transcript and protein) has already been defined earlier herein. It corresponds to the first way of assessing an activity of the antisense oligonucleotide of the invention.

The normalization, correction, or reversion of abnormal Aβ42/38 and/or Aβ43/40 and/or Aβ43/37 and/or Aβ42/40 ratios (preferably Aβ42/38 and/or Aβ43/40 ratios, more preferably the Aβ42/38 ratio) may mean a decrease of the Aβ42/38 and/or Aβ43/40 and/or Aβ43/37 and/or Aβ42/40 ratios (preferably Aβ42/38 and/or Aβ43/40 ratios, more preferably the Aβ42/38 ratio) as earlier defined herein. It corresponds to the second way of assessing an activity of the oligonucleotide of the invention.

The alleviation of one or more symptom(s) and/or characteristic(s) and/or the improvement of the value of a parameter linked with or associated with a disease or condition associated with an abnormal processing of the Amyloid Precursor Protein (APP) (preferably familiar Alzheimer disease (FAD)) may refer to at least one of the following:
a decreased level of Tau and/or phosphorylated Tau 181 in the cerebrospinal fluid and plasma,
a metabolism of glucose in the cortex which is increased,
a neuronal degeneration which is delayed or reversed and especially a neuronal degeneration of at least one of the following brain regions is delayed or reversed: hippocampus, amygdala, cortex, cerebellum, frontal lobe, parietal lobe, temporal lobe, occipital lobe, frontal lobe, corpus callosum and the thalamus,
an decreased SNAP25 level in cerebrospinal fluid,
a decreased level of NFL in cerebrospinal fluid and/or plasma,
cognitive impairments that are reversed that are improved.
a decrease/slowdown the formation of Aβ plaques formation in the brain.

In this context, a decreased level of Tau and/or phosphorylated Tau 181 in the cerebrospinal fluid and plasma may mean a decreased level of Tau of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level at the onset of the treatment. The level of Tau and phosphorylated Tau 181 may be assessed by techniques known to the skilled person as earlier defined herein.

An atrophy or degeneration of a brain region and especially of at least one of the brain regions listed above may be assessed as earlier defined herein.

In this context, a decreased level of NFL may mean a decrease of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level at the onset of the treatment. The level of NFL may be assessed by techniques known to the skilled person as earlier defined herein.

In this context, a decreased level of SNAP25 may be of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level at the onset of the treatment. The level of SNAP25 may be assessed by techniques known to the skilled person as earlier defined herein.

In this context, a decrease/slowdown of the formation of Aβ plaques formation in the brain may mean a decrease of at least 1%, 5%, 10%, 15%, 20%, 25%, 30%, 35%, 40%, 45%, 50%, 55%, 60%, 65%, 70%, 75%, 80%, 85%, 90%, 95%, 100% compared to its level at the onset of the treatment. Aβ plaques formation in the brain may be monitored using techniques known to the skilled person. Preferred techniques have been earlier defined herein.

An antisense oligonucleotide as defined herein is able to improve one parameter or reduce a symptom or characteristic if after at least one week, one month, six month, one year or more of treatment using a dose of said antisense oligonucleotide of the invention as identified herein said parameter is said to have been improved or said symptom or characteristic is said to have been reduced.

Improvement in this context may mean that said parameter had been significantly changed towards a value of said parameter for a healthy person and/or towards a value of said parameter that corresponds to the value of said parameter in the same individual at the onset of the treatment.

Reduction or alleviation in this context may mean that said symptom or characteristic had been significantly changed towards the absence of said symptom or characteristic which is characteristic for a healthy person and/or towards a change of said symptom or characteristic that corresponds to the state of the same individual at the onset of the treatment.

In a further aspect, there is provided the use of a composition or an antisense oligonucleotide as described in the previous sections for use as a medicament or part of therapy, or applications in which said oligonucleotide exerts its activity intracellularly.

Preferably, an oligonucleotide or composition of the invention is for use as a medicament or part of a therapy for preventing, delaying, curing, ameliorating and/or treating a disease or condition associated with an abnormal processing of the Amyloid Precursor Protein (APP), preferably familiar Alzheimer disease (FAD).

In a further aspect there is provided a use of a composition or an antisense oligonucleotide as described in the previous sections, for the manufacture of a medicament for preventing, delaying, curing, ameliorating and/or treating a disease or condition associated with an abnormal processing of the Amyloid Precursor Protein (APP), preferably familiar Alzheimer disease (FAD). In a further aspect, there is provided a method for preventing, treating, curing, ameliorating and/or delaying a condition or disease as defined in the previous section in an individual, in a cell, tissue or organ of said individual. The method comprises administering an antisense oligonucleotide or a composition of the invention to said individual or a subject in the need thereof. In some embodiments, administering an antisense oligonucleotide or a composition means administering to a subject such as a subject in need thereof. In a preferred embodiment, a therapeutically effective amount of an antisense oligonucleotide or a composition is administered.

The method according to the invention wherein an antisense oligonucleotide or a composition as defined herein may be suitable for administration to a cell, tissue and/or an organ in vivo of individuals affected by any of the herein defined diseases or at risk of developing said disease, and may be administered in vivo or in vitro. An individual or a subject in need is preferably a mammal, more preferably a human being.

In an embodiment, in an in vitro method of the invention (such as in gymnotic uptake experiment), a concentration of an oligonucleotide or composition is ranged from 0.1 nM to 5 μM or from 1 nM to 25 μM, or from 2 nM to 10 μM.

In an embodiment, in an in vivo method of the invention, a concentration of an oligonucleotide or composition is ranged from 0.1 μM to 10 mM or from 0.3 μM to 1 mM, or from 0.5 μM to 50 mM or from 1 μM to 10 mM.

Dose ranges of an antisense oligonucleotide or composition according to the invention are preferably designed on the basis of ascending dose studies in clinical trials (in vivo use) for which rigorous protocol requirements exist.

In an intrathecal (IT) or intracerebroventricular (ICV) (i.e. administration into the central nervous system) delivery, an antisense oligonucleotide as defined herein may be used at a dose which is ranged from 10 to 300 μg/dose in mice or 1-60 mg in Non-Human Primates (NHPs), 1-200 or 2-150 pr 5-120 mg in humans. The frequency of the IT or ICV dose is once weekly, biweekly, or once per 4, 8. 12, 16, 20 or 24 weeks.

In an SC (subcutaneous) or IV (intravenous) delivery, an antisense oligonucleotide as defined herein may be used at a dose which is ranged from 1 to 500 or 3-300 mg/kg in mice, 0.1 to 100 or 0.5 to 75 or 1-50 mg/kg in NHPs, 20-500 or 30-450 or 50-400 mg in humans. The frequency of the SC or IV treatment is daily, every 2 days, every 4 days, twice weekly, weekly, biweekly, or once per month.

The ranges of concentration or dose of antisense oligonucleotide or composition as given above are preferred concentrations or doses for in vitro or in vivo uses. The skilled person will understand that depending on the identity of the antisense oligonucleotide used, the target cell to be treated, the gene target and its expression levels, the medium used and the transfection and incubation conditions, the concentration or dose of antisense oligonucleotide used may further vary and may need to be optimised any further.

In this document and in its claims, the verb “to comprise” and its conjugations is used in its non-limiting sense to mean that items following the word are included, but items not specifically mentioned are not excluded. The verb “to comprise” is synonymous with the verb “to have” unless otherwise indicated. In addition the verb “to consist” may be replaced by “to consist essentially of” meaning that an antisense oligonucleotide or a composition as defined herein may comprise additional component(s) than the ones specifically identified, said additional component(s) not altering the unique characteristic of the invention.

In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each embodiment as identified herein may be combined together unless otherwise indicated. All patent and literature references cited in the present specification are hereby incorporated by reference in their entirety.

The following examples are offered for illustrative purposes only, and are not intended to limit the scope of the present invention in any way.

TABLE 1a
summary of the sequences identified in the application (part 1)

| SEQ ID NO | Type of sequences |
|---|---|
| 1-30, 32-117, 121-144, 173-200 (table 4 in | Oligonucleotides for |
| example 3, and table 5 in example 4), 218-277 | treatment of human |
| 31 (table 4 in example 3) | Scrambled |
| | oligonucleotide, control |
| 118-120, 157, 201-204 (table 2 in example 1, | Oligonucleotides for |
| and table 9 in example 6) | treatment of mouse |
| 162-164 | Immunogens |
| 205-217 | Primer sequences |

TABLE 1b
summary of the sequences identified in the application (part 2)

| | | | Mouse | | Human | |
|---|---|---|---|---|---|---|
| | | Mouse | A431E | Human | A431E | Human |
| | | PSEN1 | PSEN1 | PSEN1 | PSEN1 | PSEN2 |
| Genomic | | 145 | 148 | 151 | 154 | 165 |
| DNA | | | | | | |
| Coding | Isoform 1 | 146 | 149 | 152 | 155 | 171 |
| mRNA | Isoform 2 | | | 169 | 170 | 172 |
| Protein | Isoform 1 | 147 | 150 | 153 | 156 | 166 |
| | Isoform 2 | | | 159 | 161 | 168 |

### Example 1

### Introduction

Mutations in Psen1, like A431E, affect the activity of γ-secretase. This is a heterotetrameric complex in which presenilins act as the catalytic subunit in the cleavage of the APP C-terminal fragment (CTF). Impaired Psen1, leading to a reduced processivity of γ-secretase, results in increase generation of long ≥Aβ42 species at expenses of short ≤Aβ38 species. This leads to an abnormal Aβ profile, followed by amyloid deposition that causes synaptic dysfunction and neurodegeneration (Petit D et al., Mol Psychiatry. 2022 Apr. 1). The therapeutic strategy of this invention aims at the specific reduction of mutant Psen1 and normalization of Aβ peptide profiles. To demonstrate the effectivity of a mouse sequence-specific AON of the invention, neuronal cell cultures derived from the Psen1 A431E;APPKi knock-in mouse model (Tg/Tg) were treated with AON20 (SEQ ID NO:118) for eight days. The resulting PSEN1 protein levels were assessed, as well as the γ-secretase processivity and soluble Aβ peptides profiles.

### Material and Methods

### AONs

All antisense oligonucleotides (AONs) have a phosphorothioate (PS) backbone with a DNA gap flanked by 2′-MOE wings at 5′ and 3′ end (Table 1). The AONs were synthesized in 5 μmol scale using a MerMade 12 synthesizer (Bioautomation) and standard phosphoramidite protocols. The AONs were cleaved and deprotected in a two-step sequence (DEA followed by conc. NH₄OH treatment), purified by anion-exchange chromatography, desalted by size exclusion chromatography and lyophilized. Mass spectrometry confirmed the identity of all AONs, and purity (determined by UPLC) was found acceptable for all AONs (>80%).

TABLE 2
Mouse AON sequences

| | SEQ | |
|---|---|---|
| AON | ID | |
| ID | NO: | Sequence (5′-3′) |
| AON | 118 | eAₛeGₛeC*ₛeGₛeC*ₛdCₛdGₛdGₛdCₛdAₛdAₛ |
| 20 | | dTₛdTₛdCₛdTₛdTₛeTₛeC*ₛeTₛeTₛeG |
| AON | 120 | eAₛeGₛeC*ₛeGgeC*ₛdC*ₛdGₛdGₛdC*ₛdAₛ |
| 21 | | dAₛdTₛdTₛdC*ₛdTₛdTₛeTₛeC*ₛeTₛeTₛeG |

A = adenosine; G = guanine; T = thymine; C=cytosine; C* = 5-methylcytosine. d = DNA, e = 2′-MOE, ₛ = PS linkage.
Corresponding non modified sequence of the oligonucleotide represented by SEQ ID NO: 118 is SEQ ID NO: 119 (which is AGCGCCGGCAATTCTTTCTTG)
Corresponding non modified sequence of the oligonucleotide represented by SEQ ID NO: 120 is SEQ ID NO: 157 (which is AGCGCCGGCAATTCTTTCTTG)

### Primary Mouse Neuronal Cultures (NCs)

The Psen1 A431E (GCG>GAA) knock-in mouse model was generated using CrisprCas9 technology. Two additional silent mutations were introduced in the target region, resulting in four nucleotide differences compared to the wild-type allele sequence. Psen1 A431E mice were subsequently crossed with mice carrying two copies of humanized amyloid-beta precursor protein APPKi mouse (Serneels et al., 2020). To obtain primary neuronal cell cultures, cerebral cortices from Psen1 A431E; APPKi E14 embryos were dissected. Tissue was dissociated by 15 min incubation with 0.25% trypsin in HBSS followed by mechanical dissociation using a fire polish Pasteur pipette. Cells were then seeded in poly-L-lysine-coated 96 well plates in MEM medium with 10% Horse serum, 0.6% D-Glucose and 1% Pen-Strep. After 4 h, medium is changed to Neurobasal medium supplemented with B27, 0.5 nM Glutamine and 1% Pen-Strep. Half-medium was refreshed daily.

### Gymnotic Uptake

Primary neuronal cultures derived from PSEN1 A431E homozygous embryos (Psen1A431E/A431E: APPKi embryos) were treated daily by addition of 0-1-10 μM AON (Table 2) to the medium, starting at day one after culture initiation. Cells were harvested after 8 days of treatment.

### Protein Extraction and Western Blot

Proteins were extracted in lysis buffer containing STE (NaCl/Tris-HCl/EDTA), 1% Triton 100 and a cocktail of Proteases inhibitors. Protein concentrations were determined by the Bio-Rad DC protein assay (Bio-Rad). Samples were separated by SDSPAGE (4-12% Bis-Tris NuPAGE gels in MES running buffer, Life Technologies) and transferred onto nitrocellulose membranes (Life Technologies). After blocking in 4% non-fat milk, membranes were incubated overnight at 4° C. with primary antibodies (Table 3) followed by washing and incubation with horseradish peroxidase (HRP)-conjugated secondary antibodies (1 h, room temperature). After final washing, immunodetection was done using enhanced chemiluminescence (Western Lightning-Plus ECL, PerkinElmer), and immunoreactive protein bands were digitally captured and quantified on a Fuji MiniLAS 3000 imager (Fuji, Düsseldorf, Germany) using Image Studio Light software version 5.2 (LI-COR Biosciences, Cambridge, UK).

TABLE 3
Primary antibodies.

| Target | Host | Source | Reference | Immunogen | Publication |
|---|---|---|---|---|---|
| PS1 N- | Rabbit | Lab | B19 | Mouse PSEN1: | Annaert et al., |
| terminal | | Bart de | | SQERQQQH | 1999, J Cell Biol |
| fragment | | Strooper | | GRQRLDN | 147, 277-294 |
| | | | | (SEQ ID | |
| | | | | NO: 162) | |
| APP | Rabbit | Lab | B63 | Mouse APP: | Esselens et al., |
| (Full lenght | | Bart de | | NGYENPTY | 2004, J Cell Biol |
| and C-terminal | | Strooper | | KFFEQMQN | 166, 1041-1054 |
| fragment) | | | | (SEQ ID | |
| | | | | NO: 163) | |
| β-actin | Mouse | Sigma | A5441 | DDDIAALV | NA |
| | | | | IDNGSGK | |
| | | | | (SEQ ID | |
| | | | | NO: 164) | |

### Quantification of Soluble Aβ38, Aβ40 and Aβ42:

Aβ38, Aβ40 and Aβ42 levels were measured in conditioned medium with either single-spot or multi spot 96 well plates pre-coated with neo epitope anti-Aβ38, anti-Aβ40, or -Aβ42 antibodies using MSD technology. MSD antibody coated plates were blocked with 150 μl/well PBS buffer supplemented with 0.1% casein for 1.5 hr at room temperature (600 rpm) and rinsed 5× with 200 μl/well washing buffer (PBS+0.05% Tween-20). 25 μL of SULFO-TAG labeled detection antibody against the N-terminus of human Abeta diluted in blocking buffer was mixed with 25 μL of standards (synthetic human Aβ38, Aβ40, Aβ42 peptides) or samples diluted in blocking buffer and loaded 50 μL per well. After overnight incubation at 4° C. plates were rinsed with washing buffer and 150 μl/well of the 2× MSD Read Buffer T was added. Plates were read immediately on MSD Sector Imager 6000.

### Results

Treatment with AON 20 (SEQ OD NO: 118, Table 2) resulted in a dose-dependent reduction of full-length mutant PSEN1, which reached 56% reduction at the highest 10 μM AON concentration (FIG. 1A). To investigate whether the observed reduction in PSEN1 protein levels leads to a reduction in the γ-secretase activity, the proteolytic processing of APP was assessed by quantifying the levels of APP C-terminal fragments (CTF), which is a direct substrate of the γ-secretase complex. Western blot analysis revealed an accumulation of APP CTFs in neurons treated with AON 20 (FIG. 1B). Consequently, the levels of soluble Aβ peptides were reduced, the Aβ38 levels even down to 50% (FIG. 1C). Noteworthy, the Aβ42/Aβ38 ratio was not modified by the AON treatment (FIG. 1D).

### Conclusions

AON 20, an AON of the invention, is effective in reducing mouse A431E mutant Psen1 protein levels. The downstream effect is shown by an increase in APP CTF and a decrease in Aβ peptide levels.

### Example 2

### Introduction

Mutations in Psen1, like A431E, reduce the functionality of γ-secretase and increase the production of long Aβ species (≥Aβ42) which leads to an abnormal Aβ profile with increased Aβ42/40 and Aβ42/38 ratios (Szaruga M et al., Cell. 2017 Jul. 27; 170(3): 443-456.e14, Petit D et al., Mol Psychiatry. 2022 Apr. 1). The majority of FAD patients carry one Psen1 mutant allele and one Wt allele. The therapeutic strategy of this invention aims at the specific reduction of mutant Psen1 to allow replacement by Wt Psen1 in the γ-secretase complex, and to restore normal Aβ peptides profiles. To investigate if the presence of the Wt Psen1 protein can indeed normalize the AB peptide profile and reduce the Aβ42/Aβ38 ratio, the Example 1 experiment was repeated, but with a different AON of the invention (AON 21, SEQ ID NO: 120 of Table 2) and using primary neurons derived from Psen1 A431E heterozygous mouse embryos (Psen1^{wt/A431E}; APPKi). Neurons were treated with AON 21 for 7 consecutive days and the levels of soluble Aβ38, Aβ40, and Aβ42 peptides in the medium were measured as a read-out of γ-secretase processivity.

### Material and Methods

See Example 1.

### Results

Treatment with AON 21 reduced the levels of the three Aβ peptides analysed (27% reduction of Aβ38, 50% of Aβ40, and 60% of Aβ42 compared to the untreated condition (FIG. 2A). Importantly, these changes in Aβ levels also translated into a 45% reduction of the Aβ42/Aβ38 ratio in treated heterozygous neurons (FIG. 2B), which suggests a shift in the production of Aβ species towards the generation of shorter Aβ38 peptides instead of Aβ42 peptides. This was not observed in the Psen1^{A431/A431E}; APPKi cells (Example 1), which is hypothesized to relate to the presence of Wt Psen1 protein, replacing the mutant Psen1 in the γ-secretase complex. Since Aβ plaques are mainly formed by Aβ42 peptide accumulation (Gravina et al., JBC. 1995: 270(13):7013-7016), such corrective effect is anticipated to slow down the progression of the disease.

### Conclusions

AON21, an AON of the invention, is effective in reducing long Aβ peptide levels and improving the Aβ42/Aβ38 ratio.

### Example 3

### Introduction

The majority of FAD patients carry one PSEN1 mutant allele and one Wt allele. The therapeutic strategy of this invention aims at the specific reduction of mutant PSEN1, with limited impact on expression of the Wt protein. The human PSEN1 A431E mutation is a C>A single nucleotide substitution. Compared to the A431E mouse target sequence of Examples 1 and 2, containing four base substitutions compared to the Wt sequence, the design of human A431E-specific AONs thus requires a higher level of fine-tuning chemical modifications and/or the position of the nucleotide opposite the mutation, to ensure maximum specificity for the mutated allele. A series of AONs was designed and initially screened for their activity versus specificity in fibroblasts transfected with plasmids containing either the Wt or A431E mutant PSEN1 cDNA construct. The goal of this experiment was to pre-select AONs with most favourable activity/specificity profiles to be further screened in iPSC derived neurons from FAD patients with the A431E mutation (see Example 5).

### Material and Methods:

### AONS

AONs specifically targeting the human A431E PSEN1 mRNA were designed (see Table 4). AONs had a backbone containing phosphorothioate (PS), a phospodiester (PO), dmi-Phosphoramidate (PNdmi) and/or a mesyl-phosphoramidate (PNms) with a DNA gap flanked by 2′-MOE wings at 5′ and 3′ end. The AONs were synthesized in 5 μmol scale by a MerMade 12 synthesizer (Bioautomation) using standard phosphoramidite protocols. The AONs were cleaved and deprotected in a two-step sequence (DEA followed by conc. NH₄OH treatment), purified by anion-exchange chromatography, desalted by size exclusion chromatography and lyophilized. Mass spectrometry confirmed the identity of all AONs, and purity (determined by UPLC) was found acceptable for all AONs (>80%).

TABLE 4
Human AON sequences

| AON | SEQ ID | |
|---|---|---|
| ID | NO | Sequence |
| AON1 | 2 | eTₛeC*ₛeTₛeTₛeTₛdCₛdTₛdTₛdGₛdAₛdAₛdAₛdAₛdTₛdGₛeGₛeC*ₛ |
| | | eAₛeAₛeG |
| AON2 | 4 | eAₛeTₛeTₛeC*ₛeTₛdTₛdTₛdCₛdTₛdTₛdGₛdAₛdAₛdAₛdAₛeTₛeGₛe |
| | | GₛeC*ₛeA |
| AON3 | 6 | eC*ₛeAₛeAₛeTₛeTₛdCₛdTₛdTₛdTₛdCₛdTₛdTₛdGₛdAₛdAₛeAₛeAₛe |
| | | TₛeGₛeG |
| AON4 | 8 | eGₛeC*ₛeAₛeAₛeTₛdTₛdCₛdTₛdTₛdTₛdCₛdTₛdTₛdGₛdAₛeAₛeAₛe |
| | | AₛeTₛeG |
| AON5 | 10 | eGₛeGₛeC*ₛeAₛeAₛdTₛdTₛdCₛdTₛdTₛdTₛdCₛdTₛdTₛdGₛeAₛeAₛe |
| | | AₛeAₛeT |
| AON6 | 12 | eTₛeGₛeGₛeC*ₛeAₛdAₛdTₛdTₛdCₛdTₛdTₛdTₛdCₛdTₛdTₛeGₛeAₛe |
| | | AₛeAₛeA |
| AON7 | 14 | eC*ₛeTₛeGₛeGₛeC*ₛdAₛdAₛdTₛdTₛdCₛdTₛdTₛdTₛdCₛdTₛeTₛeGₛ |
| | | eAₛeAₛeA |
| AON8 | 16 | eAₛeGₛeC*ₛeTₛeGₛdGₛdCₛdAₛdAₛdTₛdTₛdCₛdTₛdTₛdTₛeC*ₛeTₛ |
| | | eTₛeGₛeA |
| AON9 | 18 | eAₛeGₛeAₛeGₛeC*ₛdTₛdGₛdGₛdCₛdAₛdAₛdTₛdTₛdCₛdTₛeTₛeTₛ |
| | | eC*ₛeTₛeT |
| AON10 | 20 | eGₛeAₛeAₛeGₛeAₛdGₛdCₛdTₛdGₛdGₛdCₛdAₛdAₛdTₛdTₛeC*ₛeTₛ |
| | | eTₛeTₛeC* |
| AON11 | 22 | eTₛeGₛeGₛeAₛeAₛdGₛdAₛdGₛdCₛdTₛdGₛdGₛdCₛdAₛdAₛeTₛeTₛe |
| | | C*ₛeTₛeT |
| AON12 | 24 | eAₛeTₛeTₛeGₛeGₛdAₛdAₛdGₛdAₛdGₛdCₛdTₛdGₛdGₛdCₛeAₛeAₛ |
| | | eTₛeTₛeC* |
| AON13 | 26 | eGₛeC*ₛeAₛeAₛeTₛdUₛdCₛdTₛdTₛdTₛdCₛdTₛdTₛdGₛdAₛeAₛeAₛe |
| | | AₛeTₛeG |
| AON14 | 28 | eGₛeGₛeC*ₛeAₛeAₛdTₛdUₛdCₛdTₛdTₛdTₛdCₛdTₛdTₛdGₛeAₛeAₛ |
| | | eAₛeAₛeT |
| AON15 | 30 | eTₛeGₛeGₛeC*ₛeAₛdAₛdTₛdUₛdCₛdTₛdTₛdTₛdCₛdTₛdTₛeGₛeAₛe |
| | | AₛeAₛeA |
| AON16 | 33 | eGₛeGₒeC*ₒeAₒeAₛdTₛdUₛdCₛdTₛdTₛdTₛdCₛdTₛdTₛdGₛe |
| | | AₒeAₒeAₒeAₛeT |
| AON17 | 35 | eGₛeGₛeC*ₛeAₛeAₛdTₛdU_{PNms}dC_{PNms}dTₛdTₛdTₛdCₛdTₛdTₛdGₛe |
| | | AₛeAₛeAₛeAₛeT |
| AON18 | 37 | eGₛeGₛeC*ₛeAₛeAₛdTₛdUₛdC_{PNms}dT_{PNms}dTₛdTₛdCₛdTₛdTₛdGₛe |
| | | AₛeAₛeAₛeAₛeT |
| AON19 | 39 | eGₛeGₒeC*ₒeAₒeAₛdTₛdUₛdC_{PNms}dT_{PNms}dTₛdTₛdCₛdTₛdTₛdGₛe |
| | | AₒeAₒeAₒeAₛeT |
| AON22 | 41 | eGₛeGₛeC*ₛeAₛeAₛdAₛdTₛdCₛdTₛdTₛdTₛdCₛdTₛdTₛdGₛeAₛeAₛe |
| | | AₛeAₛeT |
| AON23 | 43 | eGₛeGₛeC*ₛeAₛeAₛdTₛdTₛdGₛdTₛdTₛdTₛdCₛdTₛdTₛdGₛeAₛeAₛe |
| | | AₛeAₛeT |
| AON24 | 45 | eGₛeGₛeC*ₛeAₛeAₛdTₛdTₛdGₛdAₛdTₛdTₛdCₛdTₛdTₛdGₛeAₛeAₛ |
| | | eAₛeAₛeT |
| AON25 | 47 | eGseC*oeAoeAoeTsdUsdCsdTsdTsdTsdCsdTsdTsdGsdAse |
| | | AoeAoeAoeTseG |
| AON26 | 49 | eGₛeGₛeC*ₛeAₛeAₛdTₛdTₛdCₛdTₛdAₛdAₛdCₛdTₛdTₛdGₛeAₛeAₛ |
| | | eAₛeAₛeT |
| AON27 | 51 | eGₛeGₛeC*ₛeAₛeAₛdTₛdTₛdCₛdTₛdTₛdAₛdGₛdTₛdTₛdGₛeAₛeAₛ |
| | | eAₛeAₛeT |
| AON28 | 53 | eC*ₛeAₛeAₛeTₛdTₛdCₛdTₛdTₛdTₛdCₛdTₛdTₛdGₛdAₛeAₛeAₛeAₛe |
| | | T |
| AON29 | 55 | eGₛeC*ₛeAₛeAₛdTₛdTₛdCₛdTₛdTₛdTₛdCₛdTₛdTₛdGₛeAₛeAₛeAₛe |
| | | A |
| AON30 | 57 | eGₛeGₛeC*ₛeAₛdAₛdTₛdTₛdCₛdTₛdTₛdTₛdCₛdTₛdTₛeGₛeAₛeAₛe |
| | | A |
| AON31 | 59 | eAₛeAₛeTₛdTₛdCₛdTₛdTₛdTₛdCₛdTₛdTₛdGₛdAₛeAₛeAₛeA |
| AON32 | 61 | eC*ₛeAₛeAₛdTₛdTₛdCₛdTₛdTₛdTₛdCₛdTₛdTₛdGₛeAₛeAₛeA |
| AON33 | 63 | eGₛeC*ₛeAₛdAₛdTₛdTₛdCₛdTₛdTₛdTₛdCₛdTₛdTₛeGₛeAₛeA |
| AON34 | 65 | eAₛeTₒeTₒeC*ₒeTₛdTₛdTₛdCₛdTₛdTₛdGₛdAₛdAₛdAₛdAₛeTₒeGₒe |
| | | GₒeC*ₛeA |
| AON35 | 67 | eAₛeT_{PNdmi}eT_{PNdmi}eC*_{PNdmi}eTₛdTₛdTₛdCₛdTₛdTₛdGₛdAₛdAₛdAₛd |
| | | AₛeT_{PNdmi}eG_{PNdmi}eG_{PNdmi}eC*ₛeA |
| AON36 | 69 | eGₛeGₒeC*ₒeAₒeAₛdTₛdTₛdCₛdTₛdTₛdTₛdCₛdTₛdTₛdGₛeAₒeAₒ |
| | | eAₒeAₛeT |
| AON37 | 71 | eGₛeG_{PNdmi}eC*_{PNdmi}eA_{PNdmi}eAₛdTₛdTₛdCₛdTₛdTₛdTₛdCₛdTₛdTₛ |
| | | dGₛeA_{PNdmi}eA_{PNdmi}eA_{PNdmi}eAₛeT |
| AON38 | 73 | eGₛeGₒeC*ₛeAₒeAₛdTₛdTₛdCₛdTₛdTₛdTₛdCₛdTₛdTₛd |
| | | GₛeAₒeAₛeAₒeAₛeT |
| AON39 | 75 | eAₛeGₒeC*deTₒeGₛdGₛdCₛdAₛdAₛdTₛdTₛdCₛdTₛdTₛdTₛeC*ₒeTₒ |
| | | eTₒeGₛeA |
| AON40 | 77 | eAₛeG_{PNdmie}C*_{PNdmie}T_{PNdmie}GₛdGₛdCₛdAₛdAₛdTₛdTₛdCₛdTₛd |
| | | TₛdTₛeC*_{PNdmie}T_{PNdmie}T_{PNdmie}GₛeA |
| AON41 | 79 | eAₛeGₒeC*ₛeTₒeGₛdGₛdCₛdAₛdAₛdTₛdTₛdCₛdTₛdTₛdTₛeC*ₒeTₛ |
| | | eTₒeGₛeA |
| AON42 | 81 | eGₛeAₒeAₒeGₒeAₛdGₛdCₛdTₛdGₛdGₛdCₛdAₛdAₛdTₛdTₛeC*ₒeTₒe |
| | | TₒeTₛeC* |
| AON43 | 83 | eGₛeA_{PNdmie}A_{PNdmie}G_{PNdmie}AₛdGₛdCₛdTₛdGₛdGₛdCₛdAₛdAₛdT |
| | | ₛdTₛeC*_{PNdmie}T_{PNdmie}T_{PNdmie}TₛeC* |
| AON44 | 85 | eTₛeGₒeGₒeC*ₒeAₛdAₛdTₛdUₛdCₛdTₛdTₛdTₛdCₛdTₛdTₛeGₒeAₒe |
| | | AₒeAₛeA |
| AON45 | 87 | eGₛeC*_{PNdmie}AₒeA_{PNdmie}TₛdUₛdCₛdTₛdTₛdTₛdCₛdTₛdTₛdGₛdA |
| | | ₛeAₒeAₒeAₒeT_{PNdmie}G |
| AON46 | 89 | eGₛeG_{PNdmie}C*ₒeA_{PNdmie}AₛdTₛdUₛdCₛdTₛdTₛdTₛdCₛdTₛdTₛdG |
| | | ₛeAₒeAₒeAₒeA_{PNdmie}T |
| AON47 | 91 | eTₛeG_{PNdmie}GₒeC*_{PNdmie}AₛdAₛdTₛdUₛdCₛdTₛdTₛdTₛdCₛdTₛdT |
| | | ₛeGₒeAₒeAₒeA_{PNdmie}A |
| AON48 | 93 | eGₛeGₛeC*ₛeAₛdAₛdTₛdUₛdCₛdTₛdTₛdTₛdCₛdTₛdTₛeGₛeAₛe |
| | | AₛeA |
| AON49 | 95 | eGₛeG_{PNdmie}C*ₒeAₛdAₛdTₛdUₛdCₛdTₛdTₛdTₛdCₛdTₛdTₛeGₒeAₒ |
| | | eA_{PNdmie}A |
| AON50 | 97 | eGₛeC*ₒeAceAₒeTₛdU_{PNms}dC_{PNms}dTₛdTₛdTₛdCₛdTₛdTₛdGₛdAₛe |
| | | AₒeAₒeAₒeTₛeG |
| AON51 | 99 | eGₛeC*ₒeAceAₒeTₛdUₛdC_{PNms}dT_{PNms}dTₛdTₛdCₛdTₛdTₛdGₛdAₛe |
| | | AₒeAceAeTₛeG |
| AON52 | 101 | eGₛeC*_{PNms}eA_{PNms}eAₒeTₛdUₛdC_{PNms}dT_{PNms}dTₛdTₛdCₛdTₛdTₛd |
| | | GₛdAₛeAₒeAₒeAₒeTₛeG |
| AON53 | 103 | eGₛeC*ₒeAₒeAceTₛdUₛdC_{PNms}dT_{PNms}dTₛdTₛdCₛdTₛdTₛdGₛdAₛe |
| | | AₛeAₛeAₛeTₛeG |
| AON54 | 105 | eGₛeC*ₛeAₛeAₛeTₛdUₛdC_{PNms}dT_{PNms}dTₛdTₛdCₛdTₛdTₛdGₛdAₛe |
| | | AₒeAₒeAₒeTₛeG |
| AON55 | 107 | eGₛeGₒeC*ceAceAₛdTₛdU_{PNms}dC_{PNms}dTₛdTₛdTₛdCₛdTₛdTₛdGₛe |
| | | AₒeAₒeAₒeAₛeT |
| AON56 | 109 | eGₛeG_{PNms}eC*_{PNms}eAₒeAₛdTₛdUₛdC_{PNms}dT_{PNms}dTₛdTₛdCₛdTₛd |
| | | TₛdGₛeAₒeAₒeAₒeAₛeT |
| AON57 | 111 | eGₛeG_{PNms}eC*_{PNms}eAₒeAₛdTₛdUₛdCₛdTₛdTₛdTₛdCₛdTₛdTₛdGₛe |
| | | AₒeAₒeAₒeAₛeT |
| AON58 | 113 | eGₛeGₒeC*_{PNms}eA_{PNms}eAₛdTₛdUₛdCₛdTₛdTₛdTₛdCₛdTₛdTₛdGₛe |
| | | AₒeAₒeAₒeAₛeT |
| AON59 | 115 | eGₛeGₒeC*ceAₒeAₛdTₛdUₛdCₛdTₛdTₛdTₛdCₛdTₛdTₛdGₛeAₒeAₒe |
| | | A_{PNms}eA_{PNms}eT |
| AON60 | 117 | eGₛeGₒeC*ceAₒeAₛdTₛdUₛdCₛdTₛdTₛdTₛdCₛdTₛdTₛdGₛeAceA_{PN} |
| | | ₘₛeA_{PNms}eAₒeT |
| Scrambled | 31 | eAₛeTₛeGₛeC*ₛeTₛdTₛdGₛdCₛdTₛdCₛdGₛdTₛdAₛdAₛdGₛeTₛeTₛe |
| | | AₛeTₛeC* |
| AON61 | 122 | eGₛeC*ₒeAₒeAₒeTₛdUₛmCₛdTₛdTₛdTₛdCₛdTₛdTₛdGₛdAₛeAₒeAₒe |
| | | AₒeTₛeG |
| AON62 | 124 | eGₛeGₒeC*ceAceAₛdTₛmUₛdCₛdTₛdTₛdTₛdCₛdTₛdTₛdGₛeAₒeAₒe |
| | | AₒeAₛeT |
| AON63 | 126 | eGₛeC*ₒeAceAₒeTₛdUₛdCₛdTₛdTₛdTₛdCₛdTₛdTₛdGₛdAₛeAₒeAₒe |
| | | AₒeT_{PNdmie}G |
| AON64 | 128 | eGₛeGₒeC*ceAceAₛdTₛdUₛdCₛdTₛdTₛdTₛdCₛdTₛdTₛdGₛeAₒeAₒe |
| | | AₒeA_{PNdmie}T |
| AON65 | 130 | eGₛeC*ₒeAceAₒeTₛdU_{PNms}mC_{PNms}dTₛdTₛdTₛdCₛdTₛdTₛdGₛdAₛe |
| | | AₒeAₒeAₒeTₛeG |
| AON66 | 132 | eGₛeC*ₒeAceAₒeTₛdUₛmC_{PNms}dT_{PNms}dTₛdTₛdCₛdTₛdTₛdGₛdAₛe |
| | | AₒeAₒeAₒeTₛeG |
| AON67 | 134 | eGₛeGₒeC*ₒeAₒeAₛdTₛmU_{PNms}dC_{PNms}dTₛdTₛdTₛdCₛdTₛdTₛdGₛ |
| | | eAₒeAₒeAₒeAₛeT |
| AON68 | 136 | eGₛeGₒeC*ₒeAₒeAₛdTₛmUₛdC_{PNms}dT_{PNms}dTₛdTₛdCₛdTₛdTₛdGₛ |
| | | eAₒeAₒeAₒeAₛeT |
| AON69 | 138 | eGₛeC*ₒeAₒeAₒeTₛdU_{PNms}dC_{PNms}dTₛdTₛdTₛdCₛdTₛdTₛdGₛdAₛe |
| | | AₒeAₒeAₒeT_{PNdmi}eG |
| AON70 | 140 | eGₛeC*ₒeAₒeAₒeTₛdUₛdC_{PNms}dT_{PNms}dTₛdTₛdCₛdTₛdTₛdGₛdAₛe |
| | | AₒeAₒeAₒeT_{PNdmi}eG |
| AON71 | 142 | eGₛeGₒeC*ₒeAₒeAₛdTₛdU_{PNms}dC_{PNms}dTₛdTₛdTₛdCₛdTₛdTₛdGₛe |
| | | AₒeAₒeAₒeA_{PNdmi}eT |
| AON72 | 144 | eGₛeGₒeC*ₒeAₒeAₛdTₛdUₛdC_{PNms}dT_{PNms}dTₛdTₛdCₛdTₛdTₛdGₛe |
| | | AₒeAₒeAₒeA_{PNdmi}eT |
| AON89 | 219 | eGₛeC*ₒeAₛeAₒeTₛdUₛdCₛdTₛdTₛdTₛdCₛdTₛdTₛdGₛdAₛeAₒeAₛ |
| | | eAceTₛeG |
| AON90 | 22 | eGₛeGₒeC*ₛeAceAₛdTₛdUₛdCₛdTₛdTₛdTₛdCₛdTₛdTₛdGₛeAₒeAₛ |
| | | eAₒeAₛeT |
| AON91 | 223 | eTₛeGₒeGₛeC*ₒeAₛdAₛdTₛdUₛdCₛdTₛdTₛdTₛdCₛdTₛdTₛeGₒeAₛ |
| | | eAₒeAₛeA |
| AON92 | 225 | eGₛeC*ₛeAₛeAₛeTₛdUₛdC*ₛdTₛdTₛdTₛdC*ₛdTₛdTₛdGₛdAₛeAₛeA |
| | | ₛeAₛeTₛeG |
| AON93 | 227 | eGₛeGₛeC*ₛeAₛeAₛdTₛdUₛdC*ₛdTₛdTₛdTₛdC*ₛdTₛdTₛdGₛeAₛeA |
| | | ₛeAₛeAₛeT |
| AON94 | 229 | eTₛeGₛeGₛeC*ₛeAₛdAₛdTₛdUₛdC*ₛdTₛdTₛdTₛdC*ₛdTₛdTₛeGₛeA |
| | | ₛeAₛeAₛeA |
| AON95 | 23 | eGₛeC*ₒeAₒeAₒeTₛdUₛdC*ₛdTₛdTₛdTₛdC*ₛdTₛdTₛdGₛdAₛeAₒeA |
| | | ₒeAₒeTₛeG |
| AON96 | 233 | eGₛeGₒeC*ₒeAₒeAₛdTₛdUₛdC*ₛdTₛdTₛdTₛdC*ₛdTₛdTₛdGₛeAce |
| | | AₒeAₒeAₛeT |
| AON97 | 235 | eTₛeGₒeGₒeC*ₒeAₛdAₛdTₛdUₛdC*ₛdTₛdTₛdTₛdC*ₛdTₛdTₛeGₒe |
| | | AₒeAₒeAₛeA |
| AON98 | 237 | eGₛeC*ₒeAₛeAₒeTₛdUₛdC*ₛdTₛdTₛdTₛdC*ₛdTₛdTₛdGₛdAₛeAₒeA |
| | | ₛeAₒeTₛeG |
| AON99 | 239 | eGₛeGₒeC*ₛeAₒeAₛdTₛdUₛdC*ₛdTₛdTₛdTₛdC*ₛdTₛdTₛdGₛeAce |
| | | AₛeAₒeAₛeT |
| AON100 | 241 | eTₛeGₒeGₛeC*ₒeAₛdAₛdTₛdUₛdC*ₛdTₛdTₛdTₛdC*ₛdTₛdTₛeGₒeA |
| | | ₛeAₒeAₛeA |
| AON101 | 243 | eG_{PNdmi}eC*ₒeAₛeAₒeTₛdUₛdCₛdTₛdTₛdTₛdCₛdTₛdTₛdGₛdAₛeAₒ |
| | | eAₛeAₒeT_{PNdmi}eG |
| AON102 | 245 | eG_{PNdmi}eC*_{PNdmi}eA_{PNdmi}eAₒeTₛdUₛdCₛdTₛdTₛdTₛdCₛdTₛdTₛdGₛ |
| | | dAₛeAₒeA_{PNdmi}eA_{PNdmi}eT_{PNdmi}eG |
| AON103 | 247 | eG_{PNdmi}eC*ₒeA_{PNdmi}eAₒeTₛdUₛdCₛdTₛdTₛdTₛdCₛdTₛdTₛdGₛdAₛ |
| | | eAₒeA_{PNdmi}eAₒeT_{PNdmi}eG |
| AON104 | 249 | eG_{PNdmi}eC*ₒeA_{PNdmi}eAₒeT_{PNdmi}dUₛdCₛdTₛdTₛdTₛdCₛdTₛdTₛdGₛ |
| | | dA_{PNdmi}eAₒeA_{PNdmi}eAₒeT_{PNdmi}eG |
| AON105 | 251 | eG_{PNdmi}eC*_{PNdmi}eAₛeAₒeTₛdUₛdCₛdTₛdTₛdTₛdCₛdTₛdTₛdGₛdAₛ |
| | | eAₒeAₛeA_{PNdmi}eT_{PNdmi}eG |
| AON106 | 253 | eG_{PNdmi}eC*_{PNdmi}eAₛeA_{PNdmi}eTₛdUₛdCₛdTₛdTₛdTₛdCₛdTₛdTₛdGₛ |
| | | dAₛeA_{PNdmi}eAₛeA_{PNdmi}eT_{PNdmi}eG |
| AON107 | 255 | eG_{PNdmi}eGₒeC*ₛeAceAₛdTₛdUₛdCₛdTₛdTₛdTₛdCₛdTₛdTₛdGₛeAₒ |
| | | eAₛeAₒeA_{PNdmi}eT |
| AON108 | 257 | eG_{PNdmi}eG_{PNdmi}eC*_{PNdmi}eAₒeAₛdTₛdUₛdCₛdTₛdTₛdTₛdCₛdTₛdTₛ |
| | | dGₛeAₒeA_{PNdmi}eA_{PNdmi}eA_{PNdmi}eT |
| AON109 | 259 | eG_{PNdmi}eGₒeC*_{PNdmi}eAₒeAₛdTₛdUₛdCₛdTₛdTₛdTₛdCₛdTₛdTₛdGₛ |
| | | eAₒeA_{PNdmi}eAₒeA_{PNdmi}eT |
| AON110 | 261 | eG_{PNdmi}eGₒeC*_{PNdmi}eAₒeA_{PNdmi}dTₛdUₛdCₛdTₛdTₛdTₛdCₛdTₛdTₛ |
| | | dG_{PNdmi}eAₒeA_{PNdmi}eAₒeA_{PNdmi}eT |
| AON111 | 263 | eG_{PNdmi}eG_{PNdmi}eC*ₛeAₒeAₛdTₛdUₛdCₛdTₛdTₛdTₛdCₛdTₛdTₛdGₛ |
| | | eAₒeAₛeA_{PNdmi}eA_{PNdmi}eT |
| AON112 | 265 | eG_{PNdmi}eG_{PNdmi}eC*ₛeA_{PNdmi}eAₛdTₛdUₛdCₛdTₛdTₛdTₛdCₛdTₛdTₛ |
| | | dGₛeA_{PNdmi}eAₛeA_{PNdmi}eA_{PNdmi}eT |
| AON113 | 267 | eT_{PNdmi}eGₒeGₛeC*ₒeAₛdAₛdTₛdUₛdCₛdTₛdTₛdTₛdCₛdTₛdTₛeGₒ |
| | | eAₛeAₒeA_{PNdmi}eA |
| AON114 | 269 | eT_{PNdmi}eG_{PNdmi}eG_{PNdmi}eC*ₒeAₛdAₛdTₛdUₛdCₛdTₛdTₛdTₛdCₛdTₛ |
| | | dTₛeGₒeA_{PNdmi}eA_{PNdmi}eA_{PNdmi}eA |
| AON115 | 271 | eT_{PNdmi}eGₒeG_{PNdmi}eC*ₒeAₛdAₛdTₛdUₛdCₛdTₛdTₛdTₛdCₛdTₛdTₛ |
| | | eGₒeA_{PNdmi}eAₒeA_{PNdmi}eA |
| AON116 | 273 | eT_{PNdmi}eGₒeG_{PNdmi}eC*ₒeA_{PNdmi}dAₛdTₛdUₛdCₛdTₛdTₛdTₛdCₛdTₛ |
| | | dT_{PNdmi}eGₒeA_{PNdmi}eAₒeA_{PNdmi}eA |
| AON117 | 275 | eT_{PNdmi}eG_{PNdmi}eGₛeC*ₒeAₛdAₛdTₛdUₛdCₛdTₛdTₛdTₛdCₛdTₛdTₛ |
| | | eGₒeAₛeA_{PNdmi}eA_{PNdmi}eA |
| AON118 | 277 | eT_{PNdmi}eG_{PNdmi}eGₛeC*_{PNdmi}eAₛdAₛdTₛdUₛdCₛdTₛdTₛdTₛdCₛdTₛ |
| | | dTₛeG_{PNdmi}eAₛeA_{PNdmi}eA_{PNdmi}eA |

A = adenosine; G = guanine; T = thymine; C = cytosine; U = Uracil; C* = 5-methylcytosine. D = DNA, e = 2′-MOE, m = 2′-OMe, ₛ = PS linkage, ₒ = PO linkage, _{PNms} = _{PNms} linkage, _{PNdmi} = PNdmi linkage.
The corresponding sequences of the non-modified oligonucleotides are represented by SEQ ID NO: 1, 3, 5, 7, 9, 11, 13,15, 17, 19, 21, 23, 25, 27, 29, 32, 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 112, 114, 116, 121, 123, 125, 127, 129, 131, 133, 135, 137, 139, 141, 143, 218, 220, 222, 224, 226, 228, 230, 232, 234, 236, 238, 240, 242, 244, 246, 248, 250, 252, 254, 256, 258, 260,262, 264, 266, 268, 270, 272, 274, 276.

### Transfection of Human PSEN1 Expression Vectors and AONs

NIH3T3 cells were cultured to 80% confluency in 6-wells plate and transfected with 2 μg of plasmid encoding the human wild-type (Wt) PSEN1 variant 1 (NM_000021.4) or mutant A431E PSEN1 transcripts with Flag-tags at the N-terminus. These plasmids also express Neomycin Phosphotransferase II (NPT-II). Three hours after plasmid transfection, cells were transfected with 10 nM AON. Transfections were done using Lipofectamine 2000 and following manufacturer's instructions. Cells were harvested 24 hours post AON transfection.

### Protein Extraction and WES:

Proteins were extracted from cell pellets using PLB-G Lysis buffer (16% SDS, 0.07 M Tris-HCl pH 6.8, 21% Glycerol, 50 mM DTT, and 1× proteases Inhibitor Cocktail) and mechanical dissociation with a syringe. Protein concentration was measured with Pierce 660 nm Protein Assay Reagent (Thermo) supplemented with Ionic Detergent Compatibility Reagent (Thermo). Protein levels of Flag-PSEN1 and NPT-II were quantified using WES technology (Protein simple) according to the manufacturer's instructions. The Jess/Wes Separation 12-230 kDa kit (Protein simple) was used. The following primary antibodies and concentrations were used: anti Flag-tag antibody (MAβ8529, R&D Systems, 1/1000) and anti-NPT-II antibody (NB110-60487, Bio-Techne, 1/2500). For correct NPT-II detection, anti-Mouse IgG H&L (ab6708, Abcam) was added to the anti-mouse Secondary antibody at 1/100 dilution. For each sample, the area of the Flag peak was divided by the area of the NPT-II peak to normalise each sample for plasmid transfection efficacy. Subsequently, the Flag/NPT-II ratio of each treated sample was normalised to the ratio of the control untreated sample, which was transfected with the plasmid but not with the AON.

### Results

A first series of AONs was tested wherein sequence and the position of the nucleotide opposite the mutation shifted from the 5′ end to the 3′ end (Table 4, FIG. 3A). Results indicated that the best efficacy and selectivity towards the mutant A431E PSEN1 was obtained with AONs wherein the mutational position is within the 3 first positions of the central part of the antisense oligonucleotide starting from 5′ to 3′ (i.e., in position 6, 7 or 8 of the oligonucleotide sequence starting from 5′ to 3′) (AON4, AON5, AON6, SEQ ID NO:8, 10, 12). AON4 and AON5 resulted in a 70% reduction of the mutant A431E PSEN1 ratio, while this ratio is reduced up to 80% with AON6. In comparison, the reduction of the WT PSEN1 ratio reached 11% with AON4, 30% with AON5 and 26% with AON6. No significant reduction was observed in cells treated with a scrambled AON compared to the AON untreated sample (FIG. 3A).

In a second series, AON variants of AON4, AON5 and AON6 were designed containing a T (thymine) to U (uracil) substitution at the position opposite the mutation (Table 4, FIG. 3B). This resulted in a higher specificity for the mutant allele: AON13, AON14 and AON15 (SEQ ID NO:26, 28, 30) reduced PSEN1 A431E protein levels to similar levels as AON4, AON5 and AON6 (70%, 76% or 81%, respectively), but the reduction of the WT PSEN1 protein levels was less: 5% for AON13 and AON15, and 20% for AON14 (FIG. 3B).

In a third series, AON variants of AON14 (SEQ ID NO:28) were designed in which some PS linkages were substituted for PO, PNdmi, and/or PNms linkages (FIG. 3C). We hypothesized that the reduction of PS content might reduce AON in-vivo toxicity (Moazami, et al., BioRxiv 2021). The addition of PNdmi or PNms might increase AON potency and stability (Anderson B A et al., Nucleic Acids Res. 2021 Sep. 20; 49(16):9026-9041. Kandasamy P et al., Nucleic Acids Res. 2022 Feb. 2), while the incorporation of PO is predicted to increase the Tm which might result in a partial loss of AON allele specificity due to stronger binding to WT PSEN1 mRNA. Surprisingly, the introduction of three PO linkages at each wing of AON14 did not alter the AON efficacy or allele selectivity: AON16 reduces WT and A431E PSEN1 protein levels to similar levels as AON14 (85% reduction of A431E and 10% reduction of WT) (FIG. 3D). Similarly, the introduction of two PNms linkages within the positions +2 and +4 of the central part of the antisense oligonucleotide starting from 5′ to 3′ in a full PS backbone (AON17 and AON18, SEQ ID NO:35 and 37) or in a mix PO/PS backbone (AON19, SEQ ID NO:39) did not significantly impact the AON efficacy or allele selectivity: AON17, AON18 and AON19 reduce PSEN1 A431E and WT protein levels at similar levels that AON14 (for PSEN1 A431E: 79%, 82%, and 79%, respectively, while for WT PSEN1: 1%, 8%, and 24%, respectively) (FIG. 3D).

### Conclusions

These results show that the best effectivity and specificity is obtained when the position of the nucleotide opposite the mutation is at +6, +7 or +8 of the oligonucleotide sequence starting from 5′ to 3′ (i.e, in the 3 first positions of the central part of the antisense oligonucleotide starting from 5′ to 3′), and that T>U substitution at that position increases allele selectivity while keeping a similar effectivity towards the mutant PSEN1. In addition, the results show that the addition of PO and/or PNms linkages does not significantly impact the efficacy and allele selectivity.

### Example 4

### Introduction

Results in Example 3 show that AONs targeting human A431E PSEN1 have the best effectivity and allele specificity when the position of the nucleotide opposite the mutation is at position +6, +7, or +8 of the oligonucleotide sequence starting from 5′ to 3′ (i.e, in the 3 first positions of the central part of the antisense oligonucleotide starting from 5′ to 3′), and that T>U substitution at that position increases allele selectivity while keeping a similar effectivity towards the mutant PSEN1. The goal of this experiment is to determine if these criteria in AON design apply to another C/G/T>A mutation in PSEN1. More than 150 different mutations in PSEN1 have been described in FAD patients. Of those, 4% are caused by C>A mutations (like A431E), and 17% are caused by C/G/T>A mutations. A series of AONs targeting human S212Y (caused by C>A mutation) were designed and tested in fibroblasts transfected with plasmids containing either the Wt or S212Y mutant PSEN1 cDNA construct. AON efficacy and allele selectivity were asses on mRNA by Digital droplet (dd) PCR.

### Material and Methods

### AONS

AONs specifically targeting human S212Y PSEN1 mRNA were designed (see Table 5). AONs had a backbone containing phosphorothioate (PS) with a DNA gap flanked by 2′-MOE wings at 5′ and 3′ end. AONs were synthesized as explained in Example 3. Six AONs targeting A431E were also tested in this experimental set-up (AON4, AON5, AON6, AON13, AON14 and AON15; see Table 4).

TABLE 5
Human AON sequences targeting S212Y

| | SEQ | | | |
|---|---|---|---|---|
| | ID | | | |
| AON ID | NO | A | B | Sequence |
| AON73 | 174 | 2 | T | eGₛeTₛeAₛeAₛeAₛdTₛdCₛdAₛdTₛdTₛdCₛdCₛdCₛdAdCₛeC*ₛeAₛeC*ₛeAₛeC* |
| AON74 | 176 | 4 | T | eAₛeTₛeGₛeTₛeAₛdAₛdAₛdTₛdCₛdAₛdTₛdTₛdCₛdCₛdCₛeAₛeC*ₛeC*ₛeAₛeC* |
| AON75 | 178 | 6 | T | eGₛeAₛeAₛeTₛeGₛdTₛdAₛdAₛdAₛdTₛdCₛdAₛdTₛdTₛdCₛeC*ₛeC*ₛeAₛeC*ₛeC* |
| AON76 | 180 | 8 | T | eGₛeTₛeGₛeAₛeAₛdTₛdGₛdTₛdAₛdAₛdAₛdTₛdCₛdAₛdTₛeTₛeC*ₛeC*ₛeC*ₛeA |
| AON77 | 182 | 10 | T | eC*ₛeAₛeGₛeTₛeGₛdAₛdAₛdTₛdGₛdTₛdAₛdAₛdAₛdTₛdCₛeAₛeTₛeTₛeC*ₛeC* |
| AON78 | 184 | 12 | T | eTₛeC*ₛeC*ₛeAₛeGₛdTₛdGₛdAₛdAₛdTₛdGₛdTₛdAₛdAₛdAₛeTₛeC*ₛeAₛeTₛeT |
| AON79 | 186 | 14 | T | eTₛeTₛeTₛeC*ₛeC*ₛdAₛdGₛdTₛdGₛdAₛdAₛdTₛdGₛdTₛdAₛeAₛeAₛeTₛeC*ₛeA |
| AON80 | 188 | 2 | U | eGₛeUₛeAₛeAₛeAₛdTₛdCₛdAₛdTₛdTₛdCₛdCₛdCₛdAₛdCₛeC*ₛeAₛeC*ₛeAₛeC* |
| AON81 | 190 | 4 | U | eAₛeTₛeGₛeUₛeAₛdAₛdAₛdTₛdCₛdAₛdTₛdTₛdCₛdCₛdCₛeAₛeC*ₛeC*ₛeAₛeC* |
| AON82 | 192 | 6 | U | eGₛeAₛeAₛeTₛeGₛdUₛdAₛdAₛdAₛdTₛdCₛdAₛdTₛdTₛdCₛeC*ₛeC*ₛeAₛeC*ₛeC* |
| AON83 | 194 | 8 | U | eGₛeTₛeGₛeAₛeAₛdTₛdGₛdUₛdAₛdAₛdAₛdTₛdCₛdAₛdTₛeTₛeC*ₛeC*ₛeC*ₛeA |
| AON84 | 196 | 10 | U | eC*ₛeAₛeGₛeTₛeGₛdAₛdAₛdTₛdGₛdUₛdAₛdAₛdAₛdTₛdCₛeAₛeTₛeTₛeC*ₛeC* |
| AON85 | 198 | 12 | U | eTₛeC*ₛeC*ₛeAₛeGₛdTₛdGₛdAₛdAₛdTₛdGₛdUₛdAₛdAₛdAₛeTₛeC*ₛeAₛeTₛeT |
| AON86 | 200 | 14 | U | eTₛeTₛeTₛeC*ₛeC*ₛdAₛdGₛdTₛdGₛdAₛdAₛdTₛdGₛdUₛdAₛeAₛeAₛeTₛeC*ₛeA |

A = adenosine; G = guanine; T = thymine; C = cytosine; U = Uracil; C* = 5-methylcytosine. D = DNA, e = 2′-MOE, ₛ = PS linkage.
Column A = Position of mutation within the antisense oligonucleotide sequence starting from 5′ to 3′.
Column B = Nucleotide opposing the mutation.
The corresponding sequences of the non-modified oligonucleotides are represented by SEQ ID NO: 173, 175, 177, 179, 181, 183, 185, 187, 189, 191, 193, 195, 197, 199.

### Transfection of Human PSEN1 Expression Vectors and AONs

NIH3T3 cells were transfected with plasmid encoding human wild-type (Wt) PSEN1 variant 1 (NM_000021.4), mutant A431E PSEN1 or mutant S212Y PSEN1 transcripts. See Example 3 for more details on cell culture and transfection procedures.

RNA Extraction and cDNA Synthesis

Twenty-four hours after transfection cells were harvested and total RNA was extracted with the RNAse Mini Kit (Ref: 74104, Qiagen). DNAse treatment was done on the column during RNA extraction following manufacture's instructions (Rnase-Free DNase Set, Ref: 79254, Qiagen). 200 ng of RNA were used as input for the cDNA synthesis using SuperScript IV Enzyme (Thermo Fisher, Ref: 18090050) and random hexamer primers (Sigma, 11034731001).

### Digital Droplet (Dd) PCR Analysis

Specific Taqman minor groove binder (MGB) assays were designed to detect human PSEN1 and NPT-II encoded in the plasmid (Table 6). Taqman assays were purchased from Applied Biosystems. Digital droplet PCR analysis was performed on 1 μl of cDNA (cDNA was diluted 20× for PSEN1 assay and 400× for NPT-II assay) in a 20 μl reaction volume using an annealing/extension temperature of 60° C. according to the manufacturer's instructions (BioRad). mRNA copies of hPSEN1 were normalised to mRNA copies of NPT-II and expressed relative to non-AON treated samples. Data is expressed as a ratio of the relative levels of WT PSEN1 versus Mutant PSEN1.

TABLE 6
Taqman Assays hPSEN1 plasmid

| | | | | Amplicon | Seq |
|---|---|---|---|---|---|
| Assay | Target | Sequences | | length | ID No |
| hPSEN1_ | hPSEN1 | Forward | CCTTTTATGGA | 74 | 212 |
| pl | in | primer | CCAATTAGCAT | | |
| | plasmid | Reverse | TCCGGCCATCA | | 213 |
| | | primer | CCACTTTGTAC | | |
| | | Probe | AAGTCAATTTT | | 214 |
| | | (FAM) | ATATCTAGACC | | |
| | | | CAGCTT | | |
| NeoR | NPT-II | Forward | CGGCTGCATAC | 68 | 215 |
| | | primer | GCTTGATCGCT | | |
| | | Reverse | CGCTCGATGCG | | 216 |
| | | primer | ATGTTACCTGC | | |
| | | Probe | CCATTCGACCA | | 217 |
| | | (VIC) | | | |

### Results

Initially, we tested if the effect in allele selectivity and efficacy described in Example 3 on protein by WES was also detectable on mRNA by (dd) PCR (FIG. 4A). Data is shown as the ratio between the mRNA levels of WT and mutant PSEN1. If the ratio is 1, the AON has no allele selectivity. The higher the ratio is, the more allele preference towards the mutant has the AON. FIG. 4A shows that AONs containing the mutation in positions +6, +7, or +8 of the oligonucleotide sequence starting from 5′ to 3′ (i.e, in the 3 first positions of the central part of the antisense oligonucleotide starting from 5′ to 3′) have 2-3 times more preference towards mutant A431E than WT. In addition, the ratio WT/Mutant is higher in AONs that have a U opposing the mutation instead of T (Especially in positions +6 and +8 of the oligonucleotide sequence starting from 5′ to 3′).

Next, a set of AONs targeting S212Y PSEN1 with mutations in different positions and with a T>U substitution opposite to the mutation were designed and tested (Table 5). Results in FIG. 4B show that AONs with the mutation in positions +6, +8 and +10 of the oligonucleotide sequence starting from 5′ to 3′ (i.e., in positions +1, +3, +5 of the central part of the antisense oligonucleotide starting from 5′ to 3′), show the highest allele selectivity (Ratio Wt/Mutant is higher than 1.5). In addition, T to U substitution opposite to the mutation improves allele selectivity in positions +6, +8, and +12 (i.e., in positions +1, +3, +7 of the central part of the antisense oligonucleotide starting from 5′ to 3′).

### Conclusions

These results show that the best effectivity and specificity are obtained when the position of the nucleotide opposite the mutation is between position +6 and +10 of the oligonucleotide sequence starting from 5′ to 3′ (i.e., in the first five positions of the central part of the antisense oligonucleotide starting from 5′ to 3′) in 2 different PSEN1 mutations. In addition, for both PSEN1 mutations, there are specific positions where a T>U substitution opposite to the mutation increases allele selectivity. Therefore, our newly defined criteria on AON design at least apply to two PSEN1 mutations from FAD patients (A431E and S212Y).

### Example 5

### Introduction

Neurons are one of the major cell types affected in FAD patients. In Example 3, a group of efficient and allele-selective AONs targeting A431E PSEN1 was identified using mouse fibroblasts and plasmids containing either the Wt or A431E mutant PSEN1 cDNA construct. To determine if selected AONs are also efficient and selective for mutant PSEN1 in a disease-relevant cell, neurons differentiated from FAD patient-derived iPSC were treated for 10 consecutive days with AON14 (SEQ ID NO:28), AON15 (SEQ ID NO:30), and AON 16 (SEQ ID NO:33). All the selected AONs fulfil our newly established design criteria for best AON effectivity and allele selectivity: nucleotide opposite the mutation between positions +6 and +8 of the oligonucleotide sequence starting from 5′ to 3′ (i.e., first three positions of the central part of the antisense oligonucleotide starting from 5′ to 3′), and a T>U substitution on that position.

### Material and Methods

### AONS

See Table 4 in Example 3.

iPSC Differentiation to Neurons and AON Gymnotic Uptake iPSC derived from a patient carrying A431E PSEN1 mutation in heterozygosis were differentiated into neurons (cells generated by Bart de Strooper group). Briefly, iPSC were seeded as a monolayer in a Matrigel-coated 6-well plate and cultured for 12 days in Neuronal Induction Medium (NIM, see composition in Table 8). On Days in Vitro (DIV) 12, the monolayer of cells was mechanically dissociated and cells were seeded in a Matrigel-coated 6-well plate and Neuronal Maintenance Medium (N.M.M., see composition in Table 7) to induce neural progenitor differentiation. On DIV 24-26, Neural Progenitor Cells were dissociated and seeded for neuronal maturation in N.M.M medium on Poly-L-Ornithine-laminin-coated plates with N.M.M medium and treated for 48 h with 10 μM DAPT (Tocris; cat #2634) on DIV31. On DIV36, Neural progenitor cells were split and seeded in a Poly-L-Ornithine-laminin-coated 96-wells plate with N. M. M medium for further neuronal maturation (30.000 cells/well). AON treatment started on DIV62, during 10 days 10 μM of AON14, AON 15 or AON 16 (Table 4) were added daily to the medium. On DIV71 neurons were harvest.

TABLE 7
Composition of Neural induction medium

| Media component | ml |
|---|---|
| Neurobasal medium (Life technologies; cat# 21103-049) | 240 |
| DMEM:F12 Glutamax (Life Technologies; cat# 31331-028) | 240 |
| B27 supplement (Life Technologies; cat# 17504-044) | 5 |
| N2 supplement (Life Technologies; cat# 17502-048) | 2.5 |
| Glutamax (Life Technologies; cat# 35050038) | 2.5 |
| Pen/Strep (Sigma cat# P4333-100 ml) | 2.5 |
| Insulin (Sigma, I9278-5 ml) | 0.125 |
| 2-mercaptoethanol 50 mM (Life Technologies; cat# | 0.5 |
| 31350010) | |
| MEM NEAA (Life Technologies; cat# 11140-035 | 2.5 |
| sodium pyruvate (Life Technologies; cat# 58636) | 2.5 |

TABLE 8
Composition of Neuronal induction medium (NIM)

| Media component | | |
|---|---|---|
| Neuronal maintenance medium (see above) | 50 | ml |
| SB431542 10 nM (miltenyibiotec; cat# 130-106-275) | 50 | μl |
| LDN-193189 10 nM (Stemcell tech; cat# 72147) | 5 | μl |

RNA Extraction and cDNA Synthesis

Total RNA was extracted using RNeasy 96 QIAcube HT Kit (Ref: 74171, Qiagen). DNAse treatment was done on the column during RNA extraction following the manufacture's instructions (Rnase-Free DNase Set, Ref: 79254, Qiagen). A clean-up protocol was performed to detach the AON from the RNA. Briefly, RNA was mixed with RTL buffer and incubated for 5 min at 95° C. followed by cool for 2 min on ice incubation. Then, RNA was purified using the RNeasy MinElute Cleanup Kit following the manufacture's instructions (Ref: 74204, Qiagen). 200 ng of RNA were used as input for the cDNA synthesis using SuperScript IV Enzyme (Thermo Fisher, Ref: 18090050) and random hexamer primers (Sigma, 11034731001).

### Digital Droplet (Dd) PCR Analysis

SNP assays to detect human WT and A431E PSEN1 were designed by BioRad (Assay ID hPSEN1 WT: dMDW4344923033; Assay ID hPSEN1 A431E: dMDM4344923031). Taqman assays for housekeeping genes were purchased at Thermo Fisher: TFRC (Ref: Hs99999911_m1) and GAPDH (Ref: Hs04420632_g1). (dd) PCR analysis was performed on 2 μl of cDNA for PSEN1 and TFRC or 2 μl of 20× diluted cDNA for GAPDH in a 20 μl reaction volume using an annealing/extension temperature of 55° C. according to the manufacturer's instructions (BioRad). mRNA copies of WT or A431E PSEN1 were normalised to the copies of TFRC and GAPDH housekeeping genes. Data is expressed relative to the control sample with no AON treatment.

### Results

Treatment of iPSC-derived neurons during 10 days with AON14, AON15 and AON16 (Table 4) results in a selective reduction of mutant A431E PSEN1 mRNA levels. Mutant A431E mRNA levels are reduced by 49%, 62% and 63%, respectively, while WT PSEN1 mRNA levels are reduced by 17%, 22% and 22%, respectively.

### Conclusions

Our results show that three AONs of the invention (AON14, AON15 and AON16), specifically reduce the levels of mutant A431E PSEN1 in a disease-relevant cell line (neurons differentiated from FAD patient-derived iPSC).

### Example 6

### Introduction

Mutations in Psen1 (like A431E) are believed to reduce the functionality of γ-secretase and increase the production of long Aβ species (≥Aβ42), which leads to an abnormal Aβ profile with increased Aβ42/40 and Aβ42/38 ratios (Szaruga M et al., Cell. 2017 Jul. 27; 170 (3): 443-456.e14, Petit D et al., Mol Psychiatry. 2022 Apr. 1). To confirm that A431E mutation increases the production of long Aβ species (≥Aβ42) and induces an abnormal Aβ profile in neurons, we repeated the experiments from Example 2 with an AON that targets specifically mouse WT Psen1 (AON87, Table 9). Primary neurons derived from Psen1 A431E heterozygous mouse embryos (Psen1^{wt/A431E}; APPKi) were treated with AON87 for 7 consecutive days and the mRNA levels of WT and A431 Psen1, as well as, the levels of soluble Aβ38, Aβ40, and Aβ42 peptides were quantified.

### Material and Methods

### AONS

For details about AON synthesis see Example 1.

TABLE 9
Additional mouse AON sequences

| | | SEQ | |
|---|---|---|---|
| | AON | ID | |
| Target | ID | NO: | Sequence (5′-3′) |
| WT | AON87 | 202 | eAₛeGₒeGₒeGₒeC*ₛdTₛ |
| mPsen1 | | | dGₛdGₛdCₛdAₛdAₛdCₛd |
| | | | GₛdCₛdTₛdTₛeTₒeC*ₒe |
| | | | TₒeTₛeG |
| A431E | AON88 | 204 | eAₛeGₒeC*ₒeGₒeC*ₛd |
| mPsen1 | | | CₛdGₛdGₛdCₛdAₛdAₛd |
| | | | TₛdTₛdCₛdTₛdTₛeTₒe |
| | | | C*ₒeTₒeTₛeG |

A = adenosine; G = guanine; T = thymine; C = cytosine; C* = 5-methylcytosine. d = DNA, e = 2′-MOE, ₛ = PS linkage. ₒ = PO linkage.

Corresponding non modified sequence of the oligonucleotide represented by SEQ ID NO: 201, 203.

Primary mouse neuronal cultures, Gymnotic uptake, Protein extraction and Quantification of soluble Aβ38, A340 and Aβ42: see Example 1.
RNA extraction and cDNA synthesis: see Example 5

### Real-Time PCR (RT-PCR):

Specific primers were designed to amplify mouse WT Psen1, A431E Psen1, Actin and Rps23 (Table 10). RT-qPCR analysis was performed on 5 ng of cDNA in a 15 μl reaction volume using an annealing/extension temperature of 65.5° C. and PowerUP SYBR green Master mix (Thermo Fisher, A25741). RT-qPCR was performed with ViiA 7 Real-Time PCR System (Thermo Fisher).

TABLE 10
RT-qPCR primers

| Primer | | | Seq ID |
|---|---|---|---|
| name | Target | Sequence | NO |
| PS1_qPCR_F | mPsen1 | CCAGTGGAGACT | 205 |
| | | GGAACACAAC | |
| PS1_wt_ | mPsen1 | GGCTGGCAACG | 206 |
| qPCR_R | WT | CTTTCTTG | |
| PS1_A431E_ | mPsen1 | CGCCGGCAATT | 207 |
| qPCR_R | A431E | CTTTCTTGA | |
| mActinB_F | mActin | GTGACGTTGAC | 208 |
| | | ATCCGTAAAGA | |
| mActinB_R | mActin | GCCGGACTCAT | 209 |
| | | CGTACTCC | |
| Rps23-F | mRPS23 | CGTCAGGGTGC | 210 |
| | | AGCTCATTA | |
| Rps23-R | mRPS23 | GGCACGAACGC | 211 |
| | | TGTGATCTT | |

### Results

Treatment of heterozygous neurons (Psen1^{A431/Wt}; APPKi cells) with AON87 resulted in a dose-dependent reduction of WT Psen1 mRNA levels, which reached 67% reduction at the highest 10 μM AON concentration (FIG. 6A). Interestingly, the mRNA levels of A431E Psen1 show a 20% increase. To investigate whether the observed reduction in WT Psen1 and upregulation of A431E Psen1 leads to an impairment of the γ-secretase activity and an abnormal/disease-like Aβ profile the levels of soluble Aβ38, Aβ40 and Aβ42 peptides were measured in the medium. Total Aβ levels (Aβ38+Aβ40+Aβ42) were not affected by AON87 treatment (FIG. 6B). Importantly, AON87 treatment causes a 50% reduction of Aβ38 levels and a 30% increase of Aβ42 levels relative to non-treated samples (0 μM). This shift in the Aβ production results in a 2.5-fold increase in the ratio Aβ42/Aβ38.

### Conclusions

These results confirmed that A431E mutation in Psen1 reduces the functionality of γ-secretase and increases the production of long Aβ species (≥Aβ42) in primary mouse neurons, which leads to an abnormal/disease-like increase of Aβ42/38 ratio. Furthermore, the unexpected upregulation of A431E mRNA levels after the knock-down of WT Psen1 suggests that total levels of Psen1 are tightly regulated in neurons, and that compensatory mechanisms might exist. Another interesting observation is that the magnitude of change in the levels of each Aβ specie analysed is different, for example Aβ38 shows the biggest effect compared to non-treated, while Aβ40 is almost not altered. The potential compensatory mechanisms on mRNA levels and the different affectation of each Aβ species strengthens the need to use holistic readouts to evaluate the therapeutic benefit of the AON treatment.

### Example 7

### Introduction

Example 1 shows that AON20 treatment reduces Psen1 protein levels and the total production of Aβ peptides in A431E homozygous mouse primary neurons. To confirm that those changes are driven by a reduction of mutant Psen1 mRNA levels, mouse primary neurons derived from Psen1 A431E homozygous embryos (Psen1^{A431/A431E};APPKi) were treated for 7 consecutive days with AON20 (Table 2) and AON88 (Table 9). After, mutant Psen1 mRNA levels were quantified by RT-qPCR. (FIG. 7A).

Similarly, the effectivity and allele selectivity of AON20 and AON88 was asses by performing the same experiment in mouse primary neurons derived from Psen1 A431E heterozygous embryos (Psen1^{wt/A431E}; APPKi) (FIG. 7B).

### Material and Methods

See Example 1 and 6.

### Results

AON20 and AON88 treatment to primary mouse homozygous neurons (Psen1^{A431/A431E}; APPKi cells) resulted in a dose-dependent reduction of A431E PSEN1 mRNA levels, which reached 54% and 71% reduction at the highest 10 μM AON concentration, respectively (FIG. 7A).

AON20 and AON88 treatment to primary mouse heterozygous neurons (Psen1^{wt/A431E}; APPKi cells) resulted in a dose-dependent reduction of A431E PSEN1 mRNA levels, which reached, 64% and 71% reduction at the highest 10 μM AON concentration, respectively. Interestingly, the mRNA levels of WT PSEN1 showed a 12% and 19% increase at the highest AON concentration, respectively. (FIG. 7B).

### Conclusions

AON20 and AON88, AONs of the invention, are effective in reducing specifically mutant A431E Psen1 mRNA levels in mouse primary neurons. Therefore, the changes in Psen1 protein levels and Aβ peptide levels described in Example 1 are most likely driven by the AON effect on A431E Psen1 mRNA levels. Furthermore, the unexpected upregulation of WT mRNA levels observed after treatment of both AONs targeting mutant Psen1, supports the idea that Psen1 levels are tightly regulated in neurons, and reinforce the need to use holistic readouts to evaluate the effect of the AON treatment.

### Example 8

### Introduction

FIG. 7B shows that treatment with AON20 and AON88 reduces the mRNA levels of mutant Psen1 in a dose-dependent manner in primary mouse heterozygous neurons (Psen1^{wt/A431E}; APPKi cells). To evaluate if the observed changes in mRNA levels lead to a recovery of γ-secretase functionality and therefore ameliorate Aβ profile, the levels of soluble Aβ38, Aβ40, and Aβ42 peptides were quantified in the supernatant of the same cultures as in FIG. 7B (only the samples treated with 10UM of AON were analysed).

### Material and Methods

See Example 1 and 6.

### Results

AON20 and AON88 treatment to primary mouse heterozygous neurons (Psen1^{wt/A431E}; APPKi cells) does not alter total Aβ levels (FIG. 8A). Importantly, treatment with both AONs increases the levels of Aβ38 compared to the untreated samples (16% and 35%, respectively). Aβ42 levels show a tendency to decrease of 15% and 7%, respectively (Not statistically significant) (FIG. 8B). These changes lead to a decrease in the Aβ42/Aβ38 ratio, which reaches a 25% and 31% reduction relative to non-treated condition, respectively (FIG. 8C).

### Conclusions

AON20 and AON88, AONs of the invention, are effective in increasing short Aβ peptide levels and improving the Aβ42/Aβ38 ratio in primary mouse neurons. Similarly to Example 6, the changes in Aβ38 are more pronounced than in the other Aβ peptides analysed. This observation strengthens the idea that holistic readouts are needed to evaluate the therapeutic benefit of the AON treatment.

### Example 9

### Introduction

The goal of this experiment is to test if one AON of the invention (AON20, Table 2) is efficient and allele selective in-vivo when injected into the mouse brain. To this end, Psen1^{wt/A431E}; APPKi adult mice received an intracerebroventricular (ICV) single injection of AON20. Two weeks later, animals were sacrificed and the hippocampal mRNA levels of Wt and A431E Psen1 were quantified by RT-qPCR.

### Material and Methods

### Mice Treatment

Seven weeks-old Psen1^{Wt/A431E}; APPKi mice received a single ICV injection of AON20 in the right ventricle. 150 μg of AON20 were administered in 5 μl at an injection velocity of 0.3 μl/min. Mice in the vehicle group received 5 μl of artificial CSF. Animals were sacrificed 2 weeks after the injection. The right hippocampus was isolated for further analysis.

RNA Extraction and cDNA Synthesis

Tissues were homogenized in 1 ml of RNA-Bee (Bio-Connect) by grinding in a MagNa Lyser using MagNA Lyser Green Beads (Roche). Total RNA was extracted from the homogenate based on the manufacturer's instructions. RNA clean-up protocol, cDNA synthesis and RT-qPCR was performed as indicated in Example 6.

### Results

Single ICV injection of AON20 results in a 44% reduction of A431E Psen1 mRNA levels in the hippocampus. Levels of WT Psen1 mRNA are not statistically significantly changed.

### Conclusions

AON20, AON of the invention, shows efficacy and allele selectivity in-vivo when injected into the mouse brain.

1. An antisense oligonucleotide which preferentially targets a mutated allele of a protein of the γ-secretase complex when present in a cell comprising said mutated allele.

2. An antisense oligonucleotide according to claim 1, wherein the protein of the γ-secretase complex whose allele is mutated is PSEN1 or PSEN2.

3. An antisense oligonucleotide according to claim 1, wherein:
the oligonucleotide is able to preferentially silence, inactivate, break down, knock down, reduce or decrease mutated PSEN1 or PSEN2 transcripts, and
the oligonucleotide is not (or minimally) able to silence, inactivate, break down, knock down, reduce or decrease wild-type PSEN1 or PSEN2 transcripts.

4. An antisense oligonucleotide according to claim 1, wherein the oligonucleotide is able to normalize, reverse or correct abnormal Aβ42/38 and/or Aβ43/40 and/or Aβ43/37 and/or Aβ42/40 ratios.

5. An antisense oligonucleotide according to claim 1, wherein the protein of the γ-secretase complex is endogenously expressed by said cell.

6. An antisense oligonucleotide according to claim 1, wherein the cell is a neuronal cell.

7. An antisense oligonucleotide according to claim 1, wherein the protein of the γ-secretase complex whose allele is mutated is human PSEN1, and the mutation in human PSEN1 is selected from:
the group consisting of the most frequent PSEN1 mutations: P117L, M139T, M139V, M146I, H163R, G206A, P264L, E280A, L392V, A431E, and S121Y.

8. An antisense oligonucleotide according to claim 1, wherein the protein of the γ-secretase complex whose allele is mutated is human PSEN1, and the mutation in human PSEN1 is selected from the group consisting of mutations caused by a substitution of a guanine, thymine, or cytosine by an adenosine in the coding sequence, resulting in one of the following mutations in the PSEN1 protein: R35Q, V94M, F1051, R108Q, L113Q, T116N, P117T, P117Q, E120K, E123K, M139K, M139I, V142I, M146I, V151M, Y154N, L166H, L174M, 1180N, G206S, G206D, G209R, G209E, S212Y, H214N, G217D, S230N, A231T, M2331, F2371, A246E, Y256N, V261I, G266S, R269H, V272D, T274K, R278K, E280K, R358Q, A360T, S365Y, G378E, F386I, F386L, S390N, A396T, A409T, C410Y, G417S, L424H, A431E, A434T, P436Q.

9. An antisense oligonucleotide according to claim 1, wherein the antisense oligonucleotide is able to recruit RNaseH to silence, inactivate, knock down, break down, decrease or reduce the levels or expression of the targeted mutant allele.

10. An antisense oligonucleotide according to claim 1, wherein the antisense oligonucleotide preferentially targets a mutated allele or transcript of PSEN1 or PSEN2 and comprises 15 to 30 or 20 to 30 nucleotides including a central region of 5 to 15 consecutive DNA nucleotides and/or DNA nucleotides analogues flanked in each end by wing regions comprising 1 to 5 RNA nucleotides analogues.

11. An antisense oligonucleotide according to claim 10, wherein the mutation present in the mutated allele or transcript of PSEN1 or PSEN2 is targeted by the first, second, third, fourth, fifth, sixth, seventh, eighth, nineth, tenth, eleventh or twelfth nucleotide of the central region of the oligonucleotide.

12. An antisense oligonucleotide according to claim 10, wherein:
1) the DNA nucleotides in its central part have not been modified and the RNA nucleotides in each of its wings have been modified comprising a modified internucleoside linkage and/or a modified sugar and/or a modified base,
or
2) a DNA nucleotide in its central part has been modified and the backbone comprises a phosphorothioate and/or phosphoramidate, and the RNA nucleotides in each of its wings have been modified comprising a modified internucleoside linkage and/or a modified sugar and/or a modified base.

13. An antisense oligonucleotide according to claim 1, said oligonucleotide comprising SEQ ID NO: 2, 4, 6, 7, 8, 9, 10, 11, 12, 14, 16, 18, 20, 22, 24, 25, 26, 27, 28, 29, 30, 33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 111, 113, 115, 117,122, 124, 126, 128, 130, 132, 134, 136, 138, 140, 142, 144, 174, 176, 178, 180, 182, 184, 186, 188, 190, 192, 194, 196, 198, 200, 202, 204, 219, 221, 223, 225, 227, 229, 231, 233, 235, 237, 239, 241, 243, 245, 247, 249, 251, 253, 255, 257, 259, 261, 263, 265, 267, 269, 271, 273, 275, or 277.

14. A composition comprising the antisense oligonucleotide according to claim 1, wherein the composition comprises a pharmaceutically acceptable excipient.

15. (canceled)

16. A method for treating and/or delaying and/or curing and/or preventing and/or ameliorating a disease or condition associated with an abnormal processing of the Amyloid Precursor Protein (APP), said method comprises administering an antisense oligonucleotide as defined in claim 1 to an individual or a subject in need thereof.

17. The method of claim 16, wherein the antisense oligonucleotide elicits at least one of the following:
the oligonucleotide is able to preferentially silence, inactivate, break down, knock down, reduce or decrease mutated PSEN1 or PSEN2 transcripts and the oligonucleotide is not (or minimally) able to silence, inactivate, break down, knock down, reduce or decrease wild-type PSEN1 or PSEN2 transcripts,
the oligonucleotide is able to normalize, reverse, or correct abnormal Aβ42/38 and/or Aβ43/40 and/or Aβ43/37 and/or Aβ42/40 ratios, and
the oligonucleotide is able to alleviate one or more symptom(s) and/or characteristic(s) and/or to improve a parameter linked with or associated with a disease or condition associated with an abnormal processing of the Amyloid Precursor Protein (APP).

18-20. (canceled)

21. An antisense oligonucleotide according to claim 10, wherein the mutation present in the mutated allele or transcript of PSEN1 or PSEN2 is targeted by the sixth, seventh, eighth, nineth, tenth, eleventh, twelfth, thirteenth, fourteenth, fifteenth, sixteenth, seventeenth, or eighteenth nucleotide of the oligonucleotide sequence starting from 5′ to 3′.

22. (canceled)

23. An antisense oligonucleotide according to claim 10, wherein the mutation present in the mutated allele or transcript of PSEN1 or PSEN2 is targeted by the first, second, third, fourth, or fifth nucleotide of the central region of the oligonucleotide.

24. An antisense oligonucleotide according to claim 10, wherein the mutation present in the mutated allele or transcript of PSEN1 or PSEN2 is targeted by the sixth, seventh, eighth, nineth, or tenth nucleotide of the oligonucleotide sequence starting from 5′ to 3′.

25. A method for treating and/or delaying and/or curing and/or preventing and/or ameliorating a disease or condition associated with an abnormal processing of the Amyloid Precursor Protein (APP), said method comprises administering a composition as defined in claim 14 to an individual or a subject in need thereof.

26. The method of claim 25, wherein the antisense oligonucleotide comprised in the composition elicits at least one of the following:
the oligonucleotide is able to preferentially silence, inactivate, break down, knock down, reduce or decrease mutated PSEN1 or PSEN2 transcripts and the oligonucleotide is not (or minimally) able to silence, inactivate, break down, knock down, reduce or decrease wild-type PSEN1 or PSEN2 transcripts,
the oligonucleotide is able to normalize, reverse, or correct abnormal Aβ42/38 and/or Aβ43/40 and/or Aβ43/37 and/or Aβ42/40 ratios, and
the oligonucleotide is able to alleviate one or more symptom(s) and/or characteristic(s) and/or to improve a parameter linked with or associated with a disease or condition associated with an abnormal processing of the Amyloid Precursor Protein (APP).


SEQUENCE LISTING (277 sequences)

SEQ ID NO: 1 - RNA, 20 nt, synthetic construct
  source 1..20: mol_type = other RNA; note = AON1 (non-modified)
tctttcttgaaaatggcaag

SEQ ID NO: 2 - RNA, 20 nt, synthetic construct
  source 1..20: mol_type = other RNA; note = AON1
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 17: mod_base = OTHER; note = 5-methyl
  modified_base 6..15: mod_base = OTHER; note = DNA base
  modified_base 1..20: mod_base = OTHER; note = phosphorothioate linkage
tctttcttgaaaatggcaag

SEQ ID NO: 3 - RNA, 20 nt, synthetic construct
  source 1..20: mol_type = other RNA; note = AON2 (non-modified)
attctttcttgaaaatggca

SEQ ID NO: 4 - RNA, 20 nt, synthetic construct
  source 1..20: mol_type = other RNA; note = AON2
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 19: mod_base = OTHER; note = 5-methyl
  modified_base 6..15: mod_base = OTHER; note = DNA base
  modified_base 1..20: mod_base = OTHER; note = phosphorothioate linkage
attctttcttgaaaatggca

SEQ ID NO: 5 - RNA, 20 nt, synthetic construct
  source 1..20: mol_type = other RNA; note = AON3 (non-modified)
caattctttcttgaaaatgg

SEQ ID NO: 6 - RNA, 20 nt, synthetic construct
  source 1..20: mol_type = other RNA; note = AON3
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 1: mod_base = OTHER; note = 5-methyl
  modified_base 6..15: mod_base = OTHER; note = DNA base
  modified_base 1..20: mod_base = OTHER; note = phosphorothioate linkage
caattctttcttgaaaatgg

SEQ ID NO: 7 - RNA, 20 nt, synthetic construct
  source 1..20: mol_type = other RNA; note = AON4 (non-modified)
gcaattctttcttgaaaatg

SEQ ID NO: 8 - RNA, 20 nt, synthetic construct
  source 1..20: mol_type = other RNA; note = AON4
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..20: mod_base = OTHER; note = phosphorothioate linkage
gcaattctttcttgaaaatg

SEQ ID NO: 9 - RNA, 20 nt, synthetic construct
  source 1..20: mol_type = other RNA; note = AON5 (non-modified)
ggcaattctttcttgaaaat

SEQ ID NO: 10 - RNA, 20 nt, synthetic construct
  source 1..20: mol_type = other RNA; note = AON5
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattctttcttgaaaat

SEQ ID NO: 11 - RNA, 20 nt, synthetic construct
  source 1..20: mol_type = other RNA; note = AON6 (non-modified)
tggcaattctttcttgaaaa

SEQ ID NO: 12 - RNA, 20 nt, synthetic construct
  source 1..20: mol_type = other RNA; note = AON6
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1..20: mod_base = OTHER; note = phosphorothioate linkage
tggcaattctttcttgaaaa

SEQ ID NO: 13 - RNA, 20 nt, synthetic construct
  source 1..20: mol_type = other RNA; note = AON7 (non-modified)
ctggcaattctttcttgaaa

SEQ ID NO: 14 - RNA, 20 nt, synthetic construct
  source 1..20: mol_type = other RNA; note = AON7
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 1: mod_base = OTHER; note = 5-methyl
  modified_base 5: mod_base = OTHER; note = 5-methyl
  modified_base 6..15: mod_base = OTHER; note = DNA base
  modified_base 1..20: mod_base = OTHER; note = phosphorothioate linkage
ctggcaattctttcttgaaa

SEQ ID NO: 15 - RNA, 20 nt, synthetic construct
  source 1..20: mol_type = other RNA; note = AON8 (non-modified)
agctggcaattctttcttga

SEQ ID NO: 16 - RNA, 20 nt, synthetic construct
  source 1..20: mol_type = other RNA; note = AON8
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 16: mod_base = OTHER; note = 5-methyl
  modified_base 6..15: mod_base = OTHER; note = DNA base
  modified_base 1..20: mod_base = OTHER; note = phosphorothioate linkage
agctggcaattctttcttga

SEQ ID NO: 17 - RNA, 20 nt, synthetic construct
  source 1..20: mol_type = other RNA; note = AON9 (non-modified)
agagctggcaattctttctt

SEQ ID NO: 18 - RNA, 20 nt, synthetic construct
  source 1..20: mol_type = other RNA; note = AON9
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 5: mod_base = OTHER; note = 5-methyl
  modified_base 18: mod_base = OTHER; note = 5-methyl
  modified_base 6..15: mod_base = OTHER; note = DNA base
  modified_base 1..20: mod_base = OTHER; note = phosphorothioate linkage
agagctggcaattctttctt

SEQ ID NO: 19 - RNA, 20 nt, synthetic construct
  source 1..20: mol_type = other RNA; note = AON10 (non-modified)
gaagagctggcaattctttc

SEQ ID NO: 20 - RNA, 20 nt, synthetic construct
  source 1..20: mol_type = other RNA; note = AON10
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16: mod_base = OTHER; note = 5-methyl
  modified_base 20: mod_base = OTHER; note = 5-methyl
  modified_base 1..20: mod_base = OTHER; note = phosphorothioate linkage
gaagagctggcaattctttc

SEQ ID NO: 21 - RNA, 20 nt, synthetic construct
  source 1..20: mol_type = other RNA; note = AON11 (non-modified)
tggaagagctggcaattctt

SEQ ID NO: 22 - RNA, 20 nt, synthetic construct
  source 1..20: mol_type = other RNA; note = AON11
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 18: mod_base = OTHER; note = 5-methyl
  modified_base 1..20: mod_base = OTHER; note = phosphorothioate linkage
tggaagagctggcaattctt

SEQ ID NO: 23 - RNA, 20 nt, synthetic construct
  source 1..20: mol_type = other RNA; note = AON12 (non-modified)
attggaagagctggcaattc

SEQ ID NO: 24 - RNA, 20 nt, synthetic construct
  source 1..20: mol_type = other RNA; note = AON12
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 20: mod_base = OTHER; note = 5-methyl
  modified_base 1..20: mod_base = OTHER; note = phosphorothioate linkage
attggaagagctggcaattc

SEQ ID NO: 25 - RNA, 20 nt, synthetic construct
  source 1..20: mol_type = other RNA; note = AON13 (non-modified)
gcaattctttcttgaaaatg

SEQ ID NO: 26 - RNA, 20 nt, synthetic construct
  source 1..20: mol_type = other RNA; note = AON13
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..20: mod_base = OTHER; note = phosphorothioate linkage
gcaattctttcttgaaaatg

SEQ ID NO: 27 - RNA, 20 nt, synthetic construct
  source 1..20: mol_type = other RNA; note = AON14 (non-modified)
ggcaattctttcttgaaaat

SEQ ID NO: 28 - RNA, 20 nt, synthetic construct
  source 1..20: mol_type = other RNA; note = AON14
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattctttcttgaaaat

SEQ ID NO: 29 - RNA, 20 nt, synthetic construct
  source 1..20: mol_type = other RNA; note = AON15 (non-modified)
tggcaattctttcttgaaaa

SEQ ID NO: 30 - RNA, 20 nt, synthetic construct
  source 1..20: mol_type = other RNA; note = AON15
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1..20: mod_base = OTHER; note = phosphorothioate linkage
tggcaattctttcttgaaaa

SEQ ID NO: 31 - RNA, 20 nt, synthetic construct
  source 1..20: mol_type = other RNA; note = Scrambled
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 20: mod_base = OTHER; note = 5-methyl
  modified_base 6..15: mod_base = OTHER; note = DNA base
  modified_base 1..20: mod_base = OTHER; note = phosphorothioate linkage
atgcttgctcgtaagttatc

SEQ ID NO: 32 - RNA, 20 nt, synthetic construct
  source 1..20: mol_type = other RNA; note = AON16 (non-modified)
ggcaattctttcttgaaaat

SEQ ID NO: 33 - RNA, 20 nt, synthetic construct
  source 1..20: mol_type = other RNA; note = AON16
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
ggcaattctttcttgaaaat

SEQ ID NO: 34 - RNA, 20 nt, synthetic construct
  source 1..20: mol_type = other RNA; note = AON17 (non-modified)
ggcaattctttcttgaaaat

SEQ ID NO: 35 - RNA, 20 nt, synthetic construct
  source 1..20: mol_type = other RNA; note = AON17
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..7: mod_base = OTHER; note = phosphorothioate linkage
  modified_base 9..20: mod_base = OTHER; note = phosphorothioate linkage
  modified_base 7..9: mod_base = OTHER; note = mesyl-phosphoramidate linkage
ggcaattctttcttgaaaat

SEQ ID NO: 36 - RNA, 20 nt, synthetic construct
  source 1..20: mol_type = other RNA; note = AON18 (non-modified)
ggcaattctttcttgaaaat

SEQ ID NO: 37 - RNA, 20 nt, synthetic construct
  source 1..20: mol_type = other RNA; note = AON18
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..8: mod_base = OTHER; note = phosphorothioate linkage
  modified_base 10..20: mod_base = OTHER; note = phosphorothioate linkage
  modified_base 8..10: mod_base = OTHER; note = mesyl-phosphoramidate linkage
ggcaattctttcttgaaaat

SEQ ID NO: 38 - RNA, 20 nt, synthetic construct
  source 1..20: mol_type = other RNA; note = AON19 (non-modified)
ggcaattctttcttgaaaat

SEQ ID NO: 39 - RNA, 20 nt, synthetic construct
  source 1..20: mol_type = other RNA; note = AON19
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..8: mod_base = OTHER; note = phosphorothioate linkage
  modified_base 10..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 8..10: mod_base = OTHER; note = mesyl-phosphoramidate linkage
ggcaattctttcttgaaaat

SEQ ID NO: 40 - RNA, 20 nt, synthetic construct
  source 1..20: mol_type = other RNA; note = AON22 (non-modified)
ggcaaatctttcttgaaaat

SEQ ID NO: 41 - RNA, 20 nt, synthetic construct
  source 1..20: mol_type = other RNA; note = AON22
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaaatctttcttgaaaat

SEQ ID NO: 42 - RNA, 20 nt, synthetic construct
  source 1..20: mol_type = other RNA; note = AON23 (non-modified)
ggcaattgtttcttgaaaat

SEQ ID NO: 43 - RNA, 20 nt, synthetic construct
  source 1..20: mol_type = other RNA; note = AON23
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattgtttcttgaaaat

SEQ ID NO: 44 - RNA, 20 nt, synthetic construct
  source 1..20: mol_type = other RNA; note = AON24 (non-modified)
ggcaattgattcttgaaaat

SEQ ID NO: 45 - RNA, 20 nt, synthetic construct
  source 1..20: mol_type = other RNA; note = AON24
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattgattcttgaaaat

SEQ ID NO: 46 - RNA, 20 nt, synthetic construct
  source 1..20: mol_type = other RNA; note = AON25 (non-modified)
gcaattctttcttgaaaatg

SEQ ID NO: 47 - RNA, 20 nt, synthetic construct
  source 1..20: mol_type = other RNA; note = AON25
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
gcaattctttcttgaaaatg

SEQ ID NO: 48 - RNA, 20 nt, synthetic construct
  source 1..20: mol_type = other RNA; note = AON26 (non-modified)
ggcaattctaacttgaaaat

SEQ ID NO: 49 - RNA, 20 nt, synthetic construct
  source 1..20: mol_type = other RNA; note = AON26
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattctaacttgaaaat

SEQ ID NO: 50 - RNA, 20 nt, synthetic construct
  source 1..20: mol_type = other RNA; note = AON27 (non-modified)
ggcaattcttagttgaaaat

SEQ ID NO: 51 - RNA, 20 nt, synthetic construct
  source 1..20: mol_type = other RNA; note = AON27
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..20: mod_base = OTHER; note = phosphorothioate linkage
ggcaattcttagttgaaaat

SEQ ID NO: 52 - RNA, 18 nt, synthetic construct
  source 1..18: mol_type = other RNA; note = AON28 (non-modified)
caattctttcttgaaaat

SEQ ID NO: 53 - RNA, 18 nt, synthetic construct
  source 1..18: mol_type = other RNA; note = AON28
  modified_base 1..4: mod_base = OTHER; note = 2'-O-(2-methoxy)ethyl base
  modified_base 15..18: mod_base = OTHER; note = 2'-O-(2-methoxy)ethyl base
  modified_base 1: mod_base = OTHER; note = 5-methyl
  modified_base 5..14: mod_base = OTHER; note = DNA base
  modified_base 1..18: mod_base = OTHER; note = phosphorothioate linkage
caattctttcttgaaaat

SEQ ID NO: 54 - RNA, 18 nt, synthetic construct
  source 1..18: mol_type = other RNA; note = AON29 (non-modified)
gcaattctttcttgaaaa

SEQ ID NO: 55 - RNA, 18 nt, synthetic construct
  source 1..18: mol_type = other RNA; note = AON29
  modified_base 1..4: mod_base = OTHER; note = 2'-O-(2-methoxy)ethyl base
  modified_base 15..18: mod_base = OTHER; note = 2'-O-(2-methoxy)ethyl base
  modified_base 2: mod_base = OTHER; note = 5-methyl
  modified_base 5..14: mod_base = OTHER; note = DNA base
  modified_base 1..18: mod_base = OTHER; note = phosphorothioate linkage
gcaattctttcttgaaaa

SEQ ID NO: 56 - RNA, 18 nt, synthetic construct
  source 1..18: mol_type = other RNA; note = AON30 (non-modified)
ggcaattctttcttgaaa

SEQ ID NO: 57 - RNA, 18 nt, synthetic construct
  source 1..18: mol_type = other RNA; note = AON30
  modified_base 1..4: mod_base = OTHER; note = 2'-O-(2-methoxy)ethyl base
  modified_base 15..18: mod_base = OTHER; note = 2'-O-(2-methoxy)ethyl base
  modified_base 3: mod_base = OTHER; note = 5-methyl
  modified_base 5..14: mod_base = OTHER; note = DNA base
  modified_base 1..18: mod_base = OTHER; note = phosphorothioate linkage
ggcaattctttcttgaaa

SEQ ID NO: 58 - RNA, 16 nt, synthetic construct
  source 1..16: mol_type = other RNA; note = AON31 (non-modified)
aattctttcttgaaaa

SEQ ID NO: 59 - RNA, 16 nt, synthetic construct
  source 1..16: mol_type = other RNA; note = AON31
  modified_base 1..3: mod_base = OTHER; note = 2'-O-(2-methoxy)ethyl base
  modified_base 14..16: mod_base = OTHER; note = 2'-O-(2-methoxy)ethyl base
  modified_base 4..13: mod_base = OTHER; note = DNA base
  modified_base 1..16: mod_base = OTHER; note = phosphorothioate linkage
aattctttcttgaaaa

SEQ ID NO: 60 - RNA, 16 nt, synthetic construct
  source 1..16: mol_type = other RNA; note = AON32 (non-modified)
caattctttcttgaaa

SEQ ID NO: 61 - RNA, 16 nt, synthetic construct
  source 1..16: mol_type = other RNA; note = AON32
  modified_base 1..3: mod_base = OTHER; note = 2'-O-(2-methoxy)ethyl base
  modified_base 14..16: mod_base = OTHER; note = 2'-O-(2-methoxy)ethyl base
  modified_base 1: mod_base = OTHER; note = 5-methyl
  modified_base 4..13: mod_base = OTHER; note = DNA base
  modified_base 1..16: mod_base = OTHER; note = phosphorothioate linkage
caattctttcttgaaa

SEQ ID NO: 62 - RNA, 16 nt, synthetic construct
  source 1..16: mol_type = other RNA; note = AON33 (non-modified)
gcaattctttcttgaa

SEQ ID NO: 63 - RNA, 16 nt, synthetic construct
  source 1..16: mol_type = other RNA; note = AON33
  modified_base 1..3: mod_base = OTHER; note = 2'-O-(2-methoxy)ethyl base
  modified_base 14..16: mod_base = OTHER; note = 2'-O-(2-methoxy)ethyl base
  modified_base 2: mod_base = OTHER; note = 5-methyl
  modified_base 4..13: mod_base = OTHER; note = DNA base
  modified_base 1..16: mod_base = OTHER; note = phosphorothioate linkage
gcaattctttcttgaa

SEQ ID NO: 64 - RNA, 20 nt, synthetic construct
  source 1..20: mol_type = other RNA; note = AON34 (non-modified)
attctttcttgaaaatggca

SEQ ID NO: 65 - RNA, 20 nt, synthetic construct
  source 1..20: mol_type = other RNA; note = AON34
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 19: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
attctttcttgaaaatggca

SEQ ID NO: 66 - RNA, 20 nt, synthetic construct
  source 1..20: mol_type = other RNA; note = AON35 (non-modified)
attctttcttgaaaatggca

SEQ ID NO: 67 - RNA, 20 nt, synthetic construct
  source 1..20: mol_type = other RNA; note = AON35
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 19: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = dimethylimidazolidin-2-ylidene-phosphoramidate linkage
  modified_base 16..19: mod_base = OTHER; note = dimethylimidazolidin-2-ylidene-phosphoramidate linkage
attctttcttgaaaatggca

SEQ ID NO: 68 - RNA, 20 nt, synthetic construct
  source 1..20: mol_type = other RNA; note = AON36 (non-modified)
ggcaattctttcttgaaaat

SEQ ID NO: 69 - RNA, 20 nt, synthetic construct
  source 1..20: mol_type = other RNA; note = AON36
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
ggcaattctttcttgaaaat

SEQ ID NO: 70 - RNA, 20 nt, synthetic construct
  source 1..20: mol_type = other RNA; note = AON37 (non-modified)
ggcaattctttcttgaaaat

SEQ ID NO: 71 - RNA, 20 nt, synthetic construct
  source 1..20: mol_type = other RNA; note = AON37
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = dimethylimidazolidin-2-ylidene-phosphoramidate linkage
  modified_base 16..19: mod_base = OTHER; note = dimethylimidazolidin-2-ylidene-phosphoramidate linkage
ggcaattctttcttgaaaat

SEQ ID NO: 72 - RNA, 20 nt, synthetic construct
  source 1..20: mol_type = other RNA; note = AON38 (non-modified)
ggcaattctttcttgaaaat

SEQ ID NO: 73 - RNA, 20 nt, synthetic construct
  source 1..20: mol_type = other RNA; note = AON38
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
ggcaattctttcttgaaaat

SEQ ID NO: 74 - RNA, 20 nt, synthetic construct
  source 1..20: mol_type = other RNA; note = AON39 (non-modified)
agctggcaattctttcttga

SEQ ID NO: 75 - RNA, 20 nt, synthetic construct
  source 1..20: mol_type = other RNA; note = AON39
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 16: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
agctggcaattctttcttga

SEQ ID NO: 76 - RNA, 20 nt, synthetic construct
  source 1..20: mol_type = other RNA; note = AON40 (non-modified)
agctggcaattctttcttga

SEQ ID NO: 77 - RNA, 20 nt, synthetic construct
  source 1..20: mol_type = other RNA; note = AON40
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 16: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = dimethylimidazolidin-2-ylidene-phosphoramidate linkage
  modified_base 16..19: mod_base = OTHER; note = dimethylimidazolidin-2-ylidene-phosphoramidate linkage
agctggcaattctttcttga

SEQ ID NO: 78 - RNA, 20 nt, synthetic construct
  source 1..20: mol_type = other RNA; note = AON41 (non-modified)
agctggcaattctttcttga

SEQ ID NO: 79 - RNA, 20 nt, synthetic construct
  source 1..20: mol_type = other RNA; note = AON41
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 16: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
agctggcaattctttcttga

SEQ ID NO: 80 - RNA, 20 nt, synthetic construct
  source 1..20: mol_type = other RNA; note = AON42 (non-modified)
gaagagctggcaattctttc

SEQ ID NO: 81 - RNA, 20 nt, synthetic construct
  source 1..20: mol_type = other RNA; note = AON42
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16: mod_base = OTHER; note = 5-methyl
  modified_base 20: mod_base = OTHER; note = 5-methyl
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
gaagagctggcaattctttc

SEQ ID NO: 82 - RNA, 20 nt, synthetic construct
  source 1..20: mol_type = other RNA; note = AON43 (non-modified)
gaagagctggcaattctttc

SEQ ID NO: 83 - RNA, 20 nt, synthetic construct
  source 1..20: mol_type = other RNA; note = AON43
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16: mod_base = OTHER; note = 5-methyl
  modified_base 20: mod_base = OTHER; note = 5-methyl
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = dimethylimidazolidin-2-ylidene-phosphoramidate linkage
  modified_base 16..19: mod_base = OTHER; note = dimethylimidazolidin-2-ylidene-phosphoramidate linkage
gaagagctggcaattctttc

SEQ ID NO: 84 - RNA, 20 nt, synthetic construct
  source 1..20: mol_type = other RNA; note = AON44 (non-modified)
tggcaattctttcttgaaaa

SEQ ID NO: 85 - RNA, 20 nt, synthetic construct
  source 1..20: mol_type = other RNA; note = AON44
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
tggcaattctttcttgaaaa

SEQ ID NO: 86 - RNA, 20 nt, synthetic construct
  source 1..20: mol_type = other RNA; note = AON45 (non-modified)
gcaattctttcttgaaaatg

SEQ ID NO: 87 - RNA, 20 nt, synthetic construct
  source 1..20: mol_type = other RNA; note = AON45
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2^3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 3^4: mod_base = OTHER; note = phospodiester linkage
  modified_base 16..19: mod_base = OTHER; note = phospodiester linkage
gcaattctttcttgaaaatg

SEQ ID NO: 88 - RNA, 20 nt, synthetic construct
  source 1..20: mol_type = other RNA; note = AON46 (non-modified)
ggcaattctttcttgaaaat

SEQ ID NO: 89 - RNA, 20 nt, synthetic construct
  source 1..20: mol_type = other RNA; note = AON46
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2^3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 3^4: mod_base = OTHER; note = phospodiester linkage
  modified_base 16..19: mod_base = OTHER; note = phospodiester linkage
ggcaattctttcttgaaaat

SEQ ID NO: 90 - RNA, 20 nt, synthetic construct
  source 1..20: mol_type = other RNA; note = AON47 (non-modified)
tggcaattctttcttgaaaa

SEQ ID NO: 91 - RNA, 20 nt, synthetic construct
  source 1..20: mol_type = other RNA; note = AON47
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2^3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 3^4: mod_base = OTHER; note = phospodiester linkage
  modified_base 16..19: mod_base = OTHER; note = phospodiester linkage
tggcaattctttcttgaaaa

SEQ ID NO: 92 - RNA, 18 nt, synthetic construct
  source 1..18: mol_type = other RNA; note = AON48 (non-modified)
ggcaattctttcttgaaa

SEQ ID NO: 93 - RNA, 18 nt, synthetic construct
  source 1..18: mol_type = other RNA; note = AON48
  modified_base 1..4: mod_base = OTHER; note = 2'-O-(2-methoxy)ethyl base
  modified_base 15..18: mod_base = OTHER; note = 2'-O-(2-methoxy)ethyl base
  modified_base 3: mod_base = OTHER; note = 5-methyl
  modified_base 5..14: mod_base = OTHER; note = DNA base
  modified_base 1..18: mod_base = OTHER; note = phosphorothioate linkage
ggcaattctttcttgaaa

SEQ ID NO: 94 - RNA, 18 nt, synthetic construct
  source 1..18: mol_type = other RNA; note = AON49 (non-modified)
ggcaattctttcttgaaa

SEQ ID NO: 95 - RNA, 18 nt, synthetic construct
  source 1..18: mol_type = other RNA; note = AON49
  modified_base 1..4: mod_base = OTHER; note = 2'-O-(2-methoxy)ethyl base
  modified_base 15..18: mod_base = OTHER; note = 2'-O-(2-methoxy)ethyl base
  modified_base 3: mod_base = OTHER; note = 5-methyl
  modified_base 5..14: mod_base = OTHER; note = DNA base
  modified_base 1^2: mod_base = OTHER; note = phosphorothioate linkage
  modified_base 4..15: mod_base = OTHER; note = phosphorothioate linkage
  modified_base 2^3: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 3^4: mod_base = OTHER; note = phospodiester linkage
  modified_base 15..17: mod_base = OTHER; note = phospodiester linkage
ggcaattctttcttgaaa

SEQ ID NO: 96 - RNA, 20 nt, synthetic construct
  source 1..20: mol_type = other RNA; note = AON50 (non-modified)
gcaattctttcttgaaaatg

SEQ ID NO: 97 - RNA, 20 nt, synthetic construct
  source 1..20: mol_type = other RNA; note = AON50
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5^6: mod_base = OTHER; note = phosphorothioate linkage
  modified_base 8..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 6..8: mod_base = OTHER; note = mesyl-phosphoramidate linkage
gcaattctttcttgaaaatg

SEQ ID NO: 98 - RNA, 20 nt, synthetic construct
  source 1..20: mol_type = other RNA; note = AON51 (non-modified)
gcaattctttcttgaaaatg

SEQ ID NO: 99 - RNA, 20 nt, synthetic construct
  source 1..20: mol_type = other RNA; note = AON51
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
gcaattctttcttgaaaatg

SEQ ID NO: 100 - RNA, 20 nt, synthetic construct
  source 1..20: mol_type = other RNA; note = AON52 (non-modified)
gcaattctttcttgaaaatg

SEQ ID NO: 101 - RNA, 20 nt, synthetic construct
  source 1..20: mol_type = other RNA; note = AON52
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 2..4: mod_base = OTHER; note = mesyl-phosphoramidate linkage
  modified_base 7..9: mod_base = OTHER; note = mesyl-phosphoramidate linkage
  modified_base 4^5: mod_base = OTHER; note = phospodiester linkage
  modified_base 16..19: mod_base = OTHER; note = phospodiester linkage
gcaattctttcttgaaaatg

SEQ ID NO: 102 - RNA, 20 nt, synthetic construct
  source 1..20: mol_type = other RNA; note = AON53 (non-modified)
gcaattctttcttgaaaatg

SEQ ID NO: 103 - RNA, 20 nt, synthetic construct
  source 1..20: mol_type = other RNA; note = AON53
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..20: mod_base = OTHER; note = phosphorothioate linkage
  modified_base 2..5: mod_base = OTHER; note = phospodiester linkage
  modified_base 7..9: mod_base = OTHER; note = mesyl-phosphoramidate linkage
gcaattctttcttgaaaatg

SEQ ID NO: 104 - RNA, 20 nt, synthetic construct
  source 1..20: mol_type = other RNA; note = AON54 (non-modified)
gcaattctttcttgaaaatg

SEQ ID NO: 105 - RNA, 20 nt, synthetic construct
  source 1..20: mol_type = other RNA; note = AON54
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 7..9: mod_base = OTHER; note = mesyl-phosphoramidate linkage
  modified_base 16..19: mod_base = OTHER; note = phospodiester linkage
gcaattctttcttgaaaatg

SEQ ID NO: 106 - RNA, 20 nt, synthetic construct
  source 1..20: mol_type = other RNA; note = AON55 (non-modified)
ggcaattctttcttgaaaat

SEQ ID NO: 107 - RNA, 20 nt, synthetic construct
  source 1..20: mol_type = other RNA; note = AON55
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
ggcaattctttcttgaaaat

SEQ ID NO: 108 - RNA, 20 nt, synthetic construct
  source 1..20: mol_type = other RNA; note = AON56 (non-modified)
ggcaattctttcttgaaaat

SEQ ID NO: 109 - RNA, 20 nt, synthetic construct
  source 1..20: mol_type = other RNA; note = AON56
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..8: mod_base = OTHER; note = phosphorothioate linkage
  modified_base 10..16: mod_base = OTHER; note = phosphorothioate linkage
  modified_base 19^20: mod_base = OTHER; note = phosphorothioate linkage
  modified_base 2..4: mod_base = OTHER; note = mesyl-phosphoramidate linkage
  modified_base 8..10: mod_base = OTHER; note = mesyl-phosphoramidate linkage
  modified_base 4^5: mod_base = OTHER; note = phospodiester linkage
  modified_base 16..19: mod_base = OTHER; note = phospodiester linkage
ggcaattctttcttgaaaat

SEQ ID NO: 110 - RNA, 20 nt, synthetic construct
  source 1..20: mol_type = other RNA; note = AON57 (non-modified)
ggcaattctttcttgaaaat

SEQ ID NO: 111 - RNA, 20 nt, synthetic construct
  source 1..20: mol_type = other RNA; note = AON57
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..4: mod_base = OTHER; note = mesyl-phosphoramidate linkage
  modified_base 4^5: mod_base = OTHER; note = phospodiester linkage
  modified_base 16..19: mod_base = OTHER; note = phospodiester linkage
ggcaattctttcttgaaaat

SEQ ID NO: 112 - RNA, 20 nt, synthetic construct
  source 1..20: mol_type = other RNA; note = AON58 (non-modified)
ggcaattctttcttgaaaat

SEQ ID NO: 113 - RNA, 20 nt, synthetic construct
  source 1..20: mol_type = other RNA; note = AON58
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 16..19: mod_base = OTHER; note = phospodiester linkage
  modified_base 3..5: mod_base = OTHER; note = mesyl-phosphoramidate linkage
ggcaattctttcttgaaaat

SEQ ID NO: 114 - RNA, 20 nt, synthetic construct
  source 1..20: mol_type = other RNA; note = AON59 (non-modified)
ggcaattctttcttgaaaat

SEQ ID NO: 115 - RNA, 20 nt, synthetic construct
  source 1..20: mol_type = other RNA; note = AON59
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2..5: mod_base = OTHER; note = phospodiester linkage
  modified_base 16..18: mod_base = OTHER; note = phospodiester linkage
  modified_base 18..20: mod_base = OTHER; note = mesyl-phosphoramidate linkage
ggcaattctttcttgaaaat

SEQ ID NO: 116 - RNA, 20 nt, synthetic construct
  source 1..20: mol_type = other RNA; note = AON60 (non-modified)
ggcaattctttcttgaaaat

SEQ ID NO: 117 - RNA, 20 nt, synthetic construct
  source 1..20: mol_type = other RNA; note = AON60
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2..5: mod_base = OTHER; note = phospodiester linkage
  modified_base 16^17: mod_base = OTHER; note = phospodiester linkage
  modified_base 19^20: mod_base = OTHER; note = phospodiester linkage
  modified_base 17..19: mod_base = OTHER; note = mesyl-phosphoramidate linkage
ggcaattctttcttgaaaat

SEQ ID NO: 118 - RNA, 21 nt, synthetic construct
  source 1..21: mol_type = other RNA; note = AON20
  modified_base 1..5: mod_base = OTHER; note = 2'-O-(2-methoxy)ethyl base
  modified_base 17..21: mod_base = OTHER; note = 2'-O-(2-methoxy)ethyl base
  modified_base 3: mod_base = OTHER; note = 5-methyl
  modified_base 5: mod_base = OTHER; note = 5-methyl
  modified_base 18: mod_base = OTHER; note = 5-methyl
  modified_base 6..16: mod_base = OTHER; note = DNA base
  modified_base 1..21: mod_base = OTHER; note = phosphorothioate linkage
agcgccggcaattctttcttg

SEQ ID NO: 119 - RNA, 21 nt, synthetic construct
  source 1..21: mol_type = other RNA; note = AON20 (non-modified)
agcgccggcaattctttcttg

SEQ ID NO: 120 - RNA, 21 nt, synthetic construct
  source 1..21: mol_type = other RNA; note = AON21
  modified_base 1..5: mod_base = OTHER; note = 2'-O-(2-methoxy)ethyl base
  modified_base 17..21: mod_base = OTHER; note = 2'-O-(2-methoxy)ethyl base
  modified_base 3: mod_base = OTHER; note = 5-methyl
  modified_base 5..6: mod_base = OTHER; note = 5-methyl
  modified_base 9: mod_base = OTHER; note = 5-methyl
  modified_base 14: mod_base = OTHER; note = 5-methyl
  modified_base 18: mod_base = OTHER; note = 5-methyl
  modified_base 6..16: mod_base = OTHER; note = DNA base
  modified_base 1..21: mod_base = OTHER; note = phosphorothioate linkage
agcgccggcaattctttcttg

SEQ ID NO: 121 - RNA, 20 nt, synthetic construct
  source 1..20: mol_type = other RNA; note = AON61 (non-modified)
gcaattctttcttgaaaatg

SEQ ID NO: 122 - RNA, 20 nt, synthetic construct
  source 1..20: mol_type = other RNA; note = AON61
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
gcaattctttcttgaaaatg

SEQ ID NO: 123 - RNA, 20 nt, synthetic construct
  source 1..20: mol_type = other RNA; note = AON62 (non-modified)
ggcaattctttcttgaaaat

SEQ ID NO: 124 - RNA, 20 nt, synthetic construct
  source 1..20: mol_type = other RNA; note = AON62
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
ggcaattctttcttgaaaat

SEQ ID NO: 125 - RNA, 20 nt, synthetic construct
  source 1..20: mol_type = other RNA; note = AON63 (non-modified)
gcaattctttcttgaaaatg

SEQ ID NO: 126 - RNA, 20 nt, synthetic construct
  source 1..20: mol_type = other RNA; note = AON63
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 19^20: mod_base = OTHER; note = dimethylimidazolidin-2-ylidene-phosphoramidate linkage
gcaattctttcttgaaaatg

SEQ ID NO: 127 - RNA, 20 nt, synthetic construct
  source 1..20: mol_type = other RNA; note = AON64 (non-modified)
ggcaattctttcttgaaaat

SEQ ID NO: 128 - RNA, 20 nt, synthetic construct
  source 1..20: mol_type = other RNA; note = AON64
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 19^20: mod_base = OTHER; note = dimethylimidazolidin-2-ylidene-phosphoramidate linkage
ggcaattctttcttgaaaat

SEQ ID NO: 129 - RNA, 20 nt, synthetic construct
  source 1..20: mol_type = other RNA; note = AON65 (non-modified)
gcaattctttcttgaaaatg

SEQ ID NO: 130 - RNA, 20 nt, synthetic construct
  source 1..20: mol_type = other RNA; note = AON65
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5^6: mod_base = OTHER; note = phosphorothioate linkage
  modified_base 8..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 6..8: mod_base = OTHER; note = mesyl-phosphoramidate linkage
gcaattctttcttgaaaatg

SEQ ID NO: 131 - RNA, 20 nt, synthetic construct
  source 1..20: mol_type = other RNA; note = AON66 (non-modified)
gcaattctttcttgaaaatg

SEQ ID NO: 132 - RNA, 20 nt, synthetic construct
  source 1..20: mol_type = other RNA; note = AON66
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
gcaattctttcttgaaaatg

SEQ ID NO: 133 - RNA, 20 nt, synthetic construct
  source 1..20: mol_type = other RNA; note = AON67 (non-modified)
ggcaattctttcttgaaaat

SEQ ID NO: 134 - RNA, 20 nt, synthetic construct
  source 1..20: mol_type = other RNA; note = AON67
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
ggcaattctttcttgaaaat

SEQ ID NO: 135 - RNA, 20 nt, synthetic construct
  source 1..20: mol_type = other RNA; note = AON68 (non-modified)
ggcaattctttcttgaaaat

SEQ ID NO: 136 - RNA, 20 nt, synthetic construct
  source 1..20: mol_type = other RNA; note = AON68
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6: mod_base = OTHER; note = DNA base
  modified_base 8..15: mod_base = OTHER; note = DNA base
  modified_base 7: mod_base = OTHER; note = 2'-O-methyl base
  modified_base 1^2: mod_base = OTHER; note = phosphorothioate linkage
  modified_base 5..8: mod_base = OTHER; note = phosphorothioate linkage
  modified_base 10..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 8..10: mod_base = OTHER; note = mesyl-phosphoramidate linkage
ggcaattctttcttgaaaat

SEQ ID NO: 137 - RNA, 20 nt, synthetic construct
  source 1..20: mol_type = other RNA; note = AON69 (non-modified)
gcaattctttcttgaaaatg

SEQ ID NO: 138 - RNA, 20 nt, synthetic construct
  source 1..20: mol_type = other RNA; note = AON69
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5^6: mod_base = OTHER; note = phosphorothioate linkage
  modified_base 8..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 6..8: mod_base = OTHER; note = mesyl-phosphoramidate linkage
  modified_base 19^20: mod_base = OTHER; note = dimethylimidazolidin-2-ylidene-phosphoramidate linkage
gcaattctttcttgaaaatg

SEQ ID NO: 139 - RNA, 20 nt, synthetic construct
  source 1..20: mol_type = other RNA; note = AON70 (non-modified)
gcaattctttcttgaaaatg

SEQ ID NO: 140 - RNA, 20 nt, synthetic construct
  source 1..20: mol_type = other RNA; note = AON70
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
  modified_base 19^20: mod_base = OTHER; note = dimethylimidazolidin-2-ylidene-phosphoramidate linkage
gcaattctttcttgaaaatg

SEQ ID NO: 141 - RNA, 20 nt, synthetic construct
  source 1..20: mol_type = other RNA; note = AON71 (non-modified)
ggcaattctttcttgaaaat

SEQ ID NO: 142 - RNA, 20 nt, synthetic construct
  source 1..20: mol_type = other RNA; note = AON71
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..7: mod_base = OTHER; note = phosphorothioate linkage
  modified_base 9..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 7..9: mod_base = OTHER; note = mesyl-phosphoramidate linkage
  modified_base 19^20: mod_base = OTHER; note = dimethylimidazolidin-2-ylidene-phosphoramidate linkage
ggcaattctttcttgaaaat

SEQ ID NO: 143 - RNA, 20 nt, synthetic construct
  source 1..20: mol_type = other RNA; note = AON72 (non-modified)
ggcaattctttcttgaaaat

SEQ ID NO: 144 - RNA, 20 nt, synthetic construct
  source 1..20: mol_type = other RNA; note = AON72
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 5..8: mod_base = OTHER; note = phosphorothioate linkage
  modified_base 10..16: mod_base = OTHER; note = phosphorothioate linkage
  modified_base 2..5: mod_base = OTHER; note = phospodiester linkage
  modified_base 16..19: mod_base = OTHER; note = phospodiester linkage
  modified_base 8..10: mod_base = OTHER; note = mesyl-phosphoramidate linkage
  modified_base 19^20: mod_base = OTHER; note = dimethylimidazolidin-2-ylidene-phosphoramidate linkage
ggcaattctttcttgaaaat

SEQ ID NO: 145 - DNA, 2706 bp, Mus musculus
  source 1..2706: mol_type = other DNA; note = mouse PSEN1 (DNA)
agttgcgacaccggtgagacctctagggcggggcctaggacgacctgctccgtgggccgcgagtattcgtcggaaacaaaacagcggcagctgaggcggaaacctaggctgcgagccggccgcccgggcgcggagagagaaggaaccaacacaagacagcagcccttcgaggtctttaggcagcttggaggagaacacatgagagaaagaatcccaagaggttttgttttctttgagaaggtatttctgtccagctgctccaatgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagcgttgccagccctccccatctccatcaccttcgggctcgtgttctacttcgccacggattaccttgtgcagcccttcatggaccaacttgcattccatcagttttatatctagcctttctgcagttagaacatggatgtttcttctttgattatcaaaaacacaaaaacagagagcaagcccgaggaggagactggtgactttcctgtgtcctcagctaacaaaggcaggactccagctggacttctgcagcttccttccgagtctccctagccacccgcactactggactgtggaaggaagcgtctacagagaacggtttccaacatccatcgctgcagcagacggtgtccctcagtgacttgagagacaaggacaaggaaatgtgctgggccaaggagctgccgtgctctgctagctttgacccgtgggcatggagatttacccgcactgtgaactctctaaaggttaaacaaagtgaggtgaaccaaacagagctgccatcttccacaccatgttggaaataaaacacgtcctagctgaacccttactgtccaggaggttccgtgtggaggtggcactgggccgggcctccctctcaggctcctttgctgcccacttgtaagtttaaataaggacaccgccctacacaaacctcacccctgtcacatcagtgactctgaccacttttgttctcaaactctctcactattatctgtggttgccgtttcttcccaaggccagcctggacgaatttggggttgctctatcctgagagttgtaacctcaacttccaaagtttatattttcttgaaatgatggatctattgctcaacagtccctgtcatccttaagtgacttctgggtttcccacaaattcctcacttttagacacactctaagcttacttctggcctggatgcttcctctccctgtctctcccttgccccacagcggttccctgacagcagacaaggcagctctgggaggtagctagtatccaataacccaggggtttcctcatgtgatgcaaatactacgtgtccaaccaatcagtgctgtcaacgggctgccatagctccttcgatggcaaataggatgtgtgcccaaagaattaaagcgatgagtggctggtgaaaaaaaaaaaaaaaaaa

SEQ ID NO: 146 - RNA, 1404 nt, Mus musculus
  source 1..1404: mol_type = other RNA; note = mouse PSEN1 (coding RNA)
atgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagcgttgccagccctccccatctccatcaccttcgggctcgtgttctacttcgccacggattaccttgtgcagcccttcatggaccaacttgcattccatcagttttatatctag

SEQ ID NO: 147 - protein, 467 aa, Mus musculus
  source 1..467: note = mouse PSEN1 (protein)
MTEIPAPLSYFQNAQMSEDSHSSSAIRSQNDSQERQQQHDRQRLDNPEPISNGRPQSNSRQVVEQDEEEDEELTLKYGAKHVIMLFVPVTLCMVVVVATIKSVSFYTRKDGQLIYTPFTEDTETVGQRALHSILNAAIMISVIVIMTILLVVLYKYRCYKVIHAWLIISSLLLLFFFSFIYLGEVFKTYNVAVDYVTVALLIWNFGVVGMIAIHWKGPLRLQQAYLIMISALMALVFIKYLPEWTAWLILAVISVYDLVAVLCPKGPLRMLVETAQERNETLFPALIYSSTMVWLVNMAEGDPEAQRRVPKNPKYNTQRAERETQDSGSGNDDGGFSEEWEAQRDSHLGPHRSTPESRAAVQELSGSILTSEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 148 - DNA, 2706 bp, Mus musculus
  source 1..2706: mol_type = other DNA; note = mouse A431E PSEN1 (DNA)
agttgcgacaccggtgagacctctagggcggggcctaggacgacctgctccgtgggccgcgagtattcgtcggaaacaaaacagcggcagctgaggcggaaacctaggctgcgagccggccgcccgggcgcggagagagaaggaaccaacacaagacagcagcccttcgaggtctttaggcagcttggaggagaacacatgagagaaagaatcccaagaggttttgttttctttgagaaggtatttctgtccagctgctccaatgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagaattgccggcgctccccatctccatcaccttcgggctcgtgttctacttcgccacggattaccttgtgcagcccttcatggaccaacttgcattccatcagttttatatctagcctttctgcagttagaacatggatgtttcttctttgattatcaaaaacacaaaaacagagagcaagcccgaggaggagactggtgactttcctgtgtcctcagctaacaaaggcaggactccagctggacttctgcagcttccttccgagtctccctagccacccgcactactggactgtggaaggaagcgtctacagagaacggtttccaacatccatcgctgcagcagacggtgtccctcagtgacttgagagacaaggacaaggaaatgtgctgggccaaggagctgccgtgctctgctagctttgacccgtgggcatggagatttacccgcactgtgaactctctaaaggttaaacaaagtgaggtgaaccaaacagagctgccatcttccacaccatgttggaaataaaacacgtcctagctgaacccttactgtccaggaggttccgtgtggaggtggcactgggccgggcctccctctcaggctcctttgctgcccacttgtaagtttaaataaggacaccgccctacacaaacctcacccctgtcacatcagtgactctgaccacttttgttctcaaactctctcactattatctgtggttgccgtttcttcccaaggccagcctggacgaatttggggttgctctatcctgagagttgtaacctcaacttccaaagtttatattttcttgaaatgatggatctattgctcaacagtccctgtcatccttaagtgacttctgggtttcccacaaattcctcacttttagacacactctaagcttacttctggcctggatgcttcctctccctgtctctcccttgccccacagcggttccctgacagcagacaaggcagctctgggaggtagctagtatccaataacccaggggtttcctcatgtgatgcaaatactacgtgtccaaccaatcagtgctgtcaacgggctgccatagctccttcgatggcaaataggatgtgtgcccaaagaattaaagcgatgagtggctggtgaaaaaaaaaaaaaaaaaa

SEQ ID NO: 149 - RNA, 1404 nt, Mus musculus
  source 1..1404: mol_type = other RNA; note = mouse A431E PSEN1 (coding RNA)
atgacagagatacctgcacctttgtcctacttccagaatgcccagatgtctgaggacagccactccagcagcgccatccggagccagaatgacagccaagaacggcagcagcagcatgacaggcagagacttgacaaccctgagccaatatctaatgggcggccccagagtaactcaagacaggtggtggaacaagatgaggaggaagacgaagagctgacattgaaatatggagccaagcatgtcatcatgctctttgtccccgtgaccctctgcatggtcgtcgtcgtggccaccatcaaatcagtcagcttctatacccggaaggacggtcagctaatctacaccccattcacagaagacactgagactgtaggccaaagagccctgcactcgatcctgaatgcggccatcatgatcagtgtcattgtcattatgaccatcctcctggtggtcctgtataaatacaggtgctacaaggtcatccacgcctggcttattatttcatctctgttgttgctgttctttttttcgttcatttacttaggggaagtatttaagacctacaatgtcgccgtggactacgttacagtagcactcctaatctggaattttggtgtggtcgggatgattgccatccactggaaaggcccccttcgactgcagcaggcgtatctcattatgatcagtgccctcatggccctggtatttatcaagtacctccccgaatggaccgcatggctcatcttggctgtgatttcagtatatgatttggtggctgttttatgtcccaaaggcccacttcgtatgctggttgaaacagctcaggaaagaaatgagactctctttccagctcttatctattcctcaacaatggtgtggttggtgaatatggctgaaggagacccagaagcccaaaggagggtacccaagaaccccaagtataacacacaaagagcggagagagagacacaggacagtggttctgggaacgatgatggtggcttcagtgaggagtgggaggcccaaagagacagtcacctggggcctcatcgctccactcccgagtcaagagctgctgtccaggaactttctgggagcattctaacgagtgaagacccggaggaaagaggagtaaaacttggactgggagatttcattttctacagtgttctggttggtaaggcctcagcaaccgccagtggagactggaacacaaccatagcctgctttgtagccatactgatcggcctgtgccttacattactcctgctcgccattttcaagaaagaattgccggcgctccccatctccatcaccttcgggctcgtgttctacttcgccacggattaccttgtgcagcccttcatggaccaacttgcattccatcagttttatatctag

SEQ ID NO: 150 - protein, 467 aa, Mus musculus
  source 1..467: note = mouse A431E PSEN1 (protein)
MTEIPAPLSYFQNAQMSEDSHSSSAIRSQNDSQERQQQHDRQRLDNPEPISNGRPQSNSRQVVEQDEEEDEELTLKYGAKHVIMLFVPVTLCMVVVVATIKSVSFYTRKDGQLIYTPFTEDTETVGQRALHSILNAAIMISVIVIMTILLVVLYKYRCYKVIHAWLIISSLLLLFFFSFIYLGEVFKTYNVAVDYVTVALLIWNFGVVGMIAIHWKGPLRLQQAYLIMISALMALVFIKYLPEWTAWLILAVISVYDLVAVLCPKGPLRMLVETAQERNETLFPALIYSSTMVWLVNMAEGDPEAQRRVPKNPKYNTQRAERETQDSGSGNDDGGFSEEWEAQRDSHLGPHRSTPESRAAVQELSGSILTSEDPEERGVKLGLGDFIFYSVLVGKASATASGDWNTTIACFVAILIGLCLTLLLLAIFKKELPALPISITFGLVFYFATDYLVQPFMDQLAFHQFYI

SEQ ID NO: 151 - DNA, 6018 bp, Homo sapiens
  source 1..6018: mol_type = other DNA; note = human PSEN1 (DNA)
ggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcactttctgtcctggtattttgtttttgcttttgccacacagtagctcagaatttgaacaaatagccaaaagctggtggttgatgaattatgaactagttgtatcaacacaaagcaagagttggggaaagccatatttaacttggtgagctgtgggagaacctggtggcagaaggagaaccaactgccaaggggaaagagaaggggcctccagcagcgaaggggatacagtgagctaatgatgtcaaggaggagtttcaggttattctcgtcagctccacaaatgggtgctttgtggtctctgcccgcgttacctttcctctcaatgtacctttgtgtgaactgggcagtggaggtgcctgctgcagttaccatggagttcaggctctgggcagctcagtcaggcaaaacacacaaacagccatcagcctgtgtgggctcagggcacctctggacaaaggcttgtggggcataaccttctttaccacagagagcccttagctatgctgatcagaccgtaagcgtttatgagaaacttagtttcctcctgtggctgaggaggggccagctttttcttcttttgcctgctgttttctctcccaatctatgatatgatatgacctggtttggggctgtctttggtgtttagaatatttgttttctgtcccaggatatttcttataagaacctaacttcaagagtagtgtgcgagtactgatctgaatttaaattaaaattggcttatattaggcagtcacagacaggaaaaataagagctatgcaaagaaagggggatttaaagtagtaggttctatcatctcaattcatttttttccatgaaatcccttcttccaagattcattccctctctcagacatgtgctagcatgggtattatcattgagaaagcacagctacagcaaagccacctgaatagcaatttgtgattggaagcattcttgagggatccctaatctagagtaatttatttgtgtaaggatcccaaatgtgttgcacctttcatgatacatttcttctctgaagagggtacgtggggtgtgtgtatttaaatccatcctatgtattactgattgtcctgtgtagaaagatggcaattattctgtctctttctccaagtttgagccacatctcagccacattgttagacagtgtacagagaacctatctttcctttttttttttttaaaggacaggattttgctgtgttgcccaggctagacttgaactcctgggctcaagtaatccacctcagcctgagtagctgagactacagcccatcttatttctttaaatcattcatctcaggcagagaacttttccctcaaacattctttttagaattagttcagtcattcctaaaacatccaaatgctagtcttccaccatgaaaaatagattgtcactggaaagaacagtagcaatttccataaggatgtgccttcactcacacgggacaggcggtggttatagagtcgggcaaaaccagcagtagagtatgaccagccaagccaatctgcttaataaaaagatggaagacagtaaggaaggaaagtagccactaagagtctgagtctgactgggctacagaataaagggtatttatggacagaatgtcattacatgcctatgggaataccaatcatatttggaagatttgcagattttttttcagagaggaaagactcaccttcctgtttttggttctcagtaggttcgtgtgtgttcctagaatcacagctctgactccaaatgactcaatttctcaattagaaaaagtagaagctttctaagcaacttggaagaaaacagtcataagtaagcaatttgttgattttactacagaagcaacaactgaagaggcagtgtttttactttcagactccgggattcccattctgtagtctctctgcttttaaaaaccctccttttgcaatagatgcccaaacagatgatgtttattacttgttatttacgtggcctcagacagtgtatgtattctcgatataacttgtagagtgtgaaatataagtttaactaccaaataaggtctcccagggttagatgactgcgggaagcctttgatcccaacccccaaggctttgtatatttgatcatttgtgatctaaccctggaagaaaaagagctcagaaaccactatgaaaaaatttgttcagtgttttctgtgttcccgtaggttctggagtctgaggatgcaaagatgaataagataaattctcagaatgtagttataatctcttgttttctggtatatgccatctttctttaacttctctaaaatattgggtatttgtcaaataaccacttttaacagttaccattactgagggcttatacattggtgttataaaagtgacttgattcagaaatcaatccattcagtaaagtactccttctctaaatttgctgttatgtctataaggaacagtttgacctgcccttctcctcacctcctcacctgccttccaacattgaatttggaaggagacgtgaaaattggacatttggttttgcccttgggctggaaactatcatataatcataagtttgagcctagaagtgatccttgtgatcttctcacctctttaaattcccacaacacaagagattaaaaacagaggtttcagctcttcatagtgcgttgtgaaatggctggccagagtgtaccaacaaagctgtcatcgggctcacagctcagagacatctgcatgtgatcatctgcatagtcctctcctctaacgggaaacacctcagatttgcatataaaaaagcaccctggtgctgaaatgaacccctttcttgaacatcaaagctgtctcccacagccttgggcagcagggtgcctcttagtggatgtgctgggtccaccctgagccctgacatgtggtggcagcattgccagttggtctgtgtgtctgtgtagcagggacgatttcccagaaagcaattttccttttgaaatacgtaattgttgagactaggcagtttcaaagtcagctgcatatagtagcaagtacaggactgtcttgtttttggtgtccttggaggtgctggggtgagggtttcagtgggatcatttactctcacatgttgtctgccttctgcttctgtggacactgctttgtacttaattcagacagactgtgaatacaccttttttataaatacctttcaaattcttggtaagatataattttgatagctgattgcagattttctgtatttgtcagattaataaagactgcatgaatcca

SEQ ID NO: 152 - RNA, 1404 nt, Homo sapiens
  source 1..1404: mol_type = other RNA; note = human PSEN1 isoform 1 (coding RNA)
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 153 - protein, 467 aa, Homo sapiens
  source 1..467: note = human PSEN1 isoform 1 (protein)
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 154 - DNA, 6018 bp, Homo sapiens
  source 1..6018: mol_type = other DNA; note = human A431E PSEN1 (DNA)
ggaaacaaaacagcggctggtctggaaggaacctgagctacgagccgcggcggcagcggggcggcggggaagcgtatacctaatctgggagcctgcaagtgacaacagcctttgcggtccttagacagcttggcctggaggagaacacatgaaagaaagaacctcaagaggctttgttttctgtgaaacagtatttctatacagttgctccaatgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagaattgccagctcttccaatctccatcacctttgggcttgttttctactttgccacagattatcttgtacagccttttatggaccaattagcattccatcaattttatatctagcatatttgcggttagaatcccatggatgtttcttctttgactataacaaaatctggggaggacaaaggtgattttcctgtgtccacatctaacaaagtcaagattcccggctggacttttgcagcttccttccaagtcttcctgaccaccttgcactattggactttggaaggaggtgcctatagaaaacgattttgaacatacttcatcgcagtggactgtgtccctcggtgcagaaactaccagatttgagggacgaggtcaaggagatatgataggcccggaagttgctgtgccccatcagcagcttgacgcgtggtcacaggacgatttcactgacactgcgaactctcaggactaccgttaccaagaggttaggtgaagtggtttaaaccaaacggaactcttcatcttaaactacacgttgaaaatcaacccaataattctgtattaactgaattctgaacttttcaggaggtactgtgaggaagagcaggcaccagcagcagaatggggaatggagaggtgggcaggggttccagcttccctttgattttttgctgcagactcatcctttttaaatgagacttgttttcccctctctttgagtcaagtcaaatatgtagattgcctttggcaattcttcttctcaagcactgacactcattaccgtctgtgattgccatttcttcccaaggccagtctgaacctgaggttgctttatcctaaaagttttaacctcaggttccaaattcagtaaattttggaaacagtacagctatttctcatcaattctctatcatgttgaagtcaaatttggattttccaccaaattctgaatttgtagacatacttgtacgctcacttgccccagatgcctcctctgtcctcattcttctctcccacacaagcagtctttttctacagccagtaaggcagctctgtcgtggtagcagatggtcccattattctagggtcttactctttgtatgatgaaaagaatgtgttatgaatcggtgctgtcagccctgctgtcagaccttcttccacagcaaatgagatgtatgcccaaagacggtagaattaaagaagagtaaaatggctgttgaagcactttctgtcctggtattttgtttttgcttttgccacacagtagctcagaatttgaacaaatagccaaaagctggtggttgatgaattatgaactagttgtatcaacacaaagcaagagttggggaaagccatatttaacttggtgagctgtgggagaacctggtggcagaaggagaaccaactgccaaggggaaagagaaggggcctccagcagcgaaggggatacagtgagctaatgatgtcaaggaggagtttcaggttattctcgtcagctccacaaatgggtgctttgtggtctctgcccgcgttacctttcctctcaatgtacctttgtgtgaactgggcagtggaggtgcctgctgcagttaccatggagttcaggctctgggcagctcagtcaggcaaaacacacaaacagccatcagcctgtgtgggctcagggcacctctggacaaaggcttgtggggcataaccttctttaccacagagagcccttagctatgctgatcagaccgtaagcgtttatgagaaacttagtttcctcctgtggctgaggaggggccagctttttcttcttttgcctgctgttttctctcccaatctatgatatgatatgacctggtttggggctgtctttggtgtttagaatatttgttttctgtcccaggatatttcttataagaacctaacttcaagagtagtgtgcgagtactgatctgaatttaaattaaaattggcttatattaggcagtcacagacaggaaaaataagagctatgcaaagaaagggggatttaaagtagtaggttctatcatctcaattcatttttttccatgaaatcccttcttccaagattcattccctctctcagacatgtgctagcatgggtattatcattgagaaagcacagctacagcaaagccacctgaatagcaatttgtgattggaagcattcttgagggatccctaatctagagtaatttatttgtgtaaggatcccaaatgtgttgcacctttcatgatacatttcttctctgaagagggtacgtggggtgtgtgtatttaaatccatcctatgtattactgattgtcctgtgtagaaagatggcaattattctgtctctttctccaagtttgagccacatctcagccacattgttagacagtgtacagagaacctatctttcctttttttttttttaaaggacaggattttgctgtgttgcccaggctagacttgaactcctgggctcaagtaatccacctcagcctgagtagctgagactacagcccatcttatttctttaaatcattcatctcaggcagagaacttttccctcaaacattctttttagaattagttcagtcattcctaaaacatccaaatgctagtcttccaccatgaaaaatagattgtcactggaaagaacagtagcaatttccataaggatgtgccttcactcacacgggacaggcggtggttatagagtcgggcaaaaccagcagtagagtatgaccagccaagccaatctgcttaataaaaagatggaagacagtaaggaaggaaagtagccactaagagtctgagtctgactgggctacagaataaagggtatttatggacagaatgtcattacatgcctatgggaataccaatcatatttggaagatttgcagattttttttcagagaggaaagactcaccttcctgtttttggttctcagtaggttcgtgtgtgttcctagaatcacagctctgactccaaatgactcaatttctcaattagaaaaagtagaagctttctaagcaacttggaagaaaacagtcataagtaagcaatttgttgattttactacagaagcaacaactgaagaggcagtgtttttactttcagactccgggattcccattctgtagtctctctgcttttaaaaaccctccttttgcaatagatgcccaaacagatgatgtttattacttgttatttacgtggcctcagacagtgtatgtattctcgatataacttgtagagtgtgaaatataagtttaactaccaaataaggtctcccagggttagatgactgcgggaagcctttgatcccaacccccaaggctttgtatatttgatcatttgtgatctaaccctggaagaaaaagagctcagaaaccactatgaaaaaatttgttcagtgttttctgtgttcccgtaggttctggagtctgaggatgcaaagatgaataagataaattctcagaatgtagttataatctcttgttttctggtatatgccatctttctttaacttctctaaaatattgggtatttgtcaaataaccacttttaacagttaccattactgagggcttatacattggtgttataaaagtgacttgattcagaaatcaatccattcagtaaagtactccttctctaaatttgctgttatgtctataaggaacagtttgacctgcccttctcctcacctcctcacctgccttccaacattgaatttggaaggagacgtgaaaattggacatttggttttgcccttgggctggaaactatcatataatcataagtttgagcctagaagtgatccttgtgatcttctcacctctttaaattcccacaacacaagagattaaaaacagaggtttcagctcttcatagtgcgttgtgaaatggctggccagagtgtaccaacaaagctgtcatcgggctcacagctcagagacatctgcatgtgatcatctgcatagtcctctcctctaacgggaaacacctcagatttgcatataaaaaagcaccctggtgctgaaatgaacccctttcttgaacatcaaagctgtctcccacagccttgggcagcagggtgcctcttagtggatgtgctgggtccaccctgagccctgacatgtggtggcagcattgccagttggtctgtgtgtctgtgtagcagggacgatttcccagaaagcaattttccttttgaaatacgtaattgttgagactaggcagtttcaaagtcagctgcatatagtagcaagtacaggactgtcttgtttttggtgtccttggaggtgctggggtgagggtttcagtgggatcatttactctcacatgttgtctgccttctgcttctgtggacactgctttgtacttaattcagacagactgtgaatacaccttttttataaatacctttcaaattcttggtaagatataattttgatagctgattgcagattttctgtatttgtcagattaataaagactgcatgaatcca

SEQ ID NO: 155 - RNA, 1400 nt, Homo sapiens
  source 1..1400: mol_type = other RNA; note = human A431E PSEN1 isoform 1 (coding RNA)
atgacagagttacctgcaccgttgtcctacttccagaatgcacagatgtctgaggacaaccacctgagcaatactgtacgtagccagaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagaattgccagctcttccaatctccatcacctttgggcttgttttctactttgccacagattatcttgtacagccttttatggaccaattagcattccatcaattttatat

SEQ ID NO: 156 - protein, 467 aa, Homo sapiens
  source 1..467: note = human A431E PSEN1 isoform 1 (protein)
MTELPAPLSYFQNAQMSEDNHLSNTVRSQNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKELPALPISITFGLVFYFATDYLVQPFMDQLAFHQFYI

SEQ ID NO: 157 - RNA, 21 nt, synthetic construct
  source 1..21: mol_type = other RNA; note = AON21 (non-modified)
agcgccggcaattctttcttg

SEQ ID NO: 158 - skipped

SEQ ID NO: 159 - protein, 463 aa, Homo sapiens
  source 1..463: note = human PSEN1 isoform 2 (protein)
MTELPAPLSYFQNAQMSEDNHLSNTNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKALPALPISITFGLVFYFATDYLVQPFMDQLAFHQFYI

SEQ ID NO: 160 - skipped

SEQ ID NO: 161 - protein, 463 aa, Homo sapiens
  source 1..463: note = human A431E PSEN1 isoform 2 (protein)
MTELPAPLSYFQNAQMSEDNHLSNTNDNRERQEHNDRRSLGHPEPLSNGRPQGNSRQVVEQDEEEDEELTLKYGAKHVIMLFVPVTLCMVVVVATIKSVSFYTRKDGQLIYTPFTEDTETVGQRALHSILNAAIMISVIVVMTILLVVLYKYRCYKVIHAWLIISSLLLLFFFSFIYLGEVFKTYNVAVDYITVALLIWNFGVVGMISIHWKGPLRLQQAYLIMISALMALVFIKYLPEWTAWLILAVISVYDLVAVLCPKGPLRMLVETAQERNETLFPALIYSSTMVWLVNMAEGDPEAQRRVSKNSKYNAESTERESQDTVAENDDGGFSEEWEAQRDSHLGPHRSTPESRAAVQELSSSILAGEDPEERGVKLGLGDFIFYSVLVGKASATASGDWNTTIACFVAILIGLCLTLLLLAIFKKELPALPISITFGLVFYFATDYLVQPFMDQLAFHQFYI

SEQ ID NO: 162 - protein, 15 aa, synthetic construct
  source 1..15: note = PSEN1 immunogen
SQERQQQHGRQRLDN

SEQ ID NO: 163 - protein, 16 aa, synthetic construct
  source 1..16: note = APP immunogen
NGYENPTYKFFEQMQN

SEQ ID NO: 164 - protein, 15 aa, synthetic construct
  source 1..15: note = b-actin immunogen
DDDIAALVIDNGSGK

SEQ ID NO: 165 - DNA, 2249 bp, Homo sapiens
  source 1..2249: mol_type = other DNA; note = human PSEN2 (DNA)
gcgtaaactccgctggagcgcggcggcagagcaggcatttccagcagtgaggagacagccagaagcaagcttttggagctgaaggaacctgagacagaagctagtcccccctctgaattttactgatgaagaaactgaggccacagagctaaagtgacttttcccaaggtcgcccagcgaggacgtgggacttctcagacgtcaggagagtgatgtgagggagctgtgtgaccatagaaagtgacgtgttaaaaaccagcgctgccctctttgaaagccagggagcatcattcatttagcctgctgagaagaagaaaccaagtgtccgggattcagacctctctgcggccccaagtgttcgtggtgcttccagaggcagggctatgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctgagggacatggtgtgccacaggctgcaagctgcagggaattttcattggatgcagttgtatagttttacactctagtgccatatatttttaagacttttctttccttaaaaaataaagtacgtgtttacttggtgaggaggaggcagaaccagctctttggtgccagctgtttcatcaccagactttggctcccgctttggggagcgcctcgcttcacggacaggaagcacagcaggtttatccagatgaactgagaaggtcagattagggcggggagaagagcatccggcatgagggctgagatgcgcaaagagtgtgctcgggagtggcccctggcacctgggtgctctggctggagaggaaaagccagttccctacgaggagtgttcccaatgctttgtccatgatgtccttgttattttattgcctttagaaactgagtcctgttcttgttacggcagtcacactgctgggaagtggcttaatagtaatatcaataaatagatgagtcctgttagaa

SEQ ID NO: 166 - protein, 448 aa, Homo sapiens
  source 1..448: note = human PSEN2 isoform 1 (protein)
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 167 - skipped

SEQ ID NO: 168 - protein, 447 aa, Homo sapiens
  source 1..447: note = human PSEN2 isoform 2 (protein)
MLTFMASDSEEEVCDERTSLMSAESPTPRSCQEGRQGPEDGENTAQWRSQENEEDGEEDPDRYVCSGVPGRPPGLEEELTLKYGAKHVIMLFVPVTLCMIVVVATIKSVRFYTEKNGQLIYTPFTEDTPSVGQRLLNSVLNTLIMISVIVVMTIFLVVLYKYRCYKFIHGWLIMSSLMLLFLFTYIYLGEVLKTYNVAMDYPTLLLTVWNFGAVGMVCIHWKGPLVLQQAYLIMISALMALVFIKYLPEWSAWVILGAISVYDLVAVLCPKGPLRMLVETAQERNEPIFPALIYSSAMVWTVGMAKLDPSSQGALQLPYDPEMEDSYDSFGEPSYPEVFEPPLTGYPGEELEEEEERGVKLGLGDFIFYSVLVGKAAATGSGDWNTTLACFVAILIGLCLTLLLLAVFKKALPALPISITFGLIFYFSTDNLVRPFMDTLASHQLYI

SEQ ID NO: 169 - RNA, 1392 nt, Homo sapiens
  source 1..1392: mol_type = other RNA; note = human PSEN1 isoform 2 (RNA)
atgacagagttacctgcaccgttgtcctacttccagaatgcacagatgtctgaggacaaccacctgagcaatactaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagcattgccagctcttccaatctccatcacctttgggcttgttttctactttgccacagattatcttgtacagccttttatggaccaattagcattccatcaattttatatctag

SEQ ID NO: 170 - RNA, 1392 nt, Homo sapiens
  source 1..1392: mol_type = other RNA; note = human A431E PSEN1 isoform 2 (RNA)
atgacagagttacctgcaccgttgtcctacttccagaatgcacagatgtctgaggacaaccacctgagcaatactaatgacaatagagaacggcaggagcacaacgacagacggagccttggccaccctgagccattatctaatggacgaccccagggtaactcccggcaggtggtggagcaagatgaggaagaagatgaggagctgacattgaaatatggcgccaagcatgtgatcatgctctttgtccctgtgactctctgcatggtggtggtcgtggctaccattaagtcagtcagcttttatacccggaaggatgggcagctaatctataccccattcacagaagataccgagactgtgggccagagagccctgcactcaattctgaatgctgccatcatgatcagtgtcattgttgtcatgactatcctcctggtggttctgtataaatacaggtgctataaggtcatccatgcctggcttattatatcatctctattgttgctgttctttttttcattcatttacttgggggaagtgtttaaaacctataacgttgctgtggactacattactgttgcactcctgatctggaattttggtgtggtgggaatgatttccattcactggaaaggtccacttcgactccagcaggcatatctcattatgattagtgccctcatggccctggtgtttatcaagtacctccctgaatggactgcgtggctcatcttggctgtgatttcagtatatgatttagtggctgttttgtgtccgaaaggtccacttcgtatgctggttgaaacagctcaggagagaaatgaaacgctttttccagctctcatttactcctcaacaatggtgtggttggtgaatatggcagaaggagacccggaagctcaaaggagagtatccaaaaattccaagtataatgcagaaagcacagaaagggagtcacaagacactgttgcagagaatgatgatggcgggttcagtgaggaatgggaagcccagagggacagtcatctagggcctcatcgctctacacctgagtcacgagctgctgtccaggaactttccagcagtatcctcgctggtgaagacccagaggaaaggggagtaaaacttggattgggagatttcattttctacagtgttctggttggtaaagcctcagcaacagccagtggagactggaacacaaccatagcctgtttcgtagccatattaattggtttgtgccttacattattactccttgccattttcaagaaagaattgccagctcttccaatctccatcacctttgggcttgttttctactttgccacagattatcttgtacagccttttatggaccaattagcattccatcaattttatatctag

SEQ ID NO: 171 - RNA, 1347 nt, Homo sapiens
  source 1..1347: mol_type = other RNA; note = human PSEN2 isoform 1 (RNA)
atgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 172 - RNA, 1344 nt, Homo sapiens
  source 1..1344: mol_type = other RNA; note = human PSEN2 isoform 2 (RNA)
atgctcacattcatggcctctgacagcgaggaagaagtgtgtgatgagcggacgtccctaatgtcggctgagagccccacgccgcgctcctgccaggagggcaggcagggcccagaggatggagagaacactgcccagtggagaagccaggagaacgaggaggacggtgaggaggaccctgaccgctatgtctgtagtggggttcccgggcggccgccaggcctggaggaagagctgaccctcaaatacggagcgaagcacgtgatcatgctgtttgtgcctgtcactctgtgcatgatcgtggtggtagccaccatcaagtctgtgcgcttctacacagagaagaatggacagctcatctacacgccattcactgaggacacaccctcggtgggccagcgcctcctcaactccgtgctgaacaccctcatcatgatcagcgtcatcgtggttatgaccatcttcttggtggtgctctacaagtaccgctgctacaagttcatccatggctggttgatcatgtcttcactgatgctgctgttcctcttcacctatatctaccttggggaagtgctcaagacctacaatgtggccatggactaccccaccctcttgctgactgtctggaacttcggggcagtgggcatggtgtgcatccactggaagggccctctggtgctgcagcaggcctacctcatcatgatcagtgcgctcatggccctagtgttcatcaagtacctcccagagtggtccgcgtgggtcatcctgggcgccatctctgtgtatgatctcgtggctgtgctgtgtcccaaagggcctctgagaatgctggtagaaactgcccaggagagaaatgagcccatattccctgccctgatatactcatctgccatggtgtggacggttggcatggcgaagctggacccctcctctcagggtgccctccagctcccctacgacccggagatggaagactcctatgacagttttggggagccttcataccccgaagtctttgagcctcccttgactggctacccaggggaggagctggaggaagaggaggaaaggggcgtgaagcttggcctcggggacttcatcttctacagtgtgctggtgggcaaggcggctgccacgggcagcggggactggaataccacgctggcctgcttcgtggccatcctcattggcttgtgtctgaccctcctgctgcttgctgtgttcaagaaggcgctgcccgccctccccatctccatcacgttcgggctcatcttttacttctccacggacaacctggtgcggccgttcatggacaccctggcctcccatcagctctacatctga

SEQ ID NO: 173 - RNA, 20 nt, synthetic construct
  source 1..20: mol_type = other RNA; note = AON 73 (non-modified)
gtaaatcattcccaccacac

SEQ ID NO: 174 - RNA, 20 nt, synthetic construct
  source 1..20: mol_type = other RNA; note = AON 73
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16: mod_base = OTHER; note = 5-methyl
  modified_base 18: mod_base = OTHER; note = 5-methyl
  modified_base 20: mod_base = OTHER; note = 5-methyl
  modified_base 1..20: mod_base = OTHER; note = phosphorothioate linkage
gtaaatcattcccaccacac

SEQ ID NO: 175 - RNA, 20 nt, synthetic construct
  source 1..20: mol_type = other RNA; note = AON 74 (non-modified)
atgtaaatcattcccaccac

SEQ ID NO: 176 - RNA, 20 nt, synthetic construct
  source 1..20: mol_type = other RNA; note = AON 74
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 17..18: mod_base = OTHER; note = 5-methyl
  modified_base 20: mod_base = OTHER; note = 5-methyl
  modified_base 1..20: mod_base = OTHER; note = phosphorothioate linkage
atgtaaatcattcccaccac

SEQ ID NO: 177 - RNA, 20 nt, synthetic construct
  source 1..20: mol_type = other RNA; note = AON 75 (non-modified)
gaatgtaaatcattcccacc

SEQ ID NO: 178 - RNA, 20 nt, synthetic construct
  source 1..20: mol_type = other RNA; note = AON 75
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16..17: mod_base = OTHER; note = 5-methyl
  modified_base 19..20: mod_base = OTHER; note = 5-methyl
  modified_base 1..20: mod_base = OTHER; note = phosphorothioate linkage
gaatgtaaatcattcccacc

SEQ ID NO: 179 - RNA, 20 nt, synthetic construct
  source 1..20: mol_type = other RNA; note = AON 76 (non-modified)
gtgaatgtaaatcattccca

SEQ ID NO: 180 - RNA, 20 nt, synthetic construct
  source 1..20: mol_type = other RNA; note = AON 76
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 17..19: mod_base = OTHER; note = 5-methyl
  modified_base 1..20: mod_base = OTHER; note = phosphorothioate linkage
gtgaatgtaaatcattccca

SEQ ID NO: 181 - RNA, 20 nt, synthetic construct
  source 1..20: mol_type = other RNA; note = AON 77 (non-modified)
cagtgaatgtaaatcattcc

SEQ ID NO: 182 - RNA, 20 nt, synthetic construct
  source 1..20: mol_type = other RNA; note = AON 77
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 1: mod_base = OTHER; note = 5-methyl
  modified_base 19..20: mod_base = OTHER; note = 5-methyl
  modified_base 6..15: mod_base = OTHER; note = DNA base
  modified_base 1..20: mod_base = OTHER; note = phosphorothioate linkage
cagtgaatgtaaatcattcc

SEQ ID NO: 183 - RNA, 20 nt, synthetic construct
  source 1..20: mol_type = other RNA; note = AON 78 (non-modified)
tccagtgaatgtaaatcatt

SEQ ID NO: 184 - RNA, 20 nt, synthetic construct
  source 1..20: mol_type = other RNA; note = AON 78
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2..3: mod_base = OTHER; note = 5-methyl
  modified_base 17: mod_base = OTHER; note = 5-methyl
  modified_base 6..15: mod_base = OTHER; note = DNA base
  modified_base 1..20: mod_base = OTHER; note = phosphorothioate linkage
tccagtgaatgtaaatcatt

SEQ ID NO: 185 - RNA, 20 nt, synthetic construct
  source 1..20: mol_type = other RNA; note = AON 79 (non-modified)
tttccagtgaatgtaaatca

SEQ ID NO: 186 - RNA, 20 nt, synthetic construct
  source 1..20: mol_type = other RNA; note = AON 79
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4..5: mod_base = OTHER; note = 5-methyl
  modified_base 19: mod_base = OTHER; note = 5-methyl
  modified_base 6..15: mod_base = OTHER; note = DNA base
  modified_base 1..20: mod_base = OTHER; note = phosphorothioate linkage
tttccagtgaatgtaaatca

SEQ ID NO: 187 - RNA, 20 nt, synthetic construct
  source 1..20: mol_type = other RNA; note = AON 80 (non-modified)
gtaaatcattcccaccacac

SEQ ID NO: 188 - RNA, 20 nt, synthetic construct
  source 1..20: mol_type = other RNA; note = AON 80
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16: mod_base = OTHER; note = 5-methyl
  modified_base 18: mod_base = OTHER; note = 5-methyl
  modified_base 20: mod_base = OTHER; note = 5-methyl
  modified_base 1..20: mod_base = OTHER; note = phosphorothioate linkage
gtaaatcattcccaccacac

SEQ ID NO: 189 - RNA, 20 nt, synthetic construct
  source 1..20: mol_type = other RNA; note = AON 81 (non-modified)
atgtaaatcattcccaccac

SEQ ID NO: 190 - RNA, 20 nt, synthetic construct
  source 1..20: mol_type = other RNA; note = AON 81
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 17..18: mod_base = OTHER; note = 5-methyl
  modified_base 20: mod_base = OTHER; note = 5-methyl
  modified_base 1..20: mod_base = OTHER; note = phosphorothioate linkage
atgtaaatcattcccaccac

SEQ ID NO: 191 - RNA, 20 nt, synthetic construct
  source 1..20: mol_type = other RNA; note = AON 82 (non-modified)
gaatgtaaatcattcccacc

SEQ ID NO: 192 - RNA, 20 nt, synthetic construct
  source 1..20: mol_type = other RNA; note = AON 82
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 16..17: mod_base = OTHER; note = 5-methyl
  modified_base 19..20: mod_base = OTHER; note = 5-methyl
  modified_base 1..20: mod_base = OTHER; note = phosphorothioate linkage
gaatgtaaatcattcccacc

SEQ ID NO: 193 - RNA, 20 nt, synthetic construct
  source 1..20: mol_type = other RNA; note = AON 83 (non-modified)
gtgaatgtaaatcattccca

SEQ ID NO: 194 - RNA, 20 nt, synthetic construct
  source 1..20: mol_type = other RNA; note = AON 83
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 6..15: mod_base = OTHER; note = DNA base
  modified_base 17..19: mod_base = OTHER; note = 5-methyl
  modified_base 1..20: mod_base = OTHER; note = phosphorothioate linkage
gtgaatgtaaatcattccca

SEQ ID NO: 195 - RNA, 20 nt, synthetic construct
  source 1..20: mol_type = other RNA; note = AON 84 (non-modified)
cagtgaatgtaaatcattcc

SEQ ID NO: 196 - RNA, 20 nt, synthetic construct
  source 1..20: mol_type = other RNA; note = AON 84
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 1: mod_base = OTHER; note = 5-methyl
  modified_base 19: mod_base = OTHER; note = 5-methyl
  modified_base 6..15: mod_base = OTHER; note = DNA base
  modified_base 1..20: mod_base = OTHER; note = phosphorothioate linkage
cagtgaatgtaaatcattcc

SEQ ID NO: 197 - RNA, 20 nt, synthetic construct
  source 1..20: mol_type = other RNA; note = AON 85 (non-modified)
tccagtgaatgtaaatcatt

SEQ ID NO: 198 - RNA, 20 nt, synthetic construct
  source 1..20: mol_type = other RNA; note = AON 85
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2..3: mod_base = OTHER; note = 5-methyl
  modified_base 17: mod_base = OTHER; note = 5-methyl
  modified_base 6..15: mod_base = OTHER; note = DNA base
  modified_base 1..20: mod_base = OTHER; note = phosphorothioate linkage
tccagtgaatgtaaatcatt

SEQ ID NO: 199 - RNA, 20 nt, synthetic construct
  source 1..20: mol_type = other RNA; note = AON 86 (non-modified)
tttccagtgaatgtaaatca

SEQ ID NO: 200 - RNA, 20 nt, synthetic construct
  source 1..20: mol_type = other RNA; note = AON 86
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4..5: mod_base = OTHER; note = 5-methyl
  modified_base 19: mod_base = OTHER; note = 5-methyl
  modified_base 6..15: mod_base = OTHER; note = DNA base
  modified_base 1..20: mod_base = OTHER; note = phosphorothioate linkage
tttccagtgaatgtaaatca

SEQ ID NO: 201 - RNA, 21 nt, synthetic construct
  source 1..21: mol_type = other RNA; note = AON 87 (non-modified)
agggctggcaacgctttcttg

SEQ ID NO: 202 - RNA, 21 nt, synthetic construct
  source 1..21: mol_type = other RNA; note = AON 87
  modified_base 1..5: mod_base = OTHER; note = 2'-O-(2-methoxy)ethyl base
  modified_base 17..21: mod_base = OTHER; note = 2'-O-(2-methoxy)ethyl base
  modified_base 5: mod_base = OTHER; note = 5-methyl
  modified_base 18: mod_base = OTHER; note = 5-methyl
  modified_base 6..16: mod_base = OTHER; note = DNA base
  modified_base 1^2: mod_base = OTHER; note = phosphorothioate linkage
  modified_base 5..17: mod_base = OTHER; note = phosphorothioate linkage
  modified_base 20^21: mod_base = OTHER; note = phosphorothioate linkage
  modified_base 2..5: mod_base = OTHER; note = phospodiester linkage
  modified_base 17..20: mod_base = OTHER; note = phospodiester linkage
agggctggcaacgctttcttg

SEQ ID NO: 203 - RNA, 21 nt, synthetic construct
  source 1..21: mol_type = other RNA; note = AON 88 (non-modified)
agcgccggcaattctttcttg

SEQ ID NO: 204 - RNA, 21 nt, synthetic construct
  source 1..21: mol_type = other RNA; note = AON 88
  modified_base 1..5: mod_base = OTHER; note = 2'-O-(2-methoxy)ethyl base
  modified_base 17..21: mod_base = OTHER; note = 2'-O-(2-methoxy)ethyl base
  modified_base 3: mod_base = OTHER; note = 5-methyl
  modified_base 5: mod_base = OTHER; note = 5-methyl
  modified_base 18: mod_base = OTHER; note = 5-methyl
  modified_base 6..16: mod_base = OTHER; note = DNA base
  modified_base 1^2: mod_base = OTHER; note = phosphorothioate linkage
  modified_base 5..17: mod_base = OTHER; note = phosphorothioate linkage
  modified_base 20^21: mod_base = OTHER; note = phosphorothioate linkage
  modified_base 2..5: mod_base = OTHER; note = phospodiester linkage
  modified_base 17..20: mod_base = OTHER; note = phospodiester linkage
agcgccggcaattctttcttg

SEQ ID NO: 205 - DNA, 22 bp, synthetic construct
  source 1..22: mol_type = other DNA; note = mouse PSEN1 foward primer
ccagtggagactggaacacaac

SEQ ID NO: 206 - DNA, 19 bp, synthetic construct
  source 1..19: mol_type = other DNA; note = mouse wt PSEN1 reverse primer
ggctggcaacgctttcttg

SEQ ID NO: 207 - DNA, 20 bp, synthetic construct
  source 1..20: mol_type = other DNA; note = mouse mt PSEN1 reverse primer
cgccggcaattctttcttga

SEQ ID NO: 208 - DNA, 22 bp, synthetic construct
  source 1..22: mol_type = other DNA; note = mouse B-Actin forward primer
gtgacgttgacatccgtaaaga

SEQ ID NO: 209 - DNA, 19 bp, synthetic construct
  source 1..19: mol_type = other DNA; note = mouse B-Actin reverse primer
gccggactcatcgtactcc

SEQ ID NO: 210 - DNA, 20 bp, synthetic construct
  source 1..20: mol_type = other DNA; note = mouse RPS23 forward primer
  modified_base 7: mod_base = OTHER; note = 2'-O-(2-methoxy)ethyl base
cgtcagggtgcagctcatta

SEQ ID NO: 211 - DNA, 20 bp, synthetic construct
  source 1..20: mol_type = other DNA; note = mouse RPS23 reverse primer
ggcacgaacgctgtgatctt

SEQ ID NO: 212 - DNA, 25 bp, synthetic construct
  source 1..25: mol_type = other DNA; note = human PSEN1 forward primer
ccttttatggaccaattagcattcc

SEQ ID NO: 213 - DNA, 22 bp, synthetic construct
  source 1..22: mol_type = other DNA; note = human PSEN1 reverse primer
ggccatcaccactttgtacaag

SEQ ID NO: 214 - DNA, 25 bp, synthetic construct
  source 1..25: mol_type = other DNA; note = human PSEN1 Taqman probe
tcaattttatatctagacccagctt

SEQ ID NO: 215 - DNA, 19 bp, synthetic construct
  source 1..19: mol_type = other DNA; note = human NPT-II forward primer
cggctgcatacgcttgatc

SEQ ID NO: 216 - DNA, 18 bp, synthetic construct
  source 1..18: mol_type = other DNA; note = human NPT-II reverse primer
gctcgctcgatgcgatgt

SEQ ID NO: 217 - DNA, 18 bp, synthetic construct
  source 1..18: mol_type = other DNA; note = human NPT-II Taqman probe
tacctgcccattcgacca

SEQ ID NO: 218 - RNA, 20 nt, synthetic construct
  source 1..20: mol_type = other RNA; note = AON 89 (non-modified)
gcaattctttcttgaaaatg

SEQ ID NO: 219 - RNA, 20 nt, synthetic construct
  source 1..20: mol_type = other RNA; note = AON 89
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
gcaattctttcttgaaaatg

SEQ ID NO: 220 - RNA, 20 nt, synthetic construct
  source 1..20: mol_type = other RNA; note = AON 90 (non-modified)
ggcaattctttcttgaaaat

SEQ ID NO: 221 - RNA, 20 nt, synthetic construct
  source 1..20: mol_type = other RNA; note = AON 90
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
ggcaattctttcttgaaaat

SEQ ID NO: 222 - RNA, 20 nt, synthetic construct
  source 1..20: mol_type = other RNA; note = AON 91 (non-modified)
tggcaattctttcttgaaaa

SEQ ID NO: 223 - RNA, 20 nt, synthetic construct
  source 1..20: mol_type = other RNA; note = AON 91
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
tggcaattctttcttgaaaa

SEQ ID NO: 224 - RNA, 20 nt, synthetic construct
  source 1..20: mol_type = other RNA; note = AON 92 (non-modified)
gcaattctttcttgaaaatg

SEQ ID NO: 225 - RNA, 20 nt, synthetic construct
  source 1..20: mol_type = other RNA; note = AON 92
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 7: mod_base = OTHER; note = 5-methyl
  modified_base 11: mod_base = OTHER; note = 5-methyl
  modified_base 6..15: mod_base = OTHER; note = DNA base
  modified_base 1..20: mod_base = OTHER; note = phosphorothioate linkage
gcaattctttcttgaaaatg

SEQ ID NO: 226 - RNA, 20 nt, synthetic construct
  source 1..20: mol_type = other RNA; note = AON 93 (non-modified)
ggcaattctttcttgaaaat

SEQ ID NO: 227 - RNA, 20 nt, synthetic construct
  source 1..20: mol_type = other RNA; note = AON 93
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 8: mod_base = OTHER; note = 5-methyl
  modified_base 12: mod_base = OTHER; note = 5-methyl
  modified_base 6..15: mod_base = OTHER; note = DNA base
  modified_base 1..20: mod_base = OTHER; note = phosphorothioate linkage
ggcaattctttcttgaaaat

SEQ ID NO: 228 - RNA, 20 nt, synthetic construct
  source 1..20: mol_type = other RNA; note = AON 94 (non-modified)
tggcaattctttcttgaaaa

SEQ ID NO: 229 - RNA, 20 nt, synthetic construct
  source 1..20: mol_type = other RNA; note = AON 94
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 9: mod_base = OTHER; note = 5-methyl
  modified_base 13: mod_base = OTHER; note = 5-methyl
  modified_base 6..15: mod_base = OTHER; note = DNA base
  modified_base 1..20: mod_base = OTHER; note = phosphorothioate linkage
tggcaattctttcttgaaaa

SEQ ID NO: 230 - RNA, 20 nt, synthetic construct
  source 1..20: mol_type = other RNA; note = AON 95 (non-modified)
gcaattctttcttgaaaatg

SEQ ID NO: 231 - RNA, 20 nt, synthetic construct
  source 1..20: mol_type = other RNA; note = AON 95
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 7: mod_base = OTHER; note = 5-methyl
  modified_base 11: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
gcaattctttcttgaaaatg

SEQ ID NO: 232 - RNA, 20 nt, synthetic construct
  source 1..20: mol_type = other RNA; note = AON 96 (non-modified)
ggcaattctttcttgaaaat

SEQ ID NO: 233 - RNA, 20 nt, synthetic construct
  source 1..20: mol_type = other RNA; note = AON 96
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 8: mod_base = OTHER; note = 5-methyl
  modified_base 12: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
ggcaattctttcttgaaaat

SEQ ID NO: 234 - RNA, 20 nt, synthetic construct
  source 1..20: mol_type = other RNA; note = AON 97 (non-modified)
tggcaattctttcttgaaaa

SEQ ID NO: 235 - RNA, 20 nt, synthetic construct
  source 1..20: mol_type = other RNA; note = AON 97
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 9: mod_base = OTHER; note = 5-methyl
  modified_base 13: mod_base = OTHER; note = 5-methyl
  modified_base 6..15: mod_base = OTHER; note = DNA base
  modified_base 1^2: mod_base = OTHER; note = phosphorothioate linkage
  modified_base 5..16: mod_base = OTHER; note = phosphorothioate linkage
  modified_base 19^20: mod_base = OTHER; note = phosphorothioate linkage
  modified_base 2..5: mod_base = OTHER; note = phospodiester linkage
  modified_base 16..19: mod_base = OTHER; note = phospodiester linkage
tggcaattctttcttgaaaa

SEQ ID NO: 236 - RNA, 20 nt, synthetic construct
  source 1..20: mol_type = other RNA; note = AON 98 (non-modified)
gcaattctttcttgaaaatg

SEQ ID NO: 237 - RNA, 20 nt, synthetic construct
  source 1..20: mol_type = other RNA; note = AON 98
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 7: mod_base = OTHER; note = 5-methyl
  modified_base 11: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
gcaattctttcttgaaaatg

SEQ ID NO: 238 - RNA, 20 nt, synthetic construct
  source 1..20: mol_type = other RNA; note = AON 99 (non-modified)
ggcaattctttcttgaaaat

SEQ ID NO: 239 - RNA, 20 nt, synthetic construct
  source 1..20: mol_type = other RNA; note = AON 99
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 8: mod_base = OTHER; note = 5-methyl
  modified_base 12: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
ggcaattctttcttgaaaat

SEQ ID NO: 240 - RNA, 20 nt, synthetic construct
  source 1..20: mol_type = other RNA; note = AON 100 (non-modified)
tggcaattctttcttgaaaa

SEQ ID NO: 241 - RNA, 20 nt, synthetic construct
  source 1..20: mol_type = other RNA; note = AON 100
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 9: mod_base = OTHER; note = 5-methyl
  modified_base 13: mod_base = OTHER; note = 5-methyl
  modified_base 6..15: mod_base = OTHER; note = DNA base
  modified_base 1^2: mod_base = OTHER; note = phosphorothioate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 19^20: mod_base = OTHER; note = phosphorothioate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
tggcaattctttcttgaaaa

SEQ ID NO: 242 - RNA, 20 nt, synthetic construct
  source 1..20: mol_type = other RNA; note = AON 101 (non-modified)
gcaattctttcttgaaaatg

SEQ ID NO: 243 - RNA, 20 nt, synthetic construct
  source 1..20: mol_type = other RNA; note = AON 101
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
gcaattctttcttgaaaatg

SEQ ID NO: 244 - RNA, 20 nt, synthetic construct
  source 1..20: mol_type = other RNA; note = AON 102 (non-modified)
gcaattctttcttgaaaatg

SEQ ID NO: 245 - RNA, 20 nt, synthetic construct
  source 1..20: mol_type = other RNA; note = AON 102
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..4: mod_base = OTHER; note = dimethylimidazolidin-2-ylidene-phosphoramidate linkage
  modified_base 17..20: mod_base = OTHER; note = dimethylimidazolidin-2-ylidene-phosphoramidate linkage
  modified_base 4^5: mod_base = OTHER; note = phospodiester linkage
  modified_base 16^17: mod_base = OTHER; note = phospodiester linkage
  modified_base 5..16: mod_base = OTHER; note = phosphorothioate linkage
gcaattctttcttgaaaatg

SEQ ID NO: 246 - RNA, 20 nt, synthetic construct
  source 1..20: mol_type = other RNA; note = AON 103 (non-modified)
gcaattctttcttgaaaatg

SEQ ID NO: 247 - RNA, 20 nt, synthetic construct
  source 1..20: mol_type = other RNA; note = AON 103
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 5..16: mod_base = OTHER; note = phosphorothioate linkage
gcaattctttcttgaaaatg

SEQ ID NO: 248 - RNA, 20 nt, synthetic construct
  source 1..20: mol_type = other RNA; note = AON 104 (non-modified)
gcaattctttcttgaaaatg

SEQ ID NO: 249 - RNA, 20 nt, synthetic construct
  source 1..20: mol_type = other RNA; note = AON 104
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 5^6: mod_base = OTHER; note = dimethylimidazolidin-2-ylidene-phosphoramidate linkage
  modified_base 15^16: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 6..15: mod_base = OTHER; note = phosphorothioate linkage
gcaattctttcttgaaaatg

SEQ ID NO: 250 - RNA, 20 nt, synthetic construct
  source 1..20: mol_type = other RNA; note = AON 105 (non-modified)
gcaattctttcttgaaaatg

SEQ ID NO: 251 - RNA, 20 nt, synthetic construct
  source 1..20: mol_type = other RNA; note = AON 105
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 4^5: mod_base = OTHER; note = phospodiester linkage
  modified_base 16^17: mod_base = OTHER; note = phospodiester linkage
gcaattctttcttgaaaatg

SEQ ID NO: 252 - RNA, 20 nt, synthetic construct
  source 1..20: mol_type = other RNA; note = AON 106 (non-modified)
gcaattctttcttgaaaatg

SEQ ID NO: 253 - RNA, 20 nt, synthetic construct
  source 1..20: mol_type = other RNA; note = AON 106
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 2: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 16^17: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
gcaattctttcttgaaaatg

SEQ ID NO: 254 - RNA, 20 nt, synthetic construct
  source 1..20: mol_type = other RNA; note = AON 107 (non-modified)
ggcaattctttcttgaaaat

SEQ ID NO: 255 - RNA, 20 nt, synthetic construct
  source 1..20: mol_type = other RNA; note = AON 107
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
ggcaattctttcttgaaaat

SEQ ID NO: 256 - RNA, 20 nt, synthetic construct
  source 1..20: mol_type = other RNA; note = AON 108 (non-modified)
ggcaattctttcttgaaaat

SEQ ID NO: 257 - RNA, 20 nt, synthetic construct
  source 1..20: mol_type = other RNA; note = AON 108
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..4: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 5..16: mod_base = OTHER; note = phosphorothioate linkage
  modified_base 17^18: mod_base = OTHER; note = phosphorothioate linkage
ggcaattctttcttgaaaat

SEQ ID NO: 258 - RNA, 20 nt, synthetic construct
  source 1..20: mol_type = other RNA; note = AON 109 (non-modified)
ggcaattctttcttgaaaat

SEQ ID NO: 259 - RNA, 20 nt, synthetic construct
  source 1..20: mol_type = other RNA; note = AON 109
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 5..16: mod_base = OTHER; note = phosphorothioate linkage
ggcaattctttcttgaaaat

SEQ ID NO: 260 - RNA, 20 nt, synthetic construct
  source 1..20: mol_type = other RNA; note = AON 110 (non-modified)
ggcaattctttcttgaaaat

SEQ ID NO: 261 - RNA, 20 nt, synthetic construct
  source 1..20: mol_type = other RNA; note = AON 110
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 5^6: mod_base = OTHER; note = dimethylimidazolidin-2-ylidene-phosphoramidate linkage
  modified_base 15^16: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 6..15: mod_base = OTHER; note = phosphorothioate linkage
ggcaattctttcttgaaaat

SEQ ID NO: 262 - RNA, 20 nt, synthetic construct
  source 1..20: mol_type = other RNA; note = AON 111 (non-modified)
ggcaattctttcttgaaaat

SEQ ID NO: 263 - RNA, 20 nt, synthetic construct
  source 1..20: mol_type = other RNA; note = AON 111
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 4^5: mod_base = OTHER; note = phospodiester linkage
  modified_base 16^17: mod_base = OTHER; note = phospodiester linkage
ggcaattctttcttgaaaat

SEQ ID NO: 264 - RNA, 20 nt, synthetic construct
  source 1..20: mol_type = other RNA; note = AON 112 (non-modified)
ggcaattctttcttgaaaat

SEQ ID NO: 265 - RNA, 20 nt, synthetic construct
  source 1..20: mol_type = other RNA; note = AON 112
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 3: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 16^17: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
ggcaattctttcttgaaaat

SEQ ID NO: 266 - RNA, 20 nt, synthetic construct
  source 1..20: mol_type = other RNA; note = AON 113 (non-modified)
tggcaattctttcttgaaaa

SEQ ID NO: 267 - RNA, 20 nt, synthetic construct
  source 1..20: mol_type = other RNA; note = AON 113
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
tggcaattctttcttgaaaa

SEQ ID NO: 268 - RNA, 20 nt, synthetic construct
  source 1..20: mol_type = other RNA; note = AON 114 (non-modified)
tggcaattctttcttgaaaa

SEQ ID NO: 269 - RNA, 20 nt, synthetic construct
  source 1..20: mol_type = other RNA; note = AON 114
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1..4: mod_base = OTHER; note = dimethylimidazolidin-2-ylidene-phosphoramidate linkage
  modified_base 17..20: mod_base = OTHER; note = dimethylimidazolidin-2-ylidene-phosphoramidate linkage
  modified_base 4^5: mod_base = OTHER; note = phospodiester linkage
  modified_base 16^17: mod_base = OTHER; note = phospodiester linkage
  modified_base 5..16: mod_base = OTHER; note = phosphorothioate linkage
tggcaattctttcttgaaaa

SEQ ID NO: 270 - RNA, 20 nt, synthetic construct
  source 1..20: mol_type = other RNA; note = AON 115 (non-modified)
tggcaattctttcttgaaaa

SEQ ID NO: 271 - RNA, 20 nt, synthetic construct
  source 1..20: mol_type = other RNA; note = AON 115
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 5..16: mod_base = OTHER; note = phosphorothioate linkage
tggcaattctttcttgaaaa

SEQ ID NO: 272 - RNA, 20 nt, synthetic construct
  source 1..20: mol_type = other RNA; note = AON 116 (non-modified)
tggcaattctttcttgaaaa

SEQ ID NO: 273 - RNA, 20 nt, synthetic construct
  source 1..20: mol_type = other RNA; note = AON 116
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1^2: mod_base = OTHER; note = dimethylimidazolidin-2-ylidene-phosphoramidate linkage
  modified_base 3^4: mod_base = OTHER; note = dimethylimidazolidin-2-ylidene-phosphoramidate linkage
  modified_base 5^6: mod_base = OTHER; note = dimethylimidazolidin-2-ylidene-phosphoramidate linkage
  modified_base 15^16: mod_base = OTHER; note = dimethylimidazolidin-2-ylidene-phosphoramidate linkage
  modified_base 17^18: mod_base = OTHER; note = dimethylimidazolidin-2-ylidene-phosphoramidate linkage
  modified_base 19^20: mod_base = OTHER; note = dimethylimidazolidin-2-ylidene-phosphoramidate linkage
  modified_base 2^3: mod_base = OTHER; note = phospodiester linkage
  modified_base 4^5: mod_base = OTHER; note = phospodiester linkage
  modified_base 16^17: mod_base = OTHER; note = phospodiester linkage
  modified_base 18^19: mod_base = OTHER; note = phospodiester linkage
  modified_base 6..15: mod_base = OTHER; note = phosphorothioate linkage
tggcaattctttcttgaaaa

SEQ ID NO: 274 - RNA, 20 nt, synthetic construct
  source 1..20: mol_type = other RNA; note = AON 117 (non-modified)
tggcaattctttcttgaaaa

SEQ ID NO: 275 - RNA, 20 nt, synthetic construct
  source 1..20: mol_type = other RNA; note = AON 117
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
  modified_base 4^5: mod_base = OTHER; note = phospodiester linkage
  modified_base 16^17: mod_base = OTHER; note = phospodiester linkage
tggcaattctttcttgaaaa

SEQ ID NO: 276 - RNA, 20 nt, synthetic construct
  source 1..20: mol_type = other RNA; note = AON 118 (non-modified)
tggcaattctttcttgaaaa

SEQ ID NO: 277 - RNA, 20 nt, synthetic construct
  source 1..20: mol_type = other RNA; note = AON 118
  modified_base 1..5: mod_base = OTHER; note = 2'-O-(2-methoxy)ethyl base
  modified_base 16..20: mod_base = OTHER; note = 2'-O-(2-methoxy)ethyl base
  modified_base 4: mod_base = OTHER; note = 5-methyl
  modified_base 6..15: mod_base = OTHER; note = DNA base
  modified_base 1..3: mod_base = OTHER; note = dimethylimidazolidin-2-ylidene-phosphoramidate linkage
  modified_base 4^5: mod_base = OTHER; note = dimethylimidazolidin-2-ylidene-phosphoramidate linkage
  modified_base 16^17: mod_base = OTHER; note = dimethylimidazolidin-2-ylidene-phosphoramidate linkage
  modified_base 18..20: mod_base = OTHER; note = dimethylimidazolidin-2-ylidene-phosphoramidate linkage
  modified_base 3^4: mod_base = OTHER; note = phosphorothioate linkage
  modified_base 5..16: mod_base = OTHER; note = phosphorothioate linkage
  modified_base 17^18: mod_base = OTHER; note = phosphorothioate linkage
tggcaattctttcttgaaaa